(19) United States
(12) Patent Application Publication
(10) Pub. No.: US 2025/0084387 A1
(43) Pub. Date: Mar. 13, 2025

(54) FORMATE DEHYDROGENASE VARIANTS AND METHODS OF USE

(71) Applicant: Genomatica, Inc., San Diego, CA (US)

(72) Inventors: Amit Mahendra Shah, San Diego, CA (US); Justin Robert Colquitt, San Diego, CA (US); Michael Gregory Napolitano, Quincy, MA (US); Nathan Schmidt, Roslindale, MA (US); Pichet Praveschotinunt, Cambridge, MA (US); Joseph Roy Warner, Oceanside, CA (US)

(21) Appl. No.: 18/580,585

(22) PCT Filed: Aug. 29, 2022
(86) PCT No.: PCT/US2022/075588

§ 371 Date: Jan. 18, 2024

Related U.S. Application Data
(60) Provisional application No. 63/239,231, filed on Aug. 31, 2021.

(51) Int. Cl. C12N 9/02; C12N 1/26; C12N 15/70; C12P 7/18

(52) U.S. Cl. CPC C12N 9/0093; C12N 1/26; C12N 15/70; C12P 7/18

(57) ABSTRACT

The disclosure provides polypeptides and encoding nucleic acids of engineered formate dehydrogenases. The disclosure also provides cells expressing an engineered formate dehydrogenase. The disclosure further provides methods for producing a bioderived compound comprising culturing cells expressing an engineered formate dehydrogenase. Where the engineered formate dehydrogenase is capable of catalyzing a conversion.

### CROSS-REFERENCE TO RELATED APPLICATIONS

This application claims the benefit of U.S. Provisional Application No. 63/239,231, filed Aug. 31, 2021, the entire contents of which is incorporated by reference herein.

### INCORPORATION BY REFERENCE OF SEQUENCE LISTING

The instant application contains a Sequence Listing, which has been submitted via Patent Center. The Sequence Listing titled 199683-105001_PCT.xml, which was created on Aug. 24, 2022 and is 82,608 bytes in size, is hereby incorporated by reference in its entirety.

### FIELD OF DISCLOSURE

The present disclosure relates generally to formate dehydrogenase (FDH) variants and methods of using such variants, and more specifically to formate dehydrogenase variants encoded by recombinant nucleic acids that have been introduced to a non-naturally occurring microbial organism for enhancing production of NADH, thereby increasing the production of a bioderived compound (e.g., 1,3-butanediol).

### BACKGROUND DESCRIBED HEREIN

1,3-butanediol (1,3-BDO; also referred to as 1,3-butylene glycol, 1,3-BG, butylene glycol, BG) is a four carbon diol traditionally produced from acetylene via its hydration. The resulting acetaldehyde is then converted to 3-hydroxybutyraldehdye which is subsequently reduced to form 1,3-BDO. More recently, acetylene has been replaced by the less expensive ethylene as a source of acetaldehyde. 1,3-BDO is commonly used as an organic solvent for food flavoring agents. It is also used as a co-monomer for polyurethane and polyester resins and is widely employed as a hypoglycemic agent. Optically active 1,3-BDO is a useful starting material for the synthesis of biologically active compounds and liquid crystals. Another use of 1,3-BDO is that its dehydration affords 1,3-butadiene (Ichikawa et al. Journal of Molecular Catalysis A-Chemical 256:106-112 (2006); Ichikawa et al. Journal of Molecular Catalysis A-Chemical 231:181-189 (2005), which is useful in the manufacture synthetic rubbers (e.g., tires), latex, and resins. The reliance on petroleum based feedstocks for either acetylene or ethylene warrants the development of a renewable feedstock based route to 1,3-BDO and to butadiene.

1,4-butanediol (1,4-BDO) is a valuable chemical for the production of high performance polymers, solvents, and fine chemicals. It is the basis for producing other high value chemicals such as tetrahydrofuran (THF) and gamma-butyrolactone (GBL). The value chain is comprised of three main segments including: (1) polymers, (2) THF derivatives, and (3) GBL derivatives. In the case of polymers, 1,4-BDO is a comonomer for polybutylene terephthalate (PBT) production. PBT is a medium performance engineering thermoplastic used in automotive, electrical, water systems, and small appliance applications. Conversion to THF, and subsequently to polytetramethylene ether glycol (PTMEG), provides an intermediate used to manufacture spandex products such as LYCRA® fibers. PTMEG is also combined with 1,4-BDO in the production of specialty polyester ethers (COPE). COPEs are high modulus elastomers with excellent mechanical properties and oil/environmental resistance, allowing them to operate at high and low temperature extremes. PTMEG and 1,4-BDO also make thermoplastic polyurethanes processed on standard thermoplastic extrusion, calendaring, and molding equipment, and are characterized by their outstanding toughness and abrasion resistance. The GBL produced from 1,4-BDO provides the feedstock for making pyrrolidones, as well as serving the agrochemical market. The pyrrolidones are used as high performance solvents for extraction processes of increasing use, including for example, in the electronics industry and in pharmaceutical production.

1,4-BDO is produced by two main petrochemical routes with a few additional routes also in commercial operation. One route involves reacting acetylene with formaldehyde, followed by hydrogenation. More recently 1,4-BDO processes involving butane or butadiene oxidation to maleic anhydride, followed by hydrogenation have been introduced. 1,4-BDO is used almost exclusively as an intermediate to synthesize other chemicals and polymers.

Over 25 billion pounds of butadiene (1,3-butadiene, BD) are produced annually and is applied in the manufacture of polymers such as synthetic rubbers and ABS resins, and chemicals such as hexamethylenediamine and 1,4-butanediol. For example, butadiene can be reacted with numerous other chemicals, such as other alkenes (e.g., styrene) to manufacture numerous copolymers (e.g., acrylonitrile 1,3-butadiene styrene (ABS), styrene-1,3-butadiene (SBR) rubber, styrene-1,3-butadiene latex). These materials are used in rubber, plastic, insulation, fiberglass, pipes, automobile and boat parts, food containers, and carpet backing. Butadiene is typically produced as a by-product of the steam cracking process for conversion of petroleum feedstocks such as naphtha, liquefied petroleum gas, ethane or natural gas to ethylene and other olefins. The ability to manufacture butadiene from alternative and/or renewable feedstocks would represent a major advance in the quest for more sustainable chemical production processes.

Crotyl alcohol, also referred to as 2-buten-1-ol, is a valuable chemical intermediate. It serves as a precursor to crotyl halides, esters, and ethers, which in turn are chemical intermediates in the production of monomers, fine chemicals, agricultural chemicals, and pharmaceuticals. Exemplary fine chemical products include sorbic acid, trimethylhydroquinone, crotonic acid and 3-methoxybutanol. Crotyl alcohol is also a precursor to 1,3-butadiene. Crotyl alcohol is currently produced exclusively from petroleum feedstocks. For example Japanese Patent 47-013009 and U.S. Pat. Nos. 3,090,815, 3,090,816, and 3,542,883 describe a method of producing crotyl alcohol by isomerization of 1,2-epoxybutane. The ability to manufacture crotyl alcohol from alternative and/or renewable feedstocks would represent a major advance in the quest for more sustainable chemical production processes.

3-Buten-2-ol (also referenced to as methyl vinyl carbinol (MVC)) is an intermediate that can be used to produce butadiene. There are significant advantages to use of 3-buten-2-ol over 1,3-BDO because there are fewer separation steps and only one dehydration step. 3-Buten-2-ol can also be used as a solvent, a monomer for polymer production, or a precursor to fine chemicals. Accordingly, the ability to manufacture 3-buten-2-ol from alternative and/or renewable feedstock would again present a significant advantage for sustainable chemical production processes.

Adipic acid, a dicarboxylic acid, has a molecular weight of 146.14. It can be used is to produce nylon 6,6, a linear polyamide made by condensing adipic acid with hexamethylenediamine. This is employed for manufacturing different kinds of fibers. Other uses of adipic acid include its use in plasticizers, unsaturated polyesters, and polyester polyols. Additional uses include for production of polyurethane, lubricant components, and as a food ingredient as a flavorant and gelling aid.

Historically, adipic acid was prepared from various fats using oxidation. Some current processes for adipic acid synthesis rely on the oxidation of KA oil, a mixture of cyclohexanone, the ketone or K component, and cyclohexanol, the alcohol or A component, or of pure cyclohexanol using an excess of strong nitric acid. There are several variations of this theme which differ in the routes for production of KA or cyclohexanol. For example, phenol is an alternative raw material in KA oil production, and the process for the synthesis of adipic acid from phenol has been described. The other versions of this process tend to use oxidizing agents other than nitric acid, such as hydrogen peroxide, air or oxygen.

In addition to hexamethylenediamine (HMDA) being used in the production of nylon-6,6 as described above, it is also utilized to make hexamethylene diisocyanate, a monomer feedstock used in the production of polyurethane. The diamine also serves as a cross-linking agent in epoxy resins. HMDA is presently produced by the hydrogenation of adiponitrile.

Caprolactam is an organic compound which is a lactam of 6-aminohexanoic acid (E-aminohexanoic acid, 6-aminocaproic acid). It can alternatively be considered cyclic amide of caproic acid. One use of caprolactam is as a monomer in the production of nylon-6. Caprolactam can be synthesized from cyclohexanone via an oximation process using hydroxylammonium sulfate followed by catalytic rearrangement using the Beckmann rearrangement process step.

Methylacrylic acid (MAA) is a key precursor of methyl methacrylate (MMA), a chemical intermediate with a global demand in excess of 4.5 billion pounds per year, much of which is converted to polyacrylates. The conventional process for synthesizing methyl methacrylate (i.e., the acetone cyanohydrin route) involves the conversion of hydrogen cyanide (HCN) and acetone to acetone cyanohydrin which then undergoes acid assisted hydrolysis and esterification with methanol to give MAA. Difficulties in handling potentially deadly HCN along with the high costs of byproduct disposal (1.2 tons of ammonium bisulfate are formed per ton of MAA) have sparked a great deal of research aimed at cleaner and more economical processes. As a starting material, MAA can easily be converted into MMA via esterification with methanol.

Microbial organisms can be used for the production of bioderived compounds, such as 1,3-BDO, 1,4-BDO, butadiene, crotyl alcohol, MVC, adipate, HMDA, caprolactam, and MAA. The titer, rate, and yield of such production can be limited by co-factor availability. In particular, limited cofactors, such as the reducing agents nicotinamide adenine dinucleotide, reduced (NADH) or nicotinamide adenine dinucleotide phosphate, reduced (NADPH), can result in limited redox availability. For example, NADPH provides the reducing equivalents for biosynthetic reactions, such as lipid and nucleic acid synthesis, and the oxidation-reduction involved in protecting against the toxicity of reactive oxygen species (ROS). NADPH is also used for anabolic pathways, such as cholesterol synthesis and fatty acid chain elongation. An imbalance in redox levels can lead to deleterious effects on the production of bioderived compounds, such as 1,3-BDO, 1,4-BDO, butadiene, crotyl alcohol, MVC, adipate, HMDA, caprolactam, and MAA. Also, NADH is essential for metabolism and energy production, as well as enabling the correct function of many important molecules by transferring electrons between molecules in redox reactions. The NAD⁺/NADH pair has been found to be important in microbial catabolism and cell growth. Moreover, NAD(P)+ transhydrogenases (EC 1.6.1.1—Si-specific; and EC 1.6.1.2—Re/Si-specific) can catalyze the reversible reaction of NADPH+NAD⁺⇔NADP⁺+NADH, thus an increase in production of NADH can translate to an increase in production of NADPH. Accordingly, increased availability of co-factors, such as NADH, can help to increase the titer, rate, and yield of bioderived compounds.

Formate dehydrogenase (FDH; EC 1.2.1.2) is a common enzyme found in nature that catalyzes the oxidation of formate (i.e., formate ion) to carbon dioxide with concomitant reduction of nicotinamide adenine dinucleotide in its oxidized form (NAD⁺) to its reduced form (NADH). Alternatively, an FDH (EC 1.17.1.9) may use a cofactor of nicotinamide adenine dinucleotide phosphate (NADP⁺) in catalyzing the oxidation of formate, thereby producing carbon dioxide and NADPH. FDH may be used as a coenzyme cycling system for the bioconversion and production of optically active compounds, including but not limited to, most amino acids, chiral compounds (e.g., chiral alcohols), and hydroxy acids. FDH plays an important role as a catalyst in organic acid syntheses for producing desired products, for example, pharmaceutical products of interest.

Thus, there exists a need for the development of methods for effectively producing commercial quantities of bioderived compounds, such as 1,3-BDO, 1,4-BDO, butadiene, crotyl alcohol, MVC, adipate, HMDA, caprolactam, and MAA, which may be improved by increasing the availability of NADH. The present disclosure satisfies this need and provides related advantages as well.

### SUMMARY OF INVENTION

In some embodiments, provided herein is an engineered formate dehydrogenase that is a variant of amino acid sequence SEQ ID NO: 1 or 2 or a functional fragment thereof. Such an engineered formate dehydrogenase includes one or more alterations at a position described in TABLE 6 and/or TABLE 7. An engineered formate dehydrogenase described herein, in some embodiments, is capable of: (a) catalyzing the conversion of formate to carbon dioxide; (b) catalyzing the conversion or reduction of NAD⁺ to NADH; or (c) catalyzing the conversion of formate to carbon dioxide and NAD⁺ to NADH. In some embodiments, an engineered formate dehydrogenase described herein is capable of catalyzing the conversion of formate to carbon dioxide and NAD⁺ to NADH.

In some embodiments, an engineered formate dehydrogenase described herein has an activity that is at least 0.5, at least 1, at least 1.5, or at least 2-fold higher than the activity of a wild-type formate dehydrogenase, such as a formate dehydrogenase having the amino acid sequence of SEQ ID NO: 1 or 2.

In some embodiments, an engineered formate dehydrogenase described herein has one or more amino acid alterations, such as one or more amino acid substitutions, as described in TABLE 6 and/or TABLE 7. In some embodiments, an engineered formate dehydrogenase described herein has one or more amino acid alterations that include one or more conservative amino acid substitutions. In some embodiments, an engineered formate dehydrogenase provided herein has one or more amino acid alterations that include one or more non-conservative amino acid substitutions. In some embodiments, the one or more amino acid alterations result in an engineered formate dehydrogenase having one or more residues at specific positions corresponding to those in SEQ ID NO: 1 or 2, including one or more of those alterations described in TABLE 6 and/or TABLE 7.

In some embodiments, an engineered formate dehydrogenase provided herein has at least one, two, three, or four amino acid alterations described herein (e.g., TABLE 6 and/or TABLE 7). Such an engineered formate dehydrogenase, in some embodiments, has one alteration or a combination of alterations in SEQ ID NO: 1 or 2 that corresponds to a variant described in TABLE 6 or TABLE 7.

In some embodiments, an engineered formate dehydrogenase provided herein does not have an amino acid sequence of SEQ ID NO: 24.

Additional engineered formate dehydrogenases provided herein include variants of homologs of SEQ ID NO: 1 and 2 as identified herein. Accordingly, in some embodiments, an engineered formate dehydrogenase provided herein has an amino acid sequence that is a variant of amino acid sequences SEQ ID NOs: 3-24. Such an engineered formate dehydrogenase, in some embodiments, include one or more alterations at a position corresponding to a position described in TABLE 6 and/or TABLE 7.

In some embodiments, provided herein is a recombinant nucleic acid encoding an engineered formate dehydrogenase described herein. In some embodiments, such a recombinant nucleic acid has a nucleotide sequence encoding an engineered formate dehydrogenase described herein operatively linked to a promoter. In some embodiments, also provided herein is a vector having such recombinant nucleic acid.

In some embodiments, provided herein is a non-naturally occurring microbial organism having a recombinant nucleic acid encoding an engineered formate dehydrogenase described herein. Such a microbial organism, in some embodiments, further includes a pathway capable of producing a bioderived compound, wherein one or more enzymes of the pathway uses NADH or NADPH as a cofactor for catalyzing its enzymatic reaction. In some embodiments, the one or more enzymes of such a pathway are encoded by an exogenous nucleic acid.

In some embodiments, a microbial organism described herein includes an exogenous nucleic acid that is heterologous to the microbial organism. In some embodiments, a microbial organism described herein includes an exogenous nucleic acid that is homologous to the microbial organism.

In some embodiments, a microbial organism described herein includes a pathway capable of producing a bioderived compound. Such a bioderived compound, in some embodiments, is an alcohol, a glycol, an organic acid, an alkene, a diene, an organic amine, an organic aldehyde, a vitamin, a nutraceutical or a pharmaceutical. Examples of alcohols include: (a) a biofuel alcohol, wherein said biofuel is a primary alcohol, a secondary alcohol, a diol or triol comprising C3 to C10 carbon atoms; (b) n-propanol or isopropanol; and (c) a fatty alcohol, wherein said fatty alcohol comprises C4 to C27 carbon atoms, C8 to C18 carbon atoms, C12 to C18 carbon atoms, or C12 to C14 carbon atoms. Examples of biofuel alcohols include: 1-propanol, isopropanol, 1-butanol, isobutanol, 1-pentanol, isopentenol, 2-methyl-1-butanol, 3-methyl-1-butanol, 1-hexanol, 3-methyl-1-pentanol, 1-heptanol, 4-methyl-1-hexanol, and 5-methyl-1-hexanol. In some embodiments, the diol is a propanediol or a butanediol, such as 1,4 butanediol, 1,3-butanediol or 2,3-butanediol. In some embodiments, the bioderived compound is selected from the group consisting of: (a) 1,4-butanediol or an intermediate thereto, wherein said intermediate is optionally 4-hydroxybutanoic acid (4-HB); (b) butadiene (1,3-butadiene) or an intermediate thereto, wherein said intermediate is optionally 1,4-butanediol, 1,3-butanediol, 2,3-butanediol, crotyl alcohol, 3-buten-2-ol (methyl vinyl carbinol) or 3-buten-1-ol; (c) 1,3-butanediol or an intermediate thereto, wherein said intermediate is optionally 3-hydroxybutyrate (3-HB), 2,4-pentadienoate, crotyl alcohol or 3-buten-1-ol; (d) adipate, 6-aminocaproic acid, caprolactam, hexamethylenediamine, levulinic acid or an intermediate thereto, wherein said intermediate is optionally adipyl-CoA or 4-aminobutyryl-CoA; (e) methacrylic acid or an ester thereof, 3-hydroxyisobutyrate, 2-hydroxyisobutyrate, or an intermediate thereto, wherein said ester is optionally methyl methacrylate or poly(methyl methacrylate); (f) 1,2-propanediol (propylene glycol), 1,3-propanediol, glycerol, ethylene glycol, diethylene glycol, triethylene glycol, dipropylene glycol, tripropylene glycol, neopentyl glycol, bisphenol A or an intermediate thereto; (g) succinic acid or an intermediate thereto; and (h) a fatty alcohol, a fatty aldehyde or a fatty acid comprising C4 to C27 carbon atoms, C8 to C18 carbon atoms, C12 to C18 carbon atoms, or C12 to C14 carbon atoms, wherein said fatty alcohol is optionally dodecanol (C12; lauryl alcohol), tridecyl alcohol (C13; 1-tridecanol, tridecanol, isotridecanol), myristyl alcohol (C14; 1-tetradecanol), pentadecyl alcohol (C15; 1-pentadecanol, pentadecanol), cetyl alcohol (C16; 1-hexadecanol), heptadecyl alcohol (C17; 1-n-heptadecanol, heptadecanol) and stearyl alcohol (C18; 1-octadecanol) or palmitoleyl alcohol (C16 unsaturated; cis-9-hexadecen-1-ol).

In some embodiments, a microbial organism described herein is in a substantially anaerobic culture medium.

In some embodiments, a microbial organism described herein is a species of bacteria, yeast, or fungus.

In some embodiments, a microbial organism described herein is capable of producing at least 10% more NADH or a bioderived compound compared to a control microbial organism that does not include a recombinant nucleic acid encoding an engineered formate dehydrogenase described herein.

In some embodiments, provided herein is a method for producing a bioderived compound described herein that includes culturing a non-naturally occurring microbial organism described herein under conditions and for a sufficient period of time to produce the bioderived compound. Such a method, in some embodiments, also includes separating the bioderived from other components in the culture. Methods for performing such separating includes extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

In some embodiments, provided herein is culture medium having the bioderived compound produced by a method provided herein, wherein the bioderived compound has a carbon-12, carbon-13 and carbon-14 isotope ratio that reflects an atmospheric carbon dioxide uptake source.

In some embodiments, provided herein is a bioderived compound produced according to a method described herein. Such a bioderived compound, in some embodiments, has an Fm value of at least 80%, at least 85%, at least 90%, at least 95% or at least 98%.

In some embodiments, provided herein is composition having a bioderived compound described herein and a compound other than the bioderived compound. Such a compound other than said bioderived compound, in some embodiments, is a trace amount of a cellular portion of a non-naturally occurring microbial organism having a bioderived compound pathway.

In some embodiments, provided herein is composition having a bioderived compound described herein of, or a cell lysate or culture supernatant thereof.

In some embodiments, provided herein is a method for increasing the availability of NADH in a non-naturally occurring microbial organism. Such a method, in some embodiments, includes culturing a non-naturally occurring microbial organism a having a recombinant nucleic acid encoding an engineered formate dehydrogenase described herein, under conditions and for a sufficient period of time to increase the availability of NADH. The increase in availability of NADH, in some embodiments, yields an increase in production of the bioderived compound as described herein.

In some embodiments, provided herein is a method for decreasing formate concentration in a non-naturally occurring microbial organism. Such a method, in some embodiments, includes culturing a non-naturally occurring microbial organism a having a recombinant nucleic acid encoding an engineered formate dehydrogenase described herein, under conditions and for a sufficient period of time to increase the conversion of formate to carbon dioxide. The decreased formate concentration in the non-naturally occurring microbial organism, in some embodiments, yields a decrease in formate as an impurity in a method for production of the bioderived compound described herein.

### BRIEF DESCRIPTION OF THE DRAWINGS

FIG. 1 shows the performance of the controls versus the library in the FDH library screen.

FIG. 2 shows an exemplary alignment between SEQ ID NO: 1 and SEQ ID NO: 2, including a consensus sequence (SEQ ID NO: 49).

### DETAILED DESCRIPTION DESCRIBED HEREIN

The subject matter described herein relates to enzyme variants that have desirable properties and are useful for producing desired products (e.g., NADH or a bioderived compound). In some embodiments, the subject matter described herein relates to engineered formate dehydrogenases, which are enzyme variants that have markedly different structural and/or functional characteristics compared to a wild-type formate dehydrogenase that occurs in nature. Thus, the engineered formate dehydrogenases provided herein are not naturally occurring enzymes. Such engineered formate dehydrogenases provided are useful in an engineered cell, such as a microbial organism, that has been engineered to produce a desired product (e.g., NADH or a bioderived compound). For example, as disclosed herein, a cell, such as a microbial organism, having a metabolic pathway can produce a desired product (e.g., NADH or a bioderived compound). Engineered formate dehydrogenases having desirable characteristics as described herein can be introduced into a cell, such as microbial organism, that has a metabolic pathway that uses formate dehydrogenase activity to produce a bioderived compound. Thus, the engineered formate dehydrogenases provided herein can be utilized in engineered cells, such as microbial organisms, to produce a desired product.

Conventions and Abbreviations

| Abbreviation | Convention |
|---|---|
| Ala; A | Alanine |
| Arg; R | Arginine |
| Asn; N | Asparagine |
| Asp; D | Aspartic acid |
| Cys; C | Cysteine |
| Glu; E | Glutamic acid |
| Gln; Q | Glutamine |
| Gly; G | Glycine |
| His; H | Histidine |
| Ile; I | Isoleucine |
| Leu; L | Leucine |
| Lys; K | Lysine |
| Met; M | Methionine |
| Phe; F | Phenylalanine |
| Pro; P | Proline |
| Ser; S | Serine |
| Thr; T | Threonine |
| Trp; W | Tryptophan |
| Tyr; Y | Tyrosine |
| Val; V | Valine |
| FDH | formate dehydrogenase |
| NAD⁺ | Nicotinamide adenine dinucleotide |
| NADH | Nicotinamide adenine dinucleotide, reduced |
| NADP⁺ | Nicotinamide adenine dinucleotide |
| | phosphate |
| NADPH | Nicotinamide adenine dinucleotide |
| | phosphate, reduced |
| Pi | Phosphate |

As used herein the term “about” means±10% of the stated value. The term “about” can mean rounded to the nearest significant digit. Thus, about 5% means 4.5% to 5.5%. Additionally, about in reference to a specific number also includes that exact number. For example, about 5% also includes exact 5%.

As used herein, the term “alteration” or grammatical equivalents thereof when used in reference to any peptide, polypeptide, protein, nucleic acid or polynucleotide described herein refers to a change in structure of an amino acid residue or nucleic acid base relative to the starting or reference residue or base. An alteration of an amino acid residue includes, for example, deletions, insertions and substituting one amino acid residue for a structurally different amino acid residue. Such substitutions can be a conservative substitution, a non-conservative substitution, a substitution to a specific sub-class of amino acids, or a combination thereof as described herein. An alteration of a nucleic acid base includes, for example, changing one naturally occurring base for a different naturally occurring base, such as changing an adenine to a thymine or a guanine to a cytosine or an adenine to a cytosine or a guanine to a thymine. An alteration of a nucleic acid base may result in an alteration of the encoding peptide, polypeptide or protein by changing the encoded amino acid residue or function of the peptide, polypeptide or protein. An alteration of a nucleic acid base may not result in an alteration of the amino acid sequence or function of encoded peptide, polypeptide or protein, also known as a silent mutation.

As used herein, the term “bioderived” means derived from or synthesized by a biological organism and can be considered a renewable resource since it can be generated by a biological organism. Such a biological organism, in particular the non-naturally occurring microbial organism disclosed herein, can utilize feedstock or biomass, such as, sugars (e.g., cellobiose, glucose, fructose, xylose, galactose (e.g., galactose from marine plant biomass), and sucrose), carbohydrates obtained from an agricultural, plant, bacterial, or animal source, and glycerol (e.g., crude glycerol byproduct from biodiesel manufacturing) for synthesis of a desired bioderived compound.

As used herein, the term “conservative substitution” refers to the replacement of one amino acid for another such that the replacement takes place within a family of amino acids that are related in their side chains. Alternatively, the term “non-conservative substitution” refers to the replacement of one amino acid residue for another such that the replaced residue is going from one family of amino acids to a different family of residues. Genetically encoded amino acids can be divided into four families: (1) acidic (negatively charged)=Asp (D), Glu (G); (2) basic (positively charged)=Lys (K), Arg (R), His (H); (3) non-polar (hydrophobic)=Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic=Ala (A), Val (V), Leu (L), Ile (I), Met (M), Phe (F); and (ii) moderately hydrophobic=Gly (G), Pro (P), Cys (C), Tyr (Y), Trp (W); and (4) uncharged polar=Asn (N), Gln (Q), Ser (S), Thr (T). In alternative fashion, the amino acid repertoire can be grouped as (1) acidic (negatively charged)=Asp (D), Glu (G); (2) basic (positively charged)=Lys (K), Arg (R), His (H), and (3) aliphatic=Gly (G), Ala (A), Val (V), Leu (L), Ile (I), Ser (S), Thr (T), with Ser (S) and Thr (T) optionally being grouped separately as aliphatic-hydroxyl; (4) aromatic=Phe (F), Tyr (Y), Trp (W); (5) amide=Asn (N), Glu (Q); and (6) sulfur-containing=Cys (C) and Met (M) (see, for example, Biochemistry, 4th ed., Ed. by L. Stryer, WH Freeman and Co., 1995, which is incorporated by reference herein in its entirety).

As used herein, the term “culture medium,” “medium,” “growth medium” or grammatical equivalents thereof refers to a liquid or solid (e.g., gelatinous) substance containing nutrients that support the growth of a cell, including a microbial organism, such as the microbial organism described herein. Nutrients that support growth include, but are not limited to, the following: a substrate that supplies carbon, such as, but are not limited to, cellobiose, galactose, glucose, xylose, ethanol, acetate, arabinose, arabitol, sorbitol and glycerol; salts that provide essential elements including magnesium, nitrogen, phosphorus, and sulfur; a source for amino acids, such as peptone or tryptone; and a source for vitamin content, such as yeast extract. Culture medium can be a defined medium, in which quantities of all ingredients are known, or an undefined medium, in which the quantities of all ingredients are not known. Culture medium can also include substances other than nutrients needed for growth, such as a substance that only allows select cells to grow (e.g., antibiotic or antifungal), which are generally found in selective medium, or a substance that allows for differentiation of one microbial organism over another when grown on the same medium, which are generally found in differential or indicator medium. Such substances are well known to a person skilled in the art.

As used herein, the term “engineered” or “variant” when used in reference to any peptide, polypeptide, protein, nucleic acid or polynucleotide described herein refers to a sequence of amino acids or nucleic acids having at least one alteration at an amino acid residue or nucleic acid base as compared to a parent sequence. Such a sequence of amino acids or nucleic acids is not naturally occurring. The parent sequence of amino acids or nucleic acids can be, for example, a wild-type sequence or a homolog thereof, or a modified variant of a wild-type sequence or homolog thereof.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid described herein can utilize either or both a heterologous or homologous encoding nucleic acid.

It is understood that, when more than one recombinant nucleic acid and/or exogenous nucleic acid is included into a microbial organism, the more than one recombinant nucleic acid and/or exogenous nucleic acid refers to the referenced encoding nucleic acid or biosynthetic activity, as discussed herein. It is further understood, as disclosed herein, that such more than one recombinant nucleic acids or exogenous nucleic acids can be introduced into the host microbial organism on separate nucleic acid molecules, on polycistronic nucleic acid molecules, or a combination thereof, and still be considered as more than one recombinant nucleic acid and/or exogenous nucleic acid. For example, as disclosed herein a microbial organism can be engineered to express two or more recombinant and/or exogenous nucleic acids encoding a desired pathway enzyme or protein. In the case where two recombinant and/or exogenous nucleic acids encoding an enzyme or protein having a desired activity are introduced into a host microbial organism, it is understood that the two recombinant and/or exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two recombinant and/or exogenous nucleic acids can be introduced into a host organism in any desired combination, for example, on a single plasmid, on separate plasmids, can be integrated into the host chromosome at a single site or multiple sites, and still be considered as two or more recombinant or exogenous nucleic acids, for example three exogenous nucleic acids. Thus, the number of referenced recombinant or exogenous nucleic acids or biosynthetic activities refers to the number of encoding nucleic acids or the number of biosynthetic activities, not the number of separate nucleic acids introduced into the host organism.

The term “Fm value” or “Fraction Modem value” when used in reference to a compound is a ratio of carbon-14 (¹⁴C) to carbon-12 (¹²C). Specifically, Fm value is computed from the expression: Fm=(S−B)/(M−B), where B, S and M represent the ¹⁴C/¹²C ratios of the blank, the sample and the modern reference, respectively. Fm value is a measurement of the deviation of the ¹⁴C/¹²C ratio of a sample from “Modem.” Modem is defined as 95% of the radiocarbon concentration (in AD 1950) of National Bureau of Standards (NBS) Oxalic Acid I (i.e., standard reference materials (SRM) 4990b) normalized to δ¹³C_{VPDB}=−19 per mil (Olsson, The use ofOxalic acid as a Standard. in, Radiocarbon Variations and Absolute Chronology, Nobel Symposium, 12th Proc., John Wiley & Sons, New York (1970)). Mass spectrometry results, for example, measured by ASM, are calculated using the internationally agreed upon definition of 0.95 times the specific activity of NBS Oxalic Acid I (SRM 4990b) normalized to δ¹³C_{VPDB}=−19 per mil. This is equivalent to an absolute (AD 1950) ¹⁴C/¹²C ratio of 1.176±0.010×10⁻¹² (Karlen et al., Arkiv Geofysik, 4:465-471 (1968)). The standard calculations take into account the differential uptake of one isotope with respect to another, for example, the preferential uptake in biological systems of C¹² over C¹³ over C¹⁴, and these corrections are reflected as a Fm corrected for δ¹³. An Fm=0% represents the entire lack of carbon-14 atoms in a material, thus indicating a fossil (for example, petroleum based) carbon source, whereas a Fm=100%, after correction for the post-1950 injection of carbon-14 into the atmosphere from nuclear bomb testing, indicates an entirely modern carbon source. The percent modern carbon (pMC) can be greater than 100% because of the continuing but diminishing effects of the 1950s nuclear testing programs, which resulted in a considerable enrichment of carbon-14 in the atmosphere. Because all sample carbon-14 activities are referenced to a “pre-bomb” standard, and because nearly all new biobased products are produced in a post-bomb environment, all pMC values (after correction for isotopic fraction) must be multiplied by 0.95 (as of 2010) to better reflect the true biobased content of the sample. A biobased content that is greater than 103% suggests that either an analytical error has occurred, or that the source of biobased carbon is more than several years old. Applications of carbon-14 dating techniques to quantify bio-based content of materials are well known in the art (see, e.g., Currie et al., Nuclear Instruments and Methods in Physics Research B, 172:281-287 (2000), and Colonna et al., Green Chemistry, 13:2543-2548 (2011)).

As used herein, the term “functional fragment” when used in reference to a peptide, polypeptide or protein is intended to refer to a portion of the peptide, polypeptide or protein that retains some or all of the activity (e.g., catalyzing the conversion of formate to carbon dioxide and/or NAD⁺ to NADH) of the original peptide, polypeptide or protein from which the fragment was derived. Such functional fragments include amino acid sequences that are about 200 to about 380, about 200 to about 370, about 200 to about 360, about 200 to about 350, about 200 to about 340, about 200 to about 330, about 200 to about 320, about 200 to about 310, about 200 to about 300, about 300 to about 380, about 300 to about 360, about 300 to about 370, about 300 to about 360, about 300 to about 350, about 300 to about 340, about 300 to about 330, about 300 to about 320, about 350 to about 380, about 350 to about 360 amino acids in length. These functional fragments can, for example, be truncations (e.g., C-terminal or N-terminal truncations) of a peptide, polypeptide, or protein. Functional fragments can also include one or more amino acid alteration described herein, such as an amino acid alteration of an engineered peptide described herein.

As used herein, the term “isolated” when used in reference to a molecule (e.g., peptide, polypeptide, protein, nucleic acid, polynucleotide, vector) or a cell (e.g., a yeast cell) refers to a molecule or cell that is substantially free of at least one component with which the referenced molecule or cell is found in nature. The term includes a molecule or cell that is removed from some or all components with which it is found in its natural environment. Therefore, an isolated molecule or cell can be partly or completely separated from other substances with which it is found in nature or with which it is grown, stored or subsisted in non-naturally occurring environments.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

As used herein, the term “non-naturally occurring” when used in reference to a microbial organism described herein is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, genetic alterations within coding regions and functional fragments thereof. Additional modifications include, for example, non-coding regulatory regions in which the modifications alter expression of a gene or operon. Exemplary metabolic polypeptides include enzymes or proteins within an acetyl-CoA or bioderived compound pathway described herein.

As use herein, the term “operatively linked” when used in reference to a nucleic acid encoding an engineered formate dehydrogenase refers to connection of a nucleotide sequence encoding an engineered formate dehydrogenase described herein to another nucleotide sequence (e.g., a promoter) is such a way as to allow for the connected nucleotide sequences to function (e.g., express the engineered formate dehydrogenase in the microbial organism).

As used herein, the term “pathway” when used in reference to production of a desired product (e.g., 1,3-BDO or a bioderived compound) refers to one or more polypeptides (e.g., proteins or enzymes) that catalyze the conversion of a substrate compound to a product compound and/or produce a co-substrate for the conversion of a substrate compound to a product compound. Such a product compound can be one of the bioderived compounds described herein, or an intermediate compound that can lead to the bioderived compound upon further conversion by other proteins or enzymes of the metabolic pathway. Accordingly, a metabolic pathway can be comprised of a series of metabolic polypeptides (e.g., two, three, four, five, six, seven, eight, nine, ten or more) that act upon a substrate compound to convert it to a given product compound through a series of intermediate compounds. The metabolic polypeptides of a metabolic pathway can be encoded by an exogenous nucleic acid as described herein or produced naturally by the host microbial organism.

As used herein, the term “recombinant” with respect to a nucleic acid, such as a nucleic acid comprising a gene that encodes a protein or polypeptide (e.g., an engineered formate dehydrogenase described herein), refers to: a nucleic acid that has been artificially supplied to a biological system; a nucleic acid that has been modified within a biological system, or a nucleic acid whose expression or regulation has been manipulated within a biological system. The recombinant nucleic acid can be supplied to the biological system, for example, by introduction of the nucleic acid into genetic material of a microbial organism, such as by integration into a microbial organism chromosome, or as non-chromosomal genetic material such as a plasmid. A recombinant nucleic acid that is introduced into or expressed in a microbial organism may be a nucleic acid that comes from a different organism or species from the microbial organism, or may be a synthetic nucleic acid, or may be a nucleic acid that is also endogenously expressed in the same organism or species as the microbial organism. A recombinant nucleic acid that is also endogenously expressed in the same organism or species as the microbial organism can be considered heterologous if: the sequence of the recombinant nucleic acid is modified relative to the endogenously expressed sequence, the sequence of a regulatory region such as a promoter that controls expression of the nucleic acid is modified relative to the regulatory region of the endogenously expressed sequence, the nucleic acid is expressed in an alternate location in the genome of the microbial organism relative to the endogenously expressed sequence, the nucleic acid is expressed in a different copy number in the microbial organism relative to the endogenously expressed sequence, and/or the nucleic acid is expressed as non-chromosomal genetic material such as a plasmid in the microbial organism.

As used herein, the term “promoter” when used in reference to a nucleic acid encoding an engineered formate dehydrogenase refers to a nucleotide sequence where transcription of a linked open reading frame (e.g., a nucleotide sequence encoding an engineered formate dehydrogenase) by an RNA polymerase begins. A promoter sequence can be located directly upstream or at the 5′ end of the transcription initiation site. RNA polymerase and the necessary transcription factors bind to a promoter sequence and initiate transcription. Promoter sequences define the direction of transcription and indicate which DNA strand will be transcribed, i.e. the sense strand.

As used herein, the term “substantially anaerobic” when used in reference to a culture or growth condition is intended to mean that the amount of dissolved oxygen in a liquid medium is less than about 10% of saturation. The term also is intended to include sealed chambers maintained with an atmosphere of less than about 1% oxygen that include liquid or solid medium.

As used herein, the term “vector” refers to a compound and/or composition that transduces, transforms, or infects a microbial organism, thereby causing the microbial organism to express nucleic acids and/or proteins other than those native to the microbial organism, or in a manner not native to the cell. Vectors can be constructed to include one or more biosynthetic pathway enzyme or protein, such as an engineered FDH described herein, encoded by a nucleotide sequence operably linked to expression control sequences (e.g., promoter) that are functional in the microbial organism (“expression vector”). Expression vectors applicable for use in the microbial organisms described herein include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more recombinant or exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of a recombinant or exogenous nucleic acid encoding an enzyme or protein involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid or its corresponding gene product (e.g., enzyme or protein). It is understood by those skilled in the art that the recombinant or exogenous nucleic acid is expressed in a sufficient amount to produce the desired product, and it is further understood that expression levels can be optimized to obtain sufficient expression using methods well known in the art and as disclosed herein.

Those skilled in the art will understand that the genetic alterations, including metabolic modifications exemplified herein, are described with reference to a suitable microbial organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. For example, mouse epoxide hydrolase and human epoxide hydrolase can be considered orthologs for the biological function of hydrolysis of epoxides. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities. Members of the serine protease family of enzymes, including tissue plasminogen activator and elastase, are considered to have arisen by vertical descent from a common ancestor.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, those skilled in the art will understand that the orthologous gene harboring the metabolic activity to be introduced or disrupted is to be chosen for construction of the non-naturally occurring microbial organism. An example of orthologs exhibiting separable activities is where distinct activities have been separated into distinct gene products between two or more species or within a single species. A specific example is the separation of elastase proteolysis and plasminogen proteolysis, two types of serine protease activity, into distinct molecules as plasminogen activator and elastase. A second example is the separation of Mycoplasma 5′-3′ exonuclease and Drosophila DNA polymerase III activity. The DNA polymerase from the first species can be considered an ortholog to either or both of the exonuclease and the polymerase from the second species and vice versa.

In contrast, paralogs are homologs related by, for example, duplication followed by evolutionary divergence and have similar or common, but not identical functions. Paralogs can originate or derive from, for example, the same species or from a different species. For example, microsomal epoxide hydrolase (epoxide hydrolase I) and soluble epoxide hydrolase (epoxide hydrolase II) can be considered paralogs because they represent two distinct enzymes, co-evolved from a common ancestor, that catalyze distinct reactions and have distinct functions in the same species. Paralogs are proteins from the same species with significant sequence similarity to each other suggesting that they are homologous, or related through co-evolution from a common ancestor. Groups of paralogous protein families include HipA homologs, luciferase genes, peptidases, and others.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

Therefore, in identifying and constructing the non-naturally occurring microbial organisms described herein having biosynthetic capability for a desired product, those skilled in the art will understand with applying the teaching and guidance provided herein to a particular species that the identification of metabolic modifications can include identification and inclusion or inactivation of orthologs. To the extent that paralogs and/or nonorthologous gene displacements are present in the referenced microbial organism that encode an enzyme catalyzing a similar or substantially similar metabolic reaction, those skilled in the art also can utilize these evolutionally related genes. Similarly, for a gene disruption, evolutionally related genes can also be disrupted or deleted in a microbial organism to reduce or eliminate functional redundancy of enzymatic activities targeted for disruption.

Orthologs, paralogs and nonorthologous gene displacements can be determined by methods well known to those skilled in the art. For example, inspection of nucleic acid or amino acid sequences for two polypeptides will reveal sequence identity and similarities between the compared sequences. Based on such similarities, one skilled in the art can determine if the similarity is sufficiently high to indicate the proteins are related through evolution from a common ancestor. Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide sequence similarity or identity. Parameters for sufficient similarity to determine relatedness are computed based on well known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 25% to 100% sequence identity. Proteins that are unrelated can have an identity which is essentially the same as would be expected to occur by chance, if a database of sufficient size is scanned (about 5%). Sequences between 5% and 24% may or may not represent sufficient homology to conclude that the compared sequences are related. Additional statistical analysis to determine the significance of such matches given the size of the data set can be carried out to determine the relevance of these sequences.

Exemplary parameters for determining relatedness of two or more sequences using the BLAST algorithm, for example, can be as set forth below. Briefly, amino acid sequence alignments can be performed using BLASTP version 2.0.8 (Jan. 5 1999) and the following parameters: Matrix: 0 BLOSUM62; gap open: 11; gap extension: 1; x_dropoff: 50; expect: 10.0; wordsize: 3; filter: on. Nucleotide sequence alignments can be performed using BLASTN version 2.0.6 (Sep. 16 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

In some embodiments, provided herein is an engineered formate dehydrogenase that is a variant of a wild-type or parent formate dehydrogenase. Such an engineered formate dehydrogenase includes one or more alterations described herein and higher catalytic activity relative to the wild-type or parent formate dehydrogenase as described herein. The engineered formate dehydrogenase provided herein is capable of catalyzing the conversion of formate to carbon dioxide and/or NAD⁺ to NADH. An exemplary enzymatic reaction catalyzed by an engineered formate dehydrogenase described herein is represented by:

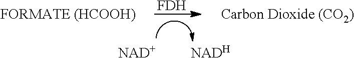

Accordingly, in some embodiments, the engineered formate dehydrogenase provided herein is capable of catalyzing the conversion of formate to carbon dioxide. In some embodiments, the engineered formate dehydrogenase provided herein is capable of catalyzing the conversion of NAD⁺ to NADH. In some embodiments, the engineered formate dehydrogenase provided herein is capable of catalyzing the conversion of catalyzing the conversion of formate to carbon dioxide and NAD⁺ to NADH.

In some embodiments, an engineered formate dehydrogenase is derived from Gibbsiella quercinecans (UniprotID: A0A250B5N7; SEQ ID NO: 1). In some embodiments, an engineered formate dehydrogenase is derived from Candida boidinii (UniprotID: 013437; SEQ ID NO: 2). Such an engineered formate dehydrogenase, in some embodiments, includes one or more alterations at a position described in TABLE 6 and/or TABLE 7. Such an engineered formate dehydrogenase provided herein can be classified as an enzyme that catalyzes the same reaction as the formate dehydrogenase of Gibbsiella quercinecans (UniprotID: A0A250B5N7; SEQ ID NO: 1) and/or Candida boidinii (UniprotID: 013437; SEQ ID NO: 2). Accordingly, in some embodiments, an engineered formate dehydrogenase provided herein is capable of forming carbon dioxide and/or NADH. Other embodiments provide an engineered formate dehydrogenase selected from or derived from any of the formate dehydrogenases described in TABLE 1, including any one of SEQ ID NOS: 3-24. Such an engineered formate dehydrogenase, in some embodiments, includes one or more alterations at a position corresponding to a position described in TABLE 6 and/or TABLE 7. Such an engineered formate dehydrogenase provided herein can be classified as an enzyme that catalyzes the same reaction as one or more of the formate dehydrogenases described in TABLE 1.

In some embodiments, provided herein is an engineered formate dehydrogenase having a variant of amino acid sequence SEQ ID NO: 1 or SEQ ID NO: 2 or a functional fragment thereof, wherein the engineered formate dehydrogenase includes one or more alterations at a position described in TABLE 6 and/or TABLE 7. In some embodiments, the engineered formate dehydrogenase includes one or more alterations at a position described in TABLE 6. In some embodiments, the engineered formate dehydrogenase comprises one or more alterations at a position described in TABLE 7. In some embodiments, an engineered formate dehydrogenase having such alterations described herein is capable of: (a) catalyzing the conversion of formate to carbon dioxide; (b) catalyzing the conversion of NAD⁺ to NADH; or (c) catalyzing the conversion of formate to carbon dioxide and NAD⁺ to NADH. Accordingly, in some embodiments, such an engineered formate dehydrogenase provided herein catalyzes the conversion of formate to carbon dioxide. In some embodiments, such an engineered formate dehydrogenase provided herein catalyzes the conversion of NAD⁺ to NADH. In some embodiments, an engineered formate dehydrogenase provided herein catalyzes the conversion of formate to carbon dioxide and NAD⁺ to NADH.

It is understood that the engineered formate dehydrogenases, such as polypeptide variants of formate dehydrogenases having the amino acid sequence of SEQ ID NO: 1 or SEQ ID NO: 2, as described herein, can carry out a similar enzymatic reaction as the parent formate dehydrogenase as discussed above. It is further understood that the polypeptide variants of the formate dehydrogenase enzyme can include variants that provide a beneficial characteristic to the engineered formate dehydrogenase, including but not limited to, increased activity (see, e.g., EXAMPLE 6). In some embodiments, the engineered formate dehydrogenase can exhibit an activity that is at least the same or higher than a wild-type or parent formate dehydrogenase, that is, it has activity that is higher than a formate dehydrogenase without the variant at the same amino acid position(s). For example, the engineered formate dehydrogenases provided here can have at least 0.5, at least 0.6, at least 0.7, at least 0.8, at least 0.9, at least 1.0, at least 1.1, at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, at least 3.0, at least 3.5, at least 4.0, at least 4.5, at least 5.0, at least 5.5, at least 6.0, at least 6.5, at least 7.0, at least 7.5, at least 8.0, at least 8.5, at least 9.0, at least 9.5, at least 10, or even higher fold activity over a wild-type or parent formate dehydrogenase (see, e.g., EXAMPLE 6). In some embodiments, an engineered formate dehydrogenase provided herein has an activity that is at least 0.5, at least 1.0, at least 1.5, or at least 2.0 fold higher than the activity of a formate dehydrogenase consisting of the amino acid sequence of SEQ ID NO: 1 or 2. In some embodiments, an engineered formate dehydrogenase provided herein has an activity that is at least 0.5 fold higher. In some embodiments, an engineered formate dehydrogenase provided herein has an activity that is at least 1.0 fold higher. In some embodiments, an engineered formate dehydrogenase provided herein has an activity that is at least 1.5 fold higher. In some embodiments, an engineered formate dehydrogenase provided herein has an activity that is at least 2.0 fold higher. It is understood that activity refers to the ability of an engineered formate dehydrogenase described herein to convert a substrate to a product relative to a wild-type or parent formate dehydrogenase under the same assay conditions, such as those described herein (see, e.g., EXAMPLE 6).

In some embodiments, the activity of a formate dehydrogenase described herein is measured as the catalytic constant (k_{cat}) value or turnover number. In some embodiments, the k_{cat} is at least 0.1 s⁻¹, at least 0.2 s⁻¹, at least 0.3 s⁻¹, at least 0.4 s⁻¹, at least 0.5 s⁻¹, at least 0.6 s⁻¹, at least 0.7 s⁻¹, at least 0.8 s⁻¹, at least 0.9 s⁻¹, at least 1 s⁻¹, at least 2 s⁻¹, at least 3 s⁻¹, at least 4 s⁻¹, at least 5 s⁻¹, at least 6 s⁻¹, at least 7 s⁻¹, at least 8 s⁻¹, at least 9 s⁻¹, at least 10 s⁻¹, at least 11 s⁻¹, at least 12 s⁻¹, at least 13 s⁻¹, at least 14 s⁻¹, at least 15 s⁻¹, at least 16 s⁻¹, at least 17 s⁻¹, at least 18 s⁻¹, at least 19 s⁻¹, at least 20 s⁻¹, at least 21 s⁻¹, at least 22 s⁻¹, at least 23 s⁻¹, at least 24 s⁻¹, at least 25 s⁻¹, at least 26 s⁻¹, at least 27 s⁻¹, at least 28 s⁻¹, at least 29 s⁻¹, at least 30 s⁻¹, at least 31 s⁻¹, at least 32 s⁻¹, at least 33 s⁻¹, at least 34 s⁻¹, at least 35 s⁻¹, at least 36 s⁻¹, at least 37 s⁻¹, at least 38 s⁻¹, at least 39 s⁻¹, at least 40 s⁻¹, at least 41 s⁻¹, at least 42 s⁻¹, at least 43 s⁻¹, at least 44 s⁻¹, at least 45 s⁻¹, at least 46 s⁻¹, at least 47 s⁻¹, at least 48 s⁻¹, at least 49 s⁻¹, at least 50 s⁻¹, at least 51 s⁻¹, at least 52 s⁻¹, at least 53 s⁻¹, at least 54 s⁻¹, at least 55 s⁻¹, at least 56 s⁻¹, at least 57 s⁻¹, at least 58 s⁻¹, at least 59 s⁻¹, at least 60 s⁻¹, at least 61 s⁻¹, at least 62 s⁻¹, at least 63 s⁻¹, at least 64 s⁻¹, at least 65 s⁻¹, at least 66 s⁻¹, at least 67 s⁻¹, at least 68 s⁻¹, at least 69 s⁻¹, at least 70 s⁻¹, at least 71 s⁻¹, at least 72 s⁻¹, at least 73 s⁻¹, at least 74 s⁻¹, at least 75 s⁻¹, at least 76 s⁻¹, at least 77 s⁻¹, at least 78 s⁻¹, at least 79 s⁻¹, at least 80 s⁻¹, at least 81 s⁻¹, at least 82 s⁻¹, at least 83 s⁻¹, at least 84 s⁻¹, at least 85 s⁻¹, at least 86 s⁻¹, at least 87 s⁻¹, at least 88 s⁻¹, at least 89 s⁻¹, at least 90 s⁻¹, at least 91 s⁻¹, at least 92 s⁻¹, at least 93 s⁻¹, at least 94 s⁻¹, at least 95 s⁻¹, at least 96 s⁻¹, at least 97 s⁻¹, at least 98 s⁻¹, at least 99 s⁻¹, at least 100 s⁻¹, at least 500 s⁻¹, at least 1000 s⁻¹, at least 2000 s⁻¹, at least 3000 s⁻¹, at least 4000 s⁻¹, at least 5000 s⁻¹, at least 6000 s⁻¹, at least 7000 s⁻¹, at least 8000 s⁻¹, at least 9000 s⁻¹, at least 10,000 s⁻¹, In some embodiments, the K_{cat} is between 1 s⁻¹ and 100 s⁻¹, between 5 s⁻¹ and 50 s⁻¹, or between 10 s⁻¹ and 50 s⁻¹.

In some embodiments, the activity of a formate dehydrogenase described herein is measured as the Michaelis constant (Kₘ). In some embodiments, the Kₘ is less than 0.005 mM, 0.006 mM, 0.007 mM, 0.008 mM, 0.009 mM, 0.01 mM, 0.02 mM, 0.03 mM, 0.04 mM, 0.05 mM, 0.06 mM, 0.07 mM, 0.08 mM, 0.09 mM, 0.1 mM, less than 0.2 mM, less than 0.3 mM, less than 0.4 mM, less than 0.5 mM, less than 0.6 mM, less than 0.7 mM, less than 0.8 mM, less than 0.9 mM, less than 1 mM, less than 2 mM, less than 3 mM, less than 4 mM, less than 5 mM, less than 6 mM, less than 7 mM, less than 8 mM, less than 9 mM, less than 10 mM, less than 11 mM, less than 12 mM, less than 13 mM, less than 14 mM, less than 15 mM, less than 16 mM, less than 17 mM, less than 18 mM, less than 19 mM, less than 20 mM, less than 21 mM, less than 22 mM, less than 23 mM, less than 24 mM, less than 25 mM, less than 26 mM, less than 27 mM, less than 28 mM, less than 29 mM, less than 30 mM, less than 31 mM, less than 32 mM, less than 33 mM, less than 34 mM, less than 35 mM, less than 36 mM, less than 37 mM, less than 38 mM, less than 39 mM, less than 40 mM, less than 41 mM, less than 42 mM, less than 43 mM, less than 44 mM, less than 45 mM, less than 46 mM, less than 47 mM, less than 48 mM, less than 49 mM, less than 50 mM, less than 51 mM, less than 52 mM, less than 53 mM, less than 54 mM, less than 55 mM, less than 56 mM, less than 57 mM, less than 58 mM, less than 59 mM, less than 60 mM, less than 61 mM, less than 62 mM, less than 63 mM, less than 64 mM, less than 65 mM, less than 66 mM, less than 67 mM, less than 68 mM, less than 69 mM, less than 70 mM, less than 71 mM, less than 72 mM, less than 73 mM, less than 74 mM, less than 75 mM, less than 76 mM, less than 77 mM, less than 78 mM, less than 79 mM, less than 80 mM, less than 81 mM, less than 82 mM, less than 83 mM, less than 84 mM, less than 85 mM, less than 86 mM, less than 87 mM, less than 88 mM, less than 89 mM, less than 90 mM, less than 91 mM, less than 92 mM, less than 93 mM, less than 94 mM, less than 95 mM, less than 96 mM, less than 97 mM, less than 98 mM, less than 99 mM, less than 100 mM, less than 500 mM, or less than 1000 mM, In some embodiments, the Kₘ is between 0.005 mM and 0.010 mM, between 0.5 mM and 10 mM, between 1 mM and 10 mM, between 2 mM and 10 mM, between 3 mM and 10 mM, between 4 mM and 10 mM, between 5 mM and 10 mM, between 6 mM and 10 mM, between 7 mM and 10 mM, between 8 mM and 10 mM, or between 9 mM and 10 mM.

In some embodiments, the activity of a formate dehydrogenase described herein is measured as the catalytic efficiency (k_{cat}/kₘ). In some embodiments, the catalytic efficiency is measured in units of liter/(millimole*second). In some embodiments, the catalytic efficiency is greater than 0.1, greater than 0.2, greater than 0.3, greater than 0.4, greater than 0.5, greater than 0.6, greater than 0.7, greater than 0.8, greater than 0.9, greater than 1, greater than 2, greater than 3, greater than 4, greater than 5, greater than 6, greater than 7, greater than 8, greater than 9, greater than 10, greater than 11, greater than 12, greater than 13, greater than 14, greater than 15, greater than 16, greater than 17, greater than 18, greater than 19, greater than 20, greater than 21, greater than 22, greater than 23, greater than 24, greater than 25, greater than 26, greater than 27, greater than 28, greater than 29, greater than 30, greater than 31, greater than 32, greater than 33, greater than 34, greater than 35, greater than 36, greater than 37, greater than 38, greater than 39, greater than 40, greater than 41, greater than 42, greater than 43, greater than 44, greater than 45, greater than 46, greater than 47, greater than 48, greater than 49, greater than 50, greater than 51, greater than 52, greater than 53, greater than 54, greater than 55, greater than 56, greater than 57, greater than 58, greater than 59, greater than 60, greater than 61, greater than 62, greater than 63, greater than 64, greater than 65, greater than 66, greater than 67, greater than 68, greater than 69, greater than 70, greater than 71, greater than 72, greater than 73, greater than 74, greater than 75, greater than 76, greater than 77, greater than 78, greater than 79, greater than 80, greater than 81, greater than 82, greater than 83, greater than 84, greater than 85, greater than 86, greater than 87, greater than 88, greater than 89, greater than 90, greater than 91, greater than 92, greater than 93, greater than 94, greater than 95, greater than 96, greater than 97, greater than 98, greater than 99, greater than 100, greater than 500, greater than 1000. In some embodiments, the catalytic efficiency (k_{cat}/kₘ) is between 1 and 30 liter/(millimole*second), between 5 and 30 liter/(millimole*second), between 1 and 10 liter/(millimole*second), between 10 and 30 liter/(millimole*second), or between 20 and 30 liter/(millimole*second).

In some embodiments, an engineered formate dehydrogenase provided herein is a variant of a reference polypeptide, wherein the reference polypeptide has an amino acid sequence of SEQ ID NO: 1 or SEQ ID NO: 2, and the engineered formate dehydrogenase has one or more alterations at a position described in TABLE 6 and/or TABLE 7 relative to SEQ ID NO: 1 or SEQ ID NO: 2. Accordingly, in some embodiments, an engineered formate dehydrogenase provided herein is a variant of SEQ ID NO: 1, and has one or more alterations at a position described in TABLE 6 relative to SEQ ID NO: 1. In some embodiments, an engineered formate dehydrogenase provided herein is a variant of SEQ ID NO: 2, and has one or more alterations at a position described in TABLE 7 relative to SEQ ID NO: 2.

In some embodiments, an engineered formate dehydrogenase provided herein has an amino acid sequence that is a variant of SEQ ID NO: 1 or SEQ ID NO: 2 that includes one or more alterations as described in TABLE 6 and/or TABLE 7, wherein the portion, other than the one or more alterations described in TABLE 6 and/or TABLE 7, of the engineered formate dehydrogenase has at least 65%, 70%, 75%, 80%, 85%, 90%, 95%, 98% or 99% sequence identity, or is identical, to an amino acid sequence referenced as SEQ ID NO: 1 or SEQ ID NO: 2. Accordingly, in some embodiments, an engineered formate dehydrogenase provided herein has an amino acid sequence that includes one or more alterations as described in TABLE 6 and the portion, other than the alteration described in Tables 1, 3, and/or 4, of the engineered formate dehydrogenase has at least 65% identical to SEQ ID NO: 1. In some embodiments, an engineered formate dehydrogenase provided herein has an amino acid sequence that includes one or more alterations as described in TABLE 6 and the portion, other than the alteration described in TABLE 6, of the engineered formate dehydrogenase has at least 70% identical to SEQ ID NO: 1. In some embodiments, an engineered formate dehydrogenase provided herein has an amino acid sequence that includes one or more alterations as described in TABLE 6 and the portion, other than the alteration described in TABLE 6, of the engineered formate dehydrogenase has at least 75% identical to SEQ ID NO: 1. In some embodiments, an engineered formate dehydrogenase provided herein has an amino acid sequence that includes one or more alterations as described in TABLE 6 and the portion, other than the alteration described in TABLE 6, of the engineered formate dehydrogenase has at least 80% identical to SEQ ID NO: 1. In some embodiments, an engineered formate dehydrogenase provided herein has an amino acid sequence that includes one or more alterations as described in TABLE 6 and the portion, other than the alteration described in TABLE 6, of the engineered formate dehydrogenase has at least 85% identical to SEQ ID NO: 1. In some embodiments, an engineered formate dehydrogenase provided herein has an amino acid sequence that includes one or more alterations as described in TABLE 6 and the portion, other than the alteration described in TABLE 6, of the engineered formate dehydrogenase has at least 90% identical to SEQ ID NO: 1. In some embodiments, an engineered formate dehydrogenase provided herein has an amino acid sequence that includes one or more alterations as described in TABLE 6 and the portion, other than the alteration described in TABLE 6, of the engineered formate dehydrogenase has at least 95% identical to SEQ ID NO: 1. In some embodiments, an engineered formate dehydrogenase provided herein has an amino acid sequence that includes one or more alterations as described in TABLE 6 and the portion, other than the alteration described in TABLE 6, of the engineered formate dehydrogenase has at least 98% identical to SEQ ID NO: 1. In some embodiments, an engineered formate dehydrogenase provided herein has an amino acid sequence that includes one or more alterations as described in TABLE 6 and the portion, other than the alteration described in TABLE 6, of the engineered formate dehydrogenase has at least 99% identical to SEQ ID NO: 1. In some embodiments, an engineered formate dehydrogenase provided herein has an amino acid sequence that includes one or more alterations as described in TABLE 7 and the portion, other than the alteration described in TABLE 7, of the engineered formate dehydrogenase has at least 65% identical to SEQ ID NO:2. In some embodiments, an engineered formate dehydrogenase provided herein has an amino acid sequence that includes one or more alterations as described in TABLE 7 and the portion, other than the alteration described in TABLE 7, of the engineered formate dehydrogenase has at least 70% identical to SEQ ID NO:2. In some embodiments, an engineered formate dehydrogenase provided herein has an amino acid sequence that includes one or more alterations as described in TABLE 7 and the portion, other than the alteration described in TABLE 7, of the engineered formate dehydrogenase has at least 75% identical to SEQ ID NO:2. In some embodiments, an engineered formate dehydrogenase provided herein has an amino acid sequence that includes one or more alterations as described in TABLE 7 and the portion, other than the alteration described in TABLE 7, of the engineered formate dehydrogenase has at least 80% identical to SEQ ID NO:2. In some embodiments, an engineered formate dehydrogenase provided herein has an amino acid sequence that includes one or more alterations as described in TABLE 7 and the portion, other than the alteration described in TABLE 7, of the engineered formate dehydrogenase has at least 85% identical to SEQ ID NO:2. In some embodiments, an engineered formate dehydrogenase provided herein has an amino acid sequence that includes one or more alterations as described in TABLE 7 and the portion, other than the alteration described in TABLE 7, of the engineered formate dehydrogenase has at least 90% identical to SEQ ID NO:2. In some embodiments, an engineered formate dehydrogenase provided herein has an amino acid sequence that includes one or more alterations as described in TABLE 7 and the portion, other than the alteration described in TABLE 7, of the engineered formate dehydrogenase has at least 95% identical to SEQ ID NO:2. In some embodiments, an engineered formate dehydrogenase provided herein has an amino acid sequence that includes one or more alterations as described in TABLE 7 and the portion, other than the alteration described in TABLE 7, of the engineered formate dehydrogenase has at least 98% identical to SEQ ID NO:2. In some embodiments, an engineered formate dehydrogenase provided herein has an amino acid sequence that includes one or more alterations as described in TABLE 7 and the portion, other than the alteration described in TABLE 7, of the engineered formate dehydrogenase has at least 99% identical to SEQ ID NO:2.

Sequence identity, homology or similarity refers to sequence similarity between two polypeptides or between two nucleic acid molecules. Identity can be determined by comparing a position in each sequence which may be aligned for purposes of comparison. When a position in the compared sequence is occupied by the same base or amino acid, then the molecules are identical at that position. A degree of homology between sequences is a function of the number of matching or homologous positions shared by the sequences. A polypeptide or polypeptide region (or a polynucleotide or polynucleotide region) has a certain percentage (for example, 65%, 70%, 75%, 80%, 85%, 90%, 95%, 98% or 99%) of “sequence identity” to another sequence means that, when aligned, that percentage of amino acids (or nucleotide bases) are the same in comparing the two sequences. The alignment of two sequences to determine their percent sequence identity can be done using software programs known in the art, such as, for example, those described in Ausubel et al., Current Protocols in Molecular Biology, John Wiley and Sons, Baltimore, MD (1999). Preferably, default parameters are used for the alignment. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information (see also Altschul et al., “J. Mol. Biol. 215:403-410 (1990)).

In some embodiments, an engineered formate dehydrogenase provided herein includes one or more amino acid substitutions at a position corresponding to position 2, 9, 16, 19, 27, 29, 30, 41, 53, 73, 97, 98, 100, 101, 120, 121, 122, 123, 124, 128, 138, 143, 144, 145, 146, 147, 149, 150, 151, 152, 153, 155, 175, 176, 191, 196, 198, 199, 203, 204, 206, 217, 218, 224, 231, 238, 256, 262, 264, 265, 266, 267, 269, 271, 284, 285, 287, 290, 291, 297, 301, 303, 313, 315, 319, 325, 329, 335, 336, 338, 339, 342, 343, 346, 350, 355, 365, 374, 381, 382, or 384, or a combination thereof, in SEQ ID NO: 1.

In some embodiments, an engineered formate dehydrogenase provided herein includes one or more amino acid substitutions at a position corresponding to position 2, 9, 16, 19, 27, 29, 30, 41, 53, 73, 97, 98, 100, 101, 120, 121, 122, 123, 124, 128, 138, 143, 144, 145, 146, 147, 149, 150, 151, 152, 153, 155, 175, 176, 191, 196, 198, 199, 203, 204, 206, 217, 218, 224, 231, 238, 256, 262, 264, 265, 266, 267, 269, 271, 284, 285, 287, 290, 291, 297, 301, 303, 313, 315, 319, 325, 329, 335, 336, 338, 339, 342, 343, 346, 350, 355, 365, 374, 381, 382, or 384, or a combination thereof, in SEQ ID NO: 1.

In some embodiments, an engineered formate dehydrogenase provided herein includes one or more amino acid substitutions at a position corresponding to position 2, 9, 16, 19, 27, 29, 30, 41, 53, 73, 97, 98, 101, 120, 122, 124, 138, 144, 145, 146, 147, 150, 151, 155, 175, 176, 191, 198, 199, 204, 206, 217, 218, 231, 238, 256, 262, 264, 265, 266, 267, 269, 271, 284, 285, 287, 290, 291, 297, 301, 303, 313, 319, 325, 329, 335, 336, 338, 339, 342, 346, 350, 355, 365, 374, 381, 382, or 384, or a combination thereof, in SEQ ID NO: 1.

In some embodiments, an engineered formate dehydrogenase provided herein includes one or more amino acid substitutions at a position corresponding to position 2, 98, 199, 206, 231, 266, or 381, or a combination thereof, in SEQ ID NO: 1.

In some embodiments, an engineered formate dehydrogenase provided herein includes one or more amino acid substitutions at a position corresponding to position 9, 16, 19, 27, 29, 30, 41, 53, 73, 97, 98, 101, 120, 122, 124, 138, 144, 145, 146, 147, 150, 151, 155, 175, 176, 191, 198, 199, 204, 217, 218, 231, 238, 256, 262, 264, 265, 266, 267, 269, 271, 284, 285, 287, 290, 291, 297, 301, 303, 313, 319, 325, 329, 335, 336, 338, 339, 342, 346, 350, 355, 365, 374, 381, 382, or 384, or a combination thereof, in SEQ ID NO: 1.

In some embodiments, an engineered formate dehydrogenase provided herein includes one or more amino acid substitutions at a position corresponding to position 36, 64, 80, 91, 97, 111, 120, 162, 164, 187, 188, 214, 229, 256, 257, 260, 312, 313, 315, 320, 323, 361, or 362, or a combination thereof, in SEQ ID NO: 2.

In some embodiments, an engineered formate dehydrogenase provided herein includes one or more amino acid substitutions at a position corresponding to position 36, 64, 80, 111, 120, 162, 214, 229, 260, 315, 320, or 361, or a combination thereof, in SEQ ID NO: 2.

In some embodiments, an engineered formate dehydrogenase provided herein includes one or more alterations at a position described in TABLE 6 and/or TABLE 7, where the one or more amino acid alterations are conservative amino acid substitutions. In some embodiments, an engineered formate dehydrogenase provided herein includes one or more conservative amino acid substitutions relative to an alteration described in TABLE 6 and/or TABLE 7. As a non-limiting example, a conservative amino acid substitution relative to the C231A substitution in SEQ ID NO: 1 may include substitution of C231 for another non-polar (hydrophobic) amino acid (e.g., Cys (C), Ala (A), Val (V), Ile (I), Pro (P), Phe (F), Met (M), Trp (W), Gly (G), or Tyr (Y)). In some embodiments, an engineered formate dehydrogenase provided herein includes one or more alterations at a position described in TABLE 6 and/or TABLE 7, wherein the one or more amino acid alterations are non-conservative amino acid substitutions. In some embodiments, an engineered formate dehydrogenase provided herein includes one or more alterations at a position described in TABLE 6. In some embodiments, an engineered formate dehydrogenase provided herein includes one or more alterations at a position described in TABLE 7. In some embodiments, an engineered formate dehydrogenase provided herein includes a conservative amino acid substitution and/or non-conservative amino acid substitution in 1 to 10 amino acid positions as set forth in TABLE 6 and/or TABLE 7.

In some embodiments, an engineered formate dehydrogenase provided herein can further include a conservative amino acid substitution in from 1 to 50 amino acid positions, or alternatively from 2 to 50 amino acid positions, or alternatively from 3 to 50 amino acid positions, or alternatively from 4 to 50 amino acid positions, or alternatively from 5 to 50 amino acid positions, or alternatively from 6 to 50 amino acid positions, or alternatively from 7 to 50 amino acid positions, or alternatively from 8 to 50 amino acid positions, or alternatively from 9 to 50 amino acid positions, or alternatively from 10 to 50 amino acid positions, or alternatively from 15 to 50 amino acid positions, or alternatively from 20 to 50 amino acid positions, or alternatively from 30 to 50 amino acid positions, or alternatively from 40 to 50 amino acid positions, or alternatively from 45 to 50 amino acid positions, or any integer therein, wherein the positions are other than the variant amino acid positions set forth in TABLE 6 and/or TABLE 7. In some aspects, such a conservative amino acid sequence is a chemically conservative or an evolutionary conservative amino acid substitution. Methods of identifying conservative amino acids are well known to one of skill in the art, any one of which can be used to generate the isolated polypeptides described herein.

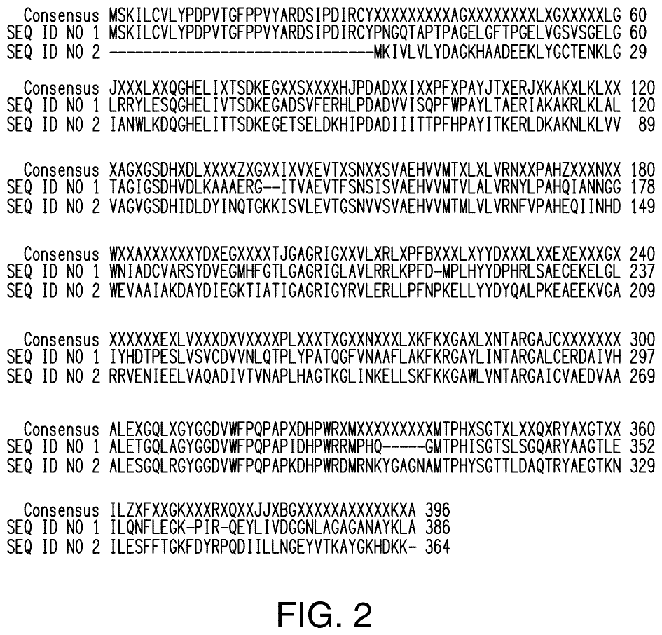

An engineered formate dehydrogenase provided herein may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, or 250 alterations relative to a wild-type or parent formate dehydrogenase. An engineered formate dehydrogenase provided herein may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 51, at most 52, at most 53, at most 54, at most 55, at most 56, at most 57, at most 58, at most 59, at most 60, at most 61, at most 62, at most 63, at most 64, at most 65, at most 66, at most 67, at most 68, at most 69, at most 70, at most 71, at most 72, at most 73, at most 74, at most 75, at most 76, at most 77, at most 78, at most 79, at most 80, at most 81, at most 82, at most 83, at most 84, at most 85, at most 86, at most 87, at most 88, at most 89, at most 90, at most 91, at most 92, at most 93, at most 94, at most 95, at most 96, at most 97, at most 98, at most 99, at most 100, at most 101, at most 102, at most 103, at most 104, at most 105, at most 106, at most 107, at most 108, at most 109, at most 110, at most 111, at most 112, at most 113, at most 114, at most 115, at most 116, at most 117, at most 118, at most 119, at most 120, at most 121, at most 122, at most 123, at most 124, at most 125, at most 126, at most 127, at most 128, at most 129, at most 130, at most 131, at most 132, at most 133, at most 134, at most 135, at most 136, at most 137, at most 138, at most 139, at most 140, at most 141, at most 142, at most 143, at most 144, at most 145, at most 146, at most 147, at most 148, at most 149, at most 150, at most 151, at most 152, at most 153, at most 154, at most 155, at most 156, at most 157, at most 158, at most 159, at most 160, at most 161, at most 162, at most 163, at most 164, at most 165, at most 166, at most 167, at most 168, at most 169, at most 170, at most 171, at most 172, at most 173, at most 174, at most 175, at most 176, at most 177, at most 178, at most 179, at most 180, at most 181, at most 182, at most 183, at most 184, at most 185, at most 186, at most 187, at most 188, at most 189, at most 190, at most 191, at most 192, at most 193, at most 194, at most 195, at most 196, at most 197, at most 198, at most 199, at most 200, at most 201, at most 202, at most 203, at most 204, at most 205, at most 206, at most 207, at most 208, at most 209, at most 210, at most 211, at most 212, at most 213, at most 214, at most 215, at most 216, at most 217, at most 218, at most 219, at most 220, at most 221, at most 222, at most 223, at most 224, at most 225, at most 226, at most 227, at most 228, at most 229, at most 230, at most 231, at most 232, at most 233, at most 234, at most 235, at most 236, at most 237, at most 238, at most 239, at most 240, at most 241, at most 242, at most 243, at most 244, at most 245, at most 246, at most 247, at most 248, at most 249, or at most 250 alterations relative to a wild-type or parent formate dehydrogenase. The one or more alterations may be located at one or more positions corresponding to the one or more positions described in TABLE 6 and/or TABLE 7. The one or more alterations may be located at one or more positions corresponding to one or more positions in SEQ ID NO: 1 and/or SEQ ID NO: 2. As used herein, the phrase “a residue corresponding to position X in SEQ ID NO: Y” refers to a residue at a corresponding position following an alignment of two sequences. For example, the residue in SEQ ID NO: 2 corresponding to the C (Cys) at position 231 in SEQ ID NO: 1 is the A (Ala) at position 203 in SEQ ID NO: 2 (see, e.g., FIG. 2). In some embodiments, a reference sequence is a formate dehydrogenase that is not SEQ ID NO: 1 or SEQ ID NO: 2.

An engineered formate dehydrogenase provided herein can include any combination of the alterations set forth in TABLE 6 and/or TABLE 7. One alteration alone, or in combination, can produce an engineered formate dehydrogenase that retains or improves the activity as described herein relative to a reference polypeptide, for example, the wild-type (native) formate dehydrogenase. In some embodiments, an engineered formate dehydrogenase provided herein includes at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 alterations as set forth in TABLE 6 and/or TABLE 7, including up to an alteration at all of the positions identified in Tables 1 and/or 2. In some embodiments, an engineered formate dehydrogenase provided herein includes at least 2 alterations as set forth in TABLE 6 and/or TABLE 7. In some embodiments, an engineered formate dehydrogenase provided herein includes at least 3 alterations as set forth in TABLE 6 and/or TABLE 7. In some embodiments, an engineered formate dehydrogenase provided herein includes at least 4 alterations as set forth in TABLE 6 and/or TABLE 7.

In some embodiments, the one or more amino acid alterations of the engineered formate dehydrogenase is an alteration described in TABLE 6. For example, in some embodiments, the one or more amino acid alterations result in an engineered formate dehydrogenase comprising: a) A at a residue corresponding to position 2 in SEQ ID NO: 1; b) F at a residue corresponding to position 9 in SEQ ID NO: 1; c) Y at a residue corresponding to position 16 in SEQ ID NO: 1; d) K or S at a residue corresponding to position 19 in SEQ ID NO: 1; e) K, E, N, A, T, or V at a residue corresponding to position 27 in SEQ ID NO: 1; f) G, E, K, N, D, A, T, or S at a residue corresponding to position 29 in SEQ ID NO: 1; g) G, S, A, R, or H at a residue corresponding to position 30 in SEQ ID NO: 1; h) K at a residue corresponding to position 41 in SEQ ID NO: 1; i) A at a residue corresponding to position 53 in SEQ ID NO: 1; j) V at a residue corresponding to position 73 in SEQ ID NO: 1; k) I or T at a residue corresponding to position 97 in SEQ ID NO: 1; l) W, S, T, or R at a residue corresponding to position 98 in SEQ ID NO: 1; m) A at a residue corresponding to position 100 in SEQ ID NO: 1; n) F at a residue corresponding to position 101 in SEQ ID NO: 1; o) C, G, A, V, H, I, S, F, or Q at a residue corresponding to position 120 in SEQ ID NO: 1; p) R at a residue corresponding to position 121 in SEQ ID NO: 1; q) S at a residue corresponding to position 122 in SEQ ID NO: 1; r) A at a residue corresponding to position 123 in SEQ ID NO: 1; s) T, A, V at a residue corresponding to position 124 in SEQ ID NO: 1; t) N, M, or S at a residue corresponding to position 128 in SEQ ID NO: 1; u) D at a residue corresponding to position 138 in SEQ ID NO: 1; v) W or Y at a residue corresponding to position 143 in SEQ ID NO: 1; w) I, C, S, A, N, or T at a residue corresponding to position 144 in SEQ ID NO: 1; x) P or S at a residue corresponding to position 145 in SEQ ID NO: 1; y) Q, N, G, P, Y, A, T, D, S, H, or V at a residue corresponding to position 146 in SEQ ID NO: 1; z) A, L, V, or C at a residue corresponding to position 147 in SEQ ID NO: 1; aa) G, A, T, or V at a residue corresponding to position 149 in SEQ ID NO: 1; bb) T, G, R, D, N, S, Q, E, V, or L at a residue corresponding to position 150 in SEQ ID NO: 1; cc) A, C, or T at a residue corresponding to position 151 in SEQ ID NO: 1; dd) A at a residue corresponding to position 152 in SEQ ID NO: 1; ee) T at a residue corresponding to position 153 in SEQ ID NO: 1; ff) F at a residue corresponding to position 155 in SEQ ID NO: 1; gg) R, I, V, A, T, or E at a residue corresponding to position 175 in SEQ ID NO: 1; hh) S at a residue corresponding to position 176 in SEQ ID NO: 1; ii) L at a residue corresponding to position 191 in SEQ ID NO: 1; jj) V at a residue corresponding to position 196 in SEQ ID NO: 1; kk) I at a residue corresponding to position 198 in SEQ ID NO: 1; ll) I or V at a residue corresponding to position 199 in SEQ ID NO: 1; mm) H at a residue corresponding to position 203 in SEQ ID NO: 1; nn) V at a residue corresponding to position 204 in SEQ ID NO: 1; oo) Q at a residue corresponding to position 206 in SEQ ID NO: 1; pp) V at a residue corresponding to position 217 in SEQ ID NO: 1; qq) T, N, R, A, E, K, G, H, R, D, S, or Q at a residue corresponding to position 218 in SEQ ID NO: 1; rr) R at a residue corresponding to position 224 in SEQ ID NO: 1; ss) D, A, K, R, V, I, L, T, Y or E at a residue corresponding to position 231 in SEQ ID NO: 1; tt) T, R, V, Q, or E at a residue corresponding to position 238 in SEQ ID NO: 1; uu) I, C, L, A, S, H, T, V, or E at a residue corresponding to position 256 in SEQ ID NO: 1; vv) E or S at a residue corresponding to position 262 in SEQ ID NO: 1; ww) E at a residue corresponding to position 264 in SEQ ID NO: 1; xx) N or H at a residue corresponding to position 265 in SEQ ID NO: 1; yy) M or L at a residue corresponding to position 266 in SEQ ID NO: 1; zz) F at a residue corresponding to position 267 in SEQ ID NO: 1; aaa) D or E at a residue corresponding to position 269 in SEQ ID NO: 1; bbb) L or M at a residue corresponding to position 271 in SEQ ID NO: 1; ccc) S, C, M, L, I, V, or A at a residue corresponding to position 284 in SEQ ID NO: 1; ddd) S or G at a residue corresponding to position 285 in SEQ ID NO: 1; eee) A at a residue corresponding to position 287 in SEQ ID NO: 1; fff) I at a residue corresponding to position 290 in SEQ ID NO: 1; ggg) D at a residue corresponding to position 291 in SEQ ID NO: 1; hhh) R, V, G, N, D, K, E, A, or Q at a residue corresponding to position 297 in SEQ ID NO: 1; iii) S, A, D, E, or N at a residue corresponding to position 301 in SEQ ID NO: 1; jjj) K at a residue corresponding to position 303 in SEQ ID NO: 1; kkk) Y at a residue corresponding to position 313 in SEQ ID NO: 1; lll) E or Y at a residue corresponding to position 315 in SEQ ID NO: 1; mmm) R, P, E, V, A, or K at a residue corresponding to position 319 in SEQ ID NO: 1; nnn) T or S at a residue corresponding to position 325 in SEQ ID NO: 1; ooo) H or N at a residue corresponding to position 329 in SEQ ID NO: 1; ppp) A, M, R, V, N, T, L, S, or Y at a residue corresponding to position 335 in SEQ ID NO: 1; qqq) A or G at a residue corresponding to position 336 in SEQ ID NO: 1; rrr) Y, F, W, S, D, V, A, L, or N at a residue corresponding to position 338 in SEQ ID NO: 1; sss) T, L, G, or A at a residue corresponding to position 339 in SEQ ID NO: 1; ttt) K, L, A, V, I, N, Y, T, E, S, M, R, C, or D at a residue corresponding to position 342 in SEQ ID NO: 1; uuu) A at a residue corresponding to position 343 in SEQ ID NO: 1; vvv) A, M, I, L, or F at a residue corresponding to position 346 in SEQ ID NO: 1; www) A at a residue corresponding to position 350 in SEQ ID NO: 1; xxx) E at a residue corresponding to position 355 in SEQ ID NO: 1; yyy) D, E, or P at a residue corresponding to position 365 in SEQ ID NO: 1; zzz) E, G, A, R, H, Q, or K at a residue corresponding to position 374 in SEQ ID NO: 1; aaaa) H, K, L, P, or R at a residue corresponding to position 381 in SEQ ID NO: 1; bbbb) S at a residue corresponding to position 382 in SEQ ID NO: 1; and/or cccc) S or T at a residue corresponding to position 384 in SEQ ID NO: 1.

In some embodiments, the one or more amino acid alterations result in an engineered formate dehydrogenase comprising: a) A at a residue corresponding to position 2 in SEQ ID NO: 1; b) F at a residue corresponding to position 9 in SEQ ID NO: 1; c) Y at a residue corresponding to position 16 in SEQ ID NO: 1; d) K or S at a residue corresponding to position 19 in SEQ ID NO: 1; e) K, E, N, A, T, or V at a residue corresponding to position 27 in SEQ ID NO: 1; f) G, E, K, N, D, A, T, or S at a residue corresponding to position 29 in SEQ ID NO: 1; g) G, S, A, R, or H at a residue corresponding to position 30 in SEQ ID NO: 1; h) K at a residue corresponding to position 41 in SEQ ID NO: 1; i) A at a residue corresponding to position 53 in SEQ ID NO: 1; j) V at a residue corresponding to position 73 in SEQ ID NO: 1; k) I or T at a residue corresponding to position 97 in SEQ ID NO: 1; l) W, R, or T at a residue corresponding to position 98 in SEQ ID NO: 1; m) F at a residue corresponding to position 101 in SEQ ID NO: 1; n) G, A, H, S, F, Q, C, V, or I at a residue corresponding to position 120 in SEQ ID NO: 1; o) S at a residue corresponding to position 122 in SEQ ID NO: 1; p) T, A, V at a residue corresponding to position 124 in SEQ ID NO: 1; q) D at a residue corresponding to position 138 in SEQ ID NO: 1; r) N, I, C, S, A, or T at a residue corresponding to position 144 in SEQ ID NO: 1; s) P or S at a residue corresponding to position 145 in SEQ ID NO: 1; t) P, D, V, Q, N, G, Y, A, T, S, or H at a residue corresponding to position 146 in SEQ ID NO: 1; u) V, L, C, A at a residue corresponding to position 147 in SEQ ID NO: 1; v) G, R, D, N, S, Q, E, L, T or V at a residue corresponding to position 150 in SEQ ID NO: 1; w) T, A, or C at a residue corresponding to position 151 in SEQ ID NO: 1; x) F at a residue corresponding to position 155 in SEQ ID NO: 1; y) R, I, V, A, T, or E at a residue corresponding to position 175 in SEQ ID NO: 1; z) S at a residue corresponding to position 176 in SEQ ID NO: 1; aa) L at a residue corresponding to position 191 in SEQ ID NO: 1; bb) I at a residue corresponding to position 198 in SEQ ID NO: 1; cc) I or V at a residue corresponding to position 199 in SEQ ID NO: 1; dd) V at a residue corresponding to position 204 in SEQ ID NO: 1; ee) Q at a residue corresponding to position 206 in SEQ ID NO: 1; ff) V at a residue corresponding to position 217 in SEQ ID NO: 1; gg) T, N, R, A, E, K, G, H, D, S, or Q at a residue corresponding to position 218 in SEQ ID NO: 1; hh) D, A, K, R, V, I, L, T, Y or E at a residue corresponding to position 231 in SEQ ID NO: 1; ii) T, R, V, Q, or E at a residue corresponding to position 238 in SEQ ID NO: 1; jj) I, C, L, H, T, V, E, A, or S at a residue corresponding to position 256 in SEQ ID NO: 1; kk) E or S at a residue corresponding to position 262 in SEQ ID NO: 1; ll) E at a residue corresponding to position 264 in SEQ ID NO: 1; mm) N or H at a residue corresponding to position 265 in SEQ ID NO: 1; nn) M or L at a residue corresponding to position 266 in SEQ ID NO: 1; oo) F at a residue corresponding to position 267 in SEQ ID NO: 1; pp) D or E at a residue corresponding to position 269 in SEQ ID NO: 1; qq) L or M at a residue corresponding to position 271 in SEQ ID NO: 1; rr) L, I, V, S, C, M, or A at a residue corresponding to position 284 in SEQ ID NO: 1; ss) S or G at a residue corresponding to position 285 in SEQ ID NO: 1; tt) A at a residue corresponding to position 287 in SEQ ID NO: 1; uu) I at a residue corresponding to position 290 in SEQ ID NO: 1; vv) D at a residue corresponding to position 291 in SEQ ID NO: 1; ww) R, V, G, N, D, K, E, A, or Q at a residue corresponding to position 297 in SEQ ID NO: 1; xx) S, A, D, E, or N at a residue corresponding to position 301 in SEQ ID NO: 1; yy) K at a residue corresponding to position 303 in SEQ ID NO: 1; zz) Y at a residue corresponding to position 313 in SEQ ID NO: 1; aaa) R, P, E, V, A, or K at a residue corresponding to position 319 in SEQ ID NO: 1; bbb) T or S at a residue corresponding to position 325 in SEQ ID NO: 1; ccc) H or N at a residue corresponding to position 329 in SEQ ID NO: 1; ddd) R, S, A, M, V, N, T, L, or Y at a residue corresponding to position 335 in SEQ ID NO: 1; eee) A or G at a residue corresponding to position 336 in SEQ ID NO: 1; fff) Y, F, W, L, S, D, V, A, or N at a residue corresponding to position 338 in SEQ ID NO: 1; ggg) L, G, A, T at a residue corresponding to position 339 in SEQ ID NO: 1; hhh) K, L, V, I, N, Y, T, E, M, R, D, A, S, or C at a residue corresponding to position 342 in SEQ ID NO: 1; iii) M, A, I, L, or F at a residue corresponding to position 346 in SEQ ID NO: 1; jjj) A at a residue corresponding to position 350 in SEQ ID NO: 1; kkk) E at a residue corresponding to position 355 in SEQ ID NO: 1; lll) D, E, or P at a residue corresponding to position 365 in SEQ ID NO: 1; mmm) E, G, A, R, H, Q, or K at a residue corresponding to position 374 in SEQ ID NO: 1; nnn) P, H, K, L, or R at a residue corresponding to position 381 in SEQ ID NO: 1; ooo) S at a residue corresponding to position 382 in SEQ ID NO: 1; and/or ppp) S or T at a residue corresponding to position 384 in SEQ ID NO: 1.

In some embodiments, the one or more amino acid alterations result in an engineered formate dehydrogenase comprising: a) A at a residue corresponding to position 2 in SEQ ID NO: 1; b) F at a residue corresponding to position 9 in SEQ ID NO: 1; c) Y at a residue corresponding to position 16 in SEQ ID NO: 1; d) K or S at a residue corresponding to position 19 in SEQ ID NO: 1; e) K, E, N, A, T, or V at a residue corresponding to position 27 in SEQ ID NO: 1; f) G, E, K, N, D, A, T, or S at a residue corresponding to position 29 in SEQ ID NO: 1; g) G, S, A, R, or H at a residue corresponding to position 30 in SEQ ID NO: 1; h) K at a residue corresponding to position 41 in SEQ ID NO: 1; i) A at a residue corresponding to position 53 in SEQ ID NO: 1; j) V at a residue corresponding to position 73 in SEQ ID NO: 1; k) I or T at a residue corresponding to position 97 in SEQ ID NO: 1; l) S or T at a residue corresponding to position 98 in SEQ ID NO: 1 m) F at a residue corresponding to position 101 in SEQ ID NO: 1; n) C, V, or I at a residue corresponding to position 120 in SEQ ID NO: 1; o) S at a residue corresponding to position 122 in SEQ ID NO: 1; p) V at a residue corresponding to position 124 in SEQ ID NO: 1; q) D at a residue corresponding to position 138 in SEQ ID NO: 1; r) I, C, S, A, or T at a residue corresponding to position 144 in SEQ ID NO: 1; s) S at a residue corresponding to position 145 in SEQ ID NO: 1; t) Q, N, G, Y, A, T, S, or H at a residue corresponding to position 146 in SEQ ID NO: 1; u) A at a residue corresponding to position 147 in SEQ ID NO: 1; v) T or V at a residue corresponding to position 150 in SEQ ID NO: 1; w) A or C at a residue corresponding to position 151 in SEQ ID NO: 1; x) F at a residue corresponding to position 155 in SEQ ID NO: 1; y) R, I, V, A, T, or E at a residue corresponding to position 175 in SEQ ID NO: 1; z) S at a residue corresponding to position 176 in SEQ ID NO: 1; aa) L at a residue corresponding to position 191 in SEQ ID NO: 1; bb) I at a residue corresponding to position 198 in SEQ ID NO: 1; cc) I or V at a residue corresponding to position 199 in SEQ ID NO: 1; dd) V at a residue corresponding to position 204 in SEQ ID NO: 1; ee) Q at a residue corresponding to position 206 in SEQ ID NO: 1; ff) V at a residue corresponding to position 217 in SEQ ID NO: 1; gg) T, N, R, A, E, K, G, H, D, S, or Q at a residue corresponding to position 218 in SEQ ID NO: 1; hh) D, A, K, R, V, I, L, T, Y or E at a residue corresponding to position 231 in SEQ ID NO: 1; ii) T, R, V, Q, or E at a residue corresponding to position 238 in SEQ ID NO: 1; jj) A or S at a residue corresponding to position 256 in SEQ ID NO: 1; kk) E or S at a residue corresponding to position 262 in SEQ ID NO: 1; ll) E at a residue corresponding to position 264 in SEQ ID NO: 1; mm) N or H at a residue corresponding to position 265 in SEQ ID NO: 1; nn) M or L at a residue corresponding to position 266 in SEQ ID NO: 1; oo) F at a residue corresponding to position 267 in SEQ ID NO: 1; pp) D or E at a residue corresponding to position 269 in SEQ ID NO: 1; qq) L or M at a residue corresponding to position 271 in SEQ ID NO: 1; rr) S, C, M, or A at a residue corresponding to position 284 in SEQ ID NO: 1; ss) G at a residue corresponding to position 285 in SEQ ID NO: 1; tt) A at a residue corresponding to position 287 in SEQ ID NO: 1; uu) I at a residue corresponding to position 290 in SEQ ID NO: 1; vv) D at a residue corresponding to position 291 in SEQ ID NO: 1; ww) R, V, G, N, D, K, E, A, or Q at a residue corresponding to position 297 in SEQ ID NO: 1; xx) S, A, D, E, or N at a residue corresponding to position 301 in SEQ ID NO: 1; yy) K at a residue corresponding to position 303 in SEQ ID NO: 1; zz) Y at a residue corresponding to position 313 in SEQ ID NO: 1; aaa) R, P, E, V, A, or K at a residue corresponding to position 319 in SEQ ID NO: 1; bbb) T or S at a residue corresponding to position 325 in SEQ ID NO: 1; ccc) H or N at a residue corresponding to position 329 in SEQ ID NO: 1; ddd) S, A, M, V, N, T, L, or Y at a residue corresponding to position 335 in SEQ ID NO: 1; eee) A or G at a residue corresponding to position 336 in SEQ ID NO: 1; fff) S, D, V, A, or N at a residue corresponding to position 338 in SEQ ID NO: 1; ggg) T at a residue corresponding to position 339 in SEQ ID NO: 1; hhh) A, S, or C at a residue corresponding to position 342 in SEQ ID NO: 1; iii) I, L, or F at a residue corresponding to position 346 in SEQ ID NO: 1; jjj) A at a residue corresponding to position 350 in SEQ ID NO: 1; kkk) E at a residue corresponding to position 355 in SEQ ID NO: 1; lll) D, E, or P at a residue corresponding to position 365 in SEQ ID NO: 1; mmm) E, G, A, R, H, Q, or K at a residue corresponding to position 374 in SEQ ID NO: 1; nnn) H, K, L, or R at a residue corresponding to position 381 in SEQ ID NO: 1; ooo) S at a residue corresponding to position 382 in SEQ ID NO: 1; and/or ppp) S or T at a residue corresponding to position 384 in SEQ ID NO: 1.

In some embodiments, the one or more amino acid alterations result in an engineered formate dehydrogenase comprising: a) A at a residue corresponding to position 2 in SEQ ID NO: 1; b) T at a residue corresponding to position 98 in SEQ ID NO: 1; c) I or V at a residue corresponding to position 199 in SEQ ID NO: 1; d) Q at a residue corresponding to position 206 in SEQ ID NO: 1; e) A, K, R, T, E, Y, V, I, or L at a residue corresponding to position 231 in SEQ ID NO: 1; f) M or L at a residue corresponding to position 266 in SEQ ID NO: 1; and/or g) P, K, L, R, or H at a residue corresponding to position 381 in SEQ ID NO: 1.

In some embodiments, the one or more amino acid alterations result in an engineered formate dehydrogenase comprising a) A at a residue corresponding to position 2 in SEQ ID NO: 1; b) T at a residue corresponding to position 98 in SEQ ID NO: 1; c) I or V at a residue corresponding to position 199 in SEQ ID NO: 1; d) Q at a residue corresponding to position 206 in SEQ ID NO: 1; e) V, I, or L at a residue corresponding to position 231 in SEQ ID NO: 1; f) M or L at a residue corresponding to position 266 in SEQ ID NO: 1; and/or g) H at a residue corresponding to position 381 in SEQ ID NO: 1.

In some embodiments, the one or more amino acid alterations of the engineered formate dehydrogenase is an alteration described in TABLE 7. For example, in some embodiments, the one or more amino acid alterations result in an engineered formate dehydrogenase comprising: a) K at a residue corresponding to position 36 in SEQ ID NO: 2; b) V at a residue corresponding to position 64 in SEQ ID NO: 2; c) E at a residue corresponding to position 80 in SEQ ID NO: 2; d) S at a residue corresponding to position 91 in SEQ ID NO: 2; e) N at a residue corresponding to position 97 in SEQ ID NO: 2; f) T at a residue corresponding to position 111 in SEQ ID NO: 2; g) I at a residue corresponding to position 120 in SEQ ID NO: 2; h) L at a residue corresponding to position 162 in SEQ ID NO: 2; i) V at a residue corresponding to position 164 in SEQ ID NO: 2; j) G at a residue corresponding to position 187 in SEQ ID NO: 2; k) C at a residue corresponding to position 188 in SEQ ID NO: 2; l) T at a residue corresponding to position 214 in SEQ ID NO: 2; m) V, T, or C at a residue corresponding to position 229 in SEQ ID NO: 2; n) C at a residue corresponding to position 256 in SEQ ID NO: 2; o) G or S at a residue corresponding to position 257 in SEQ ID NO: 2; p) G at a residue corresponding to position 260 in SEQ ID NO: 2; q) V, F, or T at a residue corresponding to position 312 in SEQ ID NO: 2; r) G or A at a residue corresponding to position 313 in SEQ ID NO: 2; s) C or S at a residue corresponding to position 315 in SEQ ID NO: 2; t) T or S at a residue corresponding to position 320 in SEQ ID NO: 2; u) M at a residue corresponding to position 323 in SEQ ID NO: 2; v) R at a residue corresponding to position 361 in SEQ ID NO: 2; and/or w) K at a residue corresponding to position 362 in SEQ ID NO: 2.

In some embodiments, the one or more amino acid alterations result in an engineered formate dehydrogenase comprising: a) K at a residue corresponding to position 36 in SEQ ID NO: 2; b) V at a residue corresponding to position 64 in SEQ ID NO: 2; c) E at a residue corresponding to position 80 in SEQ ID NO: 2; d) S at a residue corresponding to position 91 in SEQ ID NO: 2; e) N at a residue corresponding to position 97 in SEQ ID NO: 2; f) T at a residue corresponding to position 111 in SEQ ID NO: 2; g) I at a residue corresponding to position 120 in SEQ ID NO: 2; h) L at a residue corresponding to position 162 in SEQ ID NO: 2; i) V at a residue corresponding to position 164 in SEQ ID NO: 2; j) G at a residue corresponding to position 187 in SEQ ID NO: 2; k) C at a residue corresponding to position 188 in SEQ ID NO: 2; l) T at a residue corresponding to position 214 in SEQ ID NO: 2; m) T or C at a residue corresponding to position 229 in SEQ ID NO: 2; n) C at a residue corresponding to position 256 in SEQ ID NO: 2; o) G or S at a residue corresponding to position 257 in SEQ ID NO: 2; p) G at a residue corresponding to position 260 in SEQ ID NO: 2; q) V, F, or T at a residue corresponding to position 312 in SEQ ID NO: 2; r) G or A at a residue corresponding to position 313 in SEQ ID NO: 2; s) C at a residue corresponding to position 315 in SEQ ID NO: 2; t) S at a residue corresponding to position 320 in SEQ ID NO: 2; u) M at a residue corresponding to position 323 in SEQ ID NO: 2; v) R at a residue corresponding to position 361 in SEQ ID NO: 2; and/or w) K at a residue corresponding to position 362 in SEQ ID NO: 2.

In some embodiments, the one or more amino acid alterations result in an engineered formate dehydrogenase comprising: a) K at a residue corresponding to position 36 in SEQ ID NO: 2; b) V at a residue corresponding to position 64 in SEQ ID NO: 2; c) E at a residue corresponding to position 80 in SEQ ID NO: 2; d) T at a residue corresponding to position 111 in SEQ ID NO: 2; e) I at a residue corresponding to position 120 in SEQ ID NO: 2; f) L at a residue corresponding to position 162 in SEQ ID NO: 2; g) T at a residue corresponding to position 214 in SEQ ID NO: 2; h) V, T, or C at a residue corresponding to position 229 in SEQ ID NO: 2; i) G at a residue corresponding to position 260 in SEQ ID NO: 2; j) C or S at a residue corresponding to position 315 in SEQ ID NO: 2; k) T or S at a residue corresponding to position 320 in SEQ ID NO: 2; and/or l) R at a residue corresponding to position 361 in SEQ ID NO: 2.

In some embodiments, the one or more amino acid alterations result in an engineered formate dehydrogenase comprising: a) K at a residue corresponding to position 36 in SEQ ID NO: 2; b) V at a residue corresponding to position 64 in SEQ ID NO: 2; c) E at a residue corresponding to position 80 in SEQ ID NO: 2; d) T at a residue corresponding to position 111 in SEQ ID NO: 2; e) I at a residue corresponding to position 120 in SEQ ID NO: 2; f) L at a residue corresponding to position 162 in SEQ ID NO: 2; g) T at a residue corresponding to position 214 in SEQ ID NO: 2; h) T or C at a residue corresponding to position 229 in SEQ ID NO: 2; i) G at a residue corresponding to position 260 in SEQ ID NO: 2; j) C at a residue corresponding to position 315 in SEQ ID NO: 2; k) S at a residue corresponding to position 320 in SEQ ID NO: 2; and/or l) R at a residue corresponding to position 361 in SEQ ID NO: 2.

In some embodiments, the one or more amino acid alterations result in an engineered formate dehydrogenase comprising: a) H at a residue corresponding to position 381 in SEQ ID NO: 1; b) Q at a residue corresponding to position 206 and I at a residue corresponding to position 231 in SEQ ID NO: 1; c) I at a residue corresponding to position 199 in SEQ ID NO: 1; d) Q at a residue corresponding to position 206 and V at a residue corresponding to position 231 in SEQ ID NO: 1; e) I at a residue corresponding to position 199 and L at a residue corresponding to position 266 in SEQ ID NO: 1; f) Q at a residue corresponding to position 206 and L at a residue corresponding to position 231 in SEQ ID NO: 1; g) A at a residue corresponding to position 2 in SEQ ID NO: 1; h) T at a residue corresponding to position 98 in SEQ ID NO: 1; i) V at a residue corresponding to position 199 and M at a residue corresponding to position 266 in SEQ ID NO: 1; j) T at a residue corresponding to position 111 and R at a residue corresponding to position 361 in SEQ ID NO: 2; k) L at a residue corresponding to position 162 and R at a residue corresponding to position 361 in SEQ ID NO: 2; l) T at a residue corresponding to position 229 and G at a residue corresponding to position 260 in SEQ ID NO: 2; m) T at a residue corresponding to position 214 and R at a residue corresponding to position 361 in SEQ ID NO: 2; n) K at a residue corresponding to position 36, L at a residue corresponding to position 162, T at a residue corresponding to position 214, and R at a residue corresponding to position 361 in SEQ ID NO: 2; o) E at a residue corresponding to position 80 and R at a residue corresponding to position 361 in SEQ ID NO: 2; p) I at a residue corresponding to position 120 and S at a residue corresponding to position 320 in SEQ ID NO: 2; q) K at a residue corresponding to position 36 and R at a residue corresponding to position 361 in SEQ ID NO: 2; r) T at a residue corresponding to position 111 and L at a residue corresponding to position 162 in SEQ ID NO: 2; s) T at a residue corresponding to position 111, L at a residue corresponding to position 162, and R at a residue corresponding to position 361 in SEQ ID NO: 2; t) V at a residue corresponding to position 64, L at a residue corresponding to position 162, T at a residue corresponding to position 214, and R at a residue corresponding to position 361 in SEQ ID NO: 2; or u) C at a residue corresponding to position 229 and C at a residue corresponding to position 315 in SEQ ID NO: 2.

In some embodiments, the one or more alterations in the engineered formate dehydrogenase does not result in an amino acid sequence that is the same as SEQ ID NO: 24. Accordingly, in some embodiments the amino acid sequence of the engineered formate dehydrogenase described herein does not consist of the amino acid sequence of SEQ ID NO: 24. However, in some embodiments, the engineered formate dehydrogenase is a variant of a homolog of SEQ ID NO: 1 and 2 as described in TABLE 1, including SEQ ID NOS: 3-24. Such an engineered formate dehydrogenase includes one or more alterations at a position corresponding to a position described in TABLE 6 and/or TABLE 7.

Methods of generating and assaying the engineered formate dehydrogenases described herein are well known to one of skill in the art. Examples of such methods are described in EXAMPLES 1-8. Any of a variety of methods can be used to generate an engineered formate dehydrogenase disclosed herein. Such methods include, but are not limited to, site-directed mutagenesis, random mutagenesis, combinatorial libraries, and other mutagenesis methods described herein (see, e.g., Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Gillman et al., Directed Evolution Library Creation: Methods and Protocols (Methods in Molecular Biology) Springer, 2nd ed (2014)). In one non-limiting example, one skilled in the art would also be able to generate the engineered formate dehydrogenases described herein using a homolog of SEQ ID NO: 1 and 2, such as SEQ ID NOS: 3-24, which have one or more alterations at a position corresponding to a position described in TABLE 6 and/or TABLE 7, by performing sequence alignments of the target sequences with an alignment program described herein, generating the desired alteration using site-directed mutagenesis kit, such as QuikChange (Agilent, Santa Clara, CA), Q5® Site-Directed Mutagenesis Kit (New England BioLabs, Ipswich, MA), or QuikChange HT Protein Engineering System (Agilent, Santa Clara, CA), verifying the new mutant with DNA sequencing, and then assaying the new variants either with a lysate or in vivo production assay with the desired bioderived compound pathway as described in EXAMPLES 1-8. One non-limiting example of a method for preparing an engineered formate dehydrogenase is to express recombinant nucleic acids encoding the engineered formate dehydrogenase in a suitable microbial organism, such as a bacterial cell, a yeast cell, or other suitable cell, using methods well known in the art.

In some embodiments, an engineered formate dehydrogenase provided herein is an isolated formate dehydrogenase. An isolated engineered formate dehydrogenases provided herein can be isolated by a variety of methods well-known in the art, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure can be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

In some embodiments, the provided herein is a recombinant nucleic acid that has a nucleotide sequence encoding an engineered formate dehydrogenase described herein. Accordingly, in some embodiments, provided herein is a recombinant nucleic acid selected from (a) a nucleic acid molecule encoding an engineered formate dehydrogenase comprising a variant of amino acid sequence SEQ ID NO: 1 or SEQ ID NO: 2, wherein the engineered formate dehydrogenase comprises one or more alterations at a position described in Tables 1 and/or 2; (b) a recombinant nucleic acid that hybridizes to an isolated nucleic acid of (a) under highly stringent hybridization conditions; and (c) a recombinant nucleic acid that is complementary to (a) or (b).

In some embodiments, provided herein is a recombinant nucleic acid encoding an engineered formate dehydrogenase comprising a variant of amino acid sequence SEQ ID NO: 1 or SEQ ID NO: 2, wherein the engineered formate dehydrogenase comprises one or more alterations at a position described in TABLE 6 and/or TABLE 7. In some embodiments, the recombinant nucleic acid encodes an engineered formate dehydrogenase comprising one or more alterations at a position described in TABLE 6. In some embodiments, the recombinant nucleic acid encodes an engineered formate dehydrogenase comprising one or more alterations at a position described in TABLE 7.

In some embodiments, provided herein is a recombinant nucleic acid that hybridizes under highly stringent hybridization conditions to an isolated nucleic acid encoding an engineered formate dehydrogenase described herein. Accordingly, in some embodiments, the recombinant nucleic acid is an isolated nucleic acid that hybridizes under highly stringent hybridization conditions to a nucleic acid that encodes an engineered formate dehydrogenase comprising one or more alterations at a position described in TABLE 6. In some embodiments, the recombinant nucleic acid molecule is an isolated nucleic acid that hybridizes under highly stringent hybridization conditions to a nucleic acid that encodes an engineered formate dehydrogenase comprising one or more alterations at a position described in TABLE 7.

In some embodiments, provided herein is a recombinant nucleic acid encoding an engineered formate dehydrogenase comprising an amino acid sequence that is a variant of SEQ ID NO: 1 or 2 that includes one or more alterations as described in TABLE 6 and/or TABLE 7, wherein the portion, other than the alteration described in TABLE 6 and/or TABLE 7, of the engineered formate dehydrogenase has at least 65%, 70%, 75%, 80%, 85%, 90%, 95%, 98% or 99% sequence identity, or is identical, to an amino acids sequence referenced as SEQ ID NO: 1 or SEQ ID NO: 2. Accordingly, in some embodiments, a recombinant nucleic acid encodes an engineered formate dehydrogenase comprising an amino acid sequence that includes one or more alterations as described in TABLE 6 and the portion, other than the alteration described in TABLE 6, of the engineered formate dehydrogenase has at least 65% identical to SEQ ID NO: 1. In some embodiments, a recombinant nucleic acid encodes an engineered formate dehydrogenase that has an amino acid sequence that includes one or more alterations as described in TABLE 6 and the portion, other than the alteration described in TABLE 6, of the engineered formate dehydrogenase has at least 70% identical to SEQ ID NO: 1. In some embodiments, a recombinant nucleic acid encodes an engineered formate dehydrogenase that has an amino acid sequence that includes one or more alterations as described in TABLE 6 and the portion, other than the alteration described in TABLE 6, of the engineered formate dehydrogenase has at least 75% identical to SEQ ID NO: 1. In some embodiments, a recombinant nucleic acid encodes an engineered formate dehydrogenase that has an amino acid sequence that includes one or more alterations as described in TABLE 6 and the portion, other than the alteration described in TABLE 6, of the engineered formate dehydrogenase has at least 80% identical to SEQ ID NO: 1. In some embodiments, a recombinant nucleic acid encodes an engineered formate dehydrogenase that has an amino acid sequence that includes one or more alterations as described in TABLE 6 and the portion, other than the alteration described in TABLE 6, of the engineered formate dehydrogenase has at least 85% identical to SEQ ID NO: 1. In some embodiments, a recombinant nucleic acid encodes an engineered formate dehydrogenase that has an amino acid sequence that includes one or more alterations as described in TABLE 6 and the portion, other than the alteration described in TABLE 6, of the engineered formate dehydrogenase has at least 90% identical to SEQ ID NO: 1. In some embodiments, a recombinant nucleic acid encodes an engineered formate dehydrogenase that has an amino acid sequence that includes one or more alterations as described in TABLE 6 and the portion, other than the alteration described in TABLE 6, of the engineered formate dehydrogenase has at least 95% identical to SEQ ID NO: 1. In some embodiments, a recombinant nucleic acid encodes an engineered formate dehydrogenase that has an amino acid sequence that includes one or more alterations as described in TABLE 6 and the portion, other than the alteration described in TABLE 6, of the engineered formate dehydrogenase has at least 98% identical to SEQ ID NO: 1. In some embodiments, a recombinant nucleic acid encodes an engineered formate dehydrogenase that has an amino acid sequence that includes one or more alterations as described in TABLE 6 and the portion, other than the alteration described in TABLE 6, of the engineered formate dehydrogenase has at least 99% identical to SEQ ID NO: 1.

In some embodiments, a recombinant nucleic acid encodes an engineered formate dehydrogenase that has an amino acid sequence that includes one or more alterations as described in TABLE 7 and the portion, other than the alteration described in TABLE 7, of the engineered formate dehydrogenase has at least 65% identical to SEQ ID NO: 2. In some embodiments, a recombinant nucleic acid encodes an engineered formate dehydrogenase that has an amino acid sequence that includes one or more alterations as described in TABLE 7 and the portion, other than the alteration described in TABLE 7, of the engineered formate dehydrogenase has at least 70% identical to SEQ ID NO: 2. In some embodiments, a recombinant nucleic acid molecule encodes an engineered formate dehydrogenase that has an amino acid sequence that includes one or more alterations as described in TABLE 7 and the portion, other than the alteration described in TABLE 7, of the engineered formate dehydrogenase has at least 75% identical to SEQ ID NO: 2. In some embodiments, a recombinant nucleic acid encodes an engineered formate dehydrogenase that has an amino acid sequence that includes one or more alterations as described in TABLE 7 and the portion, other than the alteration described in TABLE 7, of the engineered formate dehydrogenase has at least 80% identical to SEQ ID NO: 2. In some embodiments, a recombinant nucleic acid encodes an engineered formate dehydrogenase that has an amino acid sequence that includes one or more alterations as described in TABLE 7 and the portion, other than the alteration described in TABLE 7, of the engineered formate dehydrogenase has at least 85% identical to SEQ ID NO: 2. In some embodiments, a recombinant nucleic acid encodes an engineered formate dehydrogenase that has an amino acid sequence that includes one or more alterations as described in TABLE 7 and the portion, other than the alteration described in TABLE 7, of the engineered formate dehydrogenase has at least 90% identical to SEQ ID NO: 2. In some embodiments, a recombinant nucleic acid encodes an engineered formate dehydrogenase that has an amino acid sequence that includes one or more alterations as described in TABLE 7 and the portion, other than the alteration described in TABLE 7, of the engineered formate dehydrogenase has at least 95% identical to SEQ ID NO: 2. In some embodiments, a recombinant nucleic acid encodes an engineered formate dehydrogenase that has an amino acid sequence that includes one or more alterations as described in TABLE 7 and the portion, other than the alteration described in TABLE 7, of the engineered formate dehydrogenase has at least 98% identical to SEQ ID NO: 2. In some embodiments, a recombinant nucleic acid encodes an engineered formate dehydrogenase that has an amino acid sequence that includes one or more alterations as described in TABLE 7 and the portion, other than the alteration described in TABLE 7, of the engineered formate dehydrogenase has at least 99% identical to SEQ ID NO: 2.

In some embodiments, a recombinant nucleic acid encodes an engineered formate dehydrogenase that includes one or more alterations at a position described in TABLE 6 and/or TABLE 7 wherein the one or more amino acid alterations are conservative amino acid substitutions. In some embodiments, a recombinant nucleic acid encodes an engineered formate dehydrogenase that includes one or more alterations at a position described in TABLE 6 and/or TABLE 7, wherein the one or more amino acid alterations are non-conservative amino acid substitutions. In some embodiments, a recombinant nucleic acid encodes an engineered formate dehydrogenase that includes one or more alterations at a position described in TABLE 6. In some embodiments, a recombinant nucleic acid encodes an engineered formate dehydrogenase that includes one or more alterations at a position described in TABLE 7. In some embodiments, a recombinant nucleic acid encodes an engineered formate dehydrogenase that includes a conservative amino acid substitution and/or non-conservative amino acid substitution in 1 to 10 amino acid positions as set forth in TABLE 6 and/or TABLE 7.

In some embodiments, a recombinant nucleic acid encodes an engineered formate dehydrogenase that includes a conservative amino acid substitution in from 1 to 50 amino acid positions, or alternatively from 2 to 50 amino acid positions, or alternatively from 3 to 50 amino acid positions, or alternatively from 4 to 50 amino acid positions, or alternatively from 5 to 50 amino acid positions, or alternatively from 6 to 50 amino acid positions, or alternatively from 7 to 50 amino acid positions, or alternatively from 8 to 50 amino acid positions, or alternatively from 9 to 50 amino acid positions, or alternatively from 10 to 50 amino acid positions, or alternatively from 15 to 50 amino acid positions, or alternatively from 20 to 50 amino acid positions, or alternatively from 30 to 50 amino acid positions, or alternatively from 40 to 50 amino acid positions, or alternatively from 45 to 50 amino acid positions, or any integer therein, wherein the positions are other than the variant amino acid positions set forth in TABLE 6 and/or TABLE 7. In some aspects, such a conservative amino acid sequence is a chemically conservative or an evolutionary conservative amino acid substitution. Methods of identifying conservative amino acids are well known to one of skill in the art, any one of which can be used to generate the isolated polypeptides described herein.

A recombinant nucleic acid provided herein can encode an engineered formate dehydrogenase that include any combination of the alterations set forth in TABLE 6 and/or TABLE 7. One alteration alone, or in combination, can produce an engineered formate dehydrogenase that retains or improves the activity as described herein relative to a reference polypeptide, for example, the wild-type (native) formate dehydrogenase. In some embodiments, a recombinant nucleic acid encodes an engineered formate dehydrogenase that includes at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 alterations as set forth in Tables 1, 2, 3, and/or 4, including up to an alteration at all of the positions identified in TABLE 6 and/or TABLE 7. In some embodiments, a recombinant nucleic acid encodes an engineered formate dehydrogenase that includes at least 2 alterations as set forth in TABLE 6 and/or TABLE 7. In some embodiments, a recombinant nucleic acid encodes an engineered formate dehydrogenase that includes at least 3 alterations as set forth in TABLE 6 and/or TABLE 7. In some embodiments, a recombinant nucleic acid encodes an engineered formate dehydrogenase that includes at least 4 alterations as set forth in TABLE 6 and/or TABLE 7. In some embodiments, a recombinant nucleic acid encodes an engineered formate dehydrogenase that includes at least 5 alterations as set forth in TABLE 6 and/or TABLE 7. In some embodiments, a recombinant nucleic acid encodes an engineered formate dehydrogenase that includes at least 6 alterations as set forth in TABLE 6 and/or TABLE 7. In some embodiments, a recombinant nucleic acid encodes an engineered formate dehydrogenase that includes at least 7 alterations as set forth in TABLE 6 and/or TABLE 7. In some embodiments, a recombinant nucleic acid encodes an engineered formate dehydrogenase that includes at least 8 alterations as set forth in TABLE 6 and/or TABLE 7. In some embodiments, a recombinant nucleic acid encodes an engineered formate dehydrogenase that includes at least 9 alterations as set forth in TABLE 6 and/or TABLE 7. In some embodiments, a recombinant nucleic acid encodes an engineered formate dehydrogenase that includes at least 10 alterations as set forth in TABLE 6 and/or TABLE 7.

In some embodiments, provided herein is a recombinant nucleic acid that includes a nucleotide sequence encoding an engineered formate dehydrogenase described herein that is operatively linked to a promoter. Such a promoter can express the engineered formate dehydrogenase in a microbial organism as described herein.

In some embodiments, provided herein is a vector containing a recombinant nucleic acid described herein. In some embodiments, the vector is an expression vector. In some embodiments, the vector comprises double stranded DNA.

A recombinant nucleic acid encoding an engineered formate dehydrogenase described herein also includes a nucleic acid that hybridizes to a nucleic acid disclosed herein or a nucleic acid that hybridizes to a nucleic acid that encodes an amino acid sequence disclosed. Hybridization conditions can include highly stringent, moderately stringent, or low stringency hybridization conditions that are well known to one of skill in the art such as those described herein. Similarly, a recombinant nucleic acid that can be used in the compositions and methods described herein can be described as having a certain percent sequence identity to a nucleic acid disclosed herein or a nucleic acid that hybridizes to a nucleic acid molecule that encodes an amino acid sequence disclosed herein. For example, the nucleic acid can have at least 65%, 70%, 75%, 80%, 85%, 90%, 91%, 92%, 93%, 94%, 95%, 96%, 97%, 98% or 99% sequence identity, or be identical, to a nucleotide described herein.

Stringent hybridization refers to conditions under which hybridized polynucleotides are stable. As known to those of skill in the art, the stability of hybridized polynucleotides is reflected in the melting temperature (Tₘ) of the hybrids. In general, the stability of hybridized polynucleotides is a function of the salt concentration, for example, the sodium ion concentration, and temperature. A hybridization reaction can be performed under conditions of lower stringency, followed by washes of varying, but higher, stringency. Reference to hybridization stringency relates to such washing conditions. Highly stringent hybridization includes conditions that permit hybridization of only those nucleotide sequences that form stable hybridized polynucleotides in 0.018M NaCl at 65° C., for example, if a hybrid is not stable in 0.018M NaCl at 65° C., it will not be stable under high stringency conditions, as contemplated herein. High stringency conditions can be provided, for example, by hybridization in 50% formamide, 5× Denhart's solution, 5×SSPE, 0.2% SDS at 42° C., followed by washing in 0.1×SSPE, and 0.1% SDS at 65° C. Hybridization conditions other than highly stringent hybridization conditions can also be used to describe the nucleotide sequences disclosed herein. For example, the phrase moderately stringent hybridization refers to conditions equivalent to hybridization in 50% formamide, 5× Denhart's solution, 5×SSPE, 0.2% SDS at 42° C., followed by washing in 0.2×SSPE, 0.2% SDS, at 42° C. The phrase low stringency hybridization refers to conditions equivalent to hybridization in 10% formamide, 5× Denhart's solution, 6×SSPE, 0.2% SDS at 22° C., followed by washing in 1×SSPE, 0.2% SDS, at 37° C. Denhart's solution contains 1% Ficoll, 1% polyvinylpyrolidone, and 1% bovine serum albumin (BSA). 20×SSPE (sodium chloride, sodium phosphate, ethylene diamine tetraacetic acid (EDTA)) contains 3M sodium chloride, 0.2M sodium phosphate, and 0.025 M (EDTA). Other suitable low, moderate and high stringency hybridization buffers and conditions are well known to those of skill in the art and are described, for example, in Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999).

A recombinant nucleic acid encoding an engineered formate dehydrogenase described herein can have at least a certain sequence identity to a nucleotide sequence disclosed herein. Accordingly, in some aspects described herein, a recombinant nucleic acid encoding an engineered formate dehydrogenase has a nucleotide sequence of at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity, or is identical, to a nucleic acid disclosed herein or a nucleic acid that hybridizes to a nucleic acid that encodes an amino acid sequence disclosed herein.

It is understood that a recombinant nucleic acid described herein or an engineered formate dehydrogenase described here can exclude a wild-type parental sequence, for example a parental sequence, such as SEQ ID NOS: 1 or 2. One skilled in the art will readily understand the meaning of a parental wild-type sequence based on what is well known in the art. It is further understood that such a recombinant nucleic acid described herein can exclude a nucleotide sequence encoding a naturally occurring amino acid sequence as found in nature. Similarly, an engineered formate dehydrogenase described herein can exclude an amino acid sequence as found in nature. Thus, in some embodiments, the recombinant nucleic acid or engineered formate dehydrogenase described herein is as set forth herein, with the proviso that the encoded amino acid sequence is not the wild-type parental sequence or a naturally occurring amino acid sequence and/or that the nucleotide sequence is not a wild-type or naturally occurring nucleotide sequence. A naturally occurring amino acid or nucleotide sequence is understood by those skilled in the art as relating to a sequence that is found in a naturally occurring organism as found in nature. Thus, a nucleotide or amino acid sequence that is not found in the same state or having the same nucleotide or encoded amino acid sequence as in a naturally occurring organism is included within the meaning of a recombinant nucleotide and/or amino acid sequence described herein. For example, a nucleotide or amino acid sequence that has been altered at one or more nucleotide or amino acid positions from a parent sequence, including variants as described herein, are included within the meaning of a nucleotide or amino acid sequence described herein that is not naturally occurring. A recombinant nucleic acid described herein excludes a naturally occurring chromosome that contains the nucleotide sequence, and can further exclude other molecules, as found in a naturally occurring cell, such as DNA binding proteins, for example, proteins such as histones that bind to chromosomes within a eukaryotic cell.

Thus, a recombinant nucleic acid described here has physical and chemical differences compared to a naturally occurring nucleic acid. A recombinant or non-naturally occurring nucleic acid described herein does not contain or does not necessarily have some or all of the chemical bonds, either covalent or non-covalent bonds, of a naturally occurring nucleic acid as found in nature. A recombinant nucleic acid described herein thus differs from a naturally occurring nucleic acid, for example, by having a different chemical structure than a naturally occurring nucleic acid as found in a chromosome. A different chemical structure can occur, for example, by cleavage of phosphodiester bonds that release a recombinant nucleic acid from a naturally occurring chromosome. A recombinant nucleic acid described herein can also differ from a naturally occurring nucleic acid by isolating or separating the nucleic acid from proteins that bind to chromosomal DNA in either prokaryotic or eukaryotic cells, thereby differing from a naturally occurring nucleic acid by different non-covalent bonds. With respect to nucleic acids of prokaryotic origin, a non-naturally occurring nucleic acid described herein does not necessarily have some or all of the naturally occurring chemical bonds of a chromosome, for example, binding to DNA binding proteins such as polymerases or chromosome structural proteins, or is not in a higher order structure such as being supercoiled. With respect to nucleic acids of eukaryotic origin, a non-naturally occurring nucleic acid described herein also does not contain the same internal nucleic acid chemical bonds or chemical bonds with structural proteins as found in chromatin. For example, a non-naturally occurring nucleic acid described herein is not chemically bonded to histones or scaffold proteins and is not contained in a centromere or telomere. Thus, the non-naturally occurring nucleic acids described herein are chemically distinct from a naturally occurring nucleic acid because they either lack or contain different van der Waals interactions, hydrogen bonds, ionic or electrostatic bonds, and/or covalent bonds from a nucleic acid as found in nature. Such differences in bonds can occur either internally within separate regions of the nucleic acid (that is cis) or such difference in bonds can occur in trans, for example, interactions with chromosomal proteins. In the case of a nucleic acid of eukaryotic origin, a cDNA is considered to be a recombinant or non-naturally occurring nucleic acid since the chemical bonds within a cDNA differ from the covalent bonds, that is the sequence, of a gene on chromosomal DNA. Thus, it is understood by those skilled in the art that recombinant or non-naturally occurring nucleic acid is distinct from a naturally occurring nucleic acid.

In some embodiments, provided herein is a method of constructing a host strain that can include, among other steps, introducing a vector disclosed herein into a microbial organism, for example, that is capable of expressing an amino acid sequence encoded by the vector and/or is capable of fermentation. Vectors described herein can be introduced stably or transiently into a microbial organism using techniques well known in the art including, but not limited to, conjugation, electroporation, chemical transformation, transduction, transfection, and ultrasound transformation. Additional methods are disclosed herein, any one of which can be used in the method described herein.

In some embodiments, provided herein is a microbial organism, in particular a non-naturally occurring microbial organism, that comprises a polypeptide described herein, that is, an engineered formate dehydrogenase described herein. Thus, provided herein is a non-naturally occurring microbial organism having a recombinant nucleic acid encoding an engineered formate dehydrogenase described herein. Accordingly, in some embodiments, provided herein is microbial organism (e.g., host microbial organism) that comprises a recombinant polynucleotide encoding an engineered formate dehydrogenase, wherein the engineered formate dehydrogenase comprises one or more amino acid alterations at a position corresponding to position 2, 9, 16, 19, 27, 29, 30, 41, 53, 73, 97, 98, 100, 101, 120, 121, 122, 123, 124, 128, 138, 143, 144, 145, 146, 147, 149, 150, 151, 152, 153, 155, 175, 176, 191, 196, 198, 199, 203, 204, 206, 217, 218, 224, 231, 238, 256, 262, 264, 265, 266, 267, 269, 271, 284, 285, 287, 290, 291, 297, 301, 303, 313, 315, 319, 325, 329, 335, 336, 338, 339, 342, 343, 346, 350, 355, 365, 374, 381, 382, or 384, or a combination thereof, in SEQ ID NO: 1. In some embodiments, provided herein is microbial organism (e.g., host microbial organism) that comprises a recombinant polynucleotide encoding an engineered formate dehydrogenase, wherein the engineered formate dehydrogenase comprises one or more amino acid alterations at a position corresponding to position 36, 64, 80, 91, 97, 111, 120, 162, 164, 187, 188, 214, 229, 256, 257, 260, 312, 313, 315, 320, 323, 361, or 362, or a combination thereof, in SEQ ID NO: 2.

Optionally, the non-naturally occurring microbial organism can include one or more exogenous nucleic acids encoding one or more enzymes for converting NADH to NADPH. Accordingly, in some embodiments, the non-naturally occurring microbial organism can include an exogenous nucleic acid encoding a transhydrogenase that is capable of catalyzing the conversion of NADH to NADPH. Such transhydrogenases include NAD(P)+transhydrogenases (EC 1.6.1.1—Si-specific; and EC 1.6.1.2—Re/Si-specific). Non-limiting exemplary transhydrogenases include NAD(P) transhydrogenase subunit beta of Escherichia coli, encoded by the pntB_2 gene (UniProtKB A0A377CI53), proton-translocating NAD(P)(+) transhydrogenase of Mycobacterium tuberculosis (strain ATCC 25618/H37Rv), encoded by the pntAb gene (UniProtKB P96833), NAD(P) transhydrogenase subunit alpha part 2 of Rhodospirillum rubrum, encoded by the pntAB gene (UniProtKB POC187), NAD(P) transhydrogenase alpha subunit of Coxiella burnetii (strain RSA 493 Nine Mile phase I), encoded by then pntAA gene (UniProtKB Q83AE6), soluble pyridine nucleotide transhydrogenase of Escherichia coli (strain K12), encoded by the sthA gene (UniProtKB P27306), and soluble pyridine nucleotide transhydrogenase of Pseudomonas fluorescens, encoded by the sthA gene (UniProtKB 005139). Including one or more exogenous nucleic acid encoding a transhydrogenase, in some embodiments, provides for converting the NADH generated by an engineered formate dehydrogenase described herein to a NADPH, which can be used as a cofactor for production of a bioderived compound described herein. Alternatively, or in addition, an endogenous transhydrogenase present in the non-naturally occurring microbial organism can be relied upon for converting the increase amount of NADH generated by an engineered formate dehydrogenase described herein to an increased amount of NADPH. In some embodiments, the exogenous nucleic acid is heterologous. In some embodiments, the exogenous nucleic acid is homologous.

In some embodiments, provided herein is a non-naturally occurring microbial organism as described herein that further includes a pathway capable of producing a bioderived compound as described herein. Such a pathway, in some embodiments, would benefit directly or indirectly from the production of cofactors, such as NADH produced by an engineered formate dehydrogenase described herein. A pathway capable of producing a bioderived compound as described herein that would directly benefit from the production of a cofactor includes, for example, a pathway with one or more enzymes that rely upon NADH as a cofactor in the enzymatic reaction being catalyzed. A pathway capable of producing a bioderived compound as described herein that would indirectly benefit from the production of a cofactor includes, for example, a pathway with one or more enzymes that rely upon NADPH as a cofactor in the enzymatic reaction being catalyzed, wherein the NADPH is generated by conversion of NADH to NADPH by a transhydrogenase as described herein. Moreover, because NADH can be important in microbial catabolism and cell growth in general, a non-naturally occurring microbial organism that includes a pathway capable of producing a bioderived compound that does not include an enzyme that relies upon NADH or NADPH as a cofactor, can still benefit indirectly from improvements microbial catabolism and cell growth. In some embodiments, the bioderived compound is an alcohol, a glycol, an organic acid, an alkene, a diene, an organic amine, an organic aldehyde, a vitamin, a nutraceutical or a pharmaceutical.

In some embodiments, the non-naturally occurring microbial organism described herein includes a pathway for production of an alcohol as described herein. Accordingly, in some embodiments, the alcohol is selected from: (i) a biofuel alcohol, wherein said biofuel is a primary alcohol, a secondary alcohol, a diol or triol comprising C3 to C10 carbon atoms; (ii) n-propanol or isopropanol; and (iii) a fatty alcohol, wherein said fatty alcohol comprises C4 to C27 carbon atoms, C8 to C18 carbon atoms, C12 to C18 carbon atoms, or C12 to C14 carbon atoms. In some aspects, the biofuel alcohol is selected from 1-propanol, isopropanol, 1-butanol, isobutanol, 1-pentanol, isopentenol, 2-methyl-1-butanol, 3-methyl-1-butanol, 1-hexanol, 3-methyl-1-pentanol, 1-heptanol, 4-methyl-1-hexanol, and 5-methyl-1-hexanol.

In some embodiments, the non-naturally occurring microbial organism described herein includes a pathway for production of an diol. Accordingly, in some embodiments, the diol is a propanediol or a butanediol. In some aspects, the butanediol is 1,4 butanediol, 1,3-butanediol or 2,3-butanediol.

In some embodiments, the non-naturally occurring microbial organism described herein includes a pathway for production of a bioderived compound selected from: (i) 1,4-butanediol or an intermediate thereto, wherein said intermediate is optionally 4-hydroxybutanoic acid (4-HB); (ii) butadiene (1,3-butadiene) or an intermediate thereto, wherein said intermediate is optionally 1,4-butanediol, 1,3-butanediol, 2,3-butanediol, crotyl alcohol, 3-buten-2-ol (methyl vinyl carbinol) or 3-buten-1-ol; (iii) 1,3-butanediol or an intermediate thereto, wherein said intermediate is optionally 3-hydroxybutyrate (3-HB), 2,4-pentadienoate, crotyl alcohol or 3-buten-1-ol; (iv) adipate, 6-aminocaproic acid, caprolactam, hexamethylenediamine, levulinic acid or an intermediate thereto, wherein said intermediate is optionally adipyl-CoA or 4-aminobutyryl-CoA; (v) methacrylic acid or an ester thereof, 3-hydroxyisobutyrate, 2-hydroxyisobutyrate, or an intermediate thereto, wherein said ester is optionally methyl methacrylate or poly(methyl methacrylate); (vi) 1,2-propanediol (propylene glycol), 1,3-propanediol, glycerol, ethylene glycol, diethylene glycol, triethylene glycol, dipropylene glycol, tripropylene glycol, neopentyl glycol, bisphenol A or an intermediate thereto; (vii) succinic acid or an intermediate thereto; and (viii) a fatty alcohol, a fatty aldehyde or a fatty acid comprising C4 to C27 carbon atoms, C8 to C18 carbon atoms, C12 to C18 carbon atoms, or C12 to C14 carbon atoms, wherein said fatty alcohol is optionally dodecanol (C12; lauryl alcohol), tridecyl alcohol (C13; 1-tridecanol, tridecanol, isotridecanol), myristyl alcohol (C14; 1-tetradecanol), pentadecyl alcohol (C15; 1-pentadecanol, pentadecanol), cetyl alcohol (C16; 1-hexadecanol), heptadecyl alcohol (C17; 1-n-heptadecanol, heptadecanol) and stearyl alcohol (C18; 1-octadecanol) or palmitoleyl alcohol (C16 unsaturated; cis-9-hexadecen-1-ol). Accordingly, in some embodiments, the non-naturally occurring microbial organism described herein includes a pathway for production of 1,4-butanediol or an intermediate thereto, wherein said intermediate is optionally 4-hydroxybutanoic acid (4-HB). In some embodiments, the non-naturally occurring microbial organism described herein includes a pathway for production of butadiene (1,3-butadiene) or an intermediate thereto, wherein said intermediate is optionally 1,4-butanediol, 1,3-butanediol, 2,3-butanediol, crotyl alcohol, 3-buten-2-ol (methyl vinyl carbinol) or 3-buten-1-ol. In some embodiments, the non-naturally occurring microbial organism described herein includes a pathway for production of 1,3-butanediol or an intermediate thereto, wherein said intermediate is optionally 3-hydroxybutyrate (3-HB), 2,4-pentadienoate, crotyl alcohol or 3-buten-1-ol. In some embodiments, the non-naturally occurring microbial organism described herein includes a pathway for production of adipate, 6-aminocaproic acid, caprolactam, hexamethylenediamine, levulinic acid or an intermediate thereto, wherein said intermediate is optionally adipyl-CoA or 4-aminobutyryl-CoA. In some embodiments, the non-naturally occurring microbial organism described herein includes a pathway for production of methacrylic acid or an ester thereof, 3-hydroxyisobutyrate, 2-hydroxyisobutyrate, or an intermediate thereto, wherein said ester is optionally methyl methacrylate or poly(methyl methacrylate). In some embodiments, the non-naturally occurring microbial organism described herein includes a pathway for production of 1,2-propanediol (propylene glycol), 1,3-propanediol, glycerol, ethylene glycol, diethylene glycol, triethylene glycol, dipropylene glycol, tripropylene glycol, neopentyl glycol, bisphenol A or an intermediate thereto. In some embodiments, the non-naturally occurring microbial organism described herein includes a pathway for production of succinic acid or an intermediate thereto. In some embodiments, the non-naturally occurring microbial organism described herein includes a pathway for production of a fatty alcohol, a fatty aldehyde or a fatty acid comprising C4 to C27 carbon atoms, C8 to C18 carbon atoms, C12 to C18 carbon atoms, or C12 to C14 carbon atoms, wherein said fatty alcohol is optionally dodecanol (C12; lauryl alcohol), tridecyl alcohol (C13; 1-tridecanol, tridecanol, isotridecanol), myristyl alcohol (C14; 1-tetradecanol), pentadecyl alcohol (C15; 1-pentadecanol, pentadecanol), cetyl alcohol (C16; 1-hexadecanol), heptadecyl alcohol (C17; 1-n-heptadecanol, heptadecanol) and stearyl alcohol (C18; 1-octadecanol) or palmitoleyl alcohol (C16 unsaturated; cis-9-hexadecen-1-ol).

1,4-Butanediol and intermediates thereto, such as 4-hydroxybutanoic acid (4-hydroxybutanoate, 4-hydroxybutyrate, 4-HB), are bioderived compounds that can be made via enzymatic pathways described herein and in the following publications. Suitable bioderived compound pathways and enzymes, methods for screening and methods for isolating are found in: WO2008115840A2 published 25 Sep. 2008 entitled Compositions and Methods for the Biosynthesis of 1,4-Butanediol and Its Precursors; WO2010141780A1 published 9 Dec. 2010 entitled Process of Separating Components of A Fermentation Broth; WO2010141920A2 published 9 Dec. 2010 entitled Microorganisms for the Production of 1,4-Butanediol and Related Methods; WO2010030711A2 published 18 Mar. 2010 entitled Microorganisms for the Production of 1,4-Butanediol; WO2010071697A1 published 24 Jun. 2010 Microorganisms and Methods for Conversion of Syngas and Other Carbon Sources to Useful Products; WO2009094485A1 published 30 Jul. 2009 Methods and Organisms for Utilizing Synthesis Gas or Other Gaseous Carbon Sources and Methanol; WO2009023493A1 published 19 Feb. 2009 entitled Methods and Organisms for the Growth-Coupled Production of 1,4-Butanediol; and WO2008115840A2 published 25 Sep. 2008 entitled Compositions and Methods for the Biosynthesis of 1,4-Butanediol and Its Precursors, which are all incorporated herein by reference.

Butadiene and intermediates thereto, such as 1,4-butanediol, 2,3-butanediol, 1,3-butanediol, crotyl alcohol, 3-buten-2-ol (methyl vinyl carbinol) and 3-buten-1-ol, are bioderived compounds that can be made via enzymatic pathways described herein and in the following publications. In addition to direct fermentation to produce butadiene, 1,3-butanediol, 1,4-butanediol, crotyl alcohol, 3-buten-2-ol (methyl vinyl carbinol) or 3-buten-1-ol can be separated, purified (for any use), and then chemically dehydrated to butadiene by metal-based catalysis. Suitable bioderived compound pathways and enzymes, methods for screening and methods for isolating are found in: WO2011140171A2 published 10 Nov. 2011 entitled Microorganisms and Methods for the Biosynthesis of Butadiene; WO2012018624A2 published 9 Feb. 2012 entitled Microorganisms and Methods for the Biosynthesis of Aromatics, 2,4-Pentadienoate and 1,3-Butadiene; WO2011140171A2 published 10 Nov. 2011 entitled Microorganisms and Methods for the Biosynthesis of Butadiene; WO2013040383A1 published 21 Mar. 2013 entitled Microorganisms and Methods for Producing Alkenes; WO2012177710A1 published 27 Dec. 2012 entitled Microorganisms for Producing Butadiene and Methods Related thereto; WO2012106516A1 published 9 Aug. 2012 entitled Microorganisms and Methods for the Biosynthesis of Butadiene; and WO2013028519A1 published 28 Feb. 2013 entitled Microorganisms and Methods for Producing 2,4-Pentadienoate, Butadiene, Propylene, 1,3-Butanediol and Related Alcohols, which are all incorporated herein by reference.

1,3-Butanediol and intermediates thereto, such as 2,4-pentadienoate, crotyl alcohol or 3-buten-1-ol, are bioderived compounds that can be made via enzymatic pathways described herein and in the following publications. Suitable bioderived compound pathways and enzymes, methods for screening and methods for isolating are found in: WO2011071682A1 published 16 Jun. 2011 entitled Methods and Organisms for Converting Synthesis Gas or Other Gaseous Carbon Sources and Methanol to 1,3-Butanediol; WO2011031897A published 17 Mar. 2011 entitled Microorganisms and Methods for the Co-Production of Isopropanol with Primary Alcohols, Diols and Acids; WO2010127319A2 published 4 Nov. 2010 entitled Organisms for the Production of 1,3-Butanediol; WO2013071226A1 published 16 May 2013 entitled Eukaryotic Organisms and Methods for Increasing the Availability of Cytosolic Acetyl-CoA, and for Producing 1,3-Butanediol; WO2013028519A1 published 28 Feb. 2013 entitled Microorganisms and Methods for Producing 2,4-Pentadienoate, Butadiene, Propylene, 1,3-Butanediol and Related Alcohols; WO2013036764A1 published 14 Mar. 2013 entitled Eukaryotic Organisms and Methods for Producing 1,3-Butanediol; WO2013012975A1 published 24 Jan. 2013 entitled Methods for Increasing Product Yields; WO2012177619A2 published 27 Dec. 2012 entitled Microorganisms for Producing 1,3-Butanediol and Methods Related Thereto; WO2018/183664A1 published 4 Oct. 2018 entitled Aldehyde Dehydrogenase Variants and Methods of Use; WO 2018/183640A1 published 4 Oct. 2018 entitled 3-Hhydroxybutryl-CoA Dehydrogenase Variants and Methods of Use; US 2019/0345455 published 14 Nov. 2019 entitled Alcohol Dehydrogenase Mutant and Application thereof in Synthesis of Diaryl Chiral Alcohols, and US 2019/0345455 published 14 Nov. 2019 entitled Alcohol Dehydrogenase Mutant and Application thereof in Synthesis of Diaryl Chiral Alcohols, which are all incorporated herein by reference.

Adipate, 6-aminocaproic acid, caprolactam, hexamethylenediamine and levulinic acid, and their intermediates, e.g. 4-aminobutyryl-CoA, are bioderived compounds that can be made via enzymatic pathways described herein and in the following publications. Suitable bioderived compound pathways and enzymes, methods for screening and methods for isolating are found in: WO2010129936A1 published 11 Nov. 2010 entitled Microorganisms and Methods for the Biosynthesis of Adipate, Hexamethylenediamine and 6-Aminocaproic Acid; WO2013012975A1 published 24 Jan. 2013 entitled Methods for Increasing Product Yields; WO2012177721A1 published 27 Dec. 2012 entitled Microorganisms for Producing 6-Aminocaproic Acid; WO2012099621A1 published 26 Jul. 2012 entitled Methods for Increasing Product Yields; and WO2009151728 published 17 Dec. 2009 entitled Microorganisms for the production of adipic acid and other compounds, which are all incorporated herein by reference.

Methacrylic acid (2-methyl-2-propenoic acid) is used in the preparation of its esters, known collectively as methacrylates (e.g., methyl methacrylate, which is used most notably in the manufacture of polymers). Methacrylate esters such as methyl methacrylate, 3-hydroxyisobutyrate and/or 2-hydroxyisobutyrate and their intermediates are bioderived compounds that can be made via enzymatic pathways described herein and in the following publications. Suitable bioderived compound pathways and enzymes, methods for screening and methods for isolating are found in: WO2012135789A2 published 4 Oct. 2012 entitled Microorganisms for Producing Methacrylic Acid and Methacrylate Esters and Methods Related Thereto; and WO2009135074A2 published 5 Nov. 2009 entitled Microorganisms for the Production of Methacrylic Acid, which are all incorporated herein by reference.

1,2-Propanediol (propylene glycol), n-propanol, 1,3-propanediol and glycerol, and their intermediates are bioderived compounds that can be made via enzymatic pathways described herein and in the following publications. Suitable bioderived compound pathways and enzymes, methods for screening and methods for isolating are found in: WO2009111672A1 published 9 Nov. 2009 entitled Primary Alcohol Producing Organisms; WO2011031897A1 17 Mar. 2011 entitled Microorganisms and Methods for the Co-Production of Isopropanol with Primary Alcohols, Diols and Acids; WO2012177599A2 published 27 Dec. 2012 entitled Microorganisms for Producing N-Propanol 1,3-Propanediol, 1,2-Propanediol or Glycerol and Methods Related Thereto, which are all incorporated herein by referenced.

Succinic acid and intermediates thereto, which are useful to produce products including polymers (e.g., PBS), 1,4-butanediol, tetrahydrofuran, pyrrolidone, solvents, paints, deicers, plastics, fuel additives, fabrics, carpets, pigments, and detergents, are bioderived compounds that can be made via enzymatic pathways described herein and in the following publication. Suitable bioderived compound pathways and enzymes, methods for screening and methods for isolating are found in: EP1937821A2 published 2 Jul. 2008 entitled Methods and Organisms for the Growth-Coupled Production of Succinate, which is incorporated herein by reference.

Primary alcohols and fatty alcohols (also known as long chain alcohols), including fatty acids and fatty aldehydes thereof, and intermediates thereto, are bioderived compounds that can be made via enzymatic pathways in the following publications. Suitable bioderived compound pathways and enzymes, methods for screening and methods for isolating are found in: WO2009111672 published 11 Sep. 2009 entitled Primary Alcohol Producing Organisms; WO2012177726 published 27 Dec. 2012 entitled Microorganism for Producing Primary Alcohols and Related Compounds and Methods Related Thereto, which are all incorporated herein by reference.

Further suitable bioderived compounds that the microbial organisms described herein can be used to produce via acetyl-CoA, including optionally further through acetoacetyl-CoA and/or succinyl-CoA, are included as part of the present disclosure. Exemplary well known bioderived compounds, their pathways and enzymes for production, methods for screening and methods for isolating are found in the following patents and publications: succinate (U.S. publication 2007/0111294, WO 2007/030830, WO 2013/003432), 3-hydroxypropionic acid (3-hydroxypropionate) (U.S. publication 2008/0199926, WO 2008/091627, U.S. publication 2010/0021978), 1,4-butanediol (U.S. Pat. No. 8,067,214, WO 2008/115840, U.S. Pat. No. 7,947,483, WO 2009/023493, U.S. Pat. No. 7,858,350, WO 2010/030711, U.S. publication 2011/0003355, WO 2010/141780, U.S. Pat. No. 8,129,169, WO 2010/141920, U.S. publication 2011/0201068, WO 2011/031897, U.S. Pat. No. 8,377,666, WO 2011/047101, U.S. publication 2011/0217742, WO 2011/066076, U.S. publication 2013/0034884, WO 2012/177943), 4-hydroxybutanoic acid (4-hydroxybutanoate, 4-hydroxybutyrate, 4-hydroxybutryate) (U.S. Pat. No. 8,067,214, WO 2008/115840, U.S. Pat. No. 7,947,483, WO 2009/023493, U.S. Pat. No. 7,858,350, WO 2010/030711, U.S. publication 2011/0003355, WO 2010/141780, U.S. Pat. No. 8,129,155, WO 2010/071697), γ-butyrolactone (U.S. Pat. No. 8,067,214, WO 2008/115840, U.S. Pat. No. 7,947,483, WO 2009/023493, U.S. Pat. No. 7,858,350, WO 2010/030711, U.S. publication 2011/0003355, WO 2010/141780, U.S. publication 2011/0217742, WO 2011/066076), 4-hydroxybutyryl-CoA (U.S. publication 2011/0003355, WO 2010/141780, U.S. publication 2013/0034884, WO 2012/177943), 4-hydroxybutanal (U.S. publication 2011/0003355, WO 2010/141780, U.S. publication 2013/0034884, WO 2012/177943), putrescine (U.S. publication 2011/0003355, WO 2010/141780, U.S. publication 2013/0034884, WO 2012/177943), Olefins (such as acrylic acid and acrylate ester) (U.S. Pat. No. 8,026,386, WO 2009/045637), acetyl-CoA (U.S. Pat. No. 8,323,950, WO 2009/094485), methyl tetrahydrofolate (U.S. Pat. No. 8,323,950, WO 2009/094485), ethanol (U.S. Pat. No. 8,129,155, WO 2010/071697), isopropanol (U.S. Pat. No. 8,129,155, WO 2010/071697, U.S. publication 2010/0323418, WO 2010/127303, U.S. publication 2011/0201068, WO 2011/031897), n-butanol (U.S. Pat. No. 8,129,155, WO 2010/071697), isobutanol (U.S. Pat. No. 8,129,155, WO 2010/071697), n-propanol (U.S. publication 2011/0201068, WO 2011/031897), methylacrylic acid (methylacrylate) (U.S. publication 2011/0201068, WO 2011/031897), primary alcohol (U.S. Pat. No. 7,977,084, WO 2009/111672, WO 2012/177726), long chain alcohol (U.S. Pat. No. 7,977,084, WO 2009/111672, WO 2012/177726), adipate (adipic acid) (U.S. Pat. No. 8,062,871, WO 2009/151728, U.S. Pat. No. 8,377,680, WO 2010/129936, WO 2012/177721), 6-aminocaproate (6-aminocaproic acid) (U.S. Pat. No. 8,062,871, WO 2009/151728, U.S. Pat. No. 8,377,680, WO 2010/129936, WO 2012/177721), caprolactam (U.S. Pat. No. 8,062,871, WO 2009/151728, U.S. Pat. No. 8,377,680, WO 2010/129936, WO 2012/177721), hexamethylenediamine (U.S. Pat. No. 8,377,680, WO 2010/129936, WO 2012/177721), levulinic acid (U.S. Pat. No. 8,377,680, WO 2010/129936), 2-hydroxyisobutyric acid (2-hydroxyisobutyrate) (U.S. Pat. No. 8,241,877, WO 2009/135074, U.S. publication 2013/0065279, WO 2012/135789), 3-hydroxyisobutyric acid (3-hydroxyisobutyrate) (U.S. Pat. No. 8,241,877, WO 2009/135074, U.S. publication 2013/0065279, WO 2012/135789), methacrylic acid (methacrylate) (U.S. Pat. No. 8,241,877, WO 2009/135074, U.S. publication 2013/0065279, WO 2012/135789), methacrylate ester (U.S. publication 2013/0065279, WO 2012/135789), fumarate (fumaric acid) (U.S. Pat. No. 8,129,154, WO 2009/155382), malate (malic acid) (U.S. Pat. No. 8,129,154, WO 2009/155382), acrylate (carboxylic acid) (U.S. Pat. No. 8,129,154, WO 2009/155382), methyl ethyl ketone (U.S. publication 2010/0184173, WO 2010/057022, U.S. Pat. No. 8,420,375, WO 2010/144746), 2-butanol (U.S. publication 2010/0184173, WO 2010/057022, U.S. Pat. No. 8,420,375, WO 2010/144746), 1,3-butanediol (U.S. publication 2010/0330635, WO 2010/127319, U.S. publication 2011/0201068, WO 2011/031897, U.S. Pat. No. 8,268,607, WO 2011/071682, U.S. publication 2013/0109064, WO 2013/028519, U.S. publication 2013/0066035, WO 2013/036764), cyclohexanone (U.S. publication 2011/0014668, WO 2010/132845), terephthalate (terephthalic acid) (U.S. publication 2011/0124911, WO 2011/017560, U.S. publication 2011/0207185, WO 2011/094131, U.S. publication 2012/0021478, WO 2012/018624), muconate (muconic acid) (U.S. publication 2011/0124911, WO 2011/017560), aniline (U.S. publication 2011/0097767, WO 2011/050326), p-toluate (p-toluic acid) (U.S. publication 2011/0207185, WO 2011/094131, U.S. publication 2012/0021478, WO 2012/018624), (2-hydroxy-3-methyl-4-oxobutoxy)phosphonate (U.S. publication 2011/0207185, WO 2011/094131, U.S. publication 2012/0021478, WO 2012/018624), ethylene glycol (U.S. publication 2011/0312049, WO 2011/130378, WO 2012/177983), propylene (U.S. publication 2011/0269204, WO 2011/137198, U.S. publication 2012/0329119, U.S. publication 2013/0109064, WO 2013/028519), butadiene (1,3-butadiene) (U.S. publication 2011/0300597, WO 2011/140171, U.S. publication 2012/0021478, WO 2012/018624, U.S. publication 2012/0225466, WO 2012/106516, U.S. publication 2013/0011891, WO 2012/177710, U.S. publication 2013/0109064, WO 2013/028519), toluene (U.S. publication 2012/0021478, WO 2012/018624), benzene (U.S. publication 2012/0021478, WO 2012/018624), (2-hydroxy-4-oxobutoxy)phosphonate (U.S. publication 2012/0021478, WO 2012/018624), benzoate (benzoic acid) (U.S. publication 2012/0021478, WO 2012/018624), styrene (U.S. publication 2012/0021478, WO 2012/018624), 2,4-pentadienoate (U.S. publication 2012/0021478, WO 2012/018624, U.S. publication 2013/0109064, WO 2013/028519), 3-butene-1-ol (U.S. publication 2012/0021478, WO 2012/018624, U.S. publication 2013/0109064, WO 2013/028519), 3-buten-2-ol (U.S. publication 2013/0109064, WO 2013/028519), 1,4-cyclohexanedimethanol (U.S. publication 2012/0156740, WO 2012/082978), crotyl alcohol (U.S. publication 2013/0011891, WO 2012/177710, U.S. publication 2013/0109064, WO 2013/028519), alkene (U.S. publication 2013/0122563, WO 2013/040383, US 2011/0196180), hydroxyacid (WO 2012/109176), ketoacid (WO 2012/109176), wax esters (WO 2007/136762) or caprolactone (U.S. publication 2013/0144029, WO 2013/067432) pathway. The patents and patent application publications listed above that disclose bioderived compound pathways are herein incorporated herein by reference.

One skilled in the art will understand that these are merely exemplary and that any of the substrate-product pairs disclosed herein suitable to produce a desired product and for which an appropriate activity is available for the conversion of the substrate to the product can be readily determined by one skilled in the art based on the teachings herein. Thus, in some embodiments, provided herein is a non-naturally occurring microbial organism containing at least one recombinant nucleic acid encoding an engineered formate dehydrogenase, where the formate dehydrogenase functions in a pathway to produce a bioderived compound.

In some embodiments, provided herein is a non-naturally occurring microbial organism having a vector described herein comprising a nucleic acid described herein. Also provided anon-naturally occurring microbial organism having a nucleic acid described herein. In some embodiments, the nucleic acid is integrated into a chromosome of the organism. In some embodiments, the integration is site-specific. In an embodiment described herein, the nucleic acid is expressed. In some embodiments, provided herein is a non-naturally occurring microbial organism having a polypeptide described herein.

In some embodiments, the microbial organism is a species of bacteria, yeast or fungus. In some embodiments, the microbial organism is a species of bacteria, yeast or fungus. In some embodiments, the microbial organism is a species of yeast. In some embodiments, the microbial organism is a species of fungus.

In some embodiments, provided herein is a non-naturally occurring microbial organism that is a capable of producing more NADH or a bioderived compound compared to a control microbial organism that does not having a recombinant nucleic acid that encodes an engineered formate dehydrogenase described herein. Such a microbial organism, in some embodiments, is capable of producing at least 10% more NADH or a bioderived compound compared to the control microbial organism. In some embodiments, the microbial organism is capable of producing at least 20% more NADH or a bioderived compound compared to the control microbial organism. In some embodiments, the microbial organism is capable of producing at least 30% more NADH or a bioderived compound compared to the control microbial organism. In some embodiments, the microbial organism is capable of producing at least 40% more NADH or a bioderived compound compared to the control microbial organism. In some embodiments, the microbial organism is capable of producing at least 50% more NADH or a bioderived compound compared to the control microbial organism. In some embodiments, the microbial organism is capable of producing at least 60% more NADH or a bioderived compound compared to the control microbial organism. In some embodiments, the microbial organism is capable of producing at least 70% more NADH or a bioderived compound compared to the control microbial organism. In some embodiments, the microbial organism is capable of producing at least 80% more NADH or a bioderived compound compared to the control microbial organism. In some embodiments, the microbial organism is capable of producing at least 90% more NADH or a bioderived compound compared to the control microbial organism. In some embodiments, the microbial organism is capable of producing at least 1 fold more NADH or a bioderived compound compared to the control microbial organism. In some embodiments, the microbial organism is capable of producing at least 1.1 fold more NADH or a bioderived compound compared to the control microbial organism. In some embodiments, the microbial organism is capable of producing at least 1.2 fold more NADH or a bioderived compound compared to the control microbial organism. In some embodiments, the microbial organism is capable of producing at least 1.3 fold more NADH or a bioderived compound compared to the control microbial organism. In some embodiments, the microbial organism is capable of producing at least 1.4 fold more NADH or a bioderived compound compared to the control microbial organism. In some embodiments, the microbial organism is capable of producing at least 1.5 fold more NADH or a bioderived compound compared to the control microbial organism. In some embodiments, the microbial organism is capable of producing at least 1.6 fold more NADH or a bioderived compound compared to the control microbial organism. In some embodiments, the microbial organism is capable of producing at least 1.7 fold more NADH or a bioderived compound compared to the control microbial organism. In some embodiments, the microbial organism is capable of producing at least 1.8 fold more NADH or a bioderived compound compared to the control microbial organism. In some embodiments, the microbial organism is capable of producing at least 1.9 fold more NADH or a bioderived compound compared to the control microbial organism. In some embodiments, the microbial organism is capable of producing at least 2 fold more NADH or a bioderived compound compared to the control microbial organism.

The subject matter described herein includes general reference to the metabolic reaction, reactant or product thereof, or with specific reference to one or more nucleic acids or genes encoding an enzyme associated with or catalyzing, or a protein associated with, the referenced metabolic reaction, reactant or product. Unless otherwise expressly stated herein, those skilled in the art will understand that reference to a reaction also constitutes reference to the reactants and products of the reaction. Similarly, unless otherwise expressly stated herein, reference to a reactant or product also references the reaction, and reference to any of these metabolic constituents also references the gene or genes encoding the enzymes that catalyze or proteins involved in the referenced reaction, reactant or product. Likewise, given the well known fields of metabolic biochemistry, enzymology and genomics, reference herein to a gene or encoding nucleic acid also constitutes a reference to the corresponding encoded enzyme and the reaction it catalyzes or a protein associated with the reaction as well as the reactants and products of the reaction.

The non-naturally occurring microbial organisms described herein can be produced by introducing expressible nucleic acids encoding one or more of the enzymes or proteins participating in one or more bioderived compound biosynthetic pathways. Depending on the host microbial organism chosen for biosynthesis, nucleic acids for some or all of a particular a bioderived compound biosynthetic pathway can be expressed. For example, if a chosen host is deficient in one or more enzymes or proteins for a desired biosynthetic pathway, then expressible nucleic acids for the deficient enzyme(s) or protein(s) are introduced into the host for subsequent exogenous expression. Alternatively, if the chosen host exhibits endogenous expression of some pathway genes, but is deficient in others, then an encoding nucleic acid is needed for the deficient enzyme(s) or protein(s) to achieve bioderived compound biosynthesis. Thus, a non-naturally occurring microbial organism described herein can be produced by introducing exogenous enzyme or protein activities to obtain a desired biosynthetic pathway or a desired biosynthetic pathway can be obtained by introducing one or more exogenous enzyme or protein activities that, together with one or more endogenous enzymes or proteins, produces a desired product such as a bioderived compound.

Host microbial organisms can be selected from, and the non-naturally occurring microbial organisms generated in, for example, bacteria, yeast, fungus or any of a variety of other microorganisms applicable or suitable to fermentation processes. Exemplary bacteria include any species selected from the order Enterobacteriales, family Enterobacteriaceae, including the genera Escherichia and Klebsiella; the order Aeromonadales, family Succinivibrionaceae, including the genus Anaerobiospirillum; the order Pasteurellales, family Pasteurellaceae, including the genera Actinobacillus and Mannheimia; the order Rhizobiales, family Bradyrhizobiaceae, including the genus Rhizobium; the order Bacillales, family Bacillaceae, including the genus Bacillus; the order Actinomycetales, families Corynebacteriaceae and Streptomycetaceae, including the genus Corynebacterium and the genus Streptomyces, respectively; order Rhodospirillales, family Acetobacteraceae, including the genus Gluconobacter; the order Sphingomonadales, family Sphingomonadaceae, including the genus Zymomonas; the order Lactobacillales, families Lactobacillaceae and Streptococcaceae, including the genus Lactobacillus and the genus Lactococcus, respectively; the order Clostridiales, family Clostridiaceae, genus Clostridium; and the order Pseudomonadales, family Pseudomonadaceae, including the genus Pseudomonas. Non-limiting species of host bacteria include Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. Exemplarily bacterial methylotrophs include, for example, Bacillus, Methylobacterium, Methyloversatilis, Methylococcus, Methylocystis and Hyphomicrobium.

Similarly, exemplary species of yeast or fungi species include any species selected from the order Saccharomycetales, family Saccaromycetaceae, including the genera Saccharomyces, Kluyveromyces and Pichia; the order Saccharomycetales, family Dipodascaceae, including the genus Yarrowia; the order Schizosaccharomycetales, family Schizosaccaromycetaceae, including the genus Schizosaccharomyces; the order Eurotiales, family Trichocomaceae, including the genus Aspergillus; and the order Mucorales, family Mucoraceae, including the genus Rhizopus. Non-limiting species of host yeast or fungi include Saccharomyces cerevisiae, Schizosaccharomyces pombe, Kluyveromyces lactis, Kluyveromyces marxianus, Aspergillus terreus, Aspergillus niger, Pichia pastoris, Rhizopus arrhizus, Rhizopus oryzae, Yarrowia lipolytica, and the like. E. coli is a particularly useful host organism since it is a well characterized microbial organism suitable for genetic engineering. Other particularly useful host organisms include yeast such as Saccharomyces cerevisiae and yeasts or fungi selected from the genera Saccharomyces, Schizosaccharomyces, Schizochytrium, Rhodotorula, Thraustochytrium, Aspergillus, Kluyveromyces, Issatchenkia, Yarrowia, Candida, Pichia, Ogataea, Kuraishia, Hansenula and Komagataella. Useful host organisms include Saccharomyces cerevisiae, Schizosaccharomyces pombe, Hansenula polymorpha, Pichia methanolica, Candida boidinii, Kluyveromyces lactis, Kluyveromyces marxianus, Aspergillus terreus, Aspergillus niger, Pichia pastoris, Rhizopus arrhizus, Rhizopus oryzae, Yarrowia lipolytica, Issatchenkia orientalis and the like. It is understood that any suitable microbial host organism can be used to introduce metabolic and/or genetic modifications to produce a desired product.

Depending on the bioderived compound biosynthetic pathway constituents of a selected host microbial organism, the non-naturally occurring microbial organisms described herein can include at least one exogenously expressed bioderived compound pathway-encoding nucleic acid and up to all encoding nucleic acids for one or more bioderived compound biosynthetic pathways. For example, bioderived compound biosynthesis can be established in a host deficient in a pathway enzyme or protein through exogenous expression of the corresponding encoding nucleic acid. In a host deficient in all enzymes or proteins of a bioderived compound pathway, exogenous expression of all enzyme or proteins in the pathway can be included, although it is understood that all enzymes or proteins of a pathway can be expressed even if the host contains at least one of the pathway enzymes or proteins.

Given the teachings and guidance provided herein, those skilled in the art will understand that the number of encoding nucleic acids to introduce in an expressible form will, at least, parallel the bioderived compound pathway deficiencies of the selected host microbial organism. Therefore, a non-naturally occurring microbial organism described herein can have one, two, three, four, five, six, seven, eight, nine, ten, eleven, or twelve up to all nucleic acids encoding the enzymes or proteins constituting a bioderived compound biosynthetic pathway disclosed herein. In some embodiments, the non-naturally occurring microbial organisms also can include other genetic modifications that facilitate or optimize a bioderived compound biosynthesis or that confer other useful functions onto the host microbial organism. One such other functionality can include, for example, augmentation of the synthesis of one or more of the bioderived compound pathway precursors.

Generally, a host microbial organism is selected such that it produces the precursor of a bioderived compound pathway, either as a naturally produced molecule or as an engineered product that either provides de novo production of a desired precursor or increased production of a precursor naturally produced by the host microbial organism. For example, malonyl-CoA, acetoacetyl-CoA and pyruvate are produced naturally in a host organism such as E. coli. A host organism can be engineered to increase production of a precursor, as disclosed herein. In addition, a microbial organism that has been engineered to produce a desired precursor can be used as a host organism and further engineered to express enzymes or proteins of a bioderived compound pathway.

In some embodiments, a non-naturally occurring microbial organism described herein is generated from a host that contains the enzymatic capability to synthesize a bioderived compound. In this specific embodiment it can be useful to increase the synthesis or accumulation of NADH to, for example, drive a bioderived compound pathway reactions toward a bioderived compound production. Increased synthesis or accumulation can be accomplished by, for example, expression (e.g., overexpression) of nucleic acids encoding an engineered formate dehydrogenase described herein and expression (e.g., overexpression) of an enzyme or enzymes and/or protein or proteins of the bioderived compound pathway. Expression of the enzyme or enzymes and/or protein or proteins of the bioderived compound pathway can occur, for example, through exogenous expression of the endogenous gene or genes, or through exogenous expression of the heterologous gene or genes. Therefore, naturally occurring organisms can be readily generated to be non-naturally occurring microbial organisms described herein, for example, producing a bioderived compound, through overexpression of one, two, three, four, five, six, seven, eight, nine, ten, eleven, twelve, that is, up to all nucleic acids encoding a bioderived compound biosynthetic pathway enzymes or proteins. In addition, a non-naturally occurring organism can be generated by mutagenesis of an endogenous gene that results in an increase in activity of an enzyme in the bioderived compound biosynthetic pathway.

In particularly useful embodiments, exogenous expression of the encoding nucleic acids is employed. Exogenous expression confers the ability to custom tailor the expression and/or regulatory elements to the host and application to achieve a desired expression level that is controlled by the user. In some embodiments, the expression of an endogenous gene is manipulated, such as by removing a negative regulatory effector or induction of the gene's promoter when linked to an inducible promoter or other regulatory element. Thus, an endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microbial organism.

It is understood that, in methods described herein, any of the one or more recombinant and/or exogenous nucleic acids can be introduced into a microbial organism to produce a non-naturally occurring microbial organism described herein. The nucleic acids can be introduced so as to confer, for example, production of a cofactor, such as NADH and/or NADPH, or a bioderived compound biosynthetic pathway onto the microbial organism. Alternatively, encoding nucleic acids can be introduced to produce an intermediate microbial organism having the biosynthetic capability to catalyze some of the required reactions to confer cofactor production or a bioderived compound biosynthetic capability. For example, a non-naturally occurring microbial organism having NADH and a bioderived compound biosynthetic pathway can comprise at least two exogenous nucleic acids encoding desired enzymes or proteins, such as the combination of an engineered formate dehydrogenase provided herein and a 1,3-BDO pathway enzyme, or alternatively an engineered formate dehydrogenase provided herein and a HMDA pathway enzyme, or alternatively an engineered formate dehydrogenase provided herein and a MAA pathway enzyme, and the like. Thus, it is understood that any combination of two or more enzymes or proteins of a biosynthetic pathway can be included in a non-naturally occurring microbial organism described herein. Similarly, it is understood that any combination of three or more enzymes or proteins of a biosynthetic pathway can be included in a non-naturally occurring microbial organism described herein, for example, an engineered formate dehydrogenase provided herein, a transhydrogenase and a 1,3-BDO pathway enzyme, and so forth, as desired, so long as the combination of enzymes and/or proteins of the desired biosynthetic pathway results in production of the corresponding desired product. Similarly, any combination of four, five, six, seven, eight, nine, ten, eleven, twelve or more enzymes or proteins of a biosynthetic pathway as disclosed herein can be included in a non-naturally occurring microbial organism described herein, as desired, so long as the combination of enzymes and/or proteins of the desired biosynthetic pathway results in production of the corresponding desired product.

In addition to the biosynthesis of NADH or a bioderived compound as described herein, the non-naturally occurring microbial organisms and methods described herein also can be utilized in various combinations with each other and/or with other microbial organisms and methods well known in the art to achieve product biosynthesis by other routes. For example, one alternative to produce a bioderived compound other than use of the bioderived compound producers is through addition of another microbial organism capable of converting a bioderived compound pathway intermediate to a bioderived compound. One such procedure includes, for example, the fermentation of a microbial organism that produces a bioderived compound pathway intermediate. The bioderived compound pathway intermediate can then be used as a substrate for a second microbial organism that converts the bioderived compound pathway intermediate to a bioderived compound. The bioderived compound pathway intermediate can be added directly to another culture of the second organism or the original culture of the bioderived compound pathway intermediate producers can be depleted of these microbial organisms by, for example, cell separation, and then subsequent addition of the second organism to the fermentation broth can be utilized to produce the final product without intermediate purification steps.

Given the teachings and guidance provided herein, those skilled in the art will understand that a wide variety of combinations and permutations exist for the non-naturally occurring microbial organisms and methods described herein together with other microbial organisms, with the co-culture of other non-naturally occurring microbial organisms having subpathways and with combinations of other chemical and/or biochemical procedures well known in the art to produce a bioderived compound.

Similarly, it is understood by those skilled in the art that a host organism can be selected based on desired characteristics for introduction of one or more gene disruptions to increase synthesis or production of NADH or a bioderived compound. Thus, it is understood that, if a genetic modification is to be introduced into a host organism to disrupt a gene, any homologs, orthologs or paralogs that catalyze similar, yet non-identical metabolic reactions can similarly be disrupted to ensure that a desired metabolic reaction is sufficiently disrupted. Because certain differences exist among metabolic networks between different organisms, those skilled in the art will understand that the actual genes disrupted in a given organism may differ between organisms. However, given the teachings and guidance provided herein, those skilled in the art also will understand that the methods described herein can be applied to any suitable host microorganism to identify the cognate metabolic alterations needed to construct an organism in a species of interest that will increase NADH or a bioderived compound biosynthesis. In a particular embodiment, the increased production couples biosynthesis of NADH or a bioderived compound to growth of the organism, and can obligatorily couple production of NADH or a bioderived compound to growth of the organism if desired and as disclosed herein.

Sources of encoding nucleic acids for a bioderived compound pathway enzyme or protein can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, Abies grandis, Acetobacter aceti, Acetobacter pasteurians, Achromobacter denitrificans, Acidaminococcus fermentans, Acinetobacter baumannii Naval-82, Acinetobacter baylyi, Acinetobacter calcoaceticus, Acinetobacter sp. ADP1, Acinetobacter sp. Strain M-1, Actinobacillus succinogenes, Actinobacillus succinogenes 130Z, Aeropyrum pernix, Agrobacterium tumefaciens, Alkaliphilus metalliredigenes QYF, Allochromatium vinosum DSM 180, Aminomonas aminovorus, Amycolicicoccus subflavus DQS3-9A1, Anaerobiospirillum succiniciproducens, Anaerotruncus colihominis, Aquifex aeolicus VF5, Arabidopsis thaliana, Arabidopsis thaliana col, Archaeglubus fulgidus, Archaeoglobus fulgidus, Archaeoglobus fulgidus DSM 4304, Arthrobacter globiformis, Ascaris suum, Aspergillus fumigatus, Aspergillus nidulans, Aspergillus niger, Aspergillus niger CBS 513.88, Aspergillus terreus NIH2624, Atopobium parvulum DSM 20469, Azotobacter vinelandii DJ, Bacillus alcalophilus ATCC 27647, Bacillus azotoformans LMG 9581, Bacillus cereus, Bacillus cereus ATCC 14579, Bacillus coagulans 36D1, Bacillus megaterium, Bacillus methanolicus MGA3, Bacillus methanolicus PB1, Bacillus methanolicus PB-1, Bacillus selenitireducens MLS10, Bacillus smithii, Bacillus sphaericus, Bacillus subtilis, Bacteroides capillosus, Bifidobacterium animalis lactis, Bifidobacterium breve, Bifidobacterium dentium ATCC 27678, Bifidobacterium pseudolongum subsp. globosum, Bos taurus, Burkholderia cenocepacia, Burkholderia cepacia, Burkholderia multivorans, Burkholderia pyrrocinia, Burkholderia stabilis, Burkholderia thailandensis E264, Burkholderia xenovorans, butyrate-producing bacterium L2-50, Caenorhabditis elegans, Campylobacter curvus 525.92, Campylobacter jejuni, Candida albicans, Candida boidinii, Candida methylica, Candida parapsilosis, Candida tropicalis, Candida tropicalis MYA-3404, Carboxydothermus hydrogenoformans, Carboxydothermus hydrogenoformans Z-2901, Castellaniella defragrans, Caulobacter sp. APO7, Chlamydomonas reinhardtii, Chlorobium phaeobacteroides DSM266, Chlorobium limicola, Chlorobium tepidum, Chloroflexus aggregans DSM9485, Chloroflexus aurantiacus, Chloroflexus aurantiacus J-10-fl, Citrobacter koseri ATCC BAA-895, Citrobacter youngae, Citrobacter youngae ATCC 29220, Clostridium acetobutylicum, Clostridium acetobutylicum ATCC 824, Clostridium acidurici, Clostridium aminobutyricum, Clostridium asparagiforme DSM15981, Clostridium beijerinckii, Clostridium beijerinckii NCIB 8052, Clostridium beijerinckii NRRL B593, Clostridium beijerinckii, Clostridium bolteae ATCC BAA-613, Clostridium botulinum C str. Eklund, Clostridium carboxidivorans P7, Clostridium cellulolyticum H10, Clostridium cellulovorans 743B, Clostridium difficile, Clostridium difficile 630, Clostridium hiranonis DSM 13275, Clostridium hylemonae DSM 15053, Clostridium kluyveri, Clostridium kluyveri DSM 555, Clostridium ljungdahli, Clostridium ljungdahlii DSM, Clostridium ljungdahlii DSM13528, Clostridium methylpentosum DSM5476, Clostridium novyi NT, Clostridium pasteurianum, Clostridium pasteurianum DSM525, Clostridium perfringens, Clostridium perfringens ATCC 13124, Clostridium perfringens str. 13, Clostridium phytofermentans ISDg, Clostridium propionicum, Clostridium saccharobutylicum, Clostridium saccharoperbutylacetonicum, Clostridium saccharoperbutylacetonicum N1-4, Clostridium tetani, Comamonas sp. CNB-1, Comamonas sp. CNB-1, Corynebacterium glutamicum, Corynebacterium glutamicum ATCC 13032, Corynebacterium glutamicum ATCC 14067, Corynebacterium glutamicum R, Corynebacterium sp., Corynebacterium sp. U-96, Corynebacterium variabile, Cryptosporidium parvum Iowa II, Cucumis sativus, Cupriavidus necator N-1, Cyanobium PCC7001, Deinococcus radiodurans RI, Desulfatibacillum alkenivorans AK-01, Desulfitobacterium hafniense, Desulfitobacterium metallireducens DSM15288, Desulfotomaculum reducens MI-1, Desulfovibrio africanus, Desulfovibrio africanus str. Walvis Bay, DesulfoVibrio desulfuricans G20, Desulfovibrio desulfuricans subsp. desulfuricans str. ATCC 27774, Desulfovibrio fructosovorans JJ, Desulfovibrio vulgaris str. Hildenborough, Dictyostelium discoideum AX4, Elizabethkingia meningoseptica, Enterococcus faecalis, Erythrobacter sp. NAP1, Escherichia coli C, Escherichia coli K12, Escherichia coli K-12 MG1655, Escherichia coli W, Eubacterium barkeri, Eubacterium hali DSM 3353, Eubacterium rectale ATCC 33656, Euglena gracilis, Flavobacterium frigoris, Fusobacterium nucleatum, Fusobacterium nucleatum subsp. polymorphum ATCC 10953, Geobacillus sp. GHH01, Geobacillus sp. M1OEXG, Geobacillus sp. Y4.1MC1, Geobacillus stearothermophilus, Geobacillus themodenitrificans NG80-2, Geobacillus thermoglucosidasius, Geobacter bemidjiensis Bem, Geobacter metallireducens GS-15, Geobacter sulfurreducens, Geobacter sulfurreducens PCA, Haemophilus influenza, Haemophilus influenzae, Haloarcula marismortui, Haloarcula marismortui ATCC 43049, Halobacterium salinarum, Hansenula polymorpha DL-1, Helicobacter pylori, Helicobacter pylori 26695, Heliobacter pylori, Homo sapiens, human gut metagenome, Hydrogenobacter thermophilus, Hydrogenobacter thermophilus TK-6, Hyphomicrobium denitrifcans ATCC 51888, Hyphomicrobium zavarzinii, Klebsiella pneumoniae, Klebsiella pneumoniae subsp. pneumoniae MGH 78578, Kluyveromyces lactis, Kluyveromyces lactis NRRL Y-1140, Lactobacillus acidophilus, Lactobacillus brevis ATCC 367, Lactobacillus paraplantarum, Lactococcus lactis, Leuconostoc mesenteroides, Lysinibacillus fusiformis, Lysinibacillus sphaericus, Malus x domestica, Mannheimia succiniciproducens, marine gamma proteobacterium HTCC2080, Marine metagenome JCVI_SCAF_1096627185304, Mesorhizobium loti MAFF303099, Metallosphaera sedula, Metarhizium acridum CQMa 102, Methanocaldococcus janaschii, Methanocaldococcus jannaschii, Methanosarcina acetivorans, Methanosarcina acetivorans C2A, Methanosarcina barkeri, Methanosarcina mazei, Methanosarcina mazei Tuc01, Methanosarcina thermophila, Methanothermobacter thermautotrophicus, Methylibium petroleiphilum PM1, Methylobacillus fagellatus, Methylobacillus flagellatus KT, Methylobacter marinus, Methylobacterium extorquens, Methylobacterium extorquens AM1, Methylococcus capsulatas, Methylomicrobium album BG8, Methylomonas aminofaciens, Methylovorus glucosetrophus SIP3-4, Methylovorus sp. MP688, Moorella thermoacetica, Mus musculus, Mycobacter sp. strain JCI DSM 3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Mycobacterium tuberculosis, Mycoplasma pneumoniae M129, Natranaerobius thermophilus, Nectria haematococca mpVI 77-13-4, Neurospora crassa, Nitrososphaera gargensis Ga9.2, Nocardia brasiliensis, Nocardia farcinica IFM 10152, Nocardia iowensis, Nocardia iowensis (sp. NRRL 5646), Nostoc sp. PCC 7120, Ogataea parapolymorpha DL-1(Hansenula polymorpha DL-1), Organism, Oryctolagus cuniculus, Oxalobacter formigenes, Paenibacillus peoriae KCTC 3763, Paracoccus denitrificans, Pelobacter carbinolicus DSM2380, Pelotomaculum thermopropionicum, Penicillium chrysogenum, Perkinsus marinus ATCC 50983, Photobacterium profundum 3TCK, Picea abies, Pichia pastoris, Picrophilus torridus DSM9790, Pinus sabiniana, Plasmodium falciparum, Populus alba, Populus tremula x Populus alba, Porphyromonas gingivalis, Porphyromonas gingivalis W83, Propionibacterium acnes, Propionibacteriumfredenreichii sp. shermanii, Pseudomonas aeruginosa, Pseudomonas aeruginosa PA01, Pseudomonas chlororaphis, Pseudomonas fluorescens, Pseudomonas knackmussii, Pseudomonas knackmussii (B13), Pseudomonas mendocina, Pseudomonas putida, Pseudomonas sp, Pseudomonas syringae pv. syringae B728a, Psychroflexus torquis ATCC 700755, Pueraria montana, Pyrobaculum aerophilum str. IM2, Pyrobaculum islandicum DSM 4184, Pyrococcus abyssi, Pyrococcus furiosus, Pyrococcus horikoshii OT3, Ralstonia eutropha, Ralstonia eutropha H16, Rattus norvegicus, Rhizobium leguminosarum, Rhodobacter capsulatus, Rhodobacter sphaeroides, Rhodobacter sphaeroides ATCC 17025, Rhodococcus opacus B4, Rhodococcus ruber, Rhodopseudomonas palustris, Rhodopseudomonas palustris CGA009, Rhodospirillum rubrum, Rhodospirillum rubrum ATCC 11170, Roseburia intestinalis L1-82, Roseburia inulinivorans, Roseburia sp. A2-183, Roseiflexus castenholzii, Rubrivivax gelatinosus, Ruminococcus obeum ATCC 29174, Saccharomyces cerevisiae, Saccharomyces cerevisiae s288c, Saccharomyces kluyveri, Saccharomyces serevisiae, Sachharomyces cerevisiae, Salmonella enteric, Salmonella enterica, Salmonella enterica subsp. arizonae serovar, Salmonella enterica subsp. enterica serovar Typhimurium str. LT2, Salmonella enterica Typhimurium, Salmonella typhimurium, Salmonella typhimurium LT2, Schizosaccharomyces pombe, Sebaldella termitidis ATCC 33386, Serratia proteamaculans, Shewanella oneidensis MR-4, Shigella flexneri, Sinorhizobium meliloti 1021, Solanum lycopersicum, Staphylococcus aureus, Stereum hirsutum FP-91666 SS1, Streptococcus mutans, Streptococcus pneumonia, Streptococcus pneumoniae, Streptococcus pyogenes ATCC 10782, Streptomyces anulatus, Streptomyces avermitilis, Streptomyces cinnamonensis, Streptomyces clavuligerus, Streptomyces coelicolor, Streptomyces coelicolor A3(2), Streptomyces griseus, Streptomyces griseus subsp. griseus NBRC 13350, Streptomyces sp CL190, Streptomyces sp. 2065, Streptomyces sp. ACT-1, Streptomyces sp. KO-3988, Sulfolobus acidocalarius, Sulfolobus acidocaldarius, Sulfolobus solfataricus, Sulfolobus solfataricus P-2, Sulfolobus sp. strain 7, Sulfolobus tokodaii, Sulfurimonas denitrificans, Sus scrofa, Synechococcus elongatus PCC 7942, Synechococcus sp. PCC 7002, Synechocystis str. PCC 6803, Syntrophobacter fumaroxidans, Thauera aromatica, Thermoanaerobacter brockii HTD4, Thermoanaerobacter sp. X514, Thermoanaerobacter tengcongensis MB4, Thermococcus kodakaraensis, Thermococcus litoralis, Thermoplasma acidophilum, Thermoproteus neutrophilus, Thermotoga maritima, Thermotoga maritime, Thermotoga maritime MSB8, Thermus thermophilus, Thiocapsa roseopersicina, Tolumonas auensis DSM 9187, Treponema denticola, Trichomonas vaginalis G3, Triticum aestivum, Trypanosoma brucei, Tsukamurella paurometabola DSM 20162, Uncultured bacterium, uncultured organism, Vibrio cholera, Vibrio harveyi ATCC BAA-1116, Xanthobacter autotrophicus Py2, Yarrowia lipolytica, Yersinia frederiksenii, Yersinia intermedia, Yersinia intermedia ATCC 29909, Yersinia pestis, Zea mays, Zoogloea ramigera, Zymomonas mobilus, as well as other exemplary species disclosed herein or available as source organisms for corresponding genes. However, with the complete genome sequence available for now more than 550 species (with more than half of these available on public databases such as the NCBI), including 395 microorganism genomes and a variety of yeast, fungi, plant, and mammalian genomes, the identification of genes encoding the requisite a bioderived compound biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations allowing biosynthesis of a bioderived compound described herein with reference to a particular organism such as E. coli can be readily applied to other microorganisms, including prokaryotic and eukaryotic organisms alike. Given the teachings and guidance provided herein, those skilled in the art will know that a metabolic alteration exemplified in one organism can be applied equally to other organisms.

In some instances, such as when an alternative bioderived compound biosynthetic pathway exists in an unrelated species, a bioderived compound biosynthesis can be conferred onto the host species by, for example, exogenous expression of a paralog or paralogs from the unrelated species that catalyzes a similar, yet non-identical metabolic reaction to replace the referenced reaction. Because certain differences among metabolic networks exist between different organisms, those skilled in the art will understand that the actual gene usage between different organisms may differ. However, given the teachings and guidance provided herein, those skilled in the art also will understand that the teachings and methods described herein can be applied to all microbial organisms using the cognate metabolic alterations to those exemplified herein to construct a microbial organism in a species of interest that will synthesize a bioderived compound.

Methods for constructing and testing the expression levels of a non-naturally occurring bioderived compound-producing host can be performed, for example, by recombinant and detection methods well known in the art. Such methods can be found described in, for example, Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999).

A recombinant nucleic acid encoding an engineered formate dehydrogenase as described herein and/or an exogenous nucleic acid encoding one or more enzymes or proteins involved in a pathway for production of NADH or a bioderived compound as described herein can be introduced stably or transiently into a microbial organism using techniques well known in the art including, but not limited to, conjugation, electroporation, chemical transformation, transduction, transfection, and ultrasound transformation. For exogenous expression in E. coli or other prokaryotic cells, some nucleotide sequences in the genes or cDNAs of eukaryotic nucleic acids can encode targeting signals such as an N-terminal mitochondrial or other targeting signal, which can be removed before transformation into prokaryotic microbial organisms, if desired. For example, removal of a mitochondrial leader sequence led to increased expression in E. coli (Hoffmeister et al., J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the microbial organisms. Thus, it is understood that appropriate modifications to a nucleotide sequence to remove or include a targeting sequence can be incorporated into a recombinant nucleic acid or an exogenous nucleic acid to impart desirable properties. Furthermore, genes can be subjected to codon optimization with techniques well known in the art to achieve optimized expression of the proteins.

An expression vector or vectors can be constructed to include a recombinant nucleic acid encoding an engineered formate dehydrogenase as described herein and/or an exogenous nucleic acid encoding one or more enzymes or proteins of a bioderived compound biosynthetic pathway as described herein operably linked to expression control sequences functional in the host organism. Expression vectors applicable for use in the microbial host organisms described herein include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more recombinant and/or exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of a recombinant or exogenous nucleic acid involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid or its corresponding gene product. It is understood by those skilled in the art that the recombinant and/or exogenous nucleic acid is expressed in a sufficient amount to produce the desired product, and it is further understood that expression levels can be optimized to obtain sufficient expression using methods well known in the art and as disclosed herein.

In some embodiments, provided herein is a method for producing a bioderived compound described herein. Such a method can comprise culturing the non-naturally occurring microbial organism as described herein under conditions and for a sufficient period of time to produce the bioderived compound. Thus, in some embodiments, provided herein is a method for producing a bioderived compound described herein comprising culturing a host cell described herein for a sufficient period of time to produce the bioderived compound. In another embodiment, method further includes separating the bioderived compound from other components in the culture. In this aspect, separating can include extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

In some embodiments, depending on the bioderived compound, the method described herein may further include chemically converting a bioderived compound to the directed final compound. For example, in some embodiments, wherein the bioderived compound is butadiene, the method described herein can further include chemically dehydrating 1,3-butanediol, crotyl alcohol, or 3-buten-2-ol to produce the butadiene.

Suitable purification and/or assays to test for the production of NADH or a bioderived compound can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et al., Biotechnol. Bioeng. 90:775-779 (2005)), or other suitable assay and detection methods well known in the art. The individual enzyme or protein activities from the recombinant and/or exogenous nucleic acids can also be assayed using methods well known in the art.

The bioderived compound can be separated from other components in the culture using a variety of methods well known in the art. Such separation methods include, for example, extraction procedures as well as methods that include continuous liquid-liquid extraction, pervaporation, membrane filtration, membrane separation, reverse osmosis, electrodialysis, distillation, crystallization, centrifugation, extractive filtration, ion exchange chromatography, size exclusion chromatography, adsorption chromatography, and ultrafiltration. All of the above methods are well known in the art.

Any of the non-naturally occurring microbial organisms described herein can be cultured to produce and/or secrete the biosynthetic products described herein. For example, the bioderived compound producers can be cultured for the biosynthetic production of a bioderived compound disclosed herein. Accordingly, in some embodiments, provided herein is a culture medium having the bioderived compound or bioderived compound pathway intermediate described herein. In some aspects, the culture medium can also be separated from the non-naturally occurring microbial organisms described herein that produced the bioderived compound or bioderived compound pathway intermediate. Methods for separating a microbial organism from culture medium are well known in the art. Exemplary methods include filtration, flocculation, precipitation, centrifugation, sedimentation, and the like.

For the production of NADH or a bioderived compound, the recombinant strains are cultured in a medium with carbon source and other essential nutrients. It is sometimes desirable and can be highly desirable to maintain anaerobic conditions in the fermenter to reduce the cost of the overall process. Such conditions can be obtained, for example, by first sparging the medium with nitrogen and then sealing the flasks with a septum and crimp-cap. For strains where growth is not observed anaerobically, microaerobic or substantially anaerobic conditions can be applied by perforating the septum with a small hole for limited aeration. Exemplary anaerobic conditions have been described previously and are well-known in the art. Exemplary aerobic and anaerobic conditions are described, for example, in United State publication 2009/0047719, filed Aug. 10, 2007. Fermentations can be performed in a batch, fed-batch or continuous manner, as disclosed herein. Fermentations can also be conducted in two phases, if desired. The first phase can be aerobic to allow for high growth and therefore high productivity, followed by an anaerobic phase of high bioderived compound yields.

If desired, the pH of the medium can be maintained at a desired pH, in particular neutral pH, such as a pH of around 7 by addition of a base, such as NaOH or other bases, or acid, as needed to maintain the culture medium at a desirable pH. The growth rate can be determined by measuring optical density using a spectrophotometer (600 nm), and the glucose uptake rate by monitoring carbon source depletion over time.

The growth medium, can include, for example, any carbohydrate source which can supply a source of carbon to the non-naturally occurring microbial organism described herein. Such sources include, for example, sugars such as glucose, xylose, arabinose, galactose, mannose, fructose, sucrose and starch; or glycerol, alone as the sole source of carbon or in combination with other carbon sources described herein or known in the art. In one embodiment, the carbon source is a sugar. In one embodiment, the carbon source is a sugar-containing biomass. In some embodiments, the sugar is glucose. In one embodiment, the sugar is xylose. In another embodiment, the sugar is arabinose. In one embodiment, the sugar is galactose. In another embodiment, the sugar is fructose. In other embodiments, the sugar is sucrose. In one embodiment, the sugar is starch. In certain embodiments, the carbon source is glycerol. In some embodiments, the carbon source is crude glycerol. In one embodiment, the carbon source is crude glycerol without treatment. In other embodiments, the carbon source is glycerol and glucose. In another embodiment, the carbon source is methanol and glycerol. In one embodiment, the carbon source is carbon dioxide. In one embodiment, the carbon source is formate. In one embodiment, the carbon source is methane. In one embodiment, the carbon source is methanol. In certain embodiments, methanol is used alone as the sole source of carbon or in combination with other carbon sources described herein or known in the art. In a specific embodiment, the methanol is the only (sole) carbon source. In one embodiment, the carbon source is chemoelectro-generated carbon (see, e.g., Liao et al. (2012) Science 335:1596). In one embodiment, the chemoelectro-generated carbon is methanol. In one embodiment, the chemoelectro-generated carbon is formate. In one embodiment, the chemoelectro-generated carbon is formate and methanol. In one embodiment, the carbon source is a carbohydrate and methanol. In one embodiment, the carbon source is a sugar and methanol. In another embodiment, the carbon source is a sugar and glycerol. In other embodiments, the carbon source is a sugar and crude glycerol. In yet other embodiments, the carbon source is a sugar and crude glycerol without treatment. In one embodiment, the carbon source is a sugar-containing biomass and methanol. In another embodiment, the carbon source is a sugar-containing biomass and glycerol. In other embodiments, the carbon source is a sugar-containing biomass and crude glycerol. In yet other embodiments, the carbon source is a sugar-containing biomass and crude glycerol without treatment. In some embodiments, the carbon source is a sugar-containing biomass, methanol and a carbohydrate. Other sources of carbohydrate include, for example, renewable feedstocks and biomass. Exemplary types of biomasses that can be used as feedstocks in the methods provided herein include cellulosic biomass, hemicellulosic biomass and lignin feedstocks or portions of feedstocks. Such biomass feedstocks contain, for example, carbohydrate substrates useful as carbon sources such as glucose, xylose, arabinose, galactose, mannose, fructose and starch. Given the teachings and guidance provided herein, those skilled in the art will understand that renewable feedstocks and biomass other than those exemplified above also can be used for culturing the microbial organisms provided herein for the production of succinate and other pathway intermediates.

The non-naturally occurring microbial organisms described herein are constructed using methods well known in the art as exemplified herein to express a recombinant nucleic acid and/or one or more nucleic acids encoding an engineered formate dehydrogenase or a bioderived compound pathway enzyme or protein in sufficient amounts to produce NADH or a bioderived compound. It is understood that the microbial organisms described herein are cultured under conditions sufficient to produce NADH or a bioderived compound. Following the teachings and guidance provided herein, the non-naturally occurring microbial organisms described herein can achieve biosynthesis of NADH or a bioderived compound resulting in intracellular concentrations between about 0.1-200 mM or more. Generally, the intracellular concentration of NADH or a bioderived compound is between about 3-150 mM, particularly between about 5-125 mM and more particularly between about 8-100 mM, including about 10 mM, 20 mM, 50 mM, 80 mM, or more. Intracellular concentrations between and above each of these exemplary ranges also can be achieved from the non-naturally occurring microbial organisms described herein.

In some embodiments, culture conditions include anaerobic or substantially anaerobic growth or maintenance conditions. Exemplary anaerobic conditions have been described previously and are well known in the art. Exemplary anaerobic conditions for fermentation processes are described herein and are described, for example, in U.S. publication 2009/0047719, filed Aug. 10, 2007. Any of these conditions can be employed with the non-naturally occurring microbial organisms as well as other anaerobic conditions well known in the art. Under such anaerobic or substantially anaerobic conditions, the NADH or the bioderived compound producers can synthesize NADH or a bioderived compound at intracellular concentrations of 5-10 mM or more as well as all other concentrations exemplified herein. It is understood that, even though the above description refers to intracellular concentrations, a bioderived compound producing microbial organisms can produce a bioderived compound intracellularly and/or secrete the product into the culture medium.

Exemplary fermentation processes include, but are not limited to, fed-batch fermentation and batch separation; fed-batch fermentation and continuous separation; and continuous fermentation and continuous separation. In an exemplary batch fermentation protocol, the production organism is grown in a suitably sized bioreactor sparged with an appropriate gas. Under anaerobic conditions, the culture is sparged with an inert gas or combination of gases, for example, nitrogen, N₂/CO₂ mixture, argon, helium, and the like. As the cells grow and utilize the carbon source, additional carbon source(s) and/or other nutrients are fed into the bioreactor at a rate approximately balancing consumption of the carbon source and/or nutrients. The temperature of the bioreactor is maintained at a desired temperature, generally in the range of 22-37 degrees C., but the temperature can be maintained at a higher or lower temperature depending on the growth characteristics of the production organism and/or desired conditions for the fermentation process. Growth continues for a desired period of time to achieve desired characteristics of the culture in the fermenter, for example, cell density, product concentration, and the like. In a batch fermentation process, the time period for the fermentation is generally in the range of several hours to several days, for example, 8 to 24 hours, or 1, 2, 3, 4 or 5 days, or up to a week, depending on the desired culture conditions. The pH can be controlled or not, as desired, in which case a culture in which pH is not controlled will typically decrease to pH 3-6 by the end of the run. Upon completion of the cultivation period, the fermenter contents can be passed through a cell separation unit, for example, a centrifuge, filtration unit, and the like, to remove cells and cell debris. In the case where the desired product is expressed intracellularly, the cells can be lysed or disrupted enzymatically or chemically prior to or after separation of cells from the fermentation broth, as desired, in order to release additional product. The fermentation broth can be transferred to a product separations unit. Isolation of product occurs by standard separations procedures employed in the art to separate a desired product from dilute aqueous solutions. Such methods include, but are not limited to, liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like) to provide an organic solution of the product, if appropriate, standard distillation methods, and the like, depending on the chemical characteristics of the product of the fermentation process.

In an exemplary fully continuous fermentation protocol, the production organism is generally first grown up in batch mode in order to achieve a desired cell density. When the carbon source and/or other nutrients are exhausted, feed medium of the same composition is supplied continuously at a desired rate, and fermentation liquid is withdrawn at the same rate. Under such conditions, the product concentration in the bioreactor generally remains constant, as well as the cell density. The temperature of the fermenter is maintained at a desired temperature, as discussed above. During the continuous fermentation phase, it is generally desirable to maintain a suitable pH range for optimized production. The pH can be monitored and maintained using routine methods, including the addition of suitable acids or bases to maintain a desired pH range. The bioreactor is operated continuously for extended periods of time, generally at least one week to several weeks and up to one month, or longer, as appropriate and desired. The fermentation liquid and/or culture is monitored periodically, including sampling up to every day, as desired, to assure consistency of product concentration and/or cell density. In continuous mode, fermenter contents are constantly removed as new feed medium is supplied. The exit stream, containing cells, medium, and product, are generally subjected to a continuous product separations procedure, with or without removing cells and cell debris, as desired. Continuous separations methods employed in the art can be used to separate the product from dilute aqueous solutions, including but not limited to continuous liquid-liquid extraction using a water immiscible organic solvent (e.g., toluene or other suitable solvents, including but not limited to diethyl ether, ethyl acetate, tetrahydrofuran (THF), methylene chloride, chloroform, benzene, pentane, hexane, heptane, petroleum ether, methyl tertiary butyl ether (MTBE), dioxane, dimethylformamide (DMF), dimethyl sulfoxide (DMSO), and the like), standard continuous distillation methods, and the like, or other methods well known in the art.

The culture conditions can include, for example, liquid culture procedures as well as fermentation and other large scale culture procedures. As described herein, particularly useful yields of the biosynthetic products described herein can be obtained under anaerobic or substantially anaerobic culture conditions.

As described herein, one exemplary growth condition for achieving biosynthesis of NADH or a bioderived compound includes anaerobic culture or fermentation conditions. In certain embodiments, the non-naturally occurring microbial organisms described herein can be sustained, cultured or fermented under anaerobic or substantially anaerobic conditions. Briefly, an anaerobic condition refers to an environment devoid of oxygen. Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions described herein can be scaled up and grown continuously for manufacturing of NADH or a bioderived compound. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of a bioderived compound. Generally, and as with non-continuous culture procedures, the continuous and/or near-continuous production of NADH or a bioderived compound will include culturing a non-naturally occurring NADH or a bioderived compound producing organism described herein in sufficient nutrients and medium to sustain and/or nearly sustain growth in an exponential phase. Continuous culture under such conditions can include, for example, growth or culturing for 1 day, 2, 3, 4, 5, 6 or 7 days or more. Additionally, continuous culture can include longer time periods of 1 week, 2, 3, 4 or 5 or more weeks and up to several months. Alternatively, organisms described herein can be cultured for hours, if suitable for a particular application. It is to be understood that the continuous and/or near-continuous culture conditions also can include all time intervals in between these exemplary periods. It is further understood that the time of culturing the microbial organism described herein is for a sufficient period of time to produce a sufficient amount of product for a desired purpose.

Fermentation procedures are well known in the art. Briefly, fermentation for the biosynthetic production of NADH or a bioderived compound can be utilized in, for example, fed-batch fermentation and batch separation; fed-batch fermentation and continuous separation, or continuous fermentation and continuous separation. Examples of batch and continuous fermentation procedures are well known in the art.

In addition to the above fermentation procedures using the NADH or bioderived compound producers described herein for continuous production of substantial quantities of a bioderived compound, the NADH or bioderived compound producers also can be, for example, simultaneously subjected to chemical synthesis and/or enzymatic procedures to convert the product to other compounds or the product can be separated from the fermentation culture and sequentially subjected to chemical and/or enzymatic conversion to convert the product to other compounds, if desired.

In some embodiments, the carbon feedstock and other cellular uptake sources such as phosphate, ammonia, sulfate, chloride and other halogens can be chosen to alter the isotopic distribution of the atoms present in a bioderived compound or any bioderived compound pathway intermediate. The various carbon feedstock and other uptake sources enumerated above will be referred to herein, collectively, as “uptake sources.” Uptake sources can provide isotopic enrichment for any atom present in the bioderived compound or pathway intermediate, or for side products generated in reactions diverging away from a bioderived compound pathway. Isotopic enrichment can be achieved for any target atom including, for example, carbon, hydrogen, oxygen, nitrogen, sulfur, phosphorus, chloride or other halogens.

In some embodiments, the uptake sources can be selected to alter the carbon-12, carbon-13, and carbon-14 ratios. In some embodiments, the uptake sources can be selected to alter the oxygen-16, oxygen-17, and oxygen-18 ratios. In some embodiments, the uptake sources can be selected to alter the hydrogen, deuterium, and tritium ratios. In some embodiments, the uptake sources can be selected to alter the nitrogen-14 and nitrogen-15 ratios. In some embodiments, the uptake sources can be selected to alter the sulfur-32, sulfur-33, sulfur-34, and sulfur-35 ratios. In some embodiments, the uptake sources can be selected to alter the phosphorus-31, phosphorus-32, and phosphorus-33 ratios. In some embodiments, the uptake sources can be selected to alter the chlorine-35, chlorine-36, and chlorine-37 ratios.

In some embodiments, the isotopic ratio of a target atom can be varied to a desired ratio by selecting one or more uptake sources. An uptake source can be derived from a natural source, as found in nature, or from a man-made source, and one skilled in the art can select a natural source, a man-made source, or a combination thereof, to achieve a desired isotopic ratio of a target atom. An example of a man-made uptake source includes, for example, an uptake source that is at least partially derived from a chemical synthetic reaction. Such isotopically enriched uptake sources can be purchased commercially or prepared in the laboratory and/or optionally mixed with a natural source of the uptake source to achieve a desired isotopic ratio. In some embodiments, a target atom isotopic ratio of an uptake source can be achieved by selecting a desired origin of the uptake source as found in nature. For example, as discussed herein, a natural source can be a biobased derived from or synthesized by a biological organism or a source such as petroleum-based products or the atmosphere. In some such embodiments, a source of carbon, for example, can be selected from a fossil fuel-derived carbon source, which can be relatively depleted of carbon-14, or an environmental or atmospheric carbon source, such as CO₂, which can possess a larger amount of carbon-14 than its petroleum-derived counterpart.

The unstable carbon isotope carbon-14 or radiocarbon makes up for roughly 1 in 10¹² carbon atoms in the earth's atmosphere and has a half-life of about 5700 years. The stock of carbon is replenished in the upper atmosphere by a nuclear reaction involving cosmic rays and ordinary nitrogen (¹⁴N). Fossil fuels contain no carbon-14, as it decayed long ago. Burning of fossil fuels lowers the atmospheric carbon-14 fraction, the so-called “Suess effect”.

Methods of determining the isotopic ratios of atoms in a compound are well known to those skilled in the art. Isotopic enrichment is readily assessed by mass spectrometry using techniques known in the art such as accelerated mass spectrometry (AMS), Stable Isotope Ratio Mass Spectrometry (SIRMS) and Site-Specific Natural Isotopic Fractionation by Nuclear Magnetic Resonance (SNIF-NMR). Such mass spectral techniques can be integrated with separation techniques such as liquid chromatography (LC), high performance liquid chromatography (HPLC) and/or gas chromatography, and the like.

Accordingly, in some embodiments, provided herein is a bioderived compound or a bioderived compound pathway intermediate that has a carbon-12, carbon-13, and carbon-14 ratio that reflects an atmospheric carbon, also referred to as environmental carbon, uptake source. For example, in some aspects the bioderived compound or bioderived compound pathway intermediate can have an Fm value of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98% or as much as 100%. In some such embodiments, the uptake source is CO₂. In some embodiments, provided herein is a bioderived compound or a bioderived compound pathway intermediate that has a carbon-12, carbon-13, and carbon-14 ratio that reflects petroleum-based carbon uptake source. In this aspect, the bioderived compound or a bioderived compound pathway intermediate can have an Fm value of less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2% or less than 1%. In some embodiments, provided herein is an a bioderived compound or a bioderived compound pathway intermediate that has a carbon-12, carbon-13, and carbon-14 ratio that is obtained by a combination of an atmospheric carbon uptake source with a petroleum-based uptake source. Using such a combination of uptake sources is one way by which the carbon-12, carbon-13, and carbon-14 ratio can be varied, and the respective ratios would reflect the proportions of the uptake sources.

Further, the present disclosure relates to the biologically produced bioderived compound or pathway intermediate as disclosed herein, and to the products derived therefrom, wherein the bioderived compound or a bioderived compound pathway intermediate has a carbon-12, carbon-13, and carbon-14 isotope ratio of about the same value as the CO₂ that occurs in the environment. For example, in some aspects provided herein is a bioderived compound or a bioderived compound intermediate having a carbon-12 versus carbon-13 versus carbon-14 isotope ratio of about the same value as the CO₂ that occurs in the environment, or any of the other ratios disclosed herein. It is understood, as disclosed herein, that a product can have a carbon-12 versus carbon-13 versus carbon-14 isotope ratio of about the same value as the CO₂ that occurs in the environment, or any of the ratios disclosed herein, wherein the product is generated from a bioderived compound or a bioderived compound pathway intermediate as disclosed herein, wherein the bioderived product is chemically modified to generate a final product. Methods of chemically modifying a bioderived product of a bioderived compound, or an intermediate thereof, to generate a desired product are well known to those skilled in the art, as described herein. The disclosure further provides biobased products having a carbon-12 versus carbon-13 versus carbon-14 isotope ratio of about the same value as the CO₂ that occurs in the environment, wherein the biobased products are generated directly from or in combination with bioderived compound or a bioderived compound pathway intermediate as disclosed herein.

The disclosure further provides a composition comprising bioderived compound described herein and a compound other than the bioderived compound. The compound other than the bioderived product can be a cellular portion, for example, a trace amount of a cellular portion of, or can be fermentation broth or culture medium, or a purified or partially purified fraction thereof produced in the presence of, a non-naturally occurring microbial organism described herein. The composition can comprise, for example, a reduced level of a byproduct when produced by an organism having reduced byproduct formation, as disclosed herein. The composition can comprise, for example, bioderived compound, or a cell lysate or culture supernatant of a microbial organism described herein.

The disclosure further provides a method for increasing the availability of NADH in a non-naturally occurring microbial organism. Such a method, in some embodiments, includes culturing a non-naturally occurring microbial organism described herein (e.g., having a recombinant nucleic acid encoding an engineered formate dehydrogenase described herein) under conditions and for a sufficient period of time to increase the availability of NADH. It is understood that a person skilled in the art, using methods well known in the art or those described herein, can readily determine the culturing conditions and period of time needed for increasing the availability of NADH in the non-naturally occurring microbial organism. In some embodiments, such a method also includes introducing (e.g., transducing or integrating into the genome of the microbial organism) into the non-naturally occurring microbial organism a recombinant nucleic acid encoding an engineered formate dehydrogenase described herein.

Such a method for increasing the availability of NADH in a non-naturally occurring microbial organism, in some embodiments, results in at least 10% more NADH compared to culturing the same microbial organism absent the recombinant nucleic acid encoding an engineered formate dehydrogenase described herein. In some embodiments, the method results in at least 20% more NADH compared to culturing the same microbial organism absent the recombinant nucleic acid encoding an engineered formate dehydrogenase described herein. In some embodiments, the method results in at least 30% more NADH compared to culturing the same microbial organism absent the recombinant nucleic acid encoding an engineered formate dehydrogenase described herein. In some embodiments, the method results in at least 40% more NADH compared to culturing the same microbial organism absent the recombinant nucleic acid encoding an engineered formate dehydrogenase described herein. In some embodiments, the method results in at least 50% more NADH compared to culturing the same microbial organism absent the recombinant nucleic acid encoding an engineered formate dehydrogenase described herein. In some embodiments, the method results in at least 60% more NADH compared to culturing the same microbial organism absent the recombinant nucleic acid encoding an engineered formate dehydrogenase described herein. In some embodiments, the method results in at least 70% more NADH compared to culturing the same microbial organism absent the recombinant nucleic acid encoding an engineered formate dehydrogenase described herein. In some embodiments, the method results in at least 80% more NADH compared to culturing the same microbial organism absent the recombinant nucleic acid encoding an engineered formate dehydrogenase described herein. In some embodiments, the method results in at least 90% more NADH compared to culturing the same microbial organism absent the recombinant nucleic acid encoding an engineered formate dehydrogenase described herein. In some embodiments, the method results in at least 1 fold more NADH compared to culturing the same microbial organism absent the recombinant nucleic acid encoding an engineered formate dehydrogenase described herein. In some embodiments, the method results in at least 1.1 fold more NADH compared to culturing the same microbial organism absent the recombinant nucleic acid encoding an engineered formate dehydrogenase described herein. In some embodiments, the method results in at least 1.2 fold more NADH compared to culturing the same microbial organism absent the recombinant nucleic acid encoding an engineered formate dehydrogenase described herein. In some embodiments, the method results in at least 1.3 fold more NADH compared to culturing the same microbial organism absent the recombinant nucleic acid encoding an engineered formate dehydrogenase described herein. In some embodiments, the method results in at least 1.4 fold more NADH compared to culturing the same microbial organism absent the recombinant nucleic acid encoding an engineered formate dehydrogenase described herein. In some embodiments, the method results in at least 1.5 fold more NADH compared to culturing the same microbial organism absent the recombinant nucleic acid encoding an engineered formate dehydrogenase described herein. In some embodiments, the method results in at least 1.6 fold more NADH compared to culturing the same microbial organism absent the recombinant nucleic acid encoding an engineered formate dehydrogenase described herein. In some embodiments, the method results in at least 1.7 fold more NADH compared to culturing the same microbial organism absent the recombinant nucleic acid encoding an engineered formate dehydrogenase described herein. In some embodiments, the method results in at least 1.8 fold more NADH compared to culturing the same microbial organism absent the recombinant nucleic acid encoding an engineered formate dehydrogenase described herein. In some embodiments, the method results in at least 1.9 fold more NADH compared to culturing the same microbial organism absent the recombinant nucleic acid encoding an engineered formate dehydrogenase described herein. In some embodiments, the method results in at least 2 fold more NADH compared to culturing the same microbial organism absent the recombinant nucleic acid encoding an engineered formate dehydrogenase described herein.

In some embodiments, the method for increasing the availability of NADH in a non-naturally occurring microbial organism yields an increase in production of a bioderived compound described herein, wherein the non-naturally occurring microbial organism includes a pathway capable of producing a bioderived compound as described herein. Such a pathway, in some embodiments, would benefit directly or indirectly from the production of cofactors as described herein. Such a method, in some embodiments, yields an increase of at least 10% more bioderived compound compared to culturing the same microbial organism absent the recombinant nucleic acid encoding an engineered formate dehydrogenase described herein. In some embodiments, the method yields an increase of at least 20% more bioderived compound compared to culturing the same microbial organism absent the recombinant nucleic acid encoding an engineered formate dehydrogenase described herein. In some embodiments, the method yields an increase of at least 30% more bioderived compound compared to culturing the same microbial organism absent the recombinant nucleic acid encoding an engineered formate dehydrogenase described herein. In some embodiments, the method yields an increase of at least 40% more bioderived compound compared to culturing the same microbial organism absent the recombinant nucleic acid encoding an engineered formate dehydrogenase described herein. In some embodiments, the method yields an increase of at least 50% more bioderived compound compared to culturing the same microbial organism absent the recombinant nucleic acid encoding an engineered formate dehydrogenase described herein. In some embodiments, the method yields an increase of at least 60% more bioderived compound compared to culturing the same microbial organism absent the recombinant nucleic acid encoding an engineered formate dehydrogenase described herein. In some embodiments, the method yields an increase of at least 70% more bioderived compound compared to culturing the same microbial organism absent the recombinant nucleic acid encoding an engineered formate dehydrogenase described herein. In some embodiments, the method yields an increase of at least 80% more bioderived compound compared to culturing the same microbial organism absent the recombinant nucleic acid encoding an engineered formate dehydrogenase described herein. In some embodiments, the method yields an increase of at least 90% more bioderived compound compared to culturing the same microbial organism absent the recombinant nucleic acid encoding an engineered formate dehydrogenase described herein. In some embodiments, the method yields an increase of at least 1 fold more bioderived compound compared to culturing the same microbial organism absent the recombinant nucleic acid encoding an engineered formate dehydrogenase described herein. In some embodiments, the method yields an increase of at least 1.1 fold more bioderived compound compared to culturing the same microbial organism absent the recombinant nucleic acid encoding an engineered formate dehydrogenase described herein. In some embodiments, the method yields an increase of at least 1.2 fold more bioderived compound compared to culturing the same microbial organism absent the recombinant nucleic acid encoding an engineered formate dehydrogenase described herein. In some embodiments, the method yields an increase of at least 1.3 fold more bioderived compound compared to culturing the same microbial organism absent the recombinant nucleic acid encoding an engineered formate dehydrogenase described herein. In some embodiments, the method yields an increase of at least 1.4 fold more bioderived compound compared to culturing the same microbial organism absent the recombinant nucleic acid encoding an engineered formate dehydrogenase described herein. In some embodiments, the method yields an increase of at least 1.5 fold more bioderived compound compared to culturing the same microbial organism absent the recombinant nucleic acid encoding an engineered formate dehydrogenase described herein. In some embodiments, the method yields an increase of at least 1.6 fold more bioderived compound compared to culturing the same microbial organism absent the recombinant nucleic acid encoding an engineered formate dehydrogenase described herein. In some embodiments, the method yields an increase of at least 1.7 fold more bioderived compound compared to culturing the same microbial organism absent the recombinant nucleic acid encoding an engineered formate dehydrogenase described herein. In some embodiments, the method yields an increase of at least 1.8 fold more bioderived compound compared to culturing the same microbial organism absent the recombinant nucleic acid encoding an engineered formate dehydrogenase described herein. In some embodiments, the method yields an increase of at least 1.9 fold more bioderived compound compared to culturing the same microbial organism absent the recombinant nucleic acid encoding an engineered formate dehydrogenase described herein. In some embodiments, the method yields an increase of at least 2 fold more bioderived compound compared to culturing the same microbial organism absent the recombinant nucleic acid encoding an engineered formate dehydrogenase described herein.

The disclosure further provides a method for decreasing formate concentration in a non-naturally occurring microbial organism. Such a method, in some embodiments, includes culturing a non-naturally occurring microbial organism described herein (e.g., having a recombinant nucleic acid encoding an engineered formate dehydrogenase described herein) under conditions and for a sufficient period of time to increase the conversion of formate to carbon dioxide, thereby increasing carbon dioxide produced by the microbial organism and decreasing the formate concentration in the microbial organism. It is understood that a person skilled in the art, using methods well known in the art or those described herein, can readily determine the culturing conditions and period of time needed for increasing the conversion of formate to carbon dioxide in the non-naturally occurring microbial organism. In some embodiments, such a method also includes introducing (e.g., transducing or integrating into the genome of the microbial organism) into the non-naturally occurring microbial organism a recombinant nucleic acid encoding an engineered formate dehydrogenase described herein.

Such a method for decreasing formate concentration in a non-naturally occurring microbial organism, in some embodiments, results in at least 10% more carbon dioxide compared to culturing the same microbial organism absent the recombinant nucleic acid encoding an engineered formate dehydrogenase described herein. In some embodiments, the method results in at least 20% more carbon dioxide compared to culturing the same microbial organism absent the recombinant nucleic acid encoding an engineered formate dehydrogenase described herein. In some embodiments, the method results in at least 30% more carbon dioxide compared to culturing the same microbial organism absent the recombinant nucleic acid encoding an engineered formate dehydrogenase described herein. In some embodiments, the method results in at least 40% more carbon dioxide compared to culturing the same microbial organism absent the recombinant nucleic acid encoding an engineered formate dehydrogenase described herein. In some embodiments, the method results in at least 50% more carbon dioxide compared to culturing the same microbial organism absent the recombinant nucleic acid encoding an engineered formate dehydrogenase described herein. In some embodiments, the method results in at least 60% more carbon dioxide compared to culturing the same microbial organism absent the recombinant nucleic acid encoding an engineered formate dehydrogenase described herein. In some embodiments, the method results in at least 70% more carbon dioxide compared to culturing the same microbial organism absent the recombinant nucleic acid encoding an engineered formate dehydrogenase described herein. In some embodiments, the method results in at least 80% more carbon dioxide compared to culturing the same microbial organism absent the recombinant nucleic acid encoding an engineered formate dehydrogenase described herein. In some embodiments, the method results in at least 90% more carbon dioxide compared to culturing the same microbial organism absent the recombinant nucleic acid encoding an engineered formate dehydrogenase described herein. In some embodiments, the method results in at least 1 fold more carbon dioxide compared to culturing the same microbial organism absent the recombinant nucleic acid encoding an engineered formate dehydrogenase described herein. In some embodiments, the method results in at least 1.1 fold more carbon dioxide compared to culturing the same microbial organism absent the recombinant nucleic acid encoding an engineered formate dehydrogenase described herein. In some embodiments, the method results in at least 1.2 fold more carbon dioxide compared to culturing the same microbial organism absent the recombinant nucleic acid encoding an engineered formate dehydrogenase described herein. In some embodiments, the method results in at least 1.3 fold more carbon dioxide compared to culturing the same microbial organism absent the recombinant nucleic acid encoding an engineered formate dehydrogenase described herein. In some embodiments, the method results in at least 1.4 fold more carbon dioxide compared to culturing the same microbial organism absent the recombinant nucleic acid encoding an engineered formate dehydrogenase described herein. In some embodiments, the method results in at least 1.5 fold more carbon dioxide compared to culturing the same microbial organism absent the recombinant nucleic acid encoding an engineered formate dehydrogenase described herein. In some embodiments, the method results in at least 1.6 fold more carbon dioxide compared to culturing the same microbial organism absent the recombinant nucleic acid encoding an engineered formate dehydrogenase described herein. In some embodiments, the method results in at least 1.7 fold more carbon dioxide compared to culturing the same microbial organism absent the recombinant nucleic acid encoding an engineered formate dehydrogenase described herein. In some embodiments, the method results in at least 1.8 fold more carbon dioxide compared to culturing the same microbial organism absent the recombinant nucleic acid encoding an engineered formate dehydrogenase described herein. In some embodiments, the method results in at least 1.9 fold more carbon dioxide compared to culturing the same microbial organism absent the recombinant nucleic acid encoding an engineered formate dehydrogenase described herein. In some embodiments, the method results in at least 2 fold more carbon dioxide compared to culturing the same microbial organism absent the recombinant nucleic acid encoding an engineered formate dehydrogenase described herein.

In some embodiments, the method for decreasing formate concentration in a non-naturally occurring microbial organism yields a decrease in formate as an impurity in a method for production of the bioderived compound described herein, wherein the method for production of the bioderived compound includes culturing a non-naturally occurring microbial organism having a pathway capable of producing a bioderived compound as described herein. Such a method, in some embodiments, yields a decrease of formate as an impurity by at least 10% compared to culturing the same microbial organism absent the recombinant nucleic acid encoding an engineered formate dehydrogenase described herein. In some embodiments, the method yields a decrease of formate as an impurity by at least 20% compared to culturing the same microbial organism absent the recombinant nucleic acid encoding an engineered formate dehydrogenase described herein. In some embodiments, the method yields a decrease of formate as an impurity by at least 30% compared to culturing the same microbial organism absent the recombinant nucleic acid encoding an engineered formate dehydrogenase described herein. In some embodiments, the method yields a decrease of formate as an impurity by at least 40% compared to culturing the same microbial organism absent the recombinant nucleic acid encoding an engineered formate dehydrogenase described herein. In some embodiments, the method yields a decrease of formate as an impurity by at least 50% compared to culturing the same microbial organism absent the recombinant nucleic acid encoding an engineered formate dehydrogenase described herein. In some embodiments, the method yields a decrease of formate as an impurity by at least 60% compared to culturing the same microbial organism absent the recombinant nucleic acid encoding an engineered formate dehydrogenase described herein. In some embodiments, the method yields a decrease of formate as an impurity by at least 70% compared to culturing the same microbial organism absent the recombinant nucleic acid encoding an engineered formate dehydrogenase described herein. In some embodiments, the method yields a decrease of formate as an impurity by at least 80% compared to culturing the same microbial organism absent the recombinant nucleic acid encoding an engineered formate dehydrogenase described herein. In some embodiments, the method yields a decrease of formate as an impurity by at least 90% compared to culturing the same microbial organism absent the recombinant nucleic acid encoding an engineered formate dehydrogenase described herein. In some embodiments, the method yields a decrease of formate as an impurity by at least 1 fold compared to culturing the same microbial organism absent the recombinant nucleic acid encoding an engineered formate dehydrogenase described herein. In some embodiments, the method yields a decrease of formate as an impurity by at least 1.1 fold compared to culturing the same microbial organism absent the recombinant nucleic acid encoding an engineered formate dehydrogenase described herein. In some embodiments, the method yields a decrease of formate as an impurity by at least 1.2 fold compared to culturing the same microbial organism absent the recombinant nucleic acid encoding an engineered formate dehydrogenase described herein. In some embodiments, the method yields a decrease of formate as an impurity by at least 1.3 fold compared to culturing the same microbial organism absent the recombinant nucleic acid encoding an engineered formate dehydrogenase described herein. In some embodiments, the method yields a decrease of formate as an impurity by at least 1.4 fold compared to culturing the same microbial organism absent the recombinant nucleic acid encoding an engineered formate dehydrogenase described herein. In some embodiments, the method yields a decrease of formate as an impurity by at least 1.5 fold compared to culturing the same microbial organism absent the recombinant nucleic acid encoding an engineered formate dehydrogenase described herein. In some embodiments, the method yields a decrease of formate as an impurity by at least 1.6 fold compared to culturing the same microbial organism absent the recombinant nucleic acid encoding an engineered formate dehydrogenase described herein. In some embodiments, the method yields a decrease of formate as an impurity by at least 1.7 fold compared to culturing the same microbial organism absent the recombinant nucleic acid encoding an engineered formate dehydrogenase described herein. In some embodiments, the method yields a decrease of formate as an impurity by at least 1.8 fold compared to culturing the same microbial organism absent the recombinant nucleic acid encoding an engineered formate dehydrogenase described herein. In some embodiments, the method yields a decrease of formate as an impurity by at least 1.9 fold compared to culturing the same microbial organism absent the recombinant nucleic acid encoding an engineered formate dehydrogenase described herein. In some embodiments, the method yields a decrease of formate as an impurity by at least 2 fold compared to culturing the same microbial organism absent the recombinant nucleic acid encoding an engineered formate dehydrogenase described herein.

In certain embodiments, provided herein is a composition comprising a bioderived compound provided herein produced by culturing a non-naturally occurring microbial organism described herein. In some embodiments, the composition further comprises a compound other than said bioderived compound. In certain embodiments, the compound other than said bioderived compound is a trace amount of a cellular portion of a non-naturally occurring microbial organism described herein.

### SEQUENCES

The sequences in the following TABLE 1 illustrate amino acid sequences that can be used to generate the FDH sequences and/or compositions and perform the methods described herein. As needed, an RNA sequence can be readily deduced from the DNA sequence.

TABLE 1
Sequences

| SEQ | | | UniProt | |
|---|---|---|---|---|
| ID | | | ID/ENA | |
| NO: | Enzyme/Gene | Organism | Accession | Sequence |
| 1 | Formate | Gibbsiella | A0A250B5N7 | MSKILCVLYPDPVTGFPPVYARDSIPDIRCYPNGQT |
| | dehydrogenase | quercinecans | | APTPAGELGFTPGELVGSVSGELGLRRYLESQGHEL |
| | | | | IVTSDKEGADSVFERHLPDADVVISQPFWPAYLTAE |
| | | | | RIAKAKRLKLALTAGIGSDHVDLKAAAERGITVAEV |
| | | | | TFSNSISVAEHVVMTVLALVRNYLPAHQIANNGGWN |
| | | | | IADCVARSYDVEGMHFGTLGAGRIGLAVLRRLKPFD |
| | | | | MPLHYYDPHRLSAECEKELGLIYHDTPESLVSVCDV |
| | | | | VNLQTPLYPATQGFVNAAFLAKFKRGAYLINTARGA |
| | | | | LCERDAIVHALETGQLAGYGGDVWFPQPAPIDHPWR |
| | | | | RMPHQGMTPHISGTSLSGQARYAAGTLEILQNFLEG |
| | | | | KPIRQEYLIVDGGNLAGAGANAYKLA |
| 2 | Formate | Candida | O13437 | MKIVLVLYDAGKHAADEEKLYGCTENKLGIANWLKD |
| | dehydrogenase | boidinii | | QGHELITTSDKEGETSELDKHIPDADIIITTPFHPA |
| | | | | YITKERLDKAKNLKLVVVAGVGSDHIDLDYINQTGK |
| | | | | KISVLEVTGSNVVSVAEHVVMTMLVLVRNFVPAHEQ |
| | | | | IINHDWEVAAIAKDAYDIEGKTIATIGAGRIGYRVL |
| | | | | ERLLPFNPKELLYYDYQALPKEAEEKVGARRVENIE |
| | | | | ELVAQADIVTVNAPLHAGTKGLINKELLSKFKKGAW |
| | | | | LVNTARGAICVAEDVAAALESGQLRGYGGDVWFPQP |
| | | | | APKDHPWRDMRNKYGAGNAMTPHYSGTTLDAQTRYA |
| | | | | EGTKNILESFFTGKFDYRPQDIILLNGEYVTKAYGK |
| | | | | HDKK |
| 3 | Formate | Candida | A0A0AIEQY0 | MKIVLVLYDAGKHAADEEKLYGCTENKLGIANWLKD |
| | dehydrogenase | boidinii | | QGHELITTSDKEGGNSVLDQHIPDADIIITTPFHPA |
| | | | | YITKERIDKAKKLKLVVVAGVGSDHIDLDYINQTGK |
| | | | | KISVLEVTGSNVVSVAEHVLMTMLVLVRNFVPAHEQ |
| | | | | IINHDWEVAAIAKDAYDIEGKTIATIGAGRIGYRVL |
| | | | | ERLVPFNPKELLYYDYQALPKDAEEKVGARRVENIE |
| | | | | ELVAQADIVTINAPLHAGTKGLINKELLSKFKKGAW |
| | | | | LVNTARGAICVAEDVAAALESGQLRGYGGDVWFPQP |
| | | | | APKDHPWRDMRNKYGAGNAMTPHYSGTTLDAQTRYA |
| | | | | EGTKNILESFFTGKFDYRPQDIILLNGEYITKAYGK |
| | | | | HDKK |
| 4 | Formate | Clohesyomyces | A0A1Y1ZA31 | MVFLRSISRVARPASSMLSARAAPKASFGASFFRKP |
| | dehydrogenase | aquaticus | | NAYRTLTATSTQQGKVLLVLYDGGVHAEQEPQLLGT |
| | | | | TENELGIRKWLEDAGHTLITTSDKEGENSKFDQELV |
| | | | | DAEVIITTPYVEATPHYSSSVPRNRTALSPHHLATL |
| | | | | ALPFHPGYLTAERLAKAKKLKLAVTAGIGSDHIDLD |
| | | | | AANKTNGGITVAEVTGSNVVSVAEHVVMTILTLVRN |
| | | | | FVPAHEQIAAGEWDVAAVAKNEYDLENKVVGTVAVG |
| | | | | RIGERVLRRLKPFDCKELLYFDYQPLTAEKEKEIGC |
| | | | | RRVENLEEMLAQCDVVTINCPLHEKTRGLFNKDLIS |
| | | | | KMKKGSWLINTARGAIVVKEDVADALKSGHLRGYGG |
| | | | | DVWFPQPAPKEHPLRYATNPWGGGNAMVPHMSGTSI |
| | | | | DAQKRYAAGTKAILDSYFSGREDYKPEDLIVHQGDY |
| | | | | ATKAYGQREGKKHANA |
| 5 | Formate | Clavispora | C4Y770 | MSKPKVLLVLYEGKHHAKDEPKLYGCLENELGIRGF |
| | dehydrogenase | lusitaniae | | VESHGYELVSISEKDPIGDSEFDYHLADAEIVITTP |
| | | | | FFPAYLTRERIAEAPKLKLCITAGVGSDHVDLNAAN |
| | | | | ERKITVAEVTGSNVVSVAEHAVMTMLNLVRNFVPGH |
| | | | | EQAMSGGWDIAAIAKDEYDLEDKVIATVGAGRIGYR |
| | | | | ILERLVAFNPKKLLYFDYQDLPKEAVDKLNAASKLF |
| | | | | NGHDDIVERVEKLEDMLSRSDVVTINCPLHEGSKGL |
| | | | | FNKETISHMKDGAWLVNTARGAICVEKDVADAVESG |
| | | | | KLRGYGGDVWYPQPAPDHHPWRTFRNKYGGGNAMTP |
| | | | | HVSGTSLDAQERYAAGTQAILKSYFEKSFDYRPQDV |
| | | | | IVVDGEYATKAYGQRK |
| 6 | Formate | Caballeronia | A0A7H9UPL5 | MAKVLCVLYDDPVDGMPKKYARDDIPAISKYHDGQS |
| | dehydrogenase | sordidicola | | TPTPKAIDFKPGELLGSVSGELGLRKFLESRGHTLV |
| | | | | VTSDKDGPNSTFERELEDAEVVISQPFWPAYLTAER |
| | | | | IARAPKLKLALTAGIGSDHVDLQAAIDRGITVAEVT |
| | | | | FCNSISVAEHVVMMILSQVRNYLPSHEWVKKGGWNI |
| | | | | ADCVSRAYDLEGMNVGTVAAGRIGAAVLRRLKPFDV |
| | | | | KLHYTDRHRLPEQVERELGATWHPTAESMARVCDIV |
| | | | | TINCPLHPETEHLFDETMISKMKRGAYIINTARGKI |
| | | | | VDRDAVARALESGQLAGYAGDVWFPQPAPSDHPWRT |
| | | | | MPNQGMTPHTSGTTLSAQARYAAGTREILECWFAKQ |
| | | | | PIREEYLIVDGGRLAGVGAHSYSAGNATSGSEEAAR |
| | | | | FKKA |
| 7 | Formate | Coccidioides | A0A0J6ETL5 | MGKVLMVMYDGGEHAKQQPGLLGTTENELGLRKWLE |
| | dehydrogenase | posadasii | | ERGHTLVTTSDKEGSNSTFERELVDAEIIITTPFHP |
| | | RMSCC 3488 | | GYLTAERLAKAKNLKLAITAGVGSDHVDLNAANKTN |
| | | | | GGITVAEVTGCNVVSVAEHVVMTILVLVRNFVPAHQ |
| | | | | QVASGEWDVAAVAKNEYDLEGKVVGTVAVGRIGERV |
| | | | | LRRLKPFDCKELLYYDYQPLSPEVEKEIGCRRVENL |
| | | | | EEMLAQCDVVTINCPLHEKTRGLFNKELISKMKKGS |
| | | | | WLVNTARGAIVVKEDVAEAIKSGHLRGYGGDVWFPQ |
| | | | | PAPKDHPLRYVQGPWGGGNAMVPHMSGTSIDAQIRY |
| | | | | AQGTKNILESYFSGKFDYKQEDLIVHQGDYVTKAYG |
| | | | | QRKKIQEEQRN |
| 8 | Formate | Botryotinia | M7TQ78 | MVFFTRSLSRLATRPSPCARSIFTASAVARTTPTWT |
| | dehydrogenase | fuckeliana | | TRSTAFRSQKLNNIFGNVRMLTGNHREKVKVLLVLY |
| | | | | DGKKHAEEVPELLGTTENELGIRKWLEDQGHELITT |
| | | | | SDKEGENSEFDKHLVDAEIIITTPFHPGYLTAERLA |
| | | | | KAKNLKIAITAGIGSDHVDLNAANKTNGGITVAEVT |
| | | | | GSNVVSVAEHVVMTILVLVRNFVPAHEQIQAGEWDV |
| | | | | AAAAKNEFDLEGKVVGTVAVGRIGERVLRRLKPFDC |
| | | | | KELLYFDYQPLKPEVEQEIGCRRVTDLEEMLAQCDV |
| | | | | VTINCPLHEKTRGLFNKELISKMKKGSWLVNTARGA |
| | | | | IVVKEDVADALASGHLRGYGGDVWFPQPAPKDHPLR |
| | | | | YAKNPFGGGNAMVPHMSGTSLDAQKRYADGTKAILE |
| | | | | SYLSGKHDYRPEDLIVIGGDYATKAYGERAKNSGSA |
| | | | | GANKA |
| 9 | Formate | Yarrowia | Q6CDN8 | MKILLILYDAGSHAADEPKLLGCTENELGIRSWLES |
| | dehydrogenase | lipolytica | | QGHTLVTTSSKEGADSVLDKEIVDADVVITTPFHPG |
| | | | | YITRERIAKAKNLKICVTAGVGSDHVDLAAANERNI |
| | | | | AVLEVTGSNVTSVAEHVVMTMLVLVRNFVPANEQVR |
| | | | | GGGWDVAGVAKDSYDIEGKVIGTVGVGRIGKRVLQR |
| | | | | LKPFDPKELLYYDYQPLSAADEKEIGARRVEKLEDM |
| | | | | LAQCDVVTINCPLHESTKGLFNKELLSHMKKGAWLV |
| | | | | NTARGAICVKEDVAAALKSGQLRGYGGDVWFPQPAP |
| | | | | ADHPWRKMVNKYGAGNAMTPHMSGTSLDAQARYAAG |
| | | | | VKQILDEFFSGREQYRPQDIICYGGNYGTKAYGDDK |
| | | | | KVVDKK |
| 10 | Formate | Kushneria | A0A1I1JVK8 | MAKILCVLYPDPVTGYPPRYARDDIPEITGYPNGQT |
| | dehydrogenase | avicenniae | | APSPEGDPGFSPGELIGCVSGELGLRSWAQANGHEL |
| | | | | IVTSDKEGVDSVFERHLPDADVVISQPFWPAYLSAE |
| | | | | RIARAPKLKLALTAGIGSDHVDLKAAAEHGITVAEV |
| | | | | TFSNSISVAEHVVMTALTLVRNFVPSHQIAINGGWN |
| | | | | IADCVSRSYDIEGMQFGTLGAGRIGLAVLRRMKPFD |
| | | | | TPLHYYDPHRLSEDIECELGLTYHDSPESLVSVCDI |
| | | | | VNVQTPLYPSTEGFINDALLSKFKRGAYLVNTARGG |
| | | | | LCDREAVVRALESGQLAGYGGDVWYPQPAPVDHPWR |
| | | | | RMPNHAMTPHISGTSLSAQTRYAAGTLEILQNFLAG |
| | | | | KSLREEYLIVDAGNLAGAGANAYQL |
| 11 | Formate | Zea mays | A0A3L6EKZ5 | MVESTPLLVCLLDTNTKLEKHIEDMHVLITTPFHPA |
| | dehydrogenase | | | YVTAERIKKAKNLELLLTAGIGSDHIDLPAAAAAGL |
| | 1, | | | TVAEVTGSNTVSVAEDELLRILILLRNFLPGYQQVV |
| | mitochondrial | | | QGEWNVAGIAHRAYDLEGKTVGTVGAGRIGRLLLQR |
| | | | | LKPFNCNLLYHDRLQIDPELEKEIGAKFEEDLDAML |
| | | | | PKFDVIVINTPLTEKTRGMFNKERIAKMKKGVIVVN |
| | | | | NARGAIMDARAVVDACSSGHIAGYGGDVWFPQPAPK |
| | | | | DHPWRYMPNHAMTPHISRTTIDAQVRYADGVRDMLD |
| | | | | RYFKGEDFPVQNYIVKEGQLASQYK |
| 12 | Formate | Dactylellina | S8BZ87 | MVLRATSTLARCLAKPTLQASTLGARVVRPAYRQPL |
| | dehydrogenase | haptotyla | | TAIQTRKLTTTFQPQVKVLAVLYDGGKHAQDVPALL |
| | | | | GTTENELGIRKWLEDQGHTLVTTSDKEGENSKFDQE |
| | | | | LVDAEVIITTPFRPGYLTAERLAKAKKLKLAITAGI |
| | | | | GSDHVDLNAANKTNGGVTVAEVTGSNVVSVAEHVVM |
| | | | | TILILVRNFVPAHDQIRNGDWEVAAVAKDEYDLEDK |
| | | | | IVGTVGVGRIGERVLRRLKPFDCKELLYFDYQPLNA |
| | | | | AVEKEIGCRRVESLEEMVAQCDVVTINCPLHESTHG |
| | | | | LFNKNLISKMKKGSWLVNTARGAIVVKEDVAEAVKS |
| | | | | GHLRGYGGDVWFPQPAPKDHPLRYAVNPFGGGNAMV |
| | | | | PHMSGTSLDAQKRYADGTKKILESYLSGRHDYQPHD |
| | | | | LIVHKGDYATKAYGQRK |
| 13 | Formate | Segniliparus | E5XSQ8 | MAKIVAVLYPDPVDGYPTTYARDDIPVIASYPGGQS |
| | dehydrogenase | rugosus | | APTPSAIDFVPGQLLGSVTGELGLRKFLEAQGHTLV |
| | | | | VTSDKEGPDSALDRELADADVVISQPFWPAYLTAER |
| | | | | IAAAPNLKLAITAGIGSDHVDLNAAIARGITVAEVT |
| | | | | FSNSISVAEHAVMQILALVRNYLPSHAWVAEKRGWN |
| | | | | IADSVERAYDLEGMTVGVIAAGRIGLAVLRRLKPFD |
| | | | | VKLAYTDKHRLSAEVEEELGLTFYPDIPSLVRAVDI |
| | | | | VDVHAPLVPETHHLFDDALIGTMKRGSYIVNTARGA |
| | | | | IMDTGAVVRALESGQLAGYAGDVWYPQPPAPDHPWR |
| | | | | TMPWSGMTPHVSGTTLSAQARYAAGTREILESWLAG |
| | | | | APIRDEYLIVDGGKLAGTGARSYSTSGSGSSPGSSG |
| | | | | K |
| 14 | Formate | Aspergillus | A0A2V5IND1 | MVFMRSFSRQLRRPATSLLSSRGPLASAGSSPLRTS |
| | dehydrogenase | indologenus | | PLANSIAGARTLTASANLQGKILMVLYDGGEHAKQQ |
| | | CBS 114.80 | | PGLLGTTENELGLRKWLEDQGHTLVTTSDKEGENST |
| | | | | EDKELVDAEVIITTPFHPGYLTAERLAKAKNLKIAV |
| | | | | TAGVGSDHVDLNAANKTNGGITVAEVTGCNVVSVAE |
| | | | | HVVMTILTLVRNFVPAHEQIRNGEWDVAAVAKNEYD |
| | | | | LENKVVGTVAVGRIGERVLRRLKPFDCKELLYYDYQ |
| | | | | PLSPEVEKEIGCRRVDDLEEMLAQCDVVTINCPLHE |
| | | | | KTRGLFNKDLISKMKKGSWLINTARGAIVVKEDVAE |
| | | | | AVKSGHLRGYGGDVWFPQPAPKDHPLRYVQGPWGGG |
| | | | | NAMVPHMSGTSIDAQIRYANGTKAILESYFSGRHDY |
| | | | | RNEDLIVHNGDYVTRAYGQRNKA |
| 15 | Formate | Friedmanniomyces | A0A4V5N8V4 | MVFARSALRLARPASSLFSQRAAPAFAQRGVPVAKR |
| | dehydrogenase | endolithicus | | ALSATASQQGKVLLVLYDGHQHAQDEPRLLGTTENE |
| | | | | LGIRKWIEDQGHTLVTTSDKDGENSKEDQELVDAEV |
| | | | | IITTPFHPGYLTKERLAKAKKLKIAVTAGIGSDHVD |
| | | | | LDAANTTNGGITVAEVTGSNVVSVAEHVIMTMLVLV |
| | | | | RNFVPAHEMIAAGNWDVAAVAKNEYDLENKVVGTVA |
| | | | | VGRIGERVLRRLKAFDCKELLYFDYQGLTPEKEKEI |
| | | | | GCRRVENLEDMLAQCDVVTINCPLHEKTRGMFNKEL |
| | | | | LSKMKKGSWLVNTARGAIVVKEDVAEALKSGQLRGY |
| | | | | GGDVWFPQPAPKDHPLRYASYSTWGGGNAMVPHMSG |
| | | | | TSIDAQERYAAGTKAILEEYFSGRENYKPEDLIVHK |
| | | | | GDYATKAYGQREGKKHANA |
| 16 | Formate | Actinidia | A0A2R6RYX7 | MAMKRAAASAVRALASSGNSNSSILTRELHTSPGSK |
| | dehydrogenase, | chinensis var. | | KIVGVFYKANEYAAMNPNFLGCVERALGIGDWLKSQ |
| | mitochondrial | chinensis | | GHQYIVTDDKEGPDCELEKHISDMHVLITTPFHPAY |
| | | | | VTAERIKKAKNLQLLLTAGIGSDHVDLKAAAAAGLT |
| | | | | VAEVTGSNVVSVAEDELMRVLILTRNFLPGYHQVIK |
| | | | | GDWNVAAIAHRAYDLEGKTVGTVGAGRIGRLLLQRL |
| | | | | KPFNCHLLYHDRIKMDLELENQIGAKFEEDLDVMLP |
| | | | | KCDIIVINMPLTEKTKGMFNKERIAKLKKGVLIVNN |
| | | | | ARGAIMDTQAVVDACSSGHIAGYSGDVWNPQPAPKD |
| | | | | HPWRYMPNQAMTPHISGTTIDAQLRYAAGVKDMLDR |
| | | | | YFKGEEFPPQHYIVKEGPTRCGGNAIGWGQCEKVKM |
| | | | | GLLENHLGESVRVVTKCSTSPGSKKIVGVFYKANEY |
| | | | | AAMNPNFLGCVERALGIGDWLKSQGHQYIVTDDKEG |
| | | | | PDCELEKHISDMHVLITTPFHPAYVTAERIKKAKNL |
| | | | | QLLLTAGIGSDHVDLKAAAAAGLTVAEVTGSNVVSV |
| | | | | AEDELMRVLILTRNFLPGYHQVIKGDWNVAAIAHRA |
| | | | | YDLEGKTVGTVGAGRIGRLLLQRLKPFNCHLLYHDR |
| | | | | IKMDLELENQIGAKFEEDLDVMLPKCDIIVINMPLT |
| | | | | EKTKGMFNKERIAKLKKGVLIVNNARGAIMDTQAVV |
| | | | | DACSSGHIAGYSGDVWNPQPAPKDHPWRYMPNQAMT |
| | | | | PHISGTTIDAQLRYAAGVKDMLDRYFKGEEFPPQHY |
| | | | | IVKEGELASQYR |
| 17 | Formate | Aspergillus | G7XZ66 | MVFMRSFSRQLRRPATSLLSKGAFAPAASSPFRAAP |
| | dehydrogenase | kawachii | | LANSIAGARTLTASANLQGKVLMVLYDGGEHAKQQP |
| | | | | GLLGTTENELGLRKWLEEQGHTLVTTSDKEGENSTF |
| | | | | DKELVDAEVIITTPFHPGYLTAERLAKAKNLKIAVT |
| | | | | AGVGSDHVDLNAANKTNGGITVAEVTGCNVVSVAEH |
| | | | | VVMTILTLVRNFVPAHEQIRRGEWDVAAVAKNEFDL |
| | | | | EGKVVGTVAVGRIGERVLRRLKPFDCKELLYYDYQP |
| | | | | LSPEVEKEIGCRRVDDLEEMLAQCDVVTINCPLHEK |
| | | | | TRGLFNKELISKMKKGSWLVNTARGAIVVKEDVAEA |
| | | | | VKSGHLRGYGGDVWFPQPAPADHPLRTVQGPWGGGN |
| | | | | AMVPHMSGTSIDAQIRYANGTKAILESYFSGRHDYR |
| | | | | PQDLIVHGGDYVTKAYGQRNKA |
| 18 | Formate | Candidatus | A0A654LUU9 | MKIVAVLYPGGEIAKRTPAILGSAENALGLTNFLKD |
| | dehydrogenase | Nitrosocosmicus | | KGHEFIVLTDKEEELDKHISTTDILITTPFWPAYVT |
| | | oleophilus | | KERISKASNLKLILTAGVGSDHIDLAAAASSKITVA |
| | | | | EITGSNVVSVAEQVVMHILALVRNYIPAYKQVIEGR |
| | | | | WDIAEIASNAHDLEDKVVGIIGMGRIGQRVCERLKA |
| | | | | FDVKMLYHDQIPLRTAEEFVLGVRYTPRDQIVEQAD |
| | | | | VITINTPLTPETDGMFNRDLLFKMKKGAYLVNTARG |
| | | | | KIVDTHALVESLEEGHLAGYAGDVWYPQPATGDHPW |
| | | | | RHMPNHAMVPHYSGTTLEAQNRYANGIKDCLMRFLE |
| | | | | NRPLEQQYLIVDKGGVVSPSYSYAFKK |
| 19 | Formate | Blastomyces | A0A179UWZ8 | MGKVLLVLYDGGQHAKDQPGLLGTTENELGLRKWLE |
| | dehydrogenase | gilchristii | | EKGHTLVTTSDKEGANSKFDQELVDAEVIITTPFHP |
| | | | | GYLTAERLAKAKNLKLAVTAGVGSDHVDLNAANKTN |
| | | | | GGITVAEVTGCNVVSVAEHVVMTILVLVRNFVPSHE |
| | | | | QVASGDWNVAAVAKNEYDLEGKVVGTVAVGRIGERV |
| | | | | LRRLKPFDCKELLYYDYQPLTPEVEKEIGCRRVENL |
| | | | | EEMLAQCDVVTINCPLHEKTRGLFNKDLISKMKKRL |
| | | | | LAHQHRPRRHRRQRRRRRRHQIRPSARLRRRRVVPA |
| | | | | ARAQGPSAALRPGALGRRKRDGAAHVRQLHRCAGAL |
| | | | | RGGHEGDSRVVFLRQA |
| 20 | Formate | Paraburkholderia | A0A0P0RDP0 | MYHVDMSIERRHENALDQTGARRMAKVLCVLYDDPV |
| | dehydrogenase | caribensis MBA4 | | KGMPKQYARDGVPEITRYHDGQTAPTPKGIDFTPGE |
| | | | | LLGSVSGELGLRKYLESLGHTLVVTSDKDGPNSRFE |
| | | | | RELADAEVVISQPFWPAYLTAERIAKAPKLKLALTA |
| | | | | GIGSDHVDLQAAIERGVTVAEVTYCNSISVAEHVVM |
| | | | | MILGLVRNYLPSHEWVKKGGWNIADCVERAYDLEAM |
| | | | | HVGTVAAGRIGAAVLRRLKPFDVKLHYTDRHRLPLD |
| | | | | VEKELGATWHPDVESMVKACDVVTINCPLHPETENL |
| | | | | FNEKLIAKMKRGAYIVNTARGKIVDRDAIVRALESG |
| | | | | QLAGYAGDVWFPQPAPRDHPWRTMPHNGMTPHISGT |
| | | | | TLSAQARYAAGTREILECWFEKRPIRDEYLIVDGGK |
| | | | | LAGVGAHSYSAGNATSGSEEAARFKTA |
| 21 | Formate | Mesorhizobium | A0A440NN77 | MKMAKVVCVLYDDPVDGYPSNYARDGLPKLDRYPGG |
| | dehydrogenase | sp. | | QTLPTPKAIDFEPGVLLGSVTGELGLRPFLESAGHS |
| | | | | LVVTSDKDGPDSVFERELADAEIVISQPFWPAYLTN |
| | | | | ERIAKADQLKLAITAGIGSDHVDLQAAMDRGITVAE |
| | | | | VTYCNSISVSEHVVMMILALVRNYIPSYQWVIDGGW |
| | | | | NIADCVARSYDVEGMQIGTVGAGRIGSAVLHRLQPF |
| | | | | DVKLHYTDRHRLSEAVEKHLGVTFHPDAASMVGICD |
| | | | | VVTINAPLHPETENLFDEAMIARMKRGAYLVNTARG |
| | | | | KICNRDAVARALESGQLAGYAGDVWFPQPAPKDHPW |
| | | | | RSMPHHGMTPHISGSSLSAQARYAAGTREILECWFE |
| | | | | GRPIREEYLIVDGGKLAGAGAHSYSAGDATSGSEEA |
| | | | | ARFKAKA |
| 22 | Formate | Helicocarpus | A0A2B7YOK3 | MVFLRSTSRLLSRPISSLLAGRALPRASAALPLYNT |
| | dehydrogenase | griseus | | QLCGPTSRLPFTGVRTLTASPKLQGKVLLVLYDGEE |
| | | UAMH5409 | | HAKQQPGLLGTTENELGLRKWLEDQGHTLVTTSDKE |
| | | | | GENSKFDQELVDAEVIITTPFHPGYLTAERLAKAKN |
| | | | | LKLAITAGVGSDHVDLNAANKTNGGITVAEVTGCNV |
| | | | | VSVAEHVVMTILVLVRNFVPAHEQVASGDWNVAAVA |
| | | | | KNEFDLEGKVVGTVAVGRIGERVLRRLKPFDCKELL |
| | | | | YYDYQPLKPEVEKEIGCRRVENLEEMLAQCDVVTIN |
| | | | | CPLHEKTRGLFNKELISKMKKGSWLVNTARGAIVVK |
| | | | | EDVAEAVKSGHLRGYGGDVWFPQPAPKDHPLRYAQG |
| | | | | PWGGGNAMVPHMSGTSIDAQVRYAEGTKRILDSYFS |
| | | | | GRHDYRPEDLILFGGDYATKAYGQRTKA |
| 23 | Formate | Streptomyces | A0A7H9W684 | MAKIVCVLYPDPVTGFPPQYARTSLPALDGYPGGQT |
| | dehydrogenase | albidoflavus | | LPTPEGLDFTPGELVGSVSGELGLREFLESRGHTLV |
| | | | | VTSDKEGPDSELDRELADADVVISQPFWPAYLTAER |
| | | | | IARAPKLKLALTAGIGSDHVDLDAAIARGITVAEVT |
| | | | | YSNSISVAEHAVMQILALVRNYLPSHKIAAEGGWNI |
| | | | | ADCVSHAYDLEGMDVGVIAAGRIGQAVLRRLKPFGV |
| | | | | RLHYTDKRRLPREVEEELGLTFHASAQELARNIDVV |
| | | | | SIHAPLHPETQNLFDEKLLATMRPGSYIVNTARAQI |
| | | | | VDRDAIVRALESGQLAGYAGDVWYPQPAPADHPWRT |
| | | | | MPHNGMTPHISGTTLTAQARYAAGTREILEDWLQGT |
| | | | | PIREEYLIVDGGRLAGTGAASYTAGEKTAEKS |
| 24 | Formate | artificial | N/A | MKIVLVLYDCGKHAADEEKLYGCTENKLGIANWLKD |
| | dehydrogenase | | | QGHELITTSDKEGGNSVLDQHIPDADIIITTPFHPA |
| | (mutant of US | | | YITKERIDKAKKLKLVVVAGVGSDHIDLDYINQTGK |
| | Pub. 2019- | | | KISVLEVTGSNVVSVAEHVVMTMLVLVRNFVPAHEQ |
| | 0161741) | | | IINHDWEVAAIAKDAYDIEGKTIATIGAGRIGYRVL |
| | | | | ERLVPFNPKELLYYDYQALPKDAEEKVGARRVENIE |
| | | | | ELVAQADIVTVNAPLHAGTKGLINKELLSKFKKGAW |
| | | | | LVNTARGAICVAEDVAAALESGQLRGYGGDVWFPQP |
| | | | | APKDHPWRDMRNKYGAGNAMTPHYSGTTLDAQTRYA |
| | | | | QGTKNILESFFTGKFDYRPQDIILLNGEYVTKAYGK |
| | | | | HDKK |
| 25 | fdh | Gibbsiella | A0A250B5N7/ | atgtccaagattttgtgcgtattgtatcccgatcct |
| | | quercinecans | ATA21550 | gttaccggttttccgccggtttatgcccgcgacagc |
| | | | | atccctgatatccgttgctatccgaatggccagacg |
| | | | | gcgccgacgccggctggcgaattgggctttaccccc |
| | | | | ggcgagctggtcggcagcgtatctggggaactggga |
| | | | | ttacgtcgctatctggaaagtcagggccatgagttg |
| | | | | attgtgaccagtgacaaagaaggcgcggattcggta |
| | | | | tttgaacgccatttgccggatgccgatgtggtcatt |
| | | | | tcccagccgttttggccggcttacttgaccgctgaa |
| | | | | cgcattgccaaagcgaagaggcttaaactggcgctg |
| | | | | accgccggtattggttccgaccatgtcgatctcaag |
| | | | | gccgccgccgaacgcggcatcaccgtggccgaggtg |
| | | | | acgttctccaactccatcagcgtggcggagcatgtg |
| | | | | gtgatgacggtactggccctggtgcgcaactacctg |
| | | | | ccggcgcatcaaatcgccaacaacggcggttggaac |
| | | | | attgcggactgcgtggcgcgcagctatgacgtggaa |
| | | | | gggatgcatttcggcacgctgggcgccgggcgcatt |
| | | | | gggttggcggtgctgcgacgtttaaaaccctttgat |
| | | | | atgccactgcattattacgatccgcaccgtttgtcg |
| | | | | gcggagtgcgagaaggagctgggtttaatttaccac |
| | | | | gatacgccggagtcgctggtcagcgtctgcgatgtg |
| | | | | gttaacctgcagacgccgctttacccggcaacccag |
| | | | | gggttcgtcaatgcggccttcctggcgaagttcaag |
| | | | | cgtggcgcctacctgatcaataccgcccgcggcgcg |
| | | | | ctgtgtgagcgcgatgcgatcgtccacgcactggaa |
| | | | | accgggcagttggcgggctatggcggcgacgtgtgg |
| | | | | tttcctcaaccggcgccgatcgaccatccatggcgg |
| | | | | cgcatgccgcatcagggcatgacgccgcacatttcc |
| | | | | ggcacgtcactgtcggggcaggcgcgctatgcggcc |
| | | | | gggacgctggaaattctgcagaacttcctggaaggc |
| | | | | aagccgatccgccaagaatacctgattgttgatggc |
| | | | | ggcaatttggcgggtgccggcgccaacgcctataaa |
| | | | | ttggcctga |
| 26 | fdh | Candida | O13437/ | atgaagatcgttttagtcttatatgatgctggtaag |
| | | boidinii | CAA57036 | cacgctgctgatgaagaaaaattatatggttgtact |
| | | | | gaaaataaattaggtattgctaattggttaaaagat |
| | | | | caaggtcatgaactaattactacttctgataaagaa |
| | | | | ggtgaaacaagtgaattggataaacatatcccagat |
| | | | | gctgatattatcatcaccactcctttccatcctgct |
| | | | | tatatcactaaggaaagacttgacaaggctaagaac |
| | | | | ttaaaatcagtcgttgtcgctggtgttggttctgat |
| | | | | cacattgatttagattatattaatcaaacaggtaag |
| | | | | aaaatctcagtcctggaagttacaggttctaatgtt |
| | | | | gtctctgttgctgaacacgttgtcatgaccatgctt |
| | | | | gtcttggttagaaatttcgttccagcacatgaacaa |
| | | | | attattaaccacgattgggaggttgctgctatcgct |
| | | | | aaggatgcttacgatatcgaaggtaaaactatcgct |
| | | | | accattggtgctggtagaattggttacagagtcttg |
| | | | | gaaagattactcccatttaatccaaaagaattatta |
| | | | | tactacgattatcaagctttaccaaaagaagctgaa |
| | | | | gaaaaagttggtgctagaagagttgaaaatattgaa |
| | | | | gaattagttgctcaagctgatatcgttacagttaat |
| | | | | gctccattacacgcaggtacaaaaggtttaattaat |
| | | | | aaggaattattatctaaatttaaaaaaggtgcttgg |
| | | | | ttagtcaataccgcaagaggtgctatttgtgttgct |
| | | | | gaagatgttgcagcagctttagaatctggtcaatta |
| | | | | agaggttacggtggtgatgtttggttcccacaacca |
| | | | | gctccaaaggatcacccatggagagatatgagaaat |
| | | | | aaatatggtgctggtaatgccatgactcctcactac |
| | | | | tctggtactactttagacgctcaaacaagatacgct |
| | | | | gaaggtactaaaaatattttggaatcattctttacc |
| | | | | ggtaaatttgattacagaccacaagatattatctta |
| | | | | ttaaatggtgaatacgttactaaagcttacggtaaa |
| | | | | cacgataagaaataa |
| 27 | fdh | Candida | A0A0A1EQY0/ | atgaagatcgttttagtcttatacgatgctggtaaa |
| | | boidinii | AIY34662 | cacgccgccgatgaagaaaaattatacggttgtact |
| | | | | gaaaacaaattaggtattgccaattggttaaaagat |
| | | | | caaggccatgaattaatcaccacttctgataaagaa |
| | | | | ggcggaaacagtgtgttggatcaacatatcccagat |
| | | | | gctgatattatcattacaactcctttccatcctgct |
| | | | | tatatcaccaaggaaagaatcgacaaagctaaaaaa |
| | | | | ttgaaattagttgtcgtcgctggtgtcggttctgat |
| | | | | catattgatttggattacatcaaccaaaccggtaag |
| | | | | aaaatctccgttttggaagttaccggttctaatgtt |
| | | | | gtctctgttgcagaacacgttctcatgaccatgctt |
| | | | | gtcttggttagaaattttgttccagctcacgaacaa |
| | | | | atcattaaccacgattgggaagttgctgctatcgct |
| | | | | aaggatgcttacgatatcgaaggtaaaactatcgcc |
| | | | | accattggtgccggtagaattggttacagagtcttg |
| | | | | gaaagattagtcccatttaatccaaaagaattatta |
| | | | | tactacgattatcaagctttaccaaaagatgccgaa |
| | | | | gaaaaagttggtgctagaagggttgaaaatattgaa |
| | | | | gaattagttgcccaagctgatatagttacaattaat |
| | | | | gctccattacacgctggtacaaaaggtttaattaac |
| | | | | aaggaattattgtctaaattcaagaaaggtgcttgg |
| | | | | ttagtcaatactgcaagaggtgccatttgtgttgcc |
| | | | | gaagatgttgctgcagctttagaatctggtcaatta |
| | | | | agaggttacggtggtgatgtttggttcccacaacct |
| | | | | gctccaaaagatcacccatggagagatatgagaaac |
| | | | | aaatatggtgctggtaacgccatgactcctcattac |
| | | | | tctggtactactttagatgctcaaactagatacgct |
| | | | | gaaggtactaaaaatattttagagtcattctttacc |
| | | | | ggtaagtttgactacagaccacaagatatcatctta |
| | | | | ttaaatggtgaatacattaccaaagcttatggtaag |
| | | | | cacgataagaaataa |
| 28 | fdh | Clohesyomyces | A0A1Y1ZA31/ | atggtatttctccgctcaatttctcgagtcgcccgc |
| | | aquaticus | ORY07153.1 | ccggcctcctcgatgctctcagcacgcgccgctccc |
| | | | | aaagcttcctttggagcttcgttcttccgtaagccg |
| | | | | aatgcctacagaacgctaacggcgacctcaacacaa |
| | | | | cagggcaaggtccttctcgtactctacgatggcggc |
| | | | | gtccacgcggagcaggagccgcagctcctcggtact |
| | | | | actgagaacgaactgggcatccgaaaatggctcgaa |
| | | | | gatgccggccacacgctcatcacgacttcagacaag |
| | | | | gagggcgagaactccaaattcgaccaggaactggtg |
| | | | | gatgcggaggtgatcattaccacgccgtatgttgaa |
| | | | | gctaccccgcactactcgagctctgtgccccgcaac |
| | | | | cgcaccgcgctctcgccccaccatcttgccactctc |
| | | | | gcgctacccttccaccccggttatctgaccgcagag |
| | | | | cgtctggccaaggcgaagaagctcaagctcgctgtc |
| | | | | actgccggtatcggatctgatcacattgacctcgat |
| | | | | gcagccaacaagacgaatggcggcatcaccgttgcc |
| | | | | gaagtcacgggctcgaatgtcgtgtccgtagccgag |
| | | | | cacgtggttatgaccatcctcaccctcgtgcgcaac |
| | | | | ttcgttcccgcccatgagcagattgccgctggcgag |
| | | | | tgggacgtcgctgccgtcgccaagaacgagtatgat |
| | | | | ttggagaacaaggtcgtcggcaccgttgccgtcggt |
| | | | | cgcattggcgagcgtgtgctgcgccgtctcaagccc |
| | | | | ttcgactgcaaggagctgttgtacttcgactaccag |
| | | | | ccattgaccgctgaaaaggagaaggagattggctgc |
| | | | | cgaagggtggagaacctcgaggagatgctggcacag |
| | | | | tgcgacgtcgtcactatcaactgcccgctacacgaa |
| | | | | aagacacgggggctgttcaacaaggacctcatctcc |
| | | | | aagatgaagaagggctcatggctcatcaacactgcc |
| | | | | cgaggtgccattgtcgtgaaggaggatgttgctgat |
| | | | | gcgctcaagagtggccacctccgtggatacggtggt |
| | | | | gatgtctggttcccgcagccggcgcccaaggagcac |
| | | | | ccgctccgatatgccaccaacccatggggggaggca |
| | | | | acgccatggtcccccacatgtccggtacctcgatcg |
| | | | | atgcccagaagcgatatgctgctggtacgaaggcca |
| | | | | ttctcgactcttacttctccggtcgtgaagactaca |
| | | | | agcccgaggatctcatcgtgcatcaaggcgactacg |
| | | | | ccaccaaggcgtacggtcagcgcgagggaaagaagc |
| | | | | atgctaatgcataa |
| 29 | fdh | Clavispora | C4Y770/ | atgtctaaaccaaaagtgttactcgttttgtacgaa |
| | | lusitaniae | EEQ39876.1 | ggcaagcaccatgccaaagatgagcctaagctttac |
| | | | | ggatgcctcgagaacgaattgggtatcagaggtttc |
| | | | | gtggagtctcacggttacgaattggtgagcatctct |
| | | | | gaaaaggatccaattggagactctgagtttgactat |
| | | | | catttggcagacgctgagattgtcattaccacgcca |
| | | | | tttttccccgcgtacctcactcgtgagagaattgca |
| | | | | gaggcacccaagttgaagctctgcatcaccgcagga |
| | | | | gtcggatcggaccatgttgacttgaatgctgccaat |
| | | | | gaaagaaagatcactgttgcagaggttactggatcc |
| | | | | aacgttgtttctgttgctgaacacgcagtaatgaca |
| | | | | atgttaaacttggttagaaactttgtcccaggacat |
| | | | | gagcaggccatgtctggaggatgggatattgcagcc |
| | | | | attgccaaggatgaatatgacttggaggacaaggtt |
| | | | | atcgccacagttggcgctggcaggattggttaccgt |
| | | | | atcttggagcgccttgtggcattcaatccgaagaag |
| | | | | ctcttgtactttgactaccaggatttgcccaaggaa |
| | | | | gccgtggacaagttgaacgctgcctctaaacttttc |
| | | | | aatggtcacgacgacattgttgaacgcgtggaaaag |
| | | | | ttagaggatatgttatcccgttccgatgttgtgact |
| | | | | atcaattgtcctttacacgagggaagcaaaggtttg |
| | | | | ttcaataaggaaaccattagtcacatgaaggatggc |
| | | | | gcttggttggtgaacacagcccgtggtgccatttgt |
| | | | | gttgaaaaggatgtagctgatgctgtcgaatcgggc |
| | | | | aagctccgtggatacggtggagacgtatggtatcca |
| | | | | cagccagccccagaccatcatccttggagaactttc |
| | | | | cgcaacaagtatggcggtggaaatgctatgaccccc |
| | | | | cacgttagtggaacatcattggacgcccaagagaga |
| | | | | tatgctgctggtacacaagcaattttgaaatcgtac |
| | | | | tttgaaaagtcctttgattatcggccacaggatgtg |
| | | | | attgttgttgatggcgaatacgccaccaaagcctat |
| | | | | ggccagcgtaagta |
| 30 | fdh | Caballeronia | A0A7H9UPL5/ | atggccaaggttttatgcgttttatatgacgaccct |
| | | sordidicola | RSL21651.1 | gtcgacggtatgcccaagaagtacgcccgggacgac |
| | | | | attcccgccatctcgaagtatcacgacggacaaagc |
| | | | | accccgacacccaaggcaatcgacttcaagccgggc |
| | | | | gagttgctgggcagcgtgtccggcgagttgggattg |
| | | | | cgcaagttcctcgaatcacgggggcacaccctggtc |
| | | | | gttacatccgacaaggacggccccaactccacgttc |
| | | | | gagcgcgagctggaggatgccgaggttgtcatttcc |
| | | | | cagccattctggcccgcctatctgacggctgaacgc |
| | | | | atcgcccgggcgccgaagctcaagcttgcgctcacg |
| | | | | gctggcatcggctcggatcacgtcgatttgcaggcc |
| | | | | gcgatcgatcgcggcatcacggttgccgaagtgacc |
| | | | | ttctgcaatagcatcagcgtggccgagcacgtcgtc |
| | | | | atgatgatcctgtcccaggtccggaactatttgccg |
| | | | | tcgcatgaatgggtcaagaagggcggctggaatatc |
| | | | | gcggattgcgtctcgcgtgcgtacgatctcgagggg |
| | | | | atgaacgtagggaccgtggcggccggacgcatcggt |
| | | | | gcggcggtgctgagacggctcaaaccctttgacgtc |
| | | | | aagctgcactacacggaccggcatcgtctgccggaa |
| | | | | caggtcgagcgcgagcttggcgcaacctggcatccc |
| | | | | actgctgaatccatggcgcgtgtgtgcgatatcgtg |
| | | | | accatcaactgtccgttgcacccggaaacggaacat |
| | | | | ctcttcgacgagacgatgatcagcaagatgaagcgc |
| | | | | ggcgcttacatcatcaacacggcgcgcgggaagatt |
| | | | | gtcgatcgcgatgctgtcgcgcgtgcgctggaatcg |
| | | | | ggccaactggcgggttatgcaggcgacgtctggttc |
| | | | | ccgcagcctgcaccgagcgatcacccgtggcggacc |
| | | | | atgccgaatcagggcatgacgccgcacacgtcgggc |
| | | | | actacgctttcggcacaggcgcgttacgccgctggc |
| | | | | acgcgtgaaattctcgaatgctggtttgccaaacaa |
| | | | | ccgattcgtgaggagtatctgatcgtcgacggcgga |
| | | | | cgtcttgccggtgtgggtgcgcactcgtacagcgca |
| | | | | ggcaatgcgacgtcgggctcggaagaggcggcccgc |
| | | | | ttcaagaaggcataa |
| 31 | fdh | Coccidioides | A0A0J6ETL5/ | atgggtaaagttcttatggttatgtatgacggtggg |
| | | posadasii | KMM63856.1 | gagcatgcgaagcagcagcctggtttattgggaaca |
| | | RMSCC 3488 | | acagaaaatgagcttggattaagaaaatggcttgag |
| | | | | gagagaggccacaccttggtaaccacatctgacaag |
| | | | | gagggatcgaactctacctttgagcgcgagcttgtt |
| | | | | gatgcggagatcatcatcactacaccgttccatccc |
| | | | | ggttatctcaccgcagagcgtctggccaaggccaag |
| | | | | aacctcaaacttgccatcacagcgggtgttggttcc |
| | | | | gaccatgttgacctgaatgccgccaataagaccaac |
| | | | | ggaggtatcactgtcgctgaagtcactggctgcaat |
| | | | | gtcgtgtctgttgccgaacatgtcgttatgaccatc |
| | | | | ttagtcctcgtccgcaatttcgtcccggcccatcaa |
| | | | | caagttgccagcggtgaatgggacgttgctgccgtt |
| | | | | gctaaaaacgagtatgaccttgagggtaaggtcgtc |
| | | | | ggaactgtagctgttggccgcattggtgagcgtgta |
| | | | | cttcgccgtcttaagcccttcgactgcaaggagctt |
| | | | | ctttattatgactaccagcccctcagccctgaggtc |
| | | | | gagaaggaaattggatgccgtcgtgttgagaacttg |
| | | | | gaggagatgttggcacagtgtgatgttgtcactatc |
| | | | | aactgccctctccacgagaagacacgtggcttgttc |
| | | | | aacaaggaactcatttctaagatgaagaaaggctca |
| | | | | tggcttgtaaacaccgcccgtggggctatcgtcgtc |
| | | | | aaggaggacgtcgcagaagctataaaatcaggccat |
| | | | | ctccgtggatatggcggtgacgtctggttcccacaa |
| | | | | cctgctcccaaagaccacccactacgctatgtccag |
| | | | | ggcccatggggcggcggaaatgccatggtccctcac |
| | | | | atgtctggaacttccattgatgcacaaatccgctat |
| | | | | gcacagggcacgaagaacatccttgaatcgtacttc |
| | | | | tccggcaagttcgattacaaacaagaagatttgatt |
| | | | | gttcatcagggc |
| 32 | fdh | Botryotinia | M7TQ78/ | atggtatttttcactcgctccctctctcgtttggca |
| | | fuckeliana | EMR85706.1 | acaagaccgtctccctgtgctcggtcaatcttcaca |
| | | | | gcttctgctgttgcgagaactacacctacatggacg |
| | | | | acgcgatcgactgctttccgctcgcagaagctgaat |
| | | | | aacatatttgggaatgtcagaatgctaacgggtaat |
| | | | | catcgtgaaaaggtcaaggtacttcttgtattgtac |
| | | | | gacggaaagaagcacgccgaagaggtcccagagctt |
| | | | | ctcggcacaaccgagaacgagctcggtatccgcaaa |
| | | | | tggctcgaggatcaaggacatgagctcatcaccacc |
| | | | | tccgacaaggagggagagaactccgagttcgacaag |
| | | | | cacttggtagacgcagagatcatcattaccactcct |
| | | | | ttccaccctggttacctcactgccgaacgtcttgcc |
| | | | | aaggccaagaacctcaagatcgccatcactgccggt |
| | | | | attggatctgatcacgtcgatctcaatgctgccaac |
| | | | | aagaccaacggtggtatcaccgttgctgaagttact |
| | | | | ggatccaacgtcgtctccgtcgctgagcacgtcgtt |
| | | | | atgaccatcttggttctcgtccgtaactttgtccca |
| | | | | gcacacgagcaaatccaagccggagaatgggacgtt |
| | | | | gccgctgccgccaagaacgagttcgatcttgagggt |
| | | | | aaggtcgtcggtaccgttgctgtcggaagaattggt |
| | | | | gagcgtgtcttgagaagattgaagccattcgactgc |
| | | | | aaggaattgttgtacttcgattaccaacctcttaag |
| | | | | ccagaggttgagcaagagattggttgcagacgtgtt |
| | | | | accgatttggaggagatgttggctcaatgtgatgtt |
| | | | | gtcaccattaactgccctctccacgagaagacccgt |
| | | | | ggccttttcaacaaggagttgatctccaagatgaag |
| | | | | aagggatcctggttggtcaacaccgcccgtggtgcc |
| | | | | attgttgtcaaggaagatgttgccgatgccctcgca |
| | | | | tccggtcacctccgcggttacggaggagatgtttgg |
| | | | | ttccctcaaccagcaccaaaggaccacccactccgc |
| | | | | tacgcaaagaacccattcggtggtggaaacgccatg |
| | | | | gttcctcacatgtctggtacctctctggatgctcaa |
| | | | | aagagatatgccgatggtaccaaggctattctcgag |
| | | | | agctacttgtctggaaagcatgactacagaccagag |
| | | | | gatttgattgttattggtggtgactatgctaccaag |
| | | | | gcttatggtgagagagcgaagaacagtggttctgcc |
| | | | | ggtgccaacaaggcataa |
| 33 | fdh | Yarrowia | Q6CDN8/ | atgaagattctccttatcctttacgacgccggctcc |
| | | lipolytica | CAG83477.1 | cacgctgctgacgaacccaagctgctaggttgcacc |
| | | | | gagaacgagctcggcatccgatcctggctcgagtcc |
| | | | | cagggccataccctggtcaccacctcttccaaggag |
| | | | | ggtgctgactctgttctcgacaaggagattgttgat |
| | | | | gccgatgttgtcatcaccactcccttccaccccggc |
| | | | | tacatcacccgagagcgaattgccaaggccaagaac |
| | | | | ctcaagatctgtgtcactgccggtgttggctctgac |
| | | | | cacgttgatcttgctgccgccaacgagagaaacatt |
| | | | | gccgtgctcgaggtcactggttccaacgtgacttct |
| | | | | gttgctgagcacgttgtcatgaccatgcttgttctt |
| | | | | gtccgaaacttcgtgcctgccaacgagcaggttcga |
| | | | | ggcggcggctgggacgttgccggtgttgccaaggat |
| | | | | tcctacgatattgagggtaaggtcattggtaccgtc |
| | | | | ggagtcggccgaatcggcaagcgagtgctccagcga |
| | | | | ctcaagcccttcgaccccaaggagctgctctactac |
| | | | | gactatcagcctctgtctgctgccgacgagaaggag |
| | | | | attggcgcccgacgagttgagaagctcgaggacatg |
| | | | | cttgcccagtgtgatgttgtcaccatcaactgcccc |
| | | | | ctgcacgaatccaccaagggtctctttaacaaggag |
| | | | | ctgctgtcccacatgaagaagggcgcctggctcgtc |
| | | | | aacaccgctcgaggagccatctgtgtaaaggaggac |
| | | | | gttgccgccgctctcaagtctggtcagctccgaggc |
| | | | | tacggcggagatgtctggttcccccagcctgcccct |
| | | | | gctgaccacccctggagaaagatggtcaacaagtac |
| | | | | ggtgccggtaacgccatgaccccccacatgtctgga |
| | | | | acctctctggacgcccaggcccgatacgctgctggt |
| | | | | gtcaagcagattctcgacgagttcttctccggccga |
| | | | | gaacagtaccgaccccaggacattatctgctacggt |
| | | | | ggtaactacggcaccaaggcctacggagacgacaag |
| | | | | aaggtcgtcgacaagaagtaa |
| 34 | fdh | Kushneria | A0A1I1JVK8/ | atggccaagattctctgtgttctttatcccgatccg |
| | | avicenniae | SFC52556 | gtgaccggctatccaccgcgttatgcgcgtgacgat |
| | | | | atccccgaaatcacgggctatcccaacggccagaca |
| | | | | gcgccttctccggagggagatccggggttttccccc |
| | | | | ggcgaattgatcggctgcgtctccggtgagctgggg |
| | | | | ctgcgttcgtgggcgcaggccaacggtcatgagctt |
| | | | | atcgtgaccagtgacaaggagggcgtcgattcggtc |
| | | | | ttcgagcgccatctgccggacgctgatgtcgtgatt |
| | | | | tctcagccgttctggccggcctacctcagcgcggaa |
| | | | | cgcattgcccgggcaccgaagctcaagctggcgctg |
| | | | | acggccggtatcggctcggatcacgtcgatctcaag |
| | | | | gcggcggccgagcacggtatcacggtggccgaggtg |
| | | | | accttctccaactcgatcagtgtggccgagcacgtg |
| | | | | gtcatgaccgcgctgacgctggtccgcaatttcgtg |
| | | | | ccgtcgcaccagattgccatcaacggcggctggaac |
| | | | | atcgctgactgcgtatcacgcagctacgacatcgaa |
| | | | | gggatgcagtttggtaccctcggtgccggtcgtatc |
| | | | | ggccttgcggtcctgcgtcgcatgaagcccttcgat |
| | | | | acaccgctgcattactacgatccgcaccgtctgtcc |
| | | | | gaggatatcgagtgcgagctcggtctgacctatcac |
| | | | | gactcgccggaatcgctggtgagcgtctgcgacatc |
| | | | | gtcaacgttcagacgccgctttacccctcgaccgaa |
| | | | | gggttcatcaacgacgcgctgctgtcgaaattcaag |
| | | | | cgtggcgcctatctggtcaacaccgcgcgcggcggg |
| | | | | ctttgcgaccgtgaggcagtggtcagggcgctggag |
| | | | | tccgggcagctggcaggttacgggggcgatgtgtgg |
| | | | | tacccgcagcccgcgccggtggatcatccgtggcgg |
| | | | | cgcatgcccaatcatgcgatgacgccacatatctcc |
| | | | | ggcacctcgctgtcggcccagacccgctatgccgcc |
| | | | | ggaacgctggaaatcctgcagaatttcctcgccggc |
| | | | | aagtcgctgcgtgaagagtatctcatcgtcgatgcc |
| | | | | gggaacctggccggcgccggggccaatgcctatcag |
| | | | | ctttga |
| 35 | fdh | Zea mays | A0A3L6EKZ5/ | atggtagagtccacaccattgctggtttgcttgcta |
| | | | PWZ21308 | gacacaaacactaaactggagaagcacattgaagac |
| | | | | atgcatgtcctgattaccaccccattccacccagct |
| | | | | tatgtcactgcagagaggataaagaaggcgaagaat |
| | | | | cttgagcttcttctcacggcaggaatcggctccgat |
| | | | | catattgatcttccagcagctgctgctgcgggctta |
| | | | | actgtggctgaggtcactggaagcaacactgtctct |
| | | | | gtggcagaagatgagctcttgcgcattctgattctg |
| | | | | ctcaggaacttcttgcctgggtaccaacaggtagtt |
| | | | | caaggcgaatggaacgtagcaggcattgcccacaga |
| | | | | gcttatgatcttgaaggaaaaactgttgggactgtt |
| | | | | ggagctggtcgtattggcaggctcttgcttcagcgt |
| | | | | acttaagccctttaactgcaacctactttaccatga |
| | | | | cgacttcagattgacccagagcttgagaaagaaatt |
| | | | | ggggccaaatttgaggaggacctggatgctatgctt |
| | | | | ccaaagtttgatgtgattgtgatcaacacacctctt |
| | | | | acggagaaaacaagaggcatgtttaacaaagagagg |
| | | | | attgcaaagatgaagaaaggtgtaattgttgtgaat |
| | | | | aatgctcgaggagcaatcatggatgctcgagcagtc |
| | | | | gtagatgcatgttctagcggtcacattgctggatat |
| | | | | ggtggtgatgtgtggttcccacagccagcaccaaag |
| | | | | gatcacccctggcgctacatgcctaaccatgccatg |
| | | | | acccctcacatctctaggactacaattgatgcgcag |
| | | | | gtgcggtatgcggatggtgtgagggacatgctggat |
| | | | | aggtatttcaagggcgaggacttcccggtgcagaac |
| | | | | tacatcgtcaaggaaggccagctcgccagtcagtac |
| | | | | aagtga |
| 36 | fdh | Dactylellina | S8BZ87/ | atggtacttcgagccacatctacccttgcgcgttgc |
| | | haptotyla | EPS40692 | ctcgccaaaccaaccctccaggcctccacactgggt |
| | | | | gctcgtgttgttcggccagcgtatcgccagccattg |
| | | | | accgcaattcaaaccagaaaactgacaactactttc |
| | | | | caacctcaggtcaaagttcttgctgtcttgtatgat |
| | | | | ggtggcaagcacgcccaggatgttccagctctgcta |
| | | | | ggaaccacagagaacgagctcggtattagaaaatgg |
| | | | | cttgaggatcaaggacacactttggtcaccacctct |
| | | | | gacaaggaaggcgagaactcaaaattcgaccaagag |
| | | | | ttggttgatgctgaagttatcatcacaactcccttc |
| | | | | cgccctggatatttgactgctgagcgtcttgcaaaa |
| | | | | gccaagaaattgaagcttgctatcactgccggaatt |
| | | | | ggatctgaccatgtggacctcaatgctgccaataag |
| | | | | acgaatggtggagtcactgttgctgaagtcactgga |
| | | | | tcgaatgtcgtcagtgtagctgaacatgttgttatg |
| | | | | acgatcttgatcttggtcaggaattttgtcccagct |
| | | | | catgaccaaatcagaaatggtgactgggaagttgcc |
| | | | | gctgttgccaaggatgaatatgacctcgaagataag |
| | | | | attgttggaactgttggtgttggtagaatcggcgag |
| | | | | cgtgtcctgcgaagattgaagccattcgactgcaag |
| | | | | gaactgctttacttcgactaccaaccactcaatgca |
| | | | | gcagttgaaaaagaaatcggctgccgccgtgtcgag |
| | | | | agtttagaggagatggttgcacagtgcgatgttgta |
| | | | | acgatcaactgtccactccacgagtccacccacgga |
| | | | | ttattcaacaagaatttgatctccaagatgaagaag |
| | | | | ggttcttggttggttaatactgctcgtggcgccatc |
| | | | | gttgtcaaggaagacgttgccgaagccgtcaaatct |
| | | | | ggccaccttcgaggctatggtggcgatgtttggttc |
| | | | | ccacaaccggcccctaaagatcacccactgcgatac |
| | | | | gccgtaaacccattcggtggcggtaatgccatggtc |
| | | | | ccgcatatgtcaggaacttctcttgatgctcagaaa |
| | | | | cgatacgccgatggcaccaagaaaatcctcgaaagc |
| | | | | tacctttctggtcgccacgactatcagccacatgac |
| | | | | ctcattgtccacaagggagattacgcgaccaaggca |
| | | | | tacggtcagcgcaaatga |
| 37 | fdh | Segniliparus | E5XSQ8/ | atggcgaaaatcgttgccgtcctttacccggacccg |
| | | rugosus | EFV12585.1 | gtggacgggtatcccacgacgtacgcccgcgacgac |
| | | | | atcccggtcatcgcgagctaccccggagggcagagc |
| | | | | gcgccgaccccgtccgcgatcgacttcgtccccggc |
| | | | | cagctgctgggcagcgtgaccggcgagctgggcttg |
| | | | | cggaagttcctcgaagcccagggccacacgctcgtg |
| | | | | gtcacctcggacaaggaggggccggattccgcgctc |
| | | | | gaccgcgagctggccgacgcggacgtggtgatctcg |
| | | | | cagccgttctggcccgcctacctcaccgccgagcgg |
| | | | | atcgcggcggctccgaacctcaagctcgcgatcacg |
| | | | | gcgggcatcggctcggaccacgtggacctcaacgcc |
| | | | | gccatcgcgcgcggcatcaccgtcgccgaggtcacc |
| | | | | ttcagcaactcgatcagcgtcgccgaacacgcggtc |
| | | | | atgcagatccttgctttggtgcgcaactacctgccg |
| | | | | tcgcacgcgtgggtcgccgagaagcggggctggaac |
| | | | | atcgccgactcggtggagcgcgcctacgacctcgaa |
| | | | | ggcatgaccgtcggcgtgatcgcggcgggccggatc |
| | | | | ggcctcgccgtcttgcgcaggctcaagcccttcgac |
| | | | | gtgaagctggcgtacaccgacaagcaccgcctgtcc |
| | | | | gccgaagtcgaggaggagctgggcctcacgttctac |
| | | | | ccggacatcccctcgctggtgcgcgcggtggacatc |
| | | | | gtggacgtccacgcgccgctcgtgcccgagacccac |
| | | | | cacctcttcgacgacgcgctcatcggcacgatgaag |
| | | | | cgcggctcgtacatcgtgaacaccgccagaggcgcg |
| | | | | atcatggacaccggcgcggtggtccgcgcgctcgag |
| | | | | tccggccagctcgccgggtacgcgggcgacgtctgg |
| | | | | tacccgcagccgcccgcacccgaccacccgtggcgc |
| | | | | acgatgccgtggagcgggatgaccccgcacgtctcg |
| | | | | ggcacgacgctctcggcgcaggcccggtacgccgca |
| | | | | gggactcgggagatcctcgagtcctggctcgcgggc |
| | | | | gcgccgatccgcgacgagtacctcatcgtggacggc |
| | | | | ggcaagctcgcgggcaccggagcccgttcctacagc |
| | | | | accagtggcagcgggtcgagcccaggatcgagcggg |
| | | | | aagtag |
| 38 | fdh | Aspergillus | A0A2V5IND1/ | atggtatttatgcgctctttctcccgtcaacttcga |
| | | indologenus | PYI25397.1 | cggcctgcgacttctcttctttcaagcagaggccct |
| | | CBS 114.80 | | ctcgcatcggctggcagcagtcccttgcggacctcg |
| | | | | cctctggccaactccattgctggtgcacgtacgcta |
| | | | | accgcctcggccaatctacagggcaagattcttatg |
| | | | | gttctttatgacggcggtgagcacgccaagcagcag |
| | | | | cccggtcttctgggtaccaccgagaacgagcttggt |
| | | | | ttgagaaagtggctggaggaccagggccacactttg |
| | | | | gttaccacctctgacaaggagggtgagaactccact |
| | | | | ttcgacaaggagctcgttgatgccgaagtcatcatc |
| | | | | accacccccttccaccccggttaccttactgcggag |
| | | | | cgtttggccaaggccaagaaccttaagatcgccgtc |
| | | | | actgccggtgttggttccgaccacgtcgacctcaac |
| | | | | cgcgccaacaagaccaatggcggtatcactgtggcc |
| | | | | gaagtcactggctgcaatgttgtctccgttgcggag |
| | | | | cacgttgtcatgacgatcctgaccctggtccgcaac |
| | | | | ttcgttcccgcccacgagcagatccgtaacggcgag |
| | | | | tgggacgttgctgctgtcgccaagaacgagtacgat |
| | | | | ttggagaacaaggttgtcggcactgtcgctgtcggc |
| | | | | cgcatcggtgagcgtgtcctgcgccgtctcaagcct |
| | | | | ttcgactgcaaggagctgctctactacgactaccag |
| | | | | cctctctcccctgaggtcgagaaggagatcggctgc |
| | | | | cgccgtgttgacgacctcgaggagatgctcgctcag |
| | | | | tgtgatgtcgttaccatcaactgccctctacacgag |
| | | | | aagactcgtggtctgttcaacaaggacctcatctcc |
| | | | | aagatgaagaagggctcgtggctcatcaacaccgct |
| | | | | cgcggtgccatcgttgtcaaggaggacgtcgccgag |
| | | | | gccgtcaagtctggtcacttgagaggctacggaggt |
| | | | | gatgtctggttccctcagcccgcccccaaggatcac |
| | | | | cctctgcgctacgtccagggcccctggggcggcggc |
| | | | | aacgccatggttccccacatgtccggtacctcgatc |
| | | | | gacgcccagatccgttacgccaacggcaccaaggct |
| | | | | atcctcgagagctacttctccggtcgccacgactac |
| | | | | agaaacgaggacctcatcgtccacaacggtgactat |
| | | | | gtcactcgcgcctacggtcagcgcaacaaggcttaa |
| 39 | fdh | Friedmanniomyces | A0A4V5N8V4/ | atggtattcgctcgctccgcactccgcctcgcacgc |
| | | endolithicus | TKA45179.1 | ccggcgagctcgctgttctcgcagcgggcggcgcca |
| | | | | gccttcgcccagcgcggggttccagtcgccaagagg |
| | | | | gccctgtcagcgactgcgtcgcaacagggaaaagtc |
| | | | | ctcttggtgctctacgatggccaccaacacgcccag |
| | | | | gacgagcctcgattgctcgggacgaccgagaacgag |
| | | | | ctcgggatccggaagtggattgaggaccagggccac |
| | | | | actcttgtgacgacctccgacaaagacggcgagaac |
| | | | | agcaagttcgaccaagagcttgttgacgccgaggtc |
| | | | | atcatcaccacacccttccaccccggctacctcacc |
| | | | | aaggagcgcctcgccaaggcgaagaagctcaagatc |
| | | | | gccgtgacggccggtattggttccgaccacgtcgac |
| | | | | ctcgacgccgccaacacgaccaacggcggtatcacc |
| | | | | gtcgccgaagtcaccggctccaacgtggtgtccgtc |
| | | | | gccgagcacgtcatcatgaccatgctcgtcctggtc |
| | | | | cgcaacttcgtccccgcccacgagatgatcgccgcc |
| | | | | ggcaactgggacgtcgcggccgtcgccaagaacgag |
| | | | | tacgacttggagaacaaggtcgtcggtaccgtggct |
| | | | | gtcggccggatcggcgagcgtgtcctccgcagactg |
| | | | | aaggccttcgactgcaaggagctgctgtactttgac |
| | | | | taccagggtctgactcccgagaaggagaaggagatt |
| | | | | ggctgcaggcgggtggagaacctcgaggacatgctg |
| | | | | gcgcagtgcgatgtcgtcaccatcaactgcccgctg |
| | | | | cacgagaagacgcgcggcatgttcaacaaggagctg |
| | | | | ctgagcaagatgaagaagggctcctggctcgtcaac |
| | | | | acggcccgcggcgccatcgtcgtcaaggaagacgtg |
| | | | | cgagcggccctcaagagcggccagctccgcggctac |
| | | | | cggcggcgacgtctggttcccccagccagccccaag |
| | | | | gaccacccgctccgctacgcctcctactccacctgg |
| | | | | ggcggcggcaacgccatggtgccccacatgtccggc |
| | | | | acctccatcgacgcgcaggagcgctacgccgccggc |
| | | | | accaaggcgattttggaggagtacttcagcgggcgc |
| | | | | gagaactacaagcccgaggatttgattgtgcataag |
| | | | | ggggattacgccactaaggcgtatgggcagcgggag |
| | | | | gggaagaagcatgctaatgcttag |
| 40 | fdh | Actinidia | A0A2R6RYX7/ | atggcgatgaagcgtgctgctgcctctgcagtccgt |
| | | chinensis var. | PSS35217.1 | gctcttgcttcgtcggggaattcgaattcgtcgatt |
| | | chinensis | | ttgaccagagagctccatacttctcctgggagcaaa |
| | | | | aagatagttggagtgttctataaggcaaatgaatat |
| | | | | gctgcaatgaatcccaattttttgggctgtgtggaa |
| | | | | agagcattgggcataggtgactggttgaaatcacaa |
| | | | | gggcaccaatacatcgtcacagatgacaaagaggga |
| | | | | cctgattgtgaactcgagaaacacatttctgatatg |
| | | | | cacgtcctcataactaccccatttcatccggcctat |
| | | | | gttactgcggaaaggatcaagaaggctaaaaatctg |
| | | | | caactattgctcacagctggaattggctctgaccat |
| | | | | gtcgatctgaaggctgcagctgctgccgggttaaca |
| | | | | gttgctgaggtcactggaagcaatgtggtttcggta |
| | | | | gcagaggacgagctcatgagagttctcattctcact |
| | | | | cgaaatttcttacctggataccatcaggttattaaa |
| | | | | ggggactggaatgttgcagctattgctcacagggcc |
| | | | | tacgatctggaaggaaagactgttggaactgtcggt |
| | | | | gctgggcgtattggcaggctgttactccaaagactg |
| | | | | aagcctttcaactgtcatctcctctatcatgaccgg |
| | | | | atcaagatggacctggagttggagaatcaaatcggt |
| | | | | gcaaagtttgaggaggatcttgatgtgatgcttcca |
| | | | | aaatgtgacattattgtcatcaacatgcctcttact |
| | | | | gagaaaacaaaaggtatgtttaacaaggagaggatc |
| | | | | gctaagttgaagaagggagttctcattgttaacaat |
| | | | | gctagaggagctataatggatacacaagcagttgtt |
| | | | | gatgcctgttccagtggacatattgcaggctacagt |
| | | | | ggggatgtttggaatccacaaccagctccaaaggac |
| | | | | cacccatggcgatacatgccaaaccaagccatgacc |
| | | | | cctcatatttctgggactacaatcgatgcacagttg |
| | | | | cgatatgcagcaggagttaaggacatgttggatagg |
| | | | | tatttcaagggagaagagtttcctccacagcattac |
| | | | | gattgtcaaggaggggcccacccttgtggaggaaat |
| | | | | gcgattgggtgggggcagtgcgagaaagtaaaaatg |
| | | | | ggactactggagaatcacttgggagaaagtgtcaga |
| | | | | gttgtcacaaagtgctctacttctcctgggagcaaa |
| | | | | aagatagttggagtgttctataaggcaaatgaatat |
| | | | | gctgcaatgaatcccaattttttgggctgtgtggaa |
| | | | | agagcattgggcataggtgactggttgaaatcacaa |
| | | | | gggcaccaatacatcgtcacagatgacaaagaggga |
| | | | | cctgattgtgaactcgagaaacacatttctgatatg |
| | | | | cacgtcctcataactaccccatttcatccggcctat |
| | | | | gttactgcggaaaggatcaagaaggctaaaaatctg |
| | | | | caactattgctcacagctggaattggctctgaccat |
| | | | | gtcgatctgaaggctgcagctgctgctgggttaaca |
| | | | | gttgctgaggtcactggaagcaatgtggtttcggta |
| | | | | gcagaggacgagctcatgagagttctcattctcact |
| | | | | cgaaatttcttacctggataccatcaggttattaaa |
| | | | | ggggactggaatgttgcagctattgctcacagggcc |
| | | | | tacgatctggaaggaaagactgttggaactgtcggt |
| | | | | gctgggcgtattggcaggctgttactccaaagactg |
| | | | | aagcctttcaactgtcatctcctctatcatgaccgg |
| | | | | atcaagatggacctggagttggagaatcaaatcggt |
| | | | | gcaaagtttgaggaggatcttgatgtgatgcttcca |
| | | | | aaatgtgacattattgtcatcaacatgcctcttact |
| | | | | gagaaaacaaaaggtatgtttaacaaggagaggatc |
| | | | | gctaagttgaagaagggagttctcattgttaacaat |
| | | | | gctagaggagctataatggatacacaagcagttgtt |
| | | | | gatgcctgttccagtggacatattgcaggctacagt |
| | | | | ggggatgtttggaatccacaaccagctccaaaggac |
| | | | | cacccatggcgatacatgccaaaccaagccatgacc |
| | | | | cctcatatttctgggactacaatcgatgcacagttg |
| | | | | cgatatgcagcaggagttaaggacatgttggatagg |
| | | | | tatttcaagggagaagagtttcctccacagcattac |
| | | | | attgtcaaggagggtgaactcgctagccaataccgt |
| | | | | taa |
| 41 | fdh | Aspergillus | G7XZ66/ | atggtatttatgcgctctttctcccgccaacttcgg |
| | | kawachii | GAA92225.1 | cgacctgccacctctcttctctccaagggagccttc |
| | | | | gcgcccgcggccagcagcccattccgggctgctcct |
| | | | | ctggctaactcgatcgctggtgcacgtacgctaacc |
| | | | | gcctccgcgaatctgcagggcaaggttcttatggtc |
| | | | | ctctatgacggcggtgagcacgccaagcagcagccc |
| | | | | ggtcttctgggtaccaccgagaacgagctcggcttg |
| | | | | aggaagtggctcgaggagcagggtcacactctggtc |
| | | | | accacctccgacaaggagggcgagaactcgaccttc |
| | | | | gacaaggagctcgtcgacgctgaagtcatcatcacc |
| | | | | actcccttccaccccggttacctcactgctgagcgt |
| | | | | ctggccaaggccaagaacctcaagatcgccgtcact |
| | | | | gctggtgtcggttccgaccacgttgacctgaacgct |
| | | | | gccaacaagaccaatggcggtatcaccgtcgccgag |
| | | | | gtcaccggctgcaacgtcgtctccgtcgctgagcac |
| | | | | gttgtcatgaccatcctgaccctggtccgcaacttc |
| | | | | gttcccgcccacgagcagatccgccgcggcgagtgg |
| | | | | gacgttgccgctgtcgctaagaacgagttcgacctc |
| | | | | gagggtaaggtcgtcggtaccgttgccgttggccgt |
| | | | | atcggagagcgtgtcctccgccgtctcaagcccttc |
| | | | | gactgcaaggagctcctctactacgactaccagccc |
| | | | | ctttcccccgaggttgagaaggagatcggctgccgc |
| | | | | cgcgtcgacgacctcgaggagatgctcgctcagtgc |
| | | | | gatgttgtcaccatcaactgccccctgcacgagaag |
| | | | | acccgcggtctgttcaacaaggagctcatctccaag |
| | | | | atgaagaagggctcttggctcgtcaacaccgctcgt |
| | | | | ggtgccatcgttgtcaaggaggatgtcgccgaggcc |
| | | | | gtcaagtctggccacctccgcggatacggtggtgac |
| | | | | gtctggttcccccagcccgcccccgccgaccaccct |
| | | | | ctgcgcaccgtccagggcccctggggcggcggcaac |
| | | | | gccatggttcctcacatgtccggtacctccatcgat |
| | | | | gcccagatccgttacgccaacggtaccaaggccatc |
| | | | | ctcgagagctacttctccggccgccacgactaccgc |
| | | | | ccccaggacctcattgtccacggcggtgactacgtt |
| | | | | accaaggcctacggccagcgcaacaaggcctaa |
| 42 | fdh | Candidatus | A0A654LUU9/ | atgaaaattgttgcagtgttatatcctggaggagaa |
| | | Nitrosocosmicus | ALI34306.1 | atagcaaaaaggacaccggcgattcttggttcggca |
| | | oleophilus | | gagaatgctcttggcttgacaaattttctgaaagat |
| | | | | aaaggacatgaatttattgtattaacagataaagag |
| | | | | gaggaacttgataaacacatttctactactgatatt |
| | | | | ctgatcactacccccttttggccggcctatgttacc |
| | | | | aaagaaagaatttccaaagcttctaacctcaagctc |
| | | | | atcttaactgcaggcgtaggatcagatcacatagat |
| | | | | ttggctgcagcagcaagtagtaaaataactgttgca |
| | | | | gaaattacaggtagtaatgtggtaagtgtagctgaa |
| | | | | caggttgtgatgcatatcttggctttagtgcgaaac |
| | | | | tacataccagcttataaacaggttatagaaggtaga |
| | | | | tgggatatagcagagatagcatcaaacgcgcatgat |
| | | | | ctagaagacaaggttgttggaattatcggaatggga |
| | | | | agaattggacaaagggtttgtgaaaggttgaaagca |
| | | | | tttgatgttaaaatgctttatcatgatcaaattcct |
| | | | | ctacggactgcagaagaatttgtgttgggtgtcaga |
| | | | | tacacccctagagatcagatcgtagaacaggctgat |
| | | | | gtgattacaataaatacgcctttaacgccagagacc |
| | | | | gatggtatgtttaatcgagatctactttttaaaatg |
| | | | | aaaaaaggtgcatatcttgttaatacagctcgagga |
| | | | | aagatcgtagatactcatgctcttgtagaatctttg |
| | | | | gaggaaggtcatttagcaggatatgctggcgatgtt |
| | | | | tggtatccacaaccggccacaggagatcatccttgg |
| | | | | agacatatgcctaatcatgcgatggtccctcattat |
| | | | | tctggaacaaccctcgaagcacaaaacagatatgcg |
| | | | | aatggaataaaggattgtcttatgcggtttctagaa |
| | | | | aaccgtcctcttgaacaacaataccttattgtcgat |
| | | | | aaaggtggcgtggtaagcccgagttacagctacgct |
| | | | | ttcaagaaataa |
| 43 | fdh | Blastomyces | A0A179UWZ8/ | atgggtaaagtactcttggttttgtatgatggagga |
| | | gilchristii | OAT12333.1 | caacatgcgaaggaccagcctggtttgctcggaacg |
| | | | | actgagaacgagctgggcctgaggaagtggttggaa |
| | | | | gagaagggccatacccttgtaaccacctctgacaaa |
| | | | | gaaggtgccaactctaaattcgaccaggagcttgtc |
| | | | | gatgctgaggttatcatcactaccccattccaccca |
| | | | | ggatatctgaccgcagaacgcctggctaaagccaag |
| | | | | aacctgaaactggccgtcacggccggtgtgggatcc |
| | | | | gaccacgtcgacctgaacgccgccaacaagacgaat |
| | | | | ggcggcatcaccgttgccgaagtgacgggctgcaat |
| | | | | gtcgtctccgtcgccgagcacgtcgtcatgaccatt |
| | | | | ctcgtcctggtccgcaactttgtcccttcacacgag |
| | | | | caggttgccagcggcgattggaacgttgccgccgtg |
| | | | | gcgaagaacgagtatgacctcgagggaaaagtcgtc |
| | | | | ggcaccgttgccgtgggccgtattggcgagcgtgtc |
| | | | | ctgcgacgactgaagccgtttgactgcaaggagctg |
| | | | | ctgtactatgattaccagccgctgacgccagaggtc |
| | | | | gagaaggagatcggatgccgccgcgtggagaatctg |
| | | | | gaggaaatgctcgcgcagtgtgatgttgtcaccatc |
| | | | | aactgccccctgcacgagaagactcgcggcttgttt |
| | | | | aacaaagacctcatctcgaagatgaagaagaggctc |
| | | | | ctggctcatcaacaccgcccgcggcgccatcgtcgt |
| | | | | caaagaagacgtcgccgacgccatcaaatccggcca |
| | | | | tctgcgcggctacggcggcgacgtgtggttcccgca |
| | | | | gcccgcgcccaaggaccatccgctgcgctacgtcca |
| | | | | ggggccctggggcggcggaaacgcgatggtgccgca |
| | | | | catgtccggcagctccatcgatgcgcaggtgcgcta |
| | | | | cgcggagggcacgaaggcgattctcgagtcgtattt |
| | | | | ctccggcaggcatga |
| 44 | fdh | Paraburkholderia | A0A0P0RDP0/ | atgtatcacgtcgatatgtcgatagagcgccggcat |
| | | caribensis MBA4 | ALL66463.1 | gaaaatgcgctcgaccagactggagcgagacgaatg |
| | | | | gctaaggttctttgtgtgctgtatgacgaccccgtc |
| | | | | aagggaatgccgaaacagtacgcccgcgacggcgta |
| | | | | cccgaaatcacgcgttatcacgacggtcaaacggcc |
| | | | | ccgacgccgaagggcatcgacttcacgccgggcgaa |
| | | | | ttgctgggcagcgtatcgggcgaactggggctgcgg |
| | | | | aaatacctcgaatcgctagggcatacgctcgtcgtg |
| | | | | acatccgacaaggatggtccgaactcgcggttcgag |
| | | | | cgcgagcttgccgacgccgaagtcgtgatttcccaa |
| | | | | ccgttctggcccgcgtacctgacggcggaacgcatc |
| | | | | gcgaaagcgccgaagctcaagctcgcgttgacggcg |
| | | | | ggcatcggctcggatcacgtcgatttgcaggcggcc |
| | | | | atcgagcgcggcgtgacggtggccgaagtgacgtat |
| | | | | tgcaacagcatcagcgtcgccgaacacgtcgtgatg |
| | | | | atgattctcgggctggtgcgcaattacctgccgtcg |
| | | | | catgaatgggtgaagaagggcggctggaacatcgcc |
| | | | | gactgcgtcgagcgtgcttacgatctcgaagccatg |
| | | | | cacgtaggcacggttgcggcgggccggatcggcgcg |
| | | | | gccgtgttgcgcagactgaagccgttcgacgtgaag |
| | | | | ctgcactatacggatcgtcatcgcctgccgctcgat |
| | | | | gtcgagaaagaactcggcgcgacgtggcacccggac |
| | | | | gtcgagtcgatggtgaaagcgtgcgacgtggtgacg |
| | | | | atcaactgtccgctgcacccggaaacggaaaatctc |
| | | | | ttcaatgaaaagctgatcgcgaaaatgaagcgcggc |
| | | | | gcgtatatcgtgaacacggcgcgcggcaagatcgtc |
| | | | | gatcgcgatgcgattgtgcgggcgctcgaatcggga |
| | | | | caactggcgggctatgcgggcgacgtgtggttcccg |
| | | | | cagccggcgccgcgcgatcatccgtggcgcacgatg |
| | | | | ccgcacaacggcatgacgccgcatatttcgggcact |
| | | | | acgctgtcggcacaggcgcgttatgcggctggcacg |
| | | | | cgcgagatcctcgaatgctggttcgagaagcggcct |
| | | | | atccgcgacgagtatctgatcgtcgacggcggcaag |
| | | | | ctggcgggcgtcggcgcgcactcgtatagcgcgggc |
| | | | | aatgcgacgtcgggatcggaagaggcggcgcgattc |
| | | | | aagactgcgtaa |
| 45 | fdh | Mesorhizobium | A0A440NN77/ | atcaaaatggcaaaggtcgtttgcgtcctctatgac |
| | | sp. | RWF04819.1 | gatccggttgacggttatccgtcaaattatgcacgg |
| | | | | gatgggctgccgaagctcgaccgctatcccggcggc |
| | | | | cagacgcttcccacgccgaaggccatcgacttcgaa |
| | | | | ccgggggttctgctcggcagcgtgacgggcgagctc |
| | | | | ggcctgcggccatttctcgaaagcgccgggcacagt |
| | | | | ctggtcgtgacctcggacaaggacggtcccgacagc |
| | | | | gttttcgaacgcgaactcgctgacgccgaaatcgtc |
| | | | | atctcgcagccgttctggccggcctatctgaccaac |
| | | | | gaacggatcgccaaggcagaccaacttaagcttgcg |
| | | | | atcaccgccggcatcggatccgaccatgtcgatctg |
| | | | | caggccgcgatggaccgcggcatcaccgtcgccgag |
| | | | | gtgacctactgcaactcgatcagcgtgtccgagcat |
| | | | | gttgtgatgatgatcctggcgctggtgcgaaactac |
| | | | | atcccctcttaccagtgggtcatcgacggtggctgg |
| | | | | aatatcgccgactgtgtcgcgcgctcctacgatgtc |
| | | | | gaggggatgcagatcggaaccgtcggcgaccggccg |
| | | | | gtcggtagcgcggtgctgcaccggctgcagccgttc |
| | | | | gacgtcaaattgcattacaccgaccgccaccgcctg |
| | | | | tccgaggcagtcgagaaacaccttggcgtcaccttc |
| | | | | cacccagacgccgcgtccatggtcggcatctgcgat |
| | | | | gtggtgacgatcaatgcacctctgcatccggagacg |
| | | | | gagaacctgttcgacgaggcgatgatcgccaggatg |
| | | | | aaacggggcgcctatctggtgaacacggcgcgcggc |
| | | | | aagatctgcaatcgcgacgccgtcgcccgtgcgctg |
| | | | | gagagcggccagctcgccggctatgccggcgatgtg |
| | | | | tggttcccgcagccggcccccaaggaccatccctgg |
| | | | | cgttcgatgccgcatcacggcatgacgccgcatatt |
| | | | | tcaggatcttcgctctcggcccaggcgcgctacgcc |
| | | | | gccggcacccgcgaaatcctggagtgctggttcgaa |
| | | | | ggccggccgatccgggaggaatacctgatcgtcgat |
| | | | | ggcggcaagctggccggcgccggcgcgcattcctat |
| | | | | agcgcaggcgacgccacaagcggttcggaggaggcg |
| | | | | gcgcgcttcaaggccaaggcgtga |
| 46 | fdh | Helicocarpus | A0A2B7Y0K3/ | atggtatttctgcgctctacatcccgtctcctctcc |
| | | griseus | PGH14392.1 | cggccaatctcctctctgttggctggtcgagccctt |
| | | UAMH5409 | | ccccgcgcctcagctgccttgcccctatacaacacg |
| | | | | cagctctgcgggccaacgtcccgtttgcctttcact |
| | | | | ggagtcagaacactaacagcgagtcctaaactccag |
| | | | | ggtaaagttctgcttgtcttgtacgatggagaagag |
| | | | | catgcgaagcagcagcccggcctgcttggaaccact |
| | | | | gagaacgagctcggattgagaaaatggctcgaagat |
| | | | | cagggacataccctggttaccacctccgataaagag |
| | | | | ggagagaactcaaaattcgaccaggagctcgtcgac |
| | | | | gctgaagttatcatcactactcctttccaccccggt |
| | | | | tacctcactgcggagcgtctcgccaaagctaagaac |
| | | | | ctcaagcttgccatcacagctggtgttggctccgac |
| | | | | cacgtcgatctgaacgccgccaacaagactaacggc |
| | | | | ggtatcaccgtcgctgaagtcactggctgcaatgtc |
| | | | | agtctctgtcgctgagcacgtcgtcatgactatcct |
| | | | | tgttttggtccgcaacttcgtccccgcacacgagca |
| | | | | ggttgccagtggagactggaacgttgctgccgtggc |
| | | | | caagaatgagttcgacttggagggcaaggtcgttgg |
| | | | | tccgtcgctgttggccgcattggcgagcgtgttctc |
| | | | | cgacgactcaagccattcgactgcaaggagctgctc |
| | | | | tactacgactaccagcccttgaagcctgaggttgag |
| | | | | aaggagattggatgccgtcgcgtcgagaacctcgag |
| | | | | gagatgctggctcagtgtgacgttgtcaccatcaac |
| | | | | tgccctctccacgagaagacccgtggtctgttcaac |
| | | | | aaggagctcatctcgaagatgaagaaaggctcctgg |
| | | | | ctcgtcaacactgcccgtggcgccatcgtcgtcaag |
| | | | | gaggacgttgccgaggccgttaagagcggccacctc |
| | | | | cgcggctacggaggcgacgtctggttcccccagccc |
| | | | | gcgcccaaggaccacccactccgctacgcccagggc |
| | | | | ccatggggcggcggaaacgccatggttcctcacatg |
| | | | | tccggtacctccatcgacgcccaggtccgctatgct |
| | | | | gagggcaccaagaggatcttggactcgtacttctct |
| | | | | cggcagacacgactataggctgaggatttgatcctg |
| | | | | ttcggtggtgattatgctaccaaggcatatggacag |
| | | | | cggactaaggcttag |
| 47 | fdh | Streptomyces | A0A7H9W684/ | atggcgaagatcgtctgcgttctctacccggacccg |
| | | albidoflavus | RZF05900.1 | gtcaccggcttcccgccccagtacgcgcgcacctcc |
| | | | | ctccctgcgctcgacggctaccccggcggccagacc |
| | | | | ctgccgactcccgagggcctcgacttcacccccggc |
| | | | | gaactggtgggcagcgtctccggggagctggggttg |
| | | | | cgggaattcctggagtcccggggccacaccctggtc |
| | | | | gtcacctccgacaaggagggccccgactccgagctg |
| | | | | gaccgtgaattggccgacgccgacgtggtgatatcc |
| | | | | cagccgttctggcccgcctacctcaccgccgaacgc |
| | | | | atcgcccgcgccccgaagctgaagctggcgctgacc |
| | | | | gccggcatcggctccgaccacgtcgacctggacgcc |
| | | | | gccatcgcccgcggcatcaccgtcgccgaggtcacg |
| | | | | tacagcaacagcatcagcgtcgccgagcacgctgtg |
| | | | | atgcagatcctggccctggtccgcaactacctgccc |
| | | | | tcccacaagatcgcggccgagggtggctggaacatc |
| | | | | gccgactgcgtctcgcacgcctacgacctggagggc |
| | | | | atggacgtcggagtcatcgccgccggccgcatcggc |
| | | | | caggccgtgctgcgccgcctcaagcccttcggtgtc |
| | | | | cgcctccactacaccgacaagcgccggctgccgcgc |
| | | | | gaggtcgaggaggagctgggcctgaccttccacgcg |
| | | | | agtgcccaggaactcgcccgcaacatcgacgtggtc |
| | | | | tccatccacgccccgctccaccccgagacgcagaac |
| | | | | ctcttcgacgagaagctgctggcgaccatgcggccg |
| | | | | ggctcctacatcgtcaacaccgcccgcgcccagatc |
| | | | | gtcgaccgcgacgcgatcgtccgcgccctggagtcg |
| | | | | ggacagctcgccggttacgcgggcgacgtctggtac |
| | | | | ccccagcccgcccccgccgaccacccctggcgcacc |
| | | | | atgccccacaacggcatgaccccccacatctccggc |
| | | | | accaccctcaccgcccaggcccgctacgcggccggc |
| | | | | acccgcgagatcctggaggactggctgcagggcacg |
| | | | | ccgatccgcgaggaatacctcatcgtcgacggcggc |
| | | | | cgcctcgccggaacgggcgccgcgtcgtacacggcg |
| | | | | ggagaaaagacggcagaaaagagctga |
| 48 | fdh (mutant | artificial | N/A | atgaagatcgttttagtcttatacgattgtggtaag |
| | of US | | | tcacgctgccgatgaagaaaaattatacggtgtact |
| | Pub. 2019- | | | gaaaacaaattaggtattgccaattggttgaaagat |
| | 0161741) | | | caaggacatgaattaatcaccacgtctgataaagaa |
| | | | | ggcggaaacagtgtgttggatcaacatataccagat |
| | | | | gccgatattatcattacaactcctttccatcctgct |
| | | | | tatatcactaaggaaagaatcgacaaggctaaaaaa |
| | | | | ttgaaattagttgttgtcgctggtgtcggttctgat |
| | | | | catattgatttggattatatcaaccaaaccggtaag |
| | | | | aaaatctccgttttggaagttaccggttctaatgtt |
| | | | | gtctctgttgcagaacacgttgtcatgaccatgctt |
| | | | | gtcttggttagaaattttgttccagctcacgaacaa |
| | | | | atcattaaccacgattgggaggttgctgctatcgct |
| | | | | aaggatgcttacgatatcgaaggtaaaactatcgcc |
| | | | | accattggtgccggtagaattggttacagagtcttg |
| | | | | gaaagattagtcccattcaatcctaaagaattatta |
| | | | | tactacgattatcaagctttaccaaaagatgctgaa |
| | | | | gaaaaagttggtgctagaagggttgaaaatattgaa |
| | | | | gaattggttgcccaagctgatatagttacagttaat |
| | | | | gctccattacacgctggtacaaaaggtttaattaac |
| | | | | aaggaattattgtctaaattcaagaaaggtgcttgg |
| | | | | ttagtcaatactgcaagaggtgccatttgtgttgcc |
| | | | | gaagatgttgctgcagctttagaatctggtcaatta |
| | | | | agaggttatggtggtgatgtttggttcccacaacca |
| | | | | gctccaaaagatcacccatggagagatatgagaaac |
| | | | | aaatatggtgctggtaacgccatgactcctcattac |
| | | | | tctggtactactttagatgctcaaactagatacgct |
| | | | | caaggtactaaaaatatcttggagtcattctttact |
| | | | | ggtaagtttgattacagaccacaagatatcatctta |
| | | | | ttaaacggtgaatacgttaccaaagcttacggtaag |
| | | | | cacgataagaaataa |

It is understood that modifications which do not substantially affect the activity of the various embodiments of this disclosure are also provided within the definition described herein provided herein. Accordingly, the following examples are intended to illustrate but not limit the present disclosure.

### EXAMPLES

### Example 1

### Synthetic Metagenomic and Protein Engineering FDH Libraries (Primary FDH Libraries)

### Sourcing and Design of Synthetic Metagenomic FDH Library

About 2500 FDH candidates were informatically sourced from a sequence database using a seed amino acid sequence roughly corresponding to the FDH of Candida boidinii (UniprotID: 013437; SEQ ID NO: 2). From the 2500 FDH candidates, 300 sequences (plus two seed sequences) were selected for further evaluation based on sequence quality, structure and similarity, e.g., sequences having less than 50% similarity or over 95% identity were omitted.

These proteins were then recoded into DNA using tinker and a standard E. coli codon table, and cloned into a pSC101ampR vector. The promoter/RBS sequence were the same as found on the control plasmid.

### Sourcing and Design of an FDH Library Based on Template Sequence

A protein engineering FDH library was also generated by computational design from a select template sequence. Libraries were ordered from Ranomics, Inc. The mutational complexity of this library was targeted towards fewer than 7 substitutions per variant.

### Example 2

### Screening of the Enzyme Libraries

### Validation of FDH Assay

Following the design and synthesis of the FDH libraries described in Example 1, an FDH activity screening assay based on the assay described in Hopner and Knappe, Methods of Enzymatic Analysis, Volume III, 1551-1555 (1974) was optimized using known controls and scaled down to run on 384 well plates. A brief description of the optimized assay is provided below.

Thawed glycerol stocks (5 μL) of FDH library transformants was stamped into 500 μL/well of 1×LB media with 100 μg/mL Carbenicillin in half-height deepwell plates and sealed with AeraSeals. The plates were incubated at 35° C. and shaken at 1,000 revolutions per minute (RPM) in 80% humidity for 16-20 hours. 50 μL/well of the resulting cultures was stamped into 450 μL/well of 1×LB media with 100 μg/mL Carbenicillin in half-height deepwell plates and sealed with AeraSeals. The plates were incubated at 35° C. and shaken at 1,000 revolutions per minute (RPM) in 80% humidity for 16-20 hours. 10 μL/well of the resulting production cultures was stamped into 190 μL/well Phosphate Buffered Saline (PBS) in 96-well flat bottom plates. Optical measurements were taken on a plate reader, with absorbance measured at 600 nm. 125 μL/well of the production cultures was stamped into another set of half-height deepwell plates, sealed and centrifuged at 4000×g for 15 minutes. The plates were unsealed and the supernatants were removed by decanting. The resulting pellets were stored at −80° C. until the start of the assay.

On the day of the assay, frozen pellet sample plates were thawed at room temperature for at least one hour. 125 μL/well of lysis buffer (1× Bugbuster lysis reagent, 2.5 mM 1,4-Dithiothreitol (DTT), 0.2 mM Phenylmethylsulfonyl fluoride (PMSF), 3U/L rLysozyme, 0.0025 U/uL Benzonase Nuclease) was dispensed to the pellet plates. The buffer and the pellets were mixed 25 times using the repeated pipetting setup in the Hamilton STARlet liquid handler resulting in lysed cell suspensions. 5 μL/well of lysed cell suspensions was mixed into 45 μL/well of assay buffer (final concentrations: 2.5 mM Nicotinamide adenine dinucleotide (NAD), 10 mM Sodium Formate, 50 mM Tris(hydroxymethyl)aminomethane hydrochloride (Tris-HCl) pH 7.4) in 96-well flat bottom half area black plates. Continuous, kinetic absorbance measurements were taken on a plate reader, with absorbance measured at 340 nm. The measurements were taken for the time interval of 10 minutes during which the plates were continuously shaken in slow, orbital movement and the temperature was held at 28° C. The kinetic data from the plate reader were then processed using linear fit and data processing packages on Python.

### Primary Screen of Synthetic Metagenomic and Protein Engineering FDH Library

A primary screen of the variants generated in Example 1 was performed using the optimized FDH assay described above. The results from this primary screening experiment were processed as follows:
1. kinetic data for each strain replicate were plotted;
2. the rate of reaction based on the linear fit of the first 5-minute portion of the data (i.e., the linear portion) was calculated and the unit from Abs/sec to mAbs/min was adjusted;
3. the rate by OD600 data was normalized to obtain “normalized rates”;
4. “standardized rates” were obtained by dividing the normalized rates in each container with the average of the high control rates in that container;
5. standardized rates across all the samples were ranked based on a ratio of the standardized rates and the average high controls in each container; and
6. hits were called by setting a certain threshold of the ratio between standardized rates and the average high controls (i.e., 1.0× activity compared to high controls).

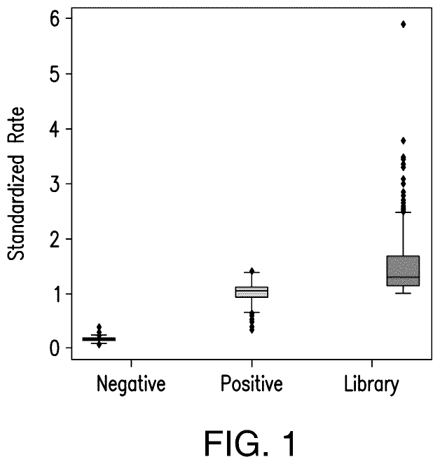

From the 1,386 strains screened, 1.0× activity was selected as the cut-off and 270 strains were identified as “hits.” The performance of the controls in the screen are found in FIG. 1. In the standardized rates of the controls and hits representation, the sample types represented from left to right are as follows: Negative control (t679853); Positive control (t594738); and the library.

Hits from the library, as defined by the criterion above, were tabulated as follows:

The number of hits with 50% cutoff was 527. The number of hits with 60% cutoff was 464. The number of hits with 70% cutoff was 415. The number of hits with 80% cutoff was 371. The number of hits with 90% cutoff was 328. The number of hits with 100% cutoff was 270. The number of hits with 110% cutoff was 225. The number of hits with 120% cutoff was 183. The number of hits with 130% cutoff was 145.

### Example 3

### Design and Synthesis of Secondary Protein Engineering Library (Secondary FDH Library)

Construction of a second generation (gen2) protein engineering library was performed leveraging the results and learning from the previous metagenomic and protein engineering FDH libraries described in Examples 1 and 2 (gen 1). In the previous screening efforts, a gene recode of the FDH from Candida boidinii (UniprotID: 013437; SEQ ID NO: 2) was identified and led to a 2-times improvement in measured activity. The metagenomic discovery library also identified an FDH from Gibbsiella quercinecans (UniprotID: A0A250B5N7; SEQ ID NO: 1) with twice the activity of the seed (i.e., FDH from Candida boidinii). In the protein engineering library, 84 out of 1176 variants had greater activity than wild-type FDH from Candida boidinii. There were over 30 beneficial point-mutations, defined as 1.5× wild-type activity, with the top 8 having >2× greater than wild-type FDH from Candida boidinii.

The gen2 protein engineering library used the FDH from Candida boidinii gene recode identified in the initial library and the discovered FDH from Gibbsiella quercinecans as sequence templates. For FDH from Candida boidinii, several design strategies were employed to generate the variant library. The top beneficial point-mutations from the first protein engineering library were combined to generate variants with 2-4 point-mutations. Homology models of wild-type FDH from Candida boidinii as well as models that incorporated point-mutations from the gen1 protein engineering library summarized in TABLE 2 were subjected to computational docking and design.

TABLE 2
Incorporated Point−Mutations from gen1 Protein Engineering Library

| Residue | Position | Alteration |
|---|---|---|
| A | 91 | S |
| H | 97 | N |
| A | 257 | S |
| T | 256 | C |
| Y | 312 | V |

The hit-recombination and computational docking and design strategies yielded 386 and 321 constructs, respectively.

Several different protein engineering design strategies were implemented using FDH from Gibbsiella quercinecans as the template sequence including: (1) multiple sequence analysis of FDH homologs; (2) consideration of commonly occurring point-mutations within 10 angstroms of the FDH active-site; (3) consideration of beneficial mutations identified in FDH from Candida boidinii; and (4) computational docking and design using a FDH Gibbsiella quercinecans homology model. These sequence bioinformatics, active-site mutagenesis, hit-transfer, and docking & design protein engineering strategies produced 150, 140, 117, and 63 constructs, respectively.

The variant protein sequences were deduplicated, put into pG_10499 plasmid, and the 1121-member library was submitted for synthesis, from which a library size of 1,061 constructs was generated.

### Example 4

### Secondary FDH Library Transformation

The initial library size of 1,061 constructs described in Example 3 were subjected to a high throughput transformation protocol. About 91% of constructs (i.e., 968 constructs) were recovered with 3 picks and 2.7% (i.e., 29) of constructs with 0 picks, likely due to extended recovery time and increased DNA amount (TABLE 3).

TABLE 3
Picking Success. Number and percentage
of samples by number of clones picked

| No. of Picks | Constructs | Percentage of Library |
|---|---|---|
| 0 | 29.0 | 2.7% |
| 1 | 34.0 | 3.2% |
| 2 | 31.0 | 2.9% |
| 3+ | 968.0 | 91.1% |
| Total | 1,062.0 | 100.0% |

Growth of the transformed constructs were further evaluated and based on OD values obtained, an additional 20 constructs (i.e., 1.9%) were eliminated after picking. As a result, 49 constructs (i.e., 4.6%) were not present in the final cryostocks with passing ODs and 918 constructs (i.e., 86.4%) were each represented by 3 clones in the final cryostocks with passing ODs (TABLE 4).

TABLE 4
Final transformant data and growth success,
specifically demonstrating the number and percentage
of picked samples with successful OD
(i.e., samples that grew) including a positive control.

| No. of Picks | Constructs | Percentage of Library |
|---|---|---|
| 0 | 49.0 | 4.6% |
| 1 | 34.0 | 3.2% |
| 2 | 61.0 | 5.7% |
| 3+ | 918.0 | 86.4% |
| Total | 1,062.0 | 100.0% |

### Example 5

### Primary Screen of Secondary FDH Library

### Definitions and Calculations of Different Rate Nomenclature

The definitions and the calculations of the different rate nomenclature provided as follows serve as a reference for the data collection. The term “raw rate” was defined by a slope of a linear regression of the kinetic data over the first five minutes of the reaction. The term “OD normalized rate” was defined as the raw rate of each sample divided by the OD of that specific sample. “Standardized rate” was defined as the OD normalized rate divided by the average OD normalized rate of selected positive controls on the specific plate that the sample is on. The positive control used in each standardized rate was described in the data collection.

### Primary Screen: Raw, OD-Normalized and Standardized Rates

A primary screening using the optimized FDH assay described in Example 2 was conducted. The following negative control and three positive controls were included in the analysis: (1) negative control t679853; and (2) the positive control Positive 1 (t594738), which was from the first generation library screen, i.e., the strain was the wild-type FDH, Positive 2 (t729843), which was a recoded wild type hit from the genI library which became one of the two generation two library templates, and Positive 3 (t730034), which was a metagenomic hit from the gen1 library. The Pearson Correlation Coefficient (R)=0.297 also indicated a slight positive correlation. The overall low R value was likely due to the high OD outliers on the right side of the plot. The data were normalized with OD to alleviate the OD dependent effects and hits that performed better than the positive controls using the OD normalized rate (Y-axis) were observed. Since the Positive 2 strain (t729843) had better correlations between the raw rates, as well as OD-normalized rates, and the average rates on each plate, the Positive 2 strain (t729843) controls were used to further normalize the OD normalized data to generate “standardized rate” that had less plate-to-plate variation.

### Primary Screen: Hit Selections

200 strains were then selected for secondary screening using the following procedure: 1) strains were ranked based solely on average standardized rates (Positive 2 strain (t728943) Normalized and OD Normalized); 2) data from A and B workcells were combined as one for this ranking process; and 3) the 150 top ranking strains that came from Positive 3 strain (t730034) (metagenomic FDH hit) template and the 50 top ranking strains that came from Positive 2 strain (t729843) (E. coli recoded, codon-optimized FDH) template were selected.

For the 200 hits selected, the number of replicates versus average standardized rates were plotted. The hits included six one-pick strains, ten two-pick strains, and 184 three-pick hit strains.

### Example 6

### Secondary Screen of Secondary FDH Library

### Secondary Screen: Raw, OD-Normalized and Standardized Rates

A secondary screening using the optimized FDH assay described in Example 2 was conducted. Preliminary analysis of the secondary screening results revealed that the minimal rates of the library samples were approximately 10 rate units, indicating that all of the primary hit members had appreciable enzymatic activities. A dot plot of rate (Y-axis) versus OD600 (X-axis) indicated a positive correlation between OD and raw rate, similar to the primary screen. In addition, the OD normalized library data rose above Positive 3 strain (t730034).

For the per container data, the distributions of the data were observed to be quite uniform. To be consistent with the primary screen and to benchmark the rates of reactions against the different positive controls, the rates in the secondary screen were standardized against all three positive controls (t594738; t729843; t730034) separately.

### Correlations Between Primary and Secondary Screens

The correlations between primary and secondary screens were evaluated and results in TABLE 5 showed that most of the rate types gave satisfactory correlations between the two screens with the Pearson correlation above 0.7, R-square above 0.5, and Spearman correlation above 0.65. Except for the standardized rate values for Positive 1 strain (t594738), results for the two screens strongly correlated indicating that the hits from both screens were probably true hits. For those hits that performed better than a specific positive control, standardized (t729843) rate or standardized (t730034) rate were good indicators to observe. Most of the data that had good positive correlations showed a strong positive slope clustering in the data supporting the correlation values.

TABLE 5
Correlations between primary and secondary screens based on various reaction rates

| | | OD | Standardized | Standardized | Standardized |
|---|---|---|---|---|---|
| | | Normalized | (t729843) | (t594738) | (t730034) |
| Correlation Coeff. | Raw Rate | Rate | Rate | Rate | Rate |
| Pearson (r) | 0.765 | 0.747 | 0.746 | 0.425 | 0.783 |
| r-square | 0.585 | 0.558 | 0.557 | 0.181 | 0.613 |
| Spearman (ρ) | 0.667 | 0.659 | 0.71 | 0.442 | 0.7 |

### Example 7

### Additional FDH Activity Analysis

Further assessment of selected variants was conducted by performing additional FDH activity screens as described below.

An E. coli strain containing a plasmid having a nucleotide sequence encoding an FDH variant on a constitutive promoter was generated. The strain was inoculated in LB with carbenicillin (100 μg/mL) and grown overnight at 35° C. in a shaking incubator. The overnight culture was diluted into fresh LB with carbenicillin grown overnight at 35° C. in a shaking incubator. Cells were collected by centrifugation and frozen at −20° C. until the day of conducting an in vitro lysate assay.

For the in vitro lysate assay, the cell pellet was thawed and resuspended in 0.1 M Tris-HCl, pH 7.0 buffer. The OD600 was measured of cell suspension and each of the candidates were normalized to an OD of 4. Pellets were prepared by centrifugation and the pellet was then lysed with a chemical lysis reagent containing nuclease and lysozyme for 30 minutes at room temperature. This lysate was used to measure the FDH activity at 35° C. as follows. An aliquot of the crude FDH lysate, a desired concentration of formate (0-100 mM), and 0.5 mM NAD were mixed in 0.04 mL of 0.1 M Tris-HCl, pH 7.4 buffer. The kinetics of the reaction was monitored by coupling the product NADH to 10 μM PMS (1-methoxy-5-methylphenazinium methyl sulfate and 2 mM XTT (2,3-Bis-(2-methoxy-4-nitro-5-sulfophenyl)-2H-tetrazolium-5-carboxanilide) using absorbance at 560 nm. Relative activity to controls (e.g., SEQ ID NOS: 1 or 2, as indicated for TABLE 6, TABLE 7, and TABLE 8) was determined.

The results of these screens, including identifying the activity of select variants, are shown in TABLE 6 and TABLE 7.

TABLE 6
Relative Activity of Exemplary FDHs Engineered using the
FDH from Gibbsiella quercinecans (SEQ ID NO: 1) as the
Template Compared to the FDH from Gibbsiella
quercinecans wild-type control

| Variant # | Residue | Position | Alteration | Activity |
|---|---|---|---|---|
| 1 | I | 335 | S | + |
| 2 | C | 231 | A | + |
| 3 | Y | 346 | A | − |
| 4 | F | 146 | Q | + |
| 5 | I | 150 | T | + |
| 6 | A | 122 | S | − |
| | T | 284 | S | |
| 7 | F | 313 | Y | − |
| | H | 128 | N | |
| 8 | T | 198 | I | − |
| | T | 284 | C | |
| 9 | Q | 98 | W | − |
| | S | 147 | V | |
| 10 | F | 100 | A | − |
| | S | 147 | A | |
| | V | 152 | A | |
| 11 | A | 122 | S | − |
| | A | 285 | S | |
| 12 | N | 175 | R | + |
| 13 | Q | 343 | A | − |
| 14 | A | 122 | S | − |
| | Y | 346 | M | |
| 15 | A | 269 | D | + |
| | H | 297 | R | |
| 16 | C | 290 | I | + |
| | F | 313 | Y | |
| 17 | Q | 303 | K | + |
| | T | 350 | A | |
| 18 | H | 128 | N | − |
| | Q | 303 | K | |
| 19 | I | 335 | A | + |
| 20 | C | 290 | I | + |
| | Q | 303 | K | |
| 21 | G | 342 | K | − |
| 22 | V | 152 | A | − |
| | Y | 346 | I | |
| 23 | G | 342 | L | − |
| 24 | Y | 346 | L | + |
| 25 | G | 342 | A | + |
| 26 | N | 381 | P | − |
| 27 | C | 30 | G | + |
| 28 | N | 176 | S | + |
| 29 | P | 218 | T | + |
| | I | 238 | T | |
| 30 | S | 147 | L | − |
| 31 | V | 144 | I | + |
| 32 | A | 122 | S | − |
| | G | 138 | D | |
| 33 | K | 384 | T | + |
| 34 | T | 338 | Y | − |
| 35 | Q | 303 | K | + |
| | T | 284 | C | |
| 36 | I | 335 | M | + |
| 37 | H | 297 | V | + |
| | T | 301 | S | |
| 38 | H | 128 | N | − |
| | Y | 346 | I | |
| 39 | S | 147 | A | − |
| | V | 152 | A | |
| | Y | 346 | L | |
| 40 | A | 122 | S | + |
| | F | 313 | Y | |
| 41 | G | 342 | V | − |
| | Q | 98 | W | |
| | S | 147 | L | |
| 42 | V | 19 | S | + |
| 43 | Q | 315 | E | − |
| 44 | H | 128 | N | − |
| | T | 284 | S | |
| 45 | D | 27 | K | + |
| 46 | I | 319 | R | + |
| 47 | Q | 98 | W | − |
| | S | 147 | L | |
| 48 | A | 262 | E | + |
| 49 | S | 339 | T | + |
| 50 | S | 151 | A | + |
| 51 | I | 150 | G | − |
| 52 | A | 285 | G | + |
| | T | 284 | M | |
| 53 | Q | 303 | K | + |
| 54 | H | 128 | M | − |
| 55 | G | 342 | I | − |
| 56 | L | 199 | I | + |
| | F | 266 | M | |
| 57 | P | 218 | N | + |
| | I | 238 | T | |
| 58 | Q | 256 | I | − |
| 59 | C | 231 | K | + |
| 60 | R | 325 | T | + |
| 61 | Q | 256 | C | − |
| 62 | Q | 256 | L | − |
| 63 | T | 338 | F | − |
| 64 | V | 191 | L | + |
| 65 | G | 138 | D | − |
| | H | 128 | N | |
| 66 | C | 231 | R | + |
| 67 | H | 297 | G | + |
| | T | 301 | S | |
| 68 | C | 231 | V | + |
| 69 | F | 100 | A | − |
| | V | 152 | A | |
| | Y | 346 | I | |
| 70 | N | 374 | E | + |
| 71 | H | 128 | S | − |
| 72 | L | 120 | C | + |
| 73 | I | 124 | T | − |
| 74 | P | 218 | R | + |
| | I | 238 | R | |
| 75 | H | 297 | R | + |
| | T | 301 | A | |
| 76 | F | 146 | N | + |
| 77 | T | 301 | S | + |
| 78 | H | 128 | M | − |
| | Y | 346 | I | |
| 79 | F | 146 | N | + |
| 80 | H | 297 | R | + |
| | T | 301 | D | |
| 81 | L | 120 | G | − |
| 82 | A | 285 | S | − |
| | T | 284 | M | |
| 83 | C | 290 | I | − |
| | T | 284 | C | |
| 84 | L | 120 | A | − |
| 85 | C | 231 | I | + |
| 86 | N | 374 | G | + |
| 87 | I | 150 | R | − |
| 88 | I | 319 | P | + |
| 89 | S | 147 | A | + |
| 90 | L | 120 | V | + |
| 91 | H | 128 | N | − |
| | T | 350 | A | |
| 92 | C | 290 | I | + |
| | T | 350 | A | |
| 93 | H | 297 | R | + |
| | T | 301 | E | |
| 94 | Q | 303 | K | + |
| | T | 198 | I | |
| 95 | Y | 9 | F | + |
| 96 | A | 269 | E | + |
| | H | 297 | R | |
| 97 | D | 27 | E | + |
| 98 | A | 285 | S | − |
| | T | 198 | I | |
| 99 | I | 319 | E | + |
| 100 | T | 338 | W | − |
| 101 | G | 342 | A | + |
| | Y | 346 | I | |
| 102 | A | 285 | S | − |
| | H | 128 | S | |
| 103 | A | 122 | S | − |
| | H | 128 | N | |
| 104 | F | 313 | Y | + |
| | Q | 303 | K | |
| 105 | T | 145 | P | − |
| 106 | G | 138 | D | + |
| 107 | N | 175 | I | + |
| 108 | P | 218 | R | + |
| 109 | N | 374 | A | + |
| 110 | S | 147 | A | − |
| | Y | 346 | L | |
| 111 | V | 144 | C | + |
| 112 | G | 342 | A | − |
| | S | 147 | A | |
| | Y | 346 | L | |
| 113 | N | 381 | H | ++ |
| 114 | V | 152 | A | − |
| | Y | 346 | A | |
| 115 | L | 206 | Q | ++ |
| | C | 231 | I | |
| 116 | P | 224 | R | − |
| 117 | G | 342 | V | − |
| | Q | 98 | W | |
| | S | 147 | V | |
| | Y | 346 | I | |
| 118 | Q | 365 | D | + |
| 119 | A | 122 | S | + |
| | A | 285 | G | |
| 120 | R | 325 | S | + |
| 121 | I | 150 | D | − |
| 122 | L | 120 | H | − |
| 123 | A | 285 | S | − |
| | T | 350 | A | |
| 124 | T | 338 | S | + |
| 125 | T | 338 | D | + |
| 126 | Q | 315 | Y | − |
| 127 | C | 231 | L | + |
| 128 | F | 146 | G | + |
| 129 | D | 27 | N | + |
| 130 | T | 284 | S | − |
| | Y | 346 | F | |
| 131 | A | 122 | S | + |
| | Q | 303 | K | |
| 132 | R | 29 | G | + |
| 133 | S | 336 | A | + |
| 134 | I | 150 | N | − |
| 135 | A | 122 | S | − |
| | T | 284 | C | |
| 136 | G | 342 | V | − |
| | S | 147 | V | |
| 137 | I | 335 | R | − |
| 138 | L | 199 | I | ++ |
| 139 | T | 284 | M | − |
| | Y | 346 | I | − |
| 140 | H | 297 | N | + |
| | T | 301 | S | |
| 141 | G | 342 | V | − |
| | Q | 98 | W | |
| | S | 147 | V | |
| 142 | H | 128 | N | − |
| | T | 198 | I | |
| 143 | T | 284 | L | |
| 144 | I | 319 | V | − |
| 145 | G | 138 | D | + |
| | T | 350 | A | |
| 146 | F | 266 | M | + |
| 147 | A | 41 | K | + |
| 148 | Q | 329 | H | + |
| 149 | G | 342 | N | − |
| 150 | P | 218 | A | + |
| | I | 238 | T | |
| 151 | A | 285 | S | − |
| | C | 290 | I | |
| 152 | I | 73 | V | + |
| 153 | P | 218 | E | + |
| | I | 238 | T | |
| 154 | P | 218 | K | + |
| | I | 238 | V | |
| 155 | N | 381 | K | + |
| 156 | F | 146 | P | − |
| 157 | P | 218 | G | + |
| 158 | A | 285 | S | − |
| | T | 284 | C | |
| 159 | N | 381 | L | + |
| 160 | F | 100 | A | − |
| | G | 342 | A | |
| | V | 152 | A | |
| | Y | 346 | I | |
| 161 | F | 100 | A | − |
| | V | 152 | A | |
| 162 | A | 285 | G | + |
| | Y | 346 | I | |
| 163 | G | 138 | D | + |
| 164 | G | 287 | A | + |
| 165 | F | 100 | A | − |
| | S | 147 | A | |
| 166 | C | 30 | S | + |
| 167 | H | 297 | D | + |
| 168 | F | 146 | Y | + |
| 169 | G | 138 | D | + |
| | T | 198 | I | |
| 170 | H | 297 | R | + |
| 171 | N | 175 | V | + |
| 172 | S | 151 | C | + |
| 173 | G | 123 | A | − |
| 174 | F | 100 | A | − |
| | V | 152 | A | |
| | Y | 346 | A | |
| 175 | F | 313 | Y | + |
| 176 | A | 285 | S | − |
| | T | 284 | S | |
| 177 | T | 284 | I | − |
| 178 | A | 285 | S | − |
| | Y | 346 | M | |
| 179 | G | 342 | A | − |
| | V | 152 | A | |
| 180 | V | 267 | F | + |
| | F | 271 | L | |
| 181 | I | 335 | V | + |
| 182 | S | 147 | C | − |
| 183 | S | 97 | I | + |
| 184 | P | 218 | H | + |
| | I | 238 | Q | |
| 185 | F | 100 | A | − |
| 186 | C | 231 | T | + |
| 187 | T | 338 | V | + |
| 188 | N | 374 | R | + |
| 189 | Q | 256 | A | + |
| 190 | P | 218 | K | + |
| | I | 238 | R | |
| 191 | M | 217 | V | + |
| 192 | P | 218 | R | + |
| | I | 238 | T | |
| 193 | I | 238 | T | + |
| 194 | T | 121 | R | − |
| 195 | P | 218 | K | + |
| 196 | Q | 256 | S | + |
| 197 | G | 342 | Y | − |
| 198 | F | 100 | A | − |
| | Y | 346 | A | |
| 199 | A | 122 | S | − |
| | H | 128 | S | |
| 200 | G | 265 | N | + |
| | E | 291 | D | |
| 201 | H | 128 | S | − |
| | Y | 346 | F | |
| 202 | S | 147 | A | − |
| | V | 152 | A | |
| 203 | R | 203 | H | − |
| 204 | F | 313 | Y | + |
| | G | 138 | D | |
| 205 | R | 29 | E | + |
| 206 | S | 149 | G | − |
| 207 | F | 146 | A | + |
| 208 | I | 319 | A | + |
| 209 | P | 218 | K | + |
| | I | 238 | E | |
| 210 | Q | 98 | S | + |
| 211 | Q | 256 | H | − |
| 212 | Q | 98 | W | − |
| | S | 147 | V | |
| | Y | 346 | I | |
| 213 | T | 284 | S | + |
| | Y | 346 | I | |
| 214 | G | 342 | T | − |
| 215 | P | 218 | T | + |
| 216 | L | 206 | Q | ++ |
| | C | 231 | V | |
| 217 | A | 285 | G | − |
| | H | 128 | S | |
| 218 | C | 231 | E | + |
| 219 | H | 297 | K | + |
| | T | 301 | S | |
| 220 | A | 122 | S | + |
| | C | 290 | I | |
| 221 | T | 338 | A | + |
| 222 | C | 290 | I | − |
| | H | 128 | N | |
| 223 | E | 143 | W | − |
| | S | 147 | L | |
| 224 | P | 218 | H | + |
| | I | 238 | R | |
| 225 | A | 285 | G | + |
| | Y | 346 | F | |
| 226 | C | 30 | A | + |
| 227 | Q | 355 | E | + |
| 228 | I | 319 | K | + |
| 229 | T | 284 | S | + |
| 230 | C | 30 | R | + |
| 231 | E | 143 | W | − |
| | Q | 98 | W | |
| | Y | 346 | I | |
| 232 | H | 297 | R | + |
| | T | 301 | N | |
| 233 | T | 284 | C | + |
| | Y | 346 | F | |
| 234 | S | 97 | T | + |
| 235 | G | 342 | V | − |
| | S | 147 | V | |
| | Y | 346 | I | |
| 236 | I | 124 | A | − |
| 237 | A | 122 | S | − |
| | H | 128 | M | |
| 238 | C | 290 | I | + |
| | T | 198 | I | |
| 239 | F | 146 | T | + |
| 240 | E | 143 | W | − |
| | Y | 346 | I | |
| 241 | G | 342 | A | − |
| | V | 152 | A | |
| | Y | 346 | I | |
| 242 | T | 284 | V | − |
| 243 | G | 342 | E | − |
| 244 | I | 124 | V | + |
| 245 | F | 271 | L | + |
| 246 | V | 144 | S | + |
| 247 | R | 29 | K | + |
| 248 | C | 290 | I | + |
| 249 | I | 335 | N | + |
| 250 | V | 19 | K | + |
| 251 | T | 284 | M | − |
| | Y | 346 | F | |
| 252 | Q | 98 | W | − |
| | Y | 346 | I | |
| 253 | F | 146 | D | − |
| 254 | I | 150 | S | − |
| 255 | V | 152 | A | − |
| | Y | 346 | L | |
| 256 | C | 30 | H | + |
| 257 | A | 285 | G | − |
| | H | 128 | N | |
| 258 | F | 16 | Y | + |
| 259 | L | 120 | I | + |
| 260 | V | 144 | A | + |
| 261 | G | 53 | A | + |
| 262 | H | 155 | F | + |
| 263 | S | 339 | L | − |
| 264 | L | 199 | I | ++ |
| | F | 266 | L | |
| 265 | Q | 256 | T | − |
| 266 | N | 175 | A | + |
| 267 | F | 313 | Y | + |
| | T | 350 | A | |
| 268 | L | 206 | Q | ++ |
| | C | 231 | L | |
| 269 | P | 218 | G | + |
| | I | 238 | T | |
| 270 | S | 336 | G | + |
| 271 | F | 100 | A | − |
| | V | 152 | A | |
| | Y | 346 | L | |
| 272 | S | 2 | A | ++ |
| 273 | Y | 346 | I | + |
| 274 | P | 218 | D | + |
| | I | 238 | T | + |
| 275 | L | 120 | S | − |
| 276 | V | 152 | A | − |
| 277 | H | 128 | M | − |
| | T | 284 | M | |
| 278 | Y | 346 | F | + |
| 279 | F | 146 | S | + |
| 280 | T | 284 | C | + |
| | Y | 346 | I | |
| 281 | Q | 256 | V | − |
| 282 | A | 122 | S | − |
| | T | 350 | A | |
| 283 | H | 128 | S | − |
| | T | 284 | S | |
| 284 | H | 297 | R | + |
| | T | 301 | S | |
| 285 | G | 342 | V | − |
| | Y | 346 | I | |
| 286 | G | 342 | S | + |
| 287 | Q | 264 | E | + |
| | G | 265 | H | |
| 288 | G | 342 | A | − |
| | V | 152 | A | |
| | Y | 346 | L | |
| 289 | A | 122 | S | − |
| | T | 284 | M | |
| 290 | Q | 98 | T | ++ |
| 291 | P | 218 | H | + |
| | I | 238 | V | |
| 292 | V | 267 | F | + |
| | F | 271 | M | |
| 293 | I | 150 | Q | − |
| 294 | F | 100 | A | − |
| | Y | 346 | L | |
| 295 | G | 138 | D | + |
| | Q | 303 | K | |
| 296 | F | 313 | Y | + |
| | T | 198 | I | |
| 297 | I | 204 | V | + |
| 298 | R | 29 | N | + |
| 299 | H | 128 | N | − |
| 300 | R | 29 | D | + |
| 301 | H | 128 | M | − |
| | T | 284 | S | |
| 302 | I | 335 | T | + |
| 303 | P | 218 | N | + |
| 304 | A | 262 | S | + |
| 305 | E | 143 | W | − |
| 306 | E | 143 | Y | − |
| | Q | 98 | W | |
| 307 | T | 284 | A | + |
| 308 | R | 29 | A | + |
| 309 | H | 297 | K | + |
| 310 | P | 218 | S | + |
| | I | 238 | T | |
| 311 | A | 285 | S | − |
| 312 | Q | 365 | E | + |
| 313 | E | 143 | W | − |
| | Q | 98 | W | |
| 314 | S | 151 | T | − |
| 329 | C | 231 | Y | + |
| 330 | I | 335 | L | + |
| 331 | I | 150 | E | − |
| 332 | A | 122 | S | + |
| 333 | G | 342 | V | − |
| | Q | 98 | W | |
| | Y | 346 | I | |
| 334 | A | 122 | S | − |
| | Y | 346 | I | |
| 335 | G | 342 | V | − |
| 336 | L | 199 | V | ++ |
| | F | 266 | M | |
| 337 | E | 143 | W | − |
| | G | 342 | V | |
| | Q | 98 | W | |
| 338 | H | 297 | D | + |
| | T | 301 | S | |
| 339 | H | 297 | E | + |
| | T | 301 | S | |
| 340 | F | 196 | V | − |
| | M | 217 | V | |
| 341 | S | 149 | A | − |
| 342 | F | 146 | H | + |
| 343 | A | 285 | S | − |
| | Y | 346 | I | |
| 344 | I | 335 | Y | + |
| 345 | G | 342 | M | − |
| 346 | S | 339 | G | − |
| 347 | G | 342 | A | − |
| | S | 147 | A | |
| | V | 152 | A | |
| 348 | H | 128 | M | − |
| | Y | 346 | F | |
| 349 | D | 27 | A | + |
| 350 | N | 374 | H | + |
| 351 | G | 342 | R | − |
| 352 | H | 128 | S | − |
| | T | 284 | M | |
| 353 | Q | 365 | P | + |
| 354 | A | 285 | S | − |
| | Y | 346 | F | |
| 355 | E | 143 | W | − |
| | G | 342 | V | |
| 356 | H | 297 | A | + |
| | T | 301 | S | |
| 357 | T | 284 | S | − |
| | Y | 346 | M | |
| 358 | P | 218 | K | + |
| | I | 238 | T | |
| 359 | T | 145 | S | + |
| 360 | H | 128 | N | − |
| | T | 284 | M | |
| 361 | D | 27 | T | + |
| 362 | L | 120 | F | − |
| 363 | H | 128 | S | − |
| | Y | 346 | I | |
| 364 | N | 374 | Q | + |
| 365 | A | 285 | G | − |
| | Y | 346 | M | |
| 366 | H | 128 | M | − |
| | T | 284 | C | |
| 367 | Q | 264 | E | + |
| | G | 265 | N | |
| 368 | H | 128 | M | − |
| | Y | 346 | M | |
| 369 | Q | 329 | N | + |
| 370 | A | 382 | S | + |
| 371 | F | 100 | A | − |
| | G | 342 | A | |
| | Y | 346 | I | |
| 372 | H | 297 | A | + |
| 373 | Q | 256 | E | − |
| 374 | I | 150 | V | + |
| 375 | S | 339 | A | − |
| 376 | H | 128 | S | − |
| | T | 284 | C | |
| 377 | P | 218 | H | + |
| | I | 238 | E | |
| 378 | E | 143 | Y | − |
| 379 | T | 198 | I | + |
| | T | 350 | A | |
| 380 | C | 231 | D | + |
| 381 | F | 146 | V | − |
| 382 | K | 384 | S | + |
| 383 | G | 342 | V | − |
| | Q | 98 | W | − |
| 384 | V | 144 | N | − |
| 385 | N | 175 | T | + |
| 386 | T | 284 | C | − |
| | T | 350 | A | |
| 387 | Q | 98 | R | − |
| 388 | Q | 264 | E | + |
| 389 | V | 144 | T | + |
| 390 | H | 297 | Q | + |
| | T | 301 | S | |
| 391 | H | 128 | N | − |
| | Y | 346 | F | |
| 392 | C | 290 | I | + |
| | G | 138 | D | |
| 393 | W | 101 | F | + |
| 394 | A | 122 | S | − |
| | Y | 346 | F | |
| 395 | G | 342 | V | − |
| | S | 147 | L | |
| 396 | P | 218 | Q | + |
| 397 | L | 199 | V | + |
| 398 | T | 338 | L | − |
| 399 | G | 265 | H | + |
| | E | 291 | D | |
| 400 | T | 338 | N | + |
| 402 | L | 120 | Q | − |
| 403 | G | 342 | A | + |
| | Y | 346 | L | |
| 404 | G | 342 | C | + |
| 405 | S | 149 | T | − |
| 406 | G | 342 | D | − |
| 407 | A | 285 | G | + |
| | T | 284 | S | |
| 408 | P | 218 | H | + |
| 409 | N | 381 | R | + |
| 410 | F | 271 | M | + |
| 411 | H | 128 | N | − |
| | Y | 346 | M | |
| 412 | S | 149 | V | − |
| 413 | R | 29 | T | + |
| 414 | S | 147 | V | − |
| | Y | 346 | I | − |
| 415 | N | 374 | K | + |
| 416 | T | 350 | A | + |
| 417 | A | 122 | S | − |
| | T | 198 | I | |
| 418 | E | 143 | W | − |
| | Q | 98 | W | |
| | S | 147 | L | |
| 419 | P | 218 | H | + |
| | I | 238 | T | |
| 420 | A | 285 | G | + |
| 421 | N | 175 | E | + |
| 422 | F | 100 | A | − |
| | Y | 346 | I | |
| 423 | I | 150 | L | − |
| 424 | R | 29 | S | + |
| 425 | A | 153 | T | − |
| 426 | D | 27 | V | + |

“+” = greater than 0.5 to 1.5 fold increase in activity relative to control
“++” = greater than 1.5 fold increase in activity relative to control

TABLE 7
Relative Activity of Exemplary FDHs Engineered using the
FDH from Candida boidinii (SEQ ID NO: 2) as the Template
Compared to the FDH of Candida boidinii wild-type control

| Variant # | Residue | Position | Alteration | Activity |
|---|---|---|---|---|
| 1 | T | 315 | C | + |
| | Y | 323 | M | |
| 2 | A | 260 | G | + |
| | A | 91 | S | |
| 3 | Q | 320 | T | + |
| | T | 315 | S | |
| 4 | H | 361 | R | + |
| | H | 97 | N | |
| | N | 214 | T | |
| 5 | D | 36 | K | + |
| | H | 361 | R | |
| | I | 162 | L | |
| | S | 111 | T | |
| 6 | A | 257 | G | + |
| | Y | 312 | V | |
| 7 | A | 257 | G | + |
| | S | 313 | G | |
| 8 | H | 361 | R | ++ |
| | S | 111 | T | |
| 9 | D | 362 | K | + |
| | N | 214 | T | |
| 10 | A | 260 | G | + |
| | S | 313 | A | |
| 11 | S | 313 | G | + |
| | T | 315 | S | |
| 12 | A | 91 | S | + |
| | Y | 312 | F | |
| 13 | H | 361 | R | ++ |
| | I | 162 | L | |
| 14 | D | 362 | K | + |
| | N | 214 | T | |
| | P | 188 | C | |
| 15 | D | 36 | K | + |
| | I | 162 | L | |
| | S | 111 | T | |
| 16 | A | 229 | T | ++ |
| | A | 260 | G | |
| 17 | H | 361 | R | ++ |
| | N | 214 | T | |
| 18 | T | 256 | C | + |
| | Y | 312 | T | |
| 19 | I | 162 | L | + |
| | N | 214 | T | |
| | S | 111 | T | |
| 20 | H | 361 | R | + |
| | H | 97 | N | |
| 21 | D | 362 | K | + |
| | P | 188 | C | |
| 22 | D | 80 | E | + |
| | I | 162 | L | |
| 23 | A | 229 | T | + |
| | V | 120 | I | |
| 24 | T | 256 | C | + |
| | Y | 312 | V | |
| 25 | D | 36 | K | ++ |
| | H | 361 | R | |
| | I | 162 | L | |
| | N | 214 | T | |
| 26 | N | 187 | G | + |
| | S | 111 | T | |
| 27 | D | 80 | E | ++ |
| | H | 361 | R | |
| 28 | I | 64 | V | + |
| | N | 214 | T | |
| 29 | Q | 320 | S | ++ |
| | V | 120 | I | |
| 30 | I | 64 | V | + |
| | N | 187 | G | |
| 31 | D | 362 | K | + |
| | I | 162 | L | |
| | S | 111 | T | |
| 32 | D | 36 | K | ++ |
| | H | 361 | R | |
| 33 | I | 162 | L | ++ |
| | S | 111 | T | |
| 34 | D | 36 | K | + |
| | I | 162 | L | |
| | I | 164 | V | |
| | N | 214 | T | |
| 49 | A | 257 | G | + |
| | A | 260 | G | |
| 50 | A | 257 | S | + |
| | A | 260 | G | |
| 51 | D | 36 | K | + |
| | I | 162 | L | |
| | I | 64 | V | |
| 52 | D | 362 | K | + |
| | I | 162 | L | |
| 53 | D | 362 | K | + |
| | I | 162 | L | |
| | I | 64 | V | |
| 54 | D | 36 | K | + |
| | D | 80 | E | |
| | I | 162 | L | |
| 55 | H | 361 | R | ++ |
| | I | 162 | L | |
| | S | 111 | T | |
| 56 | A | 229 | V | + |
| | A | 91 | S | |
| 57 | D | 362 | K | + |
| | I | 64 | V | |
| 58 | H | 361 | R | ++ |
| | I | 162 | L | |
| | I | 64 | V | |
| | N | 214 | T | |
| 59 | I | 162 | L | + |
| | I | 64 | V | |
| 60 | D | 36 | K | + |
| | D | 80 | E | |
| | H | 361 | R | |
| 61 | N | 214 | T | + |
| | S | 111 | T | |
| 62 | A | 229 | C | ++ |
| | T | 315 | C | |
| 63 | D | 362 | K | + |
| | D | 80 | E | |
| 64 | A | 229 | V | + |
| | Y | 312 | F | |

“+” = greater than 0.5- to 1.5-fold increase in FDH activity relative to control
“++” = greater than 1.5-fold increase in FDH activity relative to control

Based on these results, variants 113, 115, 138, 216, 264, 268, 272, 290 and 336 based on the FDH of Gibbsiella quercinecans (SEQ ID NO: 1) and variants 8, 13, 16, 17, 25, 27, 29, 32, 33, 55, 58, and 62 based on the FDH of Candida boidinii (SEQ ID NO: 2) were identified as having the highest increases in activity (e.g., greater than 1.5-fold increase) relative to the activity of the corresponding control FDH, whereas numerous other variants showed a modest increase in FDH activity (e.g., greater than 0.5-fold to 1.5-fold increase) relative to control.

In addition to performing the additional screening for the above identified variants, homologs of the FDH of Gibbsiella quercinecans (SEQ ID NO: 1) and the FDH of Candida boidinii (SEQ ID NO: 2) from various other organisms were identified and screened for activity using the same assays described in Example 2 and this Example. The results of this screen are provided in Table 8.

TABLE 8
Formate Dehydrogenase Homologs

| SEQ | | Activity (relative to |
|---|---|---|
| ID NO | Organism | SEQ ID NO: 2) |
| 1 | Gibbsiella quercinecans | ++ |
| 2 | Candida boidinii | Control levels |
| 3 | Candida boidinii | ++ |
| 4 | Clohesyomyces aquaticus | ++ |
| 5 | Clavispora lusitaniae | + |
| 6 | Caballeronia udeis | + |
| 7 | Coccidioides posadasii RMSCC | + |
| | 3488 | |
| 8 | Botryotinia fuckeliana | + |
| 9 | Yarrowia lipolytica | + |
| 10 | Kushneria avicenniae | + |
| 11 | Zea mays | + |
| 12 | Dactylellina haptotyla | + |
| 13 | Segniliparus rugosus | + |
| 14 | Aspergillus indologenus CBS | + |
| | 114.80 | |
| 15 | Aspergillus indologenus | + |
| 16 | Actinidia chinensis var. | + |
| | chinensis | |
| 17 | Aspergillus kawachii | + |
| 18 | Candidatus Nitrosocosmicus | + |
| | oleophilus | |
| 19 | Blastomyces gilchristii | − |
| 20 | Paraburkholderia caribensis | − |
| | MBA4 | |
| 21 | Mesorhizobium sp. | − |
| 22 | Helicocarpus griseus | − |
| | UAMH5409 | |
| 23 | Streptomyces albidoflavus | − |

“−” = no to little activity
“+” = greater than 0.5- to 1.5-fold increase in activity relative to control
“++” = greater than 1.5-fold increase in activity relative to control

Based on these results, various FDH homologs were identified as having activity that was greater than 1.5-fold that of the FDH of Candida boidinii (SEQ ID NO: 2), including the FDH from Gibbsiella quercinecans (SEQ ID NO: 1), Candida boidinii (SEQ ID NO: 3), and Clohesyomyces aquaticus (SEQ ID NO: 4).

### Example 8

Increased Production of 1,3-BDO in E. Coli with Expression of FDH Variants

In order to assess the impact of the variant FDHs generated and analyzed in EXAMPLE 1-7 on the production of a bioderived product, genes encoding select FDHs were transformed into a strain of E. coli that also included introduced genes encoding 1,3-BDO pathway enzymes: 1) a thiolase (Th1), 2) a 3-hydoxybutryl-CoA dehydrogenase (Hbd), 3) an aldehyde dehydrogenase (Ald), and 4) an alcohol dehydrogenase (Adh). The 3-hydoxybutryl-CoA dehydrogenase utilizes NADH as a cofactor. The aldehyde dehydrogenase utilizes NADH or NADPH as a cofactor, with NADH being preferred. The alcohol dehydrogenase utilizes NADPH as a cofactor. The FDHs that were introduced included the FDH of Gibbsiella quercinecans (SEQ ID NO: 1), the FDH of Candida boidinii (SEQ ID NO: 2), or an FDH variant that was identified in Example 6 as having activity that is greater than 1.5-fold than that of the wild-type FDH (i.e., relative to an FDH having the amino acid sequence of SEQ ID NO: 1).

The vectors for expressing the variant FDH genes were transformed into the Th1/Hbd/Ald/Adh E. coli strain and transformants were tested for 1,3-BDO production. The engineered E. co/i cells were fed 2% glucose in minimal media, and after an 18 h incubation at 35° C., the cells were harvested, and the supernatants were evaluated by analytical HPLC or standard LC/MS analytical method for 1,3-BDO production.

The results with the variants are shown in TABLE 9.

TABLE 9
Production of 1,3-BDO using Exemplary FDHs
Engineered using the FDH of Gibbsiella
quercinecans (SEQ ID NO: 1) as the Template.

| | | | | In vivo 1,3-BDO |
|---|---|---|---|---|
| Variant # | Residue | Position | Alteration | production |
| 113 | N | 381 | H | + |
| 115 | L | 206 | Q | + |
| | C | 231 | I | |
| 138 | L | 199 | I | + |
| 216 | L | 206 | Q | + |
| | C | 231 | V | |
| 264 | L | 199 | I | + |
| | F | 266 | L | |
| 268 | I | 206 | Q | + |
| | C | 231 | L | |
| 272 | S | 2 | A | + |
| 290 | Q | 98 | T | + |
| 336 | L | 199 | V | + |
| | F | 266 | M | |

“+” = detection of 1,3-BDO production.

Both the FDH of Gibbsiella quercinecans (SEQ ID NO: 1) and the FDH of Candida boidinii (SEQ ID NO: 2) showed production of 1,3-BDO. Moreover, all the variants that were tested showed production of 1,3-BDO. These results demonstrate that the production of NADH and/or removal of formate by conversion to carbon dioxide through activity of an engineered FDH can be used in the production of a bioderived product, such as 1,3-BDO.

Throughout this application various publications have been referenced. The disclosures of these publications in their entireties are hereby incorporated by reference in this application in order to more fully describe the state of the art to which this invention pertains. Although the invention has been described with reference to the examples provided above, it should be understood that various modifications can be made without departing from the spirit described herein.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications and variances which fall within the scope of the appended claims.

What is claimed is:

1. An engineered formate dehydrogenase comprising a variant of amino acid sequence SEQ ID NO: 1 or SEQ ID NO: 2 or a functional fragment thereof, wherein the engineered formate dehydrogenase comprises one or more alterations at a position described in TABLE 6 and/or TABLE 7.

2. The engineered formate dehydrogenase of claim 1, wherein the engineered formate dehydrogenase is capable of:
a) catalyzing the conversion of formate to carbon dioxide;
b) catalyzing the conversion of NAD⁺ to NADH; or
c) catalyzing the conversion of formate to carbon dioxide and NAD+ to NADH.

3. The engineered formate dehydrogenase of claim 1 or 2, wherein the engineered formate dehydrogenase is capable of catalyzing the conversion of formate to carbon dioxide and NAD⁺ to NADH.

4. The engineered formate dehydrogenase of any one of claims 1 to 3, wherein the engineered formate dehydrogenase comprises an activity that is at least 0.5, at least 1, at least 1.5, or at least 2-fold higher than the activity of a formate dehydrogenase consisting of the amino acid sequence of SEQ ID NO: 1 or SEQ ID NO: 2.

5. The engineered formate dehydrogenase of any one of claims 1 to 4, wherein the engineered formate dehydrogenase comprises one or more amino acid substitutions at a position corresponding to position 2, 9, 16, 19, 27, 29, 30, 41, 53, 73, 97, 98, 100, 101, 120, 121, 122, 123, 124, 128, 138, 143, 144, 145, 146, 147, 149, 150, 151, 152, 153, 155, 175, 176, 191, 196, 198, 199, 203, 204, 206, 217, 218, 224, 231, 238, 256, 262, 264, 265, 266, 267, 269, 271, 284, 285, 287, 290, 291, 297, 301, 303, 313, 315, 319, 325, 329, 335, 336, 338, 339, 342, 343, 346, 350, 355, 365, 374, 381, 382, or 384, or a combination thereof, in SEQ ID NO: 1.

6. The engineered formate dehydrogenase of any one of claims 1 to 4, wherein the engineered formate dehydrogenase comprises one or more amino acid substitutions at a position corresponding to position 2, 9, 16, 19, 27, 29, 30, 41, 53, 73, 97, 98, 101, 120, 122, 124, 138, 144, 145, 146, 147, 150, 151, 155, 175, 176, 191, 198, 199, 204, 206, 217, 218, 231, 238, 256, 262, 264, 265, 266, 267, 269, 271, 284, 285, 287, 290, 291, 297, 301, 303, 313, 319, 325, 329, 335, 336, 338, 339, 342, 346, 350, 355, 365, 374, 381, 382, or 384, or a combination thereof, in SEQ ID NO: 1.

7. The engineered formate dehydrogenase of any one of claims 1 to 4, wherein the engineered formate dehydrogenase comprises one or more amino acid substitutions at a position corresponding to position 2, 98, 199, 206, 231, 266, or 381, or a combination thereof, in SEQ ID NO: 1.

8. The engineered formate dehydrogenase of any one of claims 1 to 4, wherein the engineered formate dehydrogenase comprises one or more amino acid substitutions at a position corresponding to position 9, 16, 19, 27, 29, 30, 41, 53, 73, 97, 98, 101, 120, 122, 124, 138, 144, 145, 146, 147, 150, 151, 155, 175, 176, 191, 198, 199, 204, 217, 218, 231, 238, 256, 262, 264, 265, 266, 267, 269, 271, 284, 285, 287, 290, 291, 297, 301, 303, 313, 319, 325, 329, 335, 336, 338, 339, 342, 346, 350, 355, 365, 374, 381, 382, or 384, or a combination thereof, in SEQ ID NO: 1.

9. The engineered formate dehydrogenase of any one of claims 1 to 4, wherein the engineered formate dehydrogenase comprises one or more amino acid substitutions at a position corresponding to position 36, 64, 80, 91, 97, 111, 120, 162, 164, 187, 188, 214, 229, 256, 257, 260, 312, 313, 315, 320, 323, 361, or 362, or a combination thereof, in SEQ ID NO: 2.

10. The engineered formate dehydrogenase of any one of claims 1 to 4, wherein the engineered formate dehydrogenase comprises one or more amino acid substitutions at a position corresponding to position 36, 64, 80, 111, 120, 162, 214, 229, 260, 315, 320, or 361, or a combination thereof, in SEQ ID NO: 2.

11. The engineered formate dehydrogenase of any one of claims 1 to 10, wherein the one or more amino acid alterations are conservative amino acid substitutions.

12. The engineered formate dehydrogenase of any one of claims 1 to 10, wherein the one or more amino acid alterations are non-conservative amino acid substitutions.

13. The engineered formate dehydrogenase of any one of claims 1 to 4, wherein the one or more amino acid alterations of the engineered formate dehydrogenase is an alteration described in TABLE 6.

14. The engineered formate dehydrogenase of claim 13, wherein the one or more amino acid alterations result in an engineered formate dehydrogenase comprising:
a) A at a residue corresponding to position 2 in SEQ ID NO: 1;
b) F at a residue corresponding to position 9 in SEQ ID NO: 1;
c) Y at a residue corresponding to position 16 in SEQ ID NO: 1;
d) K or S at a residue corresponding to position 19 in SEQ ID NO: 1;
e) K, E, N, A, T, or V at a residue corresponding to position 27 in SEQ ID NO: 1;
f) G, E, K, N, D, A, T, or S at a residue corresponding to position 29 in SEQ ID NO: 1;
g) G, S, A, R, or H at a residue corresponding to position 30 in SEQ ID NO: 1;
h) K at a residue corresponding to position 41 in SEQ ID NO: 1;
i) A at a residue corresponding to position 53 in SEQ ID NO: 1;
j) V at a residue corresponding to position 73 in SEQ ID NO: 1;
k) I or T at a residue corresponding to position 97 in SEQ ID NO: 1;
l) W, S, T, or R at a residue corresponding to position 98 in SEQ ID NO: 1;
m) A at a residue corresponding to position 100 in SEQ ID NO: 1;
n) F at a residue corresponding to position 101 in SEQ ID NO: 1;
o) C, G, A, V, H, I, S, F, or Q at a residue corresponding to position 120 in SEQ ID NO: 1;
p) R at a residue corresponding to position 121 in SEQ ID NO: 1;
q) S at a residue corresponding to position 122 in SEQ ID NO: 1;
r) A at a residue corresponding to position 123 in SEQ ID NO: 1;
s) T, A, V at a residue corresponding to position 124 in SEQ ID NO: 1;
t) N, M, or S at a residue corresponding to position 128 in SEQ ID NO: 1;
u) D at a residue corresponding to position 138 in SEQ ID NO: 1;
v) W or Y at a residue corresponding to position 143 in SEQ ID NO: 1;
w) I, C, S, A, N, or T at a residue corresponding to position 144 in SEQ ID NO: 1;
x) P or S at a residue corresponding to position 145 in SEQ ID NO: 1;
y) Q, N, G, P, Y, A, T, D, S, H, or V at a residue corresponding to position 146 in SEQ ID NO: 1;
z) A, L, V, or C at a residue corresponding to position 147 in SEQ ID NO: 1;
aa) G, A, T, or V at a residue corresponding to position 149 in SEQ ID NO: 1;
bb) T, G, R, D, N, S, Q, E, V, or L at a residue corresponding to position 150 in SEQ ID NO: 1;
cc) A, C, or T at a residue corresponding to position 151 in SEQ ID NO: 1;
dd) A at a residue corresponding to position 152 in SEQ ID NO: 1;
ee) T at a residue corresponding to position 153 in SEQ ID NO: 1;
ff) F at a residue corresponding to position 155 in SEQ ID NO: 1;
gg) R, I, V, A, T, or E at a residue corresponding to position 175 in SEQ ID NO: 1;
hh) S at a residue corresponding to position 176 in SEQ ID NO: 1;
ii) L at a residue corresponding to position 191 in SEQ ID NO: 1;
jj) V at a residue corresponding to position 196 in SEQ ID NO: 1;
kk) I at a residue corresponding to position 198 in SEQ ID NO: 1;
ll) I or V at a residue corresponding to position 199 in SEQ ID NO: 1;
mm) H at a residue corresponding to position 203 in SEQ ID NO: 1;
nn) V at a residue corresponding to position 204 in SEQ ID NO: 1;
oo) Q at a residue corresponding to position 206 in SEQ ID NO: 1;
pp) V at a residue corresponding to position 217 in SEQ ID NO: 1;
qq) T, N, R, A, E, K, G, H, R, D, S, or Q at a residue corresponding to position 218 in SEQ ID NO: 1;
rr) R at a residue corresponding to position 224 in SEQ ID NO: 1;
ss) D, A, K, R, V, I, L, T, Y or E at a residue corresponding to position 231 in SEQ ID NO: 1;
tt) T, R, V, Q, or E at a residue corresponding to position 238 in SEQ ID NO: 1;
uu) I, C, L, A, S, H, T, V, or E at a residue corresponding to position 256 in SEQ ID NO: 1;
vv) E or S at a residue corresponding to position 262 in SEQ ID NO: 1;
ww) E at a residue corresponding to position 264 in SEQ ID NO: 1;
xx) N or H at a residue corresponding to position 265 in SEQ ID NO: 1;
yy) M or L at a residue corresponding to position 266 in SEQ ID NO: 1;
zz) F at a residue corresponding to position 267 in SEQ ID NO: 1;
aaa) D or E at a residue corresponding to position 269 in SEQ ID NO: 1;
bbb) L or M at a residue corresponding to position 271 in SEQ ID NO: 1;
ccc) S, C, M, L, I, V, or A at a residue corresponding to position 284 in SEQ ID NO: 1;
ddd) S or G at a residue corresponding to position 285 in SEQ ID NO: 1;
eee) A at a residue corresponding to position 287 in SEQ ID NO: 1;
fff) I at a residue corresponding to position 290 in SEQ ID NO: 1;
ggg) D at a residue corresponding to position 291 in SEQ ID NO: 1;
hhh) R, V, G, N, D, K, E, A, or Q at a residue corresponding to position 297 in SEQ ID NO: 1;
iii) S, A, D, E, or N at a residue corresponding to position 301 in SEQ ID NO: 1;
jjj) K at a residue corresponding to position 303 in SEQ ID NO: 1;
kkk) Y at a residue corresponding to position 313 in SEQ ID NO: 1;
lll) E or Y at a residue corresponding to position 315 in SEQ ID NO: 1;
mmm) R, P, E, V, A, or K at a residue corresponding to position 319 in SEQ ID NO: 1;
nnn) T or S at a residue corresponding to position 325 in SEQ ID NO: 1;
ooo) H or N at a residue corresponding to position 329 in SEQ ID NO: 1;
ppp) M, R, V, N, T, L, S, or Y at a residue corresponding to position 335 in SEQ ID NO: 1;
qqq) A or G at a residue corresponding to position 336 in SEQ ID NO: 1;
rrr) Y, F, W, S, D, V, A, L, or N at a residue corresponding to position 338 in SEQ ID NO: 1;
sss) T, L, G, or A at a residue corresponding to position 339 in SEQ ID NO: 1;
ttt) K, L, A, V, I, N, Y, T, E, S, M, R, C, or D at a residue corresponding to position 342 in SEQ ID NO: 1;
uuu) A at a residue corresponding to position 343 in SEQ ID NO: 1;
vvv) A, M, I, L, or F at a residue corresponding to position 346 in SEQ ID NO: 1;
www) A at a residue corresponding to position 350 in SEQ ID NO: 1;
xxx) E at a residue corresponding to position 355 in SEQ ID NO: 1;
yyy) D, E, or P at a residue corresponding to position 365 in SEQ ID NO: 1;
zzz) E, G, A, R, H, Q, or K at a residue corresponding to position 374 in SEQ ID NO: 1;
aaaa) H, K, L, P, or R at a residue corresponding to position 381 in SEQ ID NO: 1;
bbbb) S at a residue corresponding to position 382 in SEQ ID NO: 1; and/or
cccc) S or T at a residue corresponding to position 384 in SEQ ID NO: 1.

15. The engineered formate dehydrogenase of claim 13, wherein the one or more amino acid alterations result in an engineered formate dehydrogenase comprising:
a) A at a residue corresponding to position 2 in SEQ ID NO: 1;
b) F at a residue corresponding to position 9 in SEQ ID NO: 1;
c) Y at a residue corresponding to position 16 in SEQ ID NO: 1;
d) K or S at a residue corresponding to position 19 in SEQ ID NO: 1;
e) K, E, N, A, T, or V at a residue corresponding to position 27 in SEQ ID NO: 1;
f) G, E, K, N, D, A, T, or S at a residue corresponding to position 29 in SEQ ID NO: 1;
g) G, S, A, R, or H at a residue corresponding to position 30 in SEQ ID NO: 1;
h) K at a residue corresponding to position 41 in SEQ ID NO: 1;
i) A at a residue corresponding to position 53 in SEQ ID NO: 1;
j) V at a residue corresponding to position 73 in SEQ ID NO: 1;
k) I or T at a residue corresponding to position 97 in SEQ ID NO: 1;
l) W, R, S, or T at a residue corresponding to position 98 in SEQ ID NO: 1;
m) F at a residue corresponding to position 101 in SEQ ID NO: 1;
n) G, A, H, S, F, Q, C, V, or I at a residue corresponding to position 120 in SEQ ID NO: 1;
o) S at a residue corresponding to position 122 in SEQ ID NO: 1;
p) T, A, or V at a residue corresponding to position 124 in SEQ ID NO: 1;
q) D at a residue corresponding to position 138 in SEQ ID NO: 1;
r) N, I, C, S, A, or T at a residue corresponding to position 144 in SEQ ID NO: 1;
s) P or S at a residue corresponding to position 145 in SEQ ID NO: 1;
t) P, D, V, Q, N, G, Y, A, T, S, or H at a residue corresponding to position 146 in SEQ ID NO: 1;
u) V, L, C, or A at a residue corresponding to position 147 in SEQ ID NO: 1;
v) G, R, D, N, S, Q, E, L, T or V at a residue corresponding to position 150 in SEQ ID NO: 1;
w) T, A, or C at a residue corresponding to position 151 in SEQ ID NO: 1;
x) F at a residue corresponding to position 155 in SEQ ID NO: 1;
y) R, I, V, A, T, or E at a residue corresponding to position 175 in SEQ ID NO: 1;
z) S at a residue corresponding to position 176 in SEQ ID NO: 1;
aa) L at a residue corresponding to position 191 in SEQ ID NO: 1;
bb) I at a residue corresponding to position 198 in SEQ ID NO: 1;
cc) I or V at a residue corresponding to position 199 in SEQ ID NO: 1;
dd) V at a residue corresponding to position 204 in SEQ ID NO: 1;
ee) Q at a residue corresponding to position 206 in SEQ ID NO: 1;
ff) V at a residue corresponding to position 217 in SEQ ID NO: 1;
gg) T, N, R, A, E, K, G, H, D, S, or Q at a residue corresponding to position 218 in SEQ ID NO: 1;
hh) D, A, K, R, V, I, L, T, Y or E at a residue corresponding to position 231 in SEQ ID NO: 1;
ii) T, R, V, Q, or E at a residue corresponding to position 238 in SEQ ID NO: 1;
jj) I, C, L, H, T, V, E, A, or S at a residue corresponding to position 256 in SEQ ID NO: 1;
kk) E or S at a residue corresponding to position 262 in SEQ ID NO: 1;
ll) E at a residue corresponding to position 264 in SEQ ID NO: 1;
mm) N or H at a residue corresponding to position 265 in SEQ ID NO: 1;
nn) M or L at a residue corresponding to position 266 in SEQ ID NO: 1;
oo) F at a residue corresponding to position 267 in SEQ ID NO: 1;
pp) D or E at a residue corresponding to position 269 in SEQ ID NO: 1;
qq) L or M at a residue corresponding to position 271 in SEQ ID NO: 1;
rr) L, I, V, S, C, M, or A at a residue corresponding to position 284 in SEQ ID NO: 1;
ss) S or G at a residue corresponding to position 285 in SEQ ID NO: 1;
tt) A at a residue corresponding to position 287 in SEQ ID NO: 1;
uu) I at a residue corresponding to position 290 in SEQ ID NO: 1;
vv) D at a residue corresponding to position 291 in SEQ ID NO: 1;
ww) R, V, G, N, D, K, E, A, or Q at a residue corresponding to position 297 in SEQ ID NO: 1;
xx) S, A, D, E, or N at a residue corresponding to position 301 in SEQ ID NO: 1;
yy) K at a residue corresponding to position 303 in SEQ ID NO: 1;
zz) Y at a residue corresponding to position 313 in SEQ ID NO: 1;
aaa) R, P, E, V, A, or K at a residue corresponding to position 319 in SEQ ID NO: 1;
bbb) T or S at a residue corresponding to position 325 in SEQ ID NO: 1;
ccc) H or N at a residue corresponding to position 329 in SEQ ID NO: 1;
ddd) R, S, A, M, V, N, T, L, or Y at a residue corresponding to position 335 in SEQ ID NO: 1;
eee) A or G at a residue corresponding to position 336 in SEQ ID NO: 1;
fff) Y, F, W, L, S, D, V, A, or N at a residue corresponding to position 338 in SEQ ID NO: 1;
ggg) L, G, A, T at a residue corresponding to position 339 in SEQ ID NO: 1;
hhh) K, L, V, I, N, Y, T, E, M, R, D, A, S, or C at a residue corresponding to position 342 in SEQ ID NO: 1;
iii) M, A, I, L, or F at a residue corresponding to position 346 in SEQ ID NO: 1;
jjj) A at a residue corresponding to position 350 in SEQ ID NO: 1;
kkk) E at a residue corresponding to position 355 in SEQ ID NO: 1;
lll) D, E, or P at a residue corresponding to position 365 in SEQ ID NO: 1;
mmm) E, G, A, R, H, Q, or K at a residue corresponding to position 374 in SEQ ID NO: 1;
nnn) P, H, K, L, or R at a residue corresponding to position 381 in SEQ ID NO: 1;
ooo) S at a residue corresponding to position 382 in SEQ ID NO: 1; and/or
ppp) S or T at a residue corresponding to position 384 in SEQ ID NO: 1.

16. The engineered formate dehydrogenase of claim 13, wherein the one or more amino acid alterations result in an engineered formate dehydrogenase comprising:
a) A at a residue corresponding to position 2 in SEQ ID NO: 1;
b) F at a residue corresponding to position 9 in SEQ ID NO: 1;
c) Y at a residue corresponding to position 16 in SEQ ID NO: 1;
d) K or S at a residue corresponding to position 19 in SEQ ID NO: 1;
e) K, E, N, A, T, or V at a residue corresponding to position 27 in SEQ ID NO: 1;
f) G, E, K, N, D, A, T, or S at a residue corresponding to position 29 in SEQ ID NO: 1;
g) G, S, A, R, or H at a residue corresponding to position 30 in SEQ ID NO: 1;
h) K at a residue corresponding to position 41 in SEQ ID NO: 1;
i) A at a residue corresponding to position 53 in SEQ ID NO: 1;
j) V at a residue corresponding to position 73 in SEQ ID NO: 1;
k) I or T at a residue corresponding to position 97 in SEQ ID NO: 1;
l) S or T at a residue corresponding to position 98 in SEQ ID NO: 1;
m) F at a residue corresponding to position 101 in SEQ ID NO: 1;
n) C, V, or I at a residue corresponding to position 120 in SEQ ID NO: 1;
o) S at a residue corresponding to position 122 in SEQ ID NO: 1;
p) V at a residue corresponding to position 124 in SEQ ID NO: 1;
q) D at a residue corresponding to position 138 in SEQ ID NO: 1;
r) I, C, S, A, or T at a residue corresponding to position 144 in SEQ ID NO: 1;
s) S at a residue corresponding to position 145 in SEQ ID NO: 1;
t) Q, N, G, Y, A, T, S, or H at a residue corresponding to position 146 in SEQ ID NO: 1;
u) A at a residue corresponding to position 147 in SEQ ID NO: 1;
v) T or V at a residue corresponding to position 150 in SEQ ID NO: 1;
w) A or C at a residue corresponding to position 151 in SEQ ID NO: 1;
x) F at a residue corresponding to position 155 in SEQ ID NO: 1;
y) R, I, V, A, T, or E at a residue corresponding to position 175 in SEQ ID NO: 1;
z) S at a residue corresponding to position 176 in SEQ ID NO: 1;
aa) L at a residue corresponding to position 191 in SEQ ID NO: 1;
bb) I at a residue corresponding to position 198 in SEQ ID NO: 1;
cc) I or V at a residue corresponding to position 199 in SEQ ID NO: 1;
dd) V at a residue corresponding to position 204 in SEQ ID NO: 1;
ee) Q at a residue corresponding to position 206 in SEQ ID NO: 1;
ff) V at a residue corresponding to position 217 in SEQ ID NO: 1;
gg) T, N, R, A, E, K, G, H, D, S, or Q at a residue corresponding to position 218 in SEQ ID NO: 1;
hh) D, A, K, R, V, I, L, T, Y or E at a residue corresponding to position 231 in SEQ ID NO: 1;
ii) T, R, V, Q, or E at a residue corresponding to position 238 in SEQ ID NO: 1;
jj) A or S at a residue corresponding to position 256 in SEQ ID NO: 1;
kk) E or S at a residue corresponding to position 262 in SEQ ID NO: 1;
ll) E at a residue corresponding to position 264 in SEQ ID NO: 1;
mm) N or H at a residue corresponding to position 265 in SEQ ID NO: 1;
nn) M or L at a residue corresponding to position 266 in SEQ ID NO: 1;
oo) F at a residue corresponding to position 267 in SEQ ID NO: 1;
pp) D or E at a residue corresponding to position 269 in SEQ ID NO: 1;
qq) L or M at a residue corresponding to position 271 in SEQ ID NO: 1;
rr) S, C, M, or A at a residue corresponding to position 284 in SEQ ID NO: 1;
ss) G at a residue corresponding to position 285 in SEQ ID NO: 1;
tt) A at a residue corresponding to position 287 in SEQ ID NO: 1;
uu) I at a residue corresponding to position 290 in SEQ ID NO: 1;
vv) D at a residue corresponding to position 291 in SEQ ID NO: 1;
ww) R, V, G, N, D, K, E, A, or Q at a residue corresponding to position 297 in SEQ ID NO: 1;
xx) S, A, D, E, or N at a residue corresponding to position 301 in SEQ ID NO: 1;
yy) K at a residue corresponding to position 303 in SEQ ID NO: 1;
zz) Y at a residue corresponding to position 313 in SEQ ID NO: 1;
aaa) R, P, E, V, A, or K at a residue corresponding to position 319 in SEQ ID NO: 1;
bbb) T or S at a residue corresponding to position 325 in SEQ ID NO: 1;
ccc) H or N at a residue corresponding to position 329 in SEQ ID NO: 1;
ddd) S, A, M, V, N, T, L, or Y at a residue corresponding to position 335 in SEQ ID NO: 1;
eee) A or G at a residue corresponding to position 336 in SEQ ID NO: 1;
fff) S, D, V, A, or N at a residue corresponding to position 338 in SEQ ID NO: 1;
ggg) T at a residue corresponding to position 339 in SEQ ID NO: 1;
hhh) A, S, or C at a residue corresponding to position 342 in SEQ ID NO: 1;
iii) I, L, or F at a residue corresponding to position 346 in SEQ ID NO: 1;
jjj) A at a residue corresponding to position 350 in SEQ ID NO: 1;
kkk) E at a residue corresponding to position 355 in SEQ ID NO: 1;
lll) D, E, or P at a residue corresponding to position 365 in SEQ ID NO: 1;
mmm) E, G, A, R, H, Q, or K at a residue corresponding to position 374 in SEQ ID NO: 1;
nnn) H, K, L, or R at a residue corresponding to position 381 in SEQ ID NO: 1;
ooo) S at a residue corresponding to position 382 in SEQ ID NO: 1; and/or
ppp) S or T at a residue corresponding to position 384 in SEQ ID NO: 1.

17. The engineered formate dehydrogenase of claim 13, wherein the one or more amino acid alterations result in an engineered formate dehydrogenase comprising:
a) A at a residue corresponding to position 2 in SEQ ID NO: 1;
b) T at a residue corresponding to position 98 in SEQ ID NO: 1;
c) I or V at a residue corresponding to position 199 in SEQ ID NO: 1;
d) Q at a residue corresponding to position 206 in SEQ ID NO: 1;
e) A, K, R, T, E, Y, V, I, or L at a residue corresponding to position 231 in SEQ ID NO: 1;
f) M or L at a residue corresponding to position 266 in SEQ ID NO: 1; and/or
g) P, K, L, R, H at a residue corresponding to position 381 in SEQ ID NO: 1.

18. The engineered formate dehydrogenase of claim 13, wherein the one or more amino acid alterations result in an engineered formate dehydrogenase comprising:
a) A at a residue corresponding to position 2 in SEQ ID NO: 1;
b) T at a residue corresponding to position 98 in SEQ ID NO: 1;
c) I or V at a residue corresponding to position 199 in SEQ ID NO: 1;
d) Q at a residue corresponding to position 206 in SEQ ID NO: 1;
e) V, I, or L at a residue corresponding to position 231 in SEQ ID NO: 1;
f) M or L at a residue corresponding to position 266 in SEQ ID NO: 1; and/or
g) H at a residue corresponding to position 381 in SEQ ID NO: 1.

19. The engineered formate dehydrogenase of any one of claims 1 to 4, wherein the one or more amino acid alterations of the engineered formate dehydrogenase is an alteration described in TABLE 7.

20. The engineered formate dehydrogenase of claim 19, wherein the one or more amino acid alterations result in an engineered formate dehydrogenase comprising:
a) K at a residue corresponding to position 36 in SEQ ID NO: 2;
b) V at a residue corresponding to position 64 in SEQ ID NO: 2;
c) E at a residue corresponding to position 80 in SEQ ID NO: 2;
d) S at a residue corresponding to position 91 in SEQ ID NO: 2;
e) N at a residue corresponding to position 97 in SEQ ID NO: 2;
f) T at a residue corresponding to position 111 in SEQ ID NO: 2;
g) I at a residue corresponding to position 120 in SEQ ID NO: 2;
h) L at a residue corresponding to position 162 in SEQ ID NO: 2;
i) V at a residue corresponding to position 164 in SEQ ID NO: 2;
j) G at a residue corresponding to position 187 in SEQ ID NO: 2;
k) C at a residue corresponding to position 188 in SEQ ID NO: 2;
l) T at a residue corresponding to position 214 in SEQ ID NO: 2;
m) V, T, or C at a residue corresponding to position 229 in SEQ ID NO: 2;
n) C at a residue corresponding to position 256 in SEQ ID NO: 2;
o) G or S at a residue corresponding to position 257 in SEQ ID NO: 2;
p) G at a residue corresponding to position 260 in SEQ ID NO: 2;
q) V, F, or T at a residue corresponding to position 312 in SEQ ID NO: 2;
r) G or A at a residue corresponding to position 313 in SEQ ID NO: 2;
s) C or S at a residue corresponding to position 315 in SEQ ID NO: 2;
t) T or S at a residue corresponding to position 320 in SEQ ID NO: 2;
u) M at a residue corresponding to position 323 in SEQ ID NO: 2;
v) R at a residue corresponding to position 361 in SEQ ID NO: 2; and/or
w) K at a residue corresponding to position 362 in SEQ ID NO: 2.

21. The engineered formate dehydrogenase of claim 19, wherein the one or more amino acid alterations result in an engineered formate dehydrogenase comprising:
a) K at a residue corresponding to position 36 in SEQ ID NO: 2;
b) V at a residue corresponding to position 64 in SEQ ID NO: 2;
c) E at a residue corresponding to position 80 in SEQ ID NO: 2;
d) S at a residue corresponding to position 91 in SEQ ID NO: 2;
e) N at a residue corresponding to position 97 in SEQ ID NO: 2;
f) T at a residue corresponding to position 111 in SEQ ID NO: 2;
g) I at a residue corresponding to position 120 in SEQ ID NO: 2;
h) L at a residue corresponding to position 162 in SEQ ID NO: 2;
i) V at a residue corresponding to position 164 in SEQ ID NO: 2;
j) G at a residue corresponding to position 187 in SEQ ID NO: 2;
k) C at a residue corresponding to position 188 in SEQ ID NO: 2;
l) T at a residue corresponding to position 214 in SEQ ID NO: 2;
m) T or C at a residue corresponding to position 229 in SEQ ID NO: 2;
n) C at a residue corresponding to position 256 in SEQ ID NO: 2;
o) G or S at a residue corresponding to position 257 in SEQ ID NO: 2;
p) G at a residue corresponding to position 260 in SEQ ID NO: 2;
q) V, F, or T at a residue corresponding to position 312 in SEQ ID NO: 2;
r) G or A at a residue corresponding to position 313 in SEQ ID NO: 2;
s) C at a residue corresponding to position 315 in SEQ ID NO: 2;
t) S at a residue corresponding to position 320 in SEQ ID NO: 2;
u) M at a residue corresponding to position 323 in SEQ ID NO: 2;
v) R at a residue corresponding to position 361 in SEQ ID NO: 2; and/or
w) K at a residue corresponding to position 362 in SEQ ID NO: 2.

22. The engineered formate dehydrogenase of claim 19, wherein the one or more amino acid alterations result in an engineered formate dehydrogenase comprising:
a) K at a residue corresponding to position 36 in SEQ ID NO: 2;
b) V at a residue corresponding to position 64 in SEQ ID NO: 2;
c) E at a residue corresponding to position 80 in SEQ ID NO: 2;
d) T at a residue corresponding to position 111 in SEQ ID NO: 2;
e) I at a residue corresponding to position 120 in SEQ ID NO: 2;
f) L at a residue corresponding to position 162 in SEQ ID NO: 2;
g) T at a residue corresponding to position 214 in SEQ ID NO: 2;
h) V, T, or C at a residue corresponding to position 229 in SEQ ID NO: 2;
i) G at a residue corresponding to position 260 in SEQ ID NO: 2;
j) C or S at a residue corresponding to position 315 in SEQ ID NO: 2;
k) T or S at a residue corresponding to position 320 in SEQ ID NO: 2; and/or
l) R at a residue corresponding to position 361 in SEQ ID NO: 2.

23. The engineered formate dehydrogenase of claim 19, wherein the one or more amino acid alterations result in an engineered formate dehydrogenase comprising:
a) K at a residue corresponding to position 36 in SEQ ID NO: 2;
b) V at a residue corresponding to position 64 in SEQ ID NO: 2;
c) E at a residue corresponding to position 80 in SEQ ID NO: 2;
d) T at a residue corresponding to position 111 in SEQ ID NO: 2;
e) I at a residue corresponding to position 120 in SEQ ID NO: 2;
f) L at a residue corresponding to position 162 in SEQ ID NO: 2;
g) T at a residue corresponding to position 214 in SEQ ID NO: 2;
h) T or C at a residue corresponding to position 229 in SEQ ID NO: 2;
i) G at a residue corresponding to position 260 in SEQ ID NO: 2;
j) C at a residue corresponding to position 315 in SEQ ID NO: 2;
k) S at a residue corresponding to position 320 in SEQ ID NO: 2; and/or
l) R at a residue corresponding to position 361 in SEQ ID NO: 2.

24. The engineered formate dehydrogenase of any one of claims 1 to 23, wherein the one or more amino acid alterations comprises at least one, two, three, or four alterations.

25. The engineered formate dehydrogenase of claim 24, wherein the one or more amino acid alterations result in an engineered formate dehydrogenase comprising:
a) H at a residue corresponding to position 381 in SEQ ID NO: 1;
b) Q at a residue corresponding to position 206 and I at a residue corresponding to position 231 in SEQ ID NO: 1;
c) I at a residue corresponding to position 199 in SEQ ID NO: 1;
d) Q at a residue corresponding to position 206 and V at a residue corresponding to position 231 in SEQ ID NO: 1;
e) I at a residue corresponding to position 199 and L at a residue corresponding to position 266 in SEQ ID NO: 1;
f) Q at a residue corresponding to position 206 and L at a residue corresponding to position 231 in SEQ ID NO: 1;
g) A at a residue corresponding to position 2 in SEQ ID NO: 1;
h) T at a residue corresponding to position 98 in SEQ ID NO: 1;
i) V at a residue corresponding to position 199 and M at a residue corresponding to position 266 in SEQ ID NO: 1;
j) T at a residue corresponding to position 111 and R at a residue corresponding to position 361 in SEQ ID NO: 2;
k) L at a residue corresponding to position 162 and R at a residue corresponding to position 361 in SEQ ID NO: 2;
l) T at a residue corresponding to position 229 and G at a residue corresponding to position 260 in SEQ ID NO: 2;
m) T at a residue corresponding to position 214 and R at a residue corresponding to position 361 in SEQ ID NO: 2;
n) K at a residue corresponding to position 36, L at a residue corresponding to position 162, T at a residue corresponding to position 214, and R at a residue corresponding to position 361 in SEQ ID NO: 2;
o) E at a residue corresponding to position 80 and R at a residue corresponding to position 361 in SEQ ID NO: 2;
p) I at a residue corresponding to position 120 and S at a residue corresponding to position 320 in SEQ ID NO: 2;
q) K at a residue corresponding to position 36 and R at a residue corresponding to position 361 in SEQ ID NO: 2;
r) T at a residue corresponding to position 111 and L at a residue corresponding to position 162 in SEQ ID NO: 2;
s) T at a residue corresponding to position 111, L at a residue corresponding to position 162, and R at a residue corresponding to position 361 in SEQ ID NO: 2;
t) V at a residue corresponding to position 64, L at a residue corresponding to position 162, T at a residue corresponding to position 214, and R at a residue corresponding to position 361 in SEQ ID NO: 2; or
u) C at a residue corresponding to position 229 and C at a residue corresponding to position 315 in SEQ ID NO: 2.

26. The engineered formate dehydrogenase of any one of claims 1 to 25, wherein the amino acid sequence of the engineered formate dehydrogenase does not consist of the amino acid sequence of SEQ ID NO: 24.

27. An engineered formate dehydrogenase comprising a variant of an amino acid sequence selected from any one of SEQ ID NOs: 3-24, wherein the engineered formate dehydrogenase comprises one or more alterations at a position corresponding to a position described in TABLE 6 and/or TABLE 7.

28. A recombinant nucleic acid encoding the engineered formate dehydrogenase of any one of claims 1 to 27.

29. The recombinant nucleic acid of claim 28, wherein the nucleic acid comprises a nucleotide sequence encoding the engineered formate dehydrogenase operatively linked to a promoter.

30. A vector comprising the recombinant nucleic acid of claim 28 or 29.

31. A non-naturally occurring microbial organism comprising a recombinant nucleic acid encoding an engineered formate dehydrogenase selected from any one of claims 1 to 27.

32. The non-naturally occurring microbial organism of claim 31, wherein said non-naturally occurring microbial organism further comprises a pathway capable of producing a bioderived compound, wherein one or more enzymes of the pathway uses NADH or NADPH as a cofactor for catalyzing its enzymatic reaction.

33. The non-naturally occurring microbial organism of claim 32, wherein the one or more enzymes of the pathway are encoded by an exogenous nucleic acid.

34. The non-naturally occurring microbial organism of claim 33, wherein the exogenous nucleic acid is heterologous.

35. The non-naturally occurring microbial organism of claim 34, wherein the exogenous nucleic acid is homologous.

36. The non-naturally occurring microbial organism of any one of claims 32 to 35, wherein said bioderived compound is an alcohol, a glycol, an organic acid, an alkene, a diene, an organic amine, an organic aldehyde, a vitamin, a nutraceutical or a pharmaceutical.

37. The non-naturally occurring microbial organism of claim 36, wherein said alcohol is selected from the group consisting of:
a) a biofuel alcohol, wherein said biofuel is a primary alcohol, a secondary alcohol, a diol or triol comprising C3 to C10 carbon atoms;
b) n-propanol or isopropanol; and
c) a fatty alcohol, wherein said fatty alcohol comprises C4 to C27 carbon atoms, C8 to C18 carbon atoms, C12 to C18 carbon atoms, or C12 to C14 carbon atoms.

38. The non-naturally occurring microbial organism of claim 37, wherein said biofuel alcohol is 1-propanol, isopropanol, 1-butanol, isobutanol, 1-pentanol, isopentenol, 2-methyl-1-butanol, 3-methyl-1-butanol, 1-hexanol, 3-methyl-1-pentanol, 1-heptanol, 4-methyl-1-hexanol, and 5-methyl-1-hexanol.

39. The non-naturally occurring microbial organism of claim 37, wherein said diol is a propanediol or a butanediol.

40. The non-naturally occurring microbial organism of claim 39, wherein said butanediol is 1,4 butanediol, 1,3-butanediol or 2,3-butanediol.

41. The non-naturally occurring microbial organism of claim 39, wherein said butanediol is 1,3-butanediol.

42. The non-naturally occurring microbial organism of claim 32, wherein said bioderived compound is selected from the group consisting of:
a) 1,4-butanediol or an intermediate thereto, wherein said intermediate is optionally 4-hydroxybutanoic acid (4-HB);
b) butadiene (1,3-butadiene) or an intermediate thereto, wherein said intermediate is optionally 1,4-butanediol, 1,3-butanediol, 2,3-butanediol, crotyl alcohol, 3-buten-2-ol (methyl vinyl carbinol) or 3-buten-1-ol;
c) 1,3-butanediol or an intermediate thereto, wherein said intermediate is optionally 3-hydroxybutyrate (3-HB), 2,4-pentadienoate, crotyl alcohol or 3-buten-1-ol;
d) adipate, 6-aminocaproic acid, caprolactam, hexamethylenediamine, levulinic acid or an intermediate thereto, wherein said intermediate is optionally adipyl-CoA or 4-aminobutyryl-CoA;
e) methacrylic acid or an ester thereof, 3-hydroxyisobutyrate, 2-hydroxyisobutyrate, or an intermediate thereto, wherein said ester is optionally methyl methacrylate or poly(methyl methacrylate);
f) 1,2-propanediol (propylene glycol), 1,3-propanediol, glycerol, ethylene glycol, diethylene glycol, triethylene glycol, dipropylene glycol, tripropylene glycol, neopentyl glycol, bisphenol A or an intermediate thereto;
g) succinic acid or an intermediate thereto; and
h) a fatty alcohol, a fatty aldehyde or a fatty acid comprising C4 to C27 carbon atoms, C8 to C18 carbon atoms, C12 to C18 carbon atoms, or C12 to C14 carbon atoms, wherein said fatty alcohol is optionally dodecanol (C12; lauryl alcohol), tridecyl alcohol (C13; 1-tridecanol, tridecanol, isotridecanol), myristyl alcohol (C14; 1-tetradecanol), pentadecyl alcohol (C15; 1-pentadecanol, pentadecanol), cetyl alcohol (C16; 1-hexadecanol), heptadecyl alcohol (C17; 1-n-heptadecanol, heptadecanol) and stearyl alcohol (C18; 1-octadecanol) or palmitoleyl alcohol (C16 unsaturated; cis-9-hexadecen-1-ol).

43. The non-naturally occurring microbial organism of any one of claims 31 to 42, wherein the non-naturally occurring microbial organism is in a substantially anaerobic culture medium.

44. The non-naturally occurring microbial organism of any one of claims 31 to 43, wherein the microbial organism is a species of bacteria, yeast, or fungus.

45. The non-naturally occurring microbial organism of any one of claims 31 to 44, wherein the non-naturally occurring microbial organism is capable of producing at least 10% more NADH or a bioderived compound compared to a control microbial organism that does not comprise the nucleic acid of claim 25 or 26.

46. A method for producing a bioderived compound, comprising culturing the non-naturally occurring microbial organism of any one of claims 32 to 45 under conditions and for a sufficient period of time to produce the bioderived compound.

47. The method of claim 46, wherein said method further comprises separating the bioderived from other components in the culture.

48. The method of claim 47, wherein the separating comprises extraction, continuous liquid-liquid extraction, pervaporation, membrane filtration, membrane separation, reverse osmosis, electrodialysis, distillation, crystallization, centrifugation, extractive filtration, ion exchange chromatography, absorption chromatography, or ultrafiltration.

49. A culture medium comprising said bioderived compound produced by the method of any one of claims 46 to 48, wherein said bioderived compound has a carbon-12, carbon-13 and carbon-14 isotope ratio that reflects an atmospheric carbon dioxide uptake source.

50. A bioderived compound produced according to the method of any one of claims 46 to 48.

51. The bioderived compound of claim 50, wherein said bioderived compound has an Fm value of at least 80%, at least 85%, at least 90%, at least 95% or at least 98%.

52. A composition comprising said bioderived compound of claim 50 or 51 and a compound other than said bioderived compound.

53. The composition of claim 52, wherein said compound other than said bioderived compound is a trace amount of a cellular portion of a non-naturally occurring microbial organism having a bioderived compound pathway.

54. A composition comprising the bioderived compound of claim 50 or 51, or a cell lysate or culture supernatant thereof.

55. A method for increasing the availability of NADH in a non-naturally occurring microbial organism, comprising culturing the non-naturally occurring microbial organism of any one of claims 31 to 45, under conditions and for a sufficient period of time to increase the availability of NADH.

56. The method of claim 55, wherein increasing the availability of NADH yields an increase in production of the bioderived compound described in any one of claims 32 to 42.

57. A method for decreasing formate concentration in a non-naturally occurring microbial organism, comprising culturing the non-naturally occurring microbial organism of any one of claims 31 to 45, under conditions and for a sufficient period of time to increase the conversion of formate to carbon dioxide.

58. The method of claim 57, wherein decreasing formate concentration in the non-naturally occurring microbial organism yields a decrease in formate as an impurity in a method for production of the bioderived compound described in any one of claims 32 to 42.


SEQUENCE LISTING (49 sequences)

SEQ ID NO: 1 - protein, 386 aa, Gibbsiella quercinecans
MSKILCVLYPDPVTGFPPVYARDSIPDIRCYPNGQTAPTPAGELGFTPGELVGSVSGELGLRRYLESQGHELIVTSDKEGADSVFERHLPDADVVISQPFWPAYLTAERIAKAKRLKLALTAGIGSDHVDLKAAAERGITVAEVTFSNSISVAEHVVMTVLALVRNYLPAHQIANNGGWNIADCVARSYDVEGMHFGTLGAGRIGLAVLRRLKPFDMPLHYYDPHRLSAECEKELGLIYHDTPESLVSVCDVVNLQTPLYPATQGFVNAAFLAKFKRGAYLINTARGALCERDAIVHALETGQLAGYGGDVWFPQPAPIDHPWRRMPHQGMTPHISGTSLSGQARYAAGTLEILQNFLEGKPIRQEYLIVDGGNLAGAGANAYKLA

SEQ ID NO: 2 - protein, 364 aa, Candida boidinii
MKIVLVLYDAGKHAADEEKLYGCTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 3 - protein, 364 aa, Candida boidinii
MKIVLVLYDAGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVLMTMLVLVRNFVPAHEQIINHDWEVAAIAKDAYDIEGKTIATIGAGRIGYRVLERLVPFNPKELLYYDYQALPKDAEEKVGARRVENIEELVAQADIVTINAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYITKAYGKHDKK

SEQ ID NO: 4 - protein, 448 aa, Clohesyomyces aquaticus
MVFLRSISRVARPASSMLSARAAPKASFGASFFRKPNAYRTLTATSTQQGKVLLVLYDGGVHAEQEPQLLGTTENELGIRKWLEDAGHTLITTSDKEGENSKFDQELVDAEVIITTPYVEATPHYSSSVPRNRTALSPHHLATLALPFHPGYLTAERLAKAKKLKLAVTAGIGSDHIDLDAANKTNGGITVAEVTGSNVVSVAEHVVMTILTLVRNFVPAHEQIAAGEWDVAAVAKNEYDLENKVVGTVAVGRIGERVLRRLKPFDCKELLYFDYQPLTAEKEKEIGCRRVENLEEMLAQCDVVTINCPLHEKTRGLFNKDLISKMKKGSWLINTARGAIVVKEDVADALKSGHLRGYGGDVWFPQPAPKEHPLRYATNPWGGGNAMVPHMSGTSIDAQKRYAAGTKAILDSYFSGREDYKPEDLIVHQGDYATKAYGQREGKKHANA

SEQ ID NO: 5 - protein, 376 aa, Clavispora lusitaniae
MSKPKVLLVLYEGKHHAKDEPKLYGCLENELGIRGFVESHGYELVSISEKDPIGDSEFDYHLADAEIVITTPFFPAYLTRERIAEAPKLKLCITAGVGSDHVDLNAANERKITVAEVTGSNVVSVAEHAVMTMLNLVRNFVPGHEQAMSGGWDIAAIAKDEYDLEDKVIATVGAGRIGYRILERLVAFNPKKLLYFDYQDLPKEAVDKLNAASKLFNGHDDIVERVEKLEDMLSRSDVVTINCPLHEGSKGLFNKETISHMKDGAWLVNTARGAICVEKDVADAVESGKLRGYGGDVWYPQPAPDHHPWRTFRNKYGGGNAMTPHVSGTSLDAQERYAAGTQAILKSYFEKSFDYRPQDVIVVDGEYATKAYGQRK

SEQ ID NO: 6 - protein, 400 aa, Caballeronia sordidicola
MAKVLCVLYDDPVDGMPKKYARDDIPAISKYHDGQSTPTPKAIDFKPGELLGSVSGELGLRKFLESRGHTLVVTSDKDGPNSTFERELEDAEVVISQPFWPAYLTAERIARAPKLKLALTAGIGSDHVDLQAAIDRGITVAEVTFCNSISVAEHVVMMILSQVRNYLPSHEWVKKGGWNIADCVSRAYDLEGMNVGTVAAGRIGAAVLRRLKPFDVKLHYTDRHRLPEQVERELGATWHPTAESMARVCDIVTINCPLHPETEHLFDETMISKMKRGAYIINTARGKIVDRDAVARALESGQLAGYAGDVWFPQPAPSDHPWRTMPNQGMTPHTSGTTLSAQARYAAGTREILECWFAKQPIREEYLIVDGGRLAGVGAHSYSAGNATSGSEEAARFKKA

SEQ ID NO: 7 - protein, 371 aa, Coccidioides posadasii
MGKVLMVMYDGGEHAKQQPGLLGTTENELGLRKWLEERGHTLVTTSDKEGSNSTFERELVDAEIIITTPFHPGYLTAERLAKAKNLKLAITAGVGSDHVDLNAANKTNGGITVAEVTGCNVVSVAEHVVMTILVLVRNFVPAHQQVASGEWDVAAVAKNEYDLEGKVVGTVAVGRIGERVLRRLKPFDCKELLYYDYQPLSPEVEKEIGCRRVENLEEMLAQCDVVTINCPLHEKTRGLFNKELISKMKKGSWLVNTARGAIVVKEDVAEAIKSGHLRGYGGDVWFPQPAPKDHPLRYVQGPWGGGNAMVPHMSGTSIDAQIRYAQGTKNILESYFSGKFDYKQEDLIVHQGDYVTKAYGQRKKIQEEQRN

SEQ ID NO: 8 - protein, 437 aa, Botryotinia fuckeliana
MVFFTRSLSRLATRPSPCARSIFTASAVARTTPTWTTRSTAFRSQKLNNIFGNVRMLTGNHREKVKVLLVLYDGKKHAEEVPELLGTTENELGIRKWLEDQGHELITTSDKEGENSEFDKHLVDAEIIITTPFHPGYLTAERLAKAKNLKIAITAGIGSDHVDLNAANKTNGGITVAEVTGSNVVSVAEHVVMTILVLVRNFVPAHEQIQAGEWDVAAAAKNEFDLEGKVVGTVAVGRIGERVLRRLKPFDCKELLYFDYQPLKPEVEQEIGCRRVTDLEEMLAQCDVVTINCPLHEKTRGLFNKELISKMKKGSWLVNTARGAIVVKEDVADALASGHLRGYGGDVWFPQPAPKDHPLRYAKNPFGGGNAMVPHMSGTSLDAQKRYADGTKAILESYLSGKHDYRPEDLIVIGGDYATKAYGERAKNSGSAGANKA

SEQ ID NO: 9 - protein, 366 aa, Yarrowia lipolytica
MKILLILYDAGSHAADEPKLLGCTENELGIRSWLESQGHTLVTTSSKEGADSVLDKEIVDADVVITTPFHPGYITRERIAKAKNLKICVTAGVGSDHVDLAAANERNIAVLEVTGSNVTSVAEHVVMTMLVLVRNFVPANEQVRGGGWDVAGVAKDSYDIEGKVIGTVGVGRIGKRVLQRLKPFDPKELLYYDYQPLSAADEKEIGARRVEKLEDMLAQCDVVTINCPLHESTKGLFNKELLSHMKKGAWLVNTARGAICVKEDVAAALKSGQLRGYGGDVWFPQPAPADHPWRKMVNKYGAGNAMTPHMSGTSLDAQARYAAGVKQILDEFFSGREQYRPQDIICYGGNYGTKAYGDDKKVVDKK

SEQ ID NO: 10 - protein, 385 aa, Kushneria avicenniae
MAKILCVLYPDPVTGYPPRYARDDIPEITGYPNGQTAPSPEGDPGFSPGELIGCVSGELGLRSWAQANGHELIVTSDKEGVDSVFERHLPDADVVISQPFWPAYLSAERIARAPKLKLALTAGIGSDHVDLKAAAEHGITVAEVTFSNSISVAEHVVMTALTLVRNFVPSHQIAINGGWNIADCVSRSYDIEGMQFGTLGAGRIGLAVLRRMKPFDTPLHYYDPHRLSEDIECELGLTYHDSPESLVSVCDIVNVQTPLYPSTEGFINDALLSKFKRGAYLVNTARGGLCDREAVVRALESGQLAGYGGDVWYPQPAPVDHPWRRMPNHAMTPHISGTSLSAQTRYAAGTLEILQNFLAGKSLREEYLIVDAGNLAGAGANAYQL

SEQ ID NO: 11 - protein, 313 aa, Zea mays
MVESTPLLVCLLDTNTKLEKHIEDMHVLITTPFHPAYVTAERIKKAKNLELLLTAGIGSDHIDLPAAAAAGLTVAEVTGSNTVSVAEDELLRILILLRNFLPGYQQVVQGEWNVAGIAHRAYDLEGKTVGTVGAGRIGRLLLQRLKPFNCNLLYHDRLQIDPELEKEIGAKFEEDLDAMLPKFDVIVINTPLTEKTRGMFNKERIAKMKKGVIVVNNARGAIMDARAVVDACSSGHIAGYGGDVWFPQPAPKDHPWRYMPNHAMTPHISRTTIDAQVRYADGVRDMLDRYFKGEDFPVQNYIVKEGQLASQYK

SEQ ID NO: 12 - protein, 413 aa, Dactylellina haptotyla
MVLRATSTLARCLAKPTLQASTLGARVVRPAYRQPLTAIQTRKLTTTFQPQVKVLAVLYDGGKHAQDVPALLGTTENELGIRKWLEDQGHTLVTTSDKEGENSKFDQELVDAEVIITTPFRPGYLTAERLAKAKKLKLAITAGIGSDHVDLNAANKTNGGVTVAEVTGSNVVSVAEHVVMTILILVRNFVPAHDQIRNGDWEVAAVAKDEYDLEDKIVGTVGVGRIGERVLRRLKPFDCKELLYFDYQPLNAAVEKEIGCRRVESLEEMVAQCDVVTINCPLHESTHGLFNKNLISKMKKGSWLVNTARGAIVVKEDVAEAVKSGHLRGYGGDVWFPQPAPKDHPLRYAVNPFGGGNAMVPHMSGTSLDAQKRYADGTKKILESYLSGRHDYQPHDLIVHKGDYATKAYGQRK

SEQ ID NO: 13 - protein, 397 aa, Segniliparus rugosus
MAKIVAVLYPDPVDGYPTTYARDDIPVIASYPGGQSAPTPSAIDFVPGQLLGSVTGELGLRKFLEAQGHTLVVTSDKEGPDSALDRELADADVVISQPFWPAYLTAERIAAAPNLKLAITAGIGSDHVDLNAAIARGITVAEVTFSNSISVAEHAVMQILALVRNYLPSHAWVAEKRGWNIADSVERAYDLEGMTVGVIAAGRIGLAVLRRLKPFDVKLAYTDKHRLSAEVEEELGLTFYPDIPSLVRAVDIVDVHAPLVPETHHLFDDALIGTMKRGSYIVNTARGAIMDTGAVVRALESGQLAGYAGDVWYPQPPAPDHPWRTMPWSGMTPHVSGTTLSAQARYAAGTREILESWLAGAPIRDEYLIVDGGKLAGTGARSYSTSGSGSSPGSSGK

SEQ ID NO: 14 - protein, 419 aa, Aspergillus indologenus
MVFMRSFSRQLRRPATSLLSSRGPLASAGSSPLRTSPLANSIAGARTLTASANLQGKILMVLYDGGEHAKQQPGLLGTTENELGLRKWLEDQGHTLVTTSDKEGENSTFDKELVDAEVIITTPFHPGYLTAERLAKAKNLKIAVTAGVGSDHVDLNAANKTNGGITVAEVTGCNVVSVAEHVVMTILTLVRNFVPAHEQIRNGEWDVAAVAKNEYDLENKVVGTVAVGRIGERVLRRLKPFDCKELLYYDYQPLSPEVEKEIGCRRVDDLEEMLAQCDVVTINCPLHEKTRGLFNKDLISKMKKGSWLINTARGAIVVKEDVAEAVKSGHLRGYGGDVWFPQPAPKDHPLRYVQGPWGGGNAMVPHMSGTSIDAQIRYANGTKAILESYFSGRHDYRNEDLIVHNGDYVTRAYGQRNKA

SEQ ID NO: 15 - protein, 415 aa, Friedmanniomyces endolithicus
MVFARSALRLARPASSLFSQRAAPAFAQRGVPVAKRALSATASQQGKVLLVLYDGHQHAQDEPRLLGTTENELGIRKWIEDQGHTLVTTSDKDGENSKFDQELVDAEVIITTPFHPGYLTKERLAKAKKLKIAVTAGIGSDHVDLDAANTTNGGITVAEVTGSNVVSVAEHVIMTMLVLVRNFVPAHEMIAAGNWDVAAVAKNEYDLENKVVGTVAVGRIGERVLRRLKAFDCKELLYFDYQGLTPEKEKEIGCRRVENLEDMLAQCDVVTINCPLHEKTRGMFNKELLSKMKKGSWLVNTARGAIVVKEDVAEALKSGQLRGYGGDVWFPQPAPKDHPLRYASYSTWGGGNAMVPHMSGTSIDAQERYAAGTKAILEEYFSGRENYKPEDLIVHKGDYATKAYGQREGKKHANA

SEQ ID NO: 16 - protein, 768 aa, Actinidia chinensis
MAMKRAAASAVRALASSGNSNSSILTRELHTSPGSKKIVGVFYKANEYAAMNPNFLGCVERALGIGDWLKSQGHQYIVTDDKEGPDCELEKHISDMHVLITTPFHPAYVTAERIKKAKNLQLLLTAGIGSDHVDLKAAAAAGLTVAEVTGSNVVSVAEDELMRVLILTRNFLPGYHQVIKGDWNVAAIAHRAYDLEGKTVGTVGAGRIGRLLLQRLKPFNCHLLYHDRIKMDLELENQIGAKFEEDLDVMLPKCDIIVINMPLTEKTKGMFNKERIAKLKKGVLIVNNARGAIMDTQAVVDACSSGHIAGYSGDVWNPQPAPKDHPWRYMPNQAMTPHISGTTIDAQLRYAAGVKDMLDRYFKGEEFPPQHYIVKEGPTRCGGNAIGWGQCEKVKMGLLENHLGESVRVVTKCSTSPGSKKIVGVFYKANEYAAMNPNFLGCVERALGIGDWLKSQGHQYIVTDDKEGPDCELEKHISDMHVLITTPFHPAYVTAERIKKAKNLQLLLTAGIGSDHVDLKAAAAAGLTVAEVTGSNVVSVAEDELMRVLILTRNFLPGYHQVIKGDWNVAAIAHRAYDLEGKTVGTVGAGRIGRLLLQRLKPFNCHLLYHDRIKMDLELENQIGAKFEEDLDVMLPKCDIIVINMPLTEKTKGMFNKERIAKLKKGVLIVNNARGAIMDTQAVVDACSSGHIAGYSGDVWNPQPAPKDHPWRYMPNQAMTPHISGTTIDAQLRYAAGVKDMLDRYFKGEEFPPQHYIVKEGELASQYR

SEQ ID NO: 17 - protein, 418 aa, Aspergillus kawachii
MVFMRSFSRQLRRPATSLLSKGAFAPAASSPFRAAPLANSIAGARTLTASANLQGKVLMVLYDGGEHAKQQPGLLGTTENELGLRKWLEEQGHTLVTTSDKEGENSTFDKELVDAEVIITTPFHPGYLTAERLAKAKNLKIAVTAGVGSDHVDLNAANKTNGGITVAEVTGCNVVSVAEHVVMTILTLVRNFVPAHEQIRRGEWDVAAVAKNEFDLEGKVVGTVAVGRIGERVLRRLKPFDCKELLYYDYQPLSPEVEKEIGCRRVDDLEEMLAQCDVVTINCPLHEKTRGLFNKELISKMKKGSWLVNTARGAIVVKEDVAEAVKSGHLRGYGGDVWFPQPAPADHPLRTVQGPWGGGNAMVPHMSGTSIDAQIRYANGTKAILESYFSGRHDYRPQDLIVHGGDYVTKAYGQRNKA

SEQ ID NO: 18 - protein, 351 aa, Candidatus Nitrosocosmicus oleophilus
MKIVAVLYPGGEIAKRTPAILGSAENALGLTNFLKDKGHEFIVLTDKEEELDKHISTTDILITTPFWPAYVTKERISKASNLKLILTAGVGSDHIDLAAAASSKITVAEITGSNVVSVAEQVVMHILALVRNYIPAYKQVIEGRWDIAEIASNAHDLEDKVVGIIGMGRIGQRVCERLKAFDVKMLYHDQIPLRTAEEFVLGVRYTPRDQIVEQADVITINTPLTPETDGMFNRDLLFKMKKGAYLVNTARGKIVDTHALVESLEEGHLAGYAGDVWYPQPATGDHPWRHMPNHAMVPHYSGTTLEAQNRYANGIKDCLMRFLENRPLEQQYLIVDKGGVVSPSYSYAFKK

SEQ ID NO: 19 - protein, 340 aa, Blastomyces gilchristii
MGKVLLVLYDGGQHAKDQPGLLGTTENELGLRKWLEEKGHTLVTTSDKEGANSKFDQELVDAEVIITTPFHPGYLTAERLAKAKNLKLAVTAGVGSDHVDLNAANKTNGGITVAEVTGCNVVSVAEHVVMTILVLVRNFVPSHEQVASGDWNVAAVAKNEYDLEGKVVGTVAVGRIGERVLRRLKPFDCKELLYYDYQPLTPEVEKEIGCRRVENLEEMLAQCDVVTINCPLHEKTRGLFNKDLISKMKKRLLAHQHRPRRHRRQRRRRRRHQIRPSARLRRRRVVPAARAQGPSAALRPGALGRRKRDGAAHVRQLHRCAGALRGGHEGDSRVVFLRQA

SEQ ID NO: 20 - protein, 423 aa, Paraburkholderia caribensis
MYHVDMSIERRHENALDQTGARRMAKVLCVLYDDPVKGMPKQYARDGVPEITRYHDGQTAPTPKGIDFTPGELLGSVSGELGLRKYLESLGHTLVVTSDKDGPNSRFERELADAEVVISQPFWPAYLTAERIAKAPKLKLALTAGIGSDHVDLQAAIERGVTVAEVTYCNSISVAEHVVMMILGLVRNYLPSHEWVKKGGWNIADCVERAYDLEAMHVGTVAAGRIGAAVLRRLKPFDVKLHYTDRHRLPLDVEKELGATWHPDVESMVKACDVVTINCPLHPETENLFNEKLIAKMKRGAYIVNTARGKIVDRDAIVRALESGQLAGYAGDVWFPQPAPRDHPWRTMPHNGMTPHISGTTLSAQARYAAGTREILECWFEKRPIRDEYLIVDGGKLAGVGAHSYSAGNATSGSEEAARFKTA

SEQ ID NO: 21 - protein, 403 aa, Mesorhizobium sp.
MKMAKVVCVLYDDPVDGYPSNYARDGLPKLDRYPGGQTLPTPKAIDFEPGVLLGSVTGELGLRPFLESAGHSLVVTSDKDGPDSVFERELADAEIVISQPFWPAYLTNERIAKADQLKLAITAGIGSDHVDLQAAMDRGITVAEVTYCNSISVSEHVVMMILALVRNYIPSYQWVIDGGWNIADCVARSYDVEGMQIGTVGAGRIGSAVLHRLQPFDVKLHYTDRHRLSEAVEKHLGVTFHPDAASMVGICDVVTINAPLHPETENLFDEAMIARMKRGAYLVNTARGKICNRDAVARALESGQLAGYAGDVWFPQPAPKDHPWRSMPHHGMTPHISGSSLSAQARYAAGTREILECWFEGRPIREEYLIVDGGKLAGAGAHSYSAGDATSGSEEAARFKAKA

SEQ ID NO: 22 - protein, 424 aa, Helicocarpus griseus
MVFLRSTSRLLSRPISSLLAGRALPRASAALPLYNTQLCGPTSRLPFTGVRTLTASPKLQGKVLLVLYDGEEHAKQQPGLLGTTENELGLRKWLEDQGHTLVTTSDKEGENSKFDQELVDAEVIITTPFHPGYLTAERLAKAKNLKLAITAGVGSDHVDLNAANKTNGGITVAEVTGCNVVSVAEHVVMTILVLVRNFVPAHEQVASGDWNVAAVAKNEFDLEGKVVGTVAVGRIGERVLRRLKPFDCKELLYYDYQPLKPEVEKEIGCRRVENLEEMLAQCDVVTINCPLHEKTRGLFNKELISKMKKGSWLVNTARGAIVVKEDVAEAVKSGHLRGYGGDVWFPQPAPKDHPLRYAQGPWGGGNAMVPHMSGTSIDAQVRYAEGTKRILDSYFSGRHDYRPEDLILFGGDYATKAYGQRTKA

SEQ ID NO: 23 - protein, 392 aa, Streptomyces albidoflavus
MAKIVCVLYPDPVTGFPPQYARTSLPALDGYPGGQTLPTPEGLDFTPGELVGSVSGELGLREFLESRGHTLVVTSDKEGPDSELDRELADADVVISQPFWPAYLTAERIARAPKLKLALTAGIGSDHVDLDAAIARGITVAEVTYSNSISVAEHAVMQILALVRNYLPSHKIAAEGGWNIADCVSHAYDLEGMDVGVIAAGRIGQAVLRRLKPFGVRLHYTDKRRLPREVEEELGLTFHASAQELARNIDVVSIHAPLHPETQNLFDEKLLATMRPGSYIVNTARAQIVDRDAIVRALESGQLAGYAGDVWYPQPAPADHPWRTMPHNGMTPHISGTTLTAQARYAAGTREILEDWLQGTPIREEYLIVDGGRLAGTGAASYTAGEKTAEKS

SEQ ID NO: 24 - protein, 364 aa, synthetic construct
MKIVLVLYDCGKHAADEEKLYGCTENKLGIANWLKDQGHELITTSDKEGGNSVLDQHIPDADIIITTPFHPAYITKERIDKAKKLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLVPFNPKELLYYDYQALPKDAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAQGTKNILESFFTGKFDYRPQDIILLNGEYVTKAYGKHDKK

SEQ ID NO: 25 - DNA, 1161 bp, Gibbsiella quercinecans
  source 1..1161: mol_type = other DNA
atgtccaagattttgtgcgtattgtatcccgatcctgttaccggttttccgccggtttatgcccgcgacagcatccctgatatccgttgctatccgaatggccagacggcgccgacgccggctggcgaattgggctttacccccggcgagctggtcggcagcgtatctggggaactgggattacgtcgctatctggaaagtcagggccatgagttgattgtgaccagtgacaaagaaggcgcggattcggtatttgaacgccatttgccggatgccgatgtggtcatttcccagccgttttggccggcttacttgaccgctgaacgcattgccaaagcgaagaggcttaaactggcgctgaccgccggtattggttccgaccatgtcgatctcaaggccgccgccgaacgcggcatcaccgtggccgaggtgacgttctccaactccatcagcgtggcggagcatgtggtgatgacggtactggccctggtgcgcaactacctgccggcgcatcaaatcgccaacaacggcggttggaacattgcggactgcgtggcgcgcagctatgacgtggaagggatgcatttcggcacgctgggcgccgggcgcattgggttggcggtgctgcgacgtttaaaaccctttgatatgccactgcattattacgatccgcaccgtttgtcggcggagtgcgagaaggagctgggtttaatttaccacgatacgccggagtcgctggtcagcgtctgcgatgtggttaacctgcagacgccgctttacccggcaacccaggggttcgtcaatgcggccttcctggcgaagttcaagcgtggcgcctacctgatcaataccgcccgcggcgcgctgtgtgagcgcgatgcgatcgtccacgcactggaaaccgggcagttggcgggctatggcggcgacgtgtggtttcctcaaccggcgccgatcgaccatccatggcggcgcatgccgcatcagggcatgacgccgcacatttccggcacgtcactgtcggggcaggcgcgctatgcggccgggacgctggaaattctgcagaacttcctggaaggcaagccgatccgccaagaatacctgattgttgatggcggcaatttggcgggtgccggcgccaacgcctataaattggcctga

SEQ ID NO: 26 - DNA, 1095 bp, Candida boidinii
  source 1..1095: mol_type = other DNA
atgaagatcgttttagtcttatatgatgctggtaagcacgctgctgatgaagaaaaattatatggttgtactgaaaataaattaggtattgctaattggttaaaagatcaaggtcatgaactaattactacttctgataaagaaggtgaaacaagtgaattggataaacatatcccagatgctgatattatcatcaccactcctttccatcctgcttatatcactaaggaaagacttgacaaggctaagaacttaaaatcagtcgttgtcgctggtgttggttctgatcacattgatttagattatattaatcaaacaggtaagaaaatctcagtcctggaagttacaggttctaatgttgtctctgttgctgaacacgttgtcatgaccatgcttgtcttggttagaaatttcgttccagcacatgaacaaattattaaccacgattgggaggttgctgctatcgctaaggatgcttacgatatcgaaggtaaaactatcgctaccattggtgctggtagaattggttacagagtcttggaaagattactcccatttaatccaaaagaattattatactacgattatcaagctttaccaaaagaagctgaagaaaaagttggtgctagaagagttgaaaatattgaagaattagttgctcaagctgatatcgttacagttaatgctccattacacgcaggtacaaaaggtttaattaataaggaattattatctaaatttaaaaaaggtgcttggttagtcaataccgcaagaggtgctatttgtgttgctgaagatgttgcagcagctttagaatctggtcaattaagaggttacggtggtgatgtttggttcccacaaccagctccaaaggatcacccatggagagatatgagaaataaatatggtgctggtaatgccatgactcctcactactctggtactactttagacgctcaaacaagatacgctgaaggtactaaaaatattttggaatcattctttaccggtaaatttgattacagaccacaagatattatcttattaaatggtgaatacgttactaaagcttacggtaaacacgataagaaataa

SEQ ID NO: 27 - DNA, 1095 bp, Candida boidinii
  source 1..1095: mol_type = other DNA
atgaagatcgttttagtcttatacgatgctggtaaacacgccgccgatgaagaaaaattatacggttgtactgaaaacaaattaggtattgccaattggttaaaagatcaaggccatgaattaatcaccacttctgataaagaaggcggaaacagtgtgttggatcaacatatcccagatgctgatattatcattacaactcctttccatcctgcttatatcaccaaggaaagaatcgacaaagctaaaaaattgaaattagttgtcgtcgctggtgtcggttctgatcatattgatttggattacatcaaccaaaccggtaagaaaatctccgttttggaagttaccggttctaatgttgtctctgttgcagaacacgttctcatgaccatgcttgtcttggttagaaattttgttccagctcacgaacaaatcattaaccacgattgggaagttgctgctatcgctaaggatgcttacgatatcgaaggtaaaactatcgccaccattggtgccggtagaattggttacagagtcttggaaagattagtcccatttaatccaaaagaattattatactacgattatcaagctttaccaaaagatgccgaagaaaaagttggtgctagaagggttgaaaatattgaagaattagttgcccaagctgatatagttacaattaatgctccattacacgctggtacaaaaggtttaattaacaaggaattattgtctaaattcaagaaaggtgcttggttagtcaatactgcaagaggtgccatttgtgttgccgaagatgttgctgcagctttagaatctggtcaattaagaggttacggtggtgatgtttggttcccacaacctgctccaaaagatcacccatggagagatatgagaaacaaatatggtgctggtaacgccatgactcctcattactctggtactactttagatgctcaaactagatacgctgaaggtactaaaaatattttagagtcattctttaccggtaagtttgactacagaccacaagatatcatcttattaaatggtgaatacattaccaaagcttatggtaagcacgataagaaataa

SEQ ID NO: 28 - DNA, 1347 bp, Clohesyomyces aquaticus
  source 1..1347: mol_type = other DNA
atggtatttctccgctcaatttctcgagtcgcccgcccggcctcctcgatgctctcagcacgcgccgctcccaaagcttcctttggagcttcgttcttccgtaagccgaatgcctacagaacgctaacggcgacctcaacacaacagggcaaggtccttctcgtactctacgatggcggcgtccacgcggagcaggagccgcagctcctcggtactactgagaacgaactgggcatccgaaaatggctcgaagatgccggccacacgctcatcacgacttcagacaaggagggcgagaactccaaattcgaccaggaactggtggatgcggaggtgatcattaccacgccgtatgttgaagctaccccgcactactcgagctctgtgccccgcaaccgcaccgcgctctcgccccaccatcttgccactctcgcgctacccttccaccccggttatctgaccgcagagcgtctggccaaggcgaagaagctcaagctcgctgtcactgccggtatcggatctgatcacattgacctcgatgcagccaacaagacgaatggcggcatcaccgttgccgaagtcacgggctcgaatgtcgtgtccgtagccgagcacgtggttatgaccatcctcaccctcgtgcgcaacttcgttcccgcccatgagcagattgccgctggcgagtgggacgtcgctgccgtcgccaagaacgagtatgatttggagaacaaggtcgtcggcaccgttgccgtcggtcgcattggcgagcgtgtgctgcgccgtctcaagcccttcgactgcaaggagctgttgtacttcgactaccagccattgaccgctgaaaaggagaaggagattggctgccgaagggtggagaacctcgaggagatgctggcacagtgcgacgtcgtcactatcaactgcccgctacacgaaaagacacgggggctgttcaacaaggacctcatctccaagatgaagaagggctcatggctcatcaacactgcccgaggtgccattgtcgtgaaggaggatgttgctgatgcgctcaagagtggccacctccgtggatacggtggtgatgtctggttcccgcagccggcgcccaaggagcacccgctccgatatgccaccaacccatggggtggaggcaacgccatggtcccccacatgtccggtacctcgatcgatgcccagaagcgatatgctgctggtacgaaggccattctcgactcttacttctccggtcgtgaagactacaagcccgaggatctcatcgtgcatcaaggcgactacgccaccaaggcgtacggtcagcgcgagggaaagaagcatgctaatgcataa

SEQ ID NO: 29 - DNA, 1130 bp, Clavispora lusitaniae
  source 1..1130: mol_type = other DNA
atgtctaaaccaaaagtgttactcgttttgtacgaaggcaagcaccatgccaaagatgagcctaagctttacggatgcctcgagaacgaattgggtatcagaggtttcgtggagtctcacggttacgaattggtgagcatctctgaaaaggatccaattggagactctgagtttgactatcatttggcagacgctgagattgtcattaccacgccatttttccccgcgtacctcactcgtgagagaattgcagaggcacccaagttgaagctctgcatcaccgcaggagtcggatcggaccatgttgacttgaatgctgccaatgaaagaaagatcactgttgcagaggttactggatccaacgttgtttctgttgctgaacacgcagtaatgacaatgttaaacttggttagaaactttgtcccaggacatgagcaggccatgtctggaggatgggatattgcagccattgccaaggatgaatatgacttggaggacaaggttatcgccacagttggcgctggcaggattggttaccgtatcttggagcgccttgtggcattcaatccgaagaagctcttgtactttgactaccaggatttgcccaaggaagccgtggacaagttgaacgctgcctctaaacttttcaatggtcacgacgacattgttgaacgcgtggaaaagttagaggatatgttatcccgttccgatgttgtgactatcaattgtcctttacacgagggaagcaaaggtttgttcaataaggaaaccattagtcacatgaaggatggcgcttggttggtgaacacagcccgtggtgccatttgtgttgaaaaggatgtagctgatgctgtcgaatcgggcaagctccgtggatacggtggagacgtatggtatccacagccagccccagaccatcatccttggagaactttccgcaacaagtatggcggtggaaatgctatgaccccccacgttagtggaacatcattggacgcccaagagagatatgctgctggtacacaagcaattttgaaatcgtactttgaaaagtcctttgattatcggccacaggatgtgattgttgttgatggcgaatacgccaccaaagcctatggccagcgtaagta

SEQ ID NO: 30 - DNA, 1203 bp, Caballeronia sordidicola
  source 1..1203: mol_type = other DNA
atggccaaggttttatgcgttttatatgacgaccctgtcgacggtatgcccaagaagtacgcccgggacgacattcccgccatctcgaagtatcacgacggacaaagcaccccgacacccaaggcaatcgacttcaagccgggcgagttgctgggcagcgtgtccggcgagttgggattgcgcaagttcctcgaatcacgggggcacaccctggtcgttacatccgacaaggacggccccaactccacgttcgagcgcgagctggaggatgccgaggttgtcatttcccagccattctggcccgcctatctgacggctgaacgcatcgcccgggcgccgaagctcaagcttgcgctcacggctggcatcggctcggatcacgtcgatttgcaggccgcgatcgatcgcggcatcacggttgccgaagtgaccttctgcaatagcatcagcgtggccgagcacgtcgtcatgatgatcctgtcccaggtccggaactatttgccgtcgcatgaatgggtcaagaagggcggctggaatatcgcggattgcgtctcgcgtgcgtacgatctcgaggggatgaacgtagggaccgtggcggccggacgcatcggtgcggcggtgctgagacggctcaaaccctttgacgtcaagctgcactacacggaccggcatcgtctgccggaacaggtcgagcgcgagcttggcgcaacctggcatcccactgctgaatccatggcgcgtgtgtgcgatatcgtgaccatcaactgtccgttgcacccggaaacggaacatctcttcgacgagacgatgatcagcaagatgaagcgcggcgcttacatcatcaacacggcgcgcgggaagattgtcgatcgcgatgctgtcgcgcgtgcgctggaatcgggccaactggcgggttatgcaggcgacgtctggttcccgcagcctgcaccgagcgatcacccgtggcggaccatgccgaatcagggcatgacgccgcacacgtcgggcactacgctttcggcacaggcgcgttacgccgctggcacgcgtgaaattctcgaatgctggtttgccaaacaaccgattcgtgaggagtatctgatcgtcgacggcggacgtcttgccggtgtgggtgcgcactcgtacagcgcaggcaatgcgacgtcgggctcggaagaggcggcccgcttcaagaaggcataa

SEQ ID NO: 31 - DNA, 1056 bp, Coccidioides posadasii
  source 1..1056: mol_type = other DNA
atgggtaaagttcttatggttatgtatgacggtggggagcatgcgaagcagcagcctggtttattgggaacaacagaaaatgagcttggattaagaaaatggcttgaggagagaggccacaccttggtaaccacatctgacaaggagggatcgaactctacctttgagcgcgagcttgttgatgcggagatcatcatcactacaccgttccatcccggttatctcaccgcagagcgtctggccaaggccaagaacctcaaacttgccatcacagcgggtgttggttccgaccatgttgacctgaatgccgccaataagaccaacggaggtatcactgtcgctgaagtcactggctgcaatgtcgtgtctgttgccgaacatgtcgttatgaccatcttagtcctcgtccgcaatttcgtcccggcccatcaacaagttgccagcggtgaatgggacgttgctgccgttgctaaaaacgagtatgaccttgagggtaaggtcgtcggaactgtagctgttggccgcattggtgagcgtgtacttcgccgtcttaagcccttcgactgcaaggagcttctttattatgactaccagcccctcagccctgaggtcgagaaggaaattggatgccgtcgtgttgagaacttggaggagatgttggcacagtgtgatgttgtcactatcaactgccctctccacgagaagacacgtggcttgttcaacaaggaactcatttctaagatgaagaaaggctcatggcttgtaaacaccgcccgtggggctatcgtcgtcaaggaggacgtcgcagaagctataaaatcaggccatctccgtggatatggcggtgacgtctggttcccacaacctgctcccaaagaccacccactacgctatgtccagggcccatggggcggcggaaatgccatggtccctcacatgtctggaacttccattgatgcacaaatccgctatgcacagggcacgaagaacatccttgaatcgtacttctccggcaagttcgattacaaacaagaagatttgattgttcatcagggc

SEQ ID NO: 32 - DNA, 1314 bp, Botryotinia fuckeliana
  source 1..1314: mol_type = other DNA
atggtatttttcactcgctccctctctcgtttggcaacaagaccgtctccctgtgctcggtcaatcttcacagcttctgctgttgcgagaactacacctacatggacgacgcgatcgactgctttccgctcgcagaagctgaataacatatttgggaatgtcagaatgctaacgggtaatcatcgtgaaaaggtcaaggtacttcttgtattgtacgacggaaagaagcacgccgaagaggtcccagagcttctcggcacaaccgagaacgagctcggtatccgcaaatggctcgaggatcaaggacatgagctcatcaccacctccgacaaggagggagagaactccgagttcgacaagcacttggtagacgcagagatcatcattaccactcctttccaccctggttacctcactgccgaacgtcttgccaaggccaagaacctcaagatcgccatcactgccggtattggatctgatcacgtcgatctcaatgctgccaacaagaccaacggtggtatcaccgttgctgaagttactggatccaacgtcgtctccgtcgctgagcacgtcgttatgaccatcttggttctcgtccgtaactttgtcccagcacacgagcaaatccaagccggagaatgggacgttgccgctgccgccaagaacgagttcgatcttgagggtaaggtcgtcggtaccgttgctgtcggaagaattggtgagcgtgtcttgagaagattgaagccattcgactgcaaggaattgttgtacttcgattaccaacctcttaagccagaggttgagcaagagattggttgcagacgtgttaccgatttggaggagatgttggctcaatgtgatgttgtcaccattaactgccctctccacgagaagacccgtggccttttcaacaaggagttgatctccaagatgaagaagggatcctggttggtcaacaccgcccgtggtgccattgttgtcaaggaagatgttgccgatgccctcgcatccggtcacctccgcggttacggaggagatgtttggttccctcaaccagcaccaaaggaccacccactccgctacgcaaagaacccattcggtggtggaaacgccatggttcctcacatgtctggtacctctctggatgctcaaaagagatatgccgatggtaccaaggctattctcgagagctacttgtctggaaagcatgactacagaccagaggatttgattgttattggtggtgactatgctaccaaggcttatggtgagagagcgaagaacagtggttctgccggtgccaacaaggcataa

SEQ ID NO: 33 - DNA, 1101 bp, Yarrowia lipolytica
  source 1..1101: mol_type = other DNA
atgaagattctccttatcctttacgacgccggctcccacgctgctgacgaacccaagctgctaggttgcaccgagaacgagctcggcatccgatcctggctcgagtcccagggccataccctggtcaccacctcttccaaggagggtgctgactctgttctcgacaaggagattgttgatgccgatgttgtcatcaccactcccttccaccccggctacatcacccgagagcgaattgccaaggccaagaacctcaagatctgtgtcactgccggtgttggctctgaccacgttgatcttgctgccgccaacgagagaaacattgccgtgctcgaggtcactggttccaacgtgacttctgttgctgagcacgttgtcatgaccatgcttgttcttgtccgaaacttcgtgcctgccaacgagcaggttcgaggcggcggctgggacgttgccggtgttgccaaggattcctacgatattgagggtaaggtcattggtaccgtcggagtcggccgaatcggcaagcgagtgctccagcgactcaagcccttcgaccccaaggagctgctctactacgactatcagcctctgtctgctgccgacgagaaggagattggcgcccgacgagttgagaagctcgaggacatgcttgcccagtgtgatgttgtcaccatcaactgccccctgcacgaatccaccaagggtctctttaacaaggagctgctgtcccacatgaagaagggcgcctggctcgtcaacaccgctcgaggagccatctgtgtaaaggaggacgttgccgccgctctcaagtctggtcagctccgaggctacggcggagatgtctggttcccccagcctgcccctgctgaccacccctggagaaagatggtcaacaagtacggtgccggtaacgccatgaccccccacatgtctggaacctctctggacgcccaggcccgatacgctgctggtgtcaagcagattctcgacgagttcttctccggccgagaacagtaccgaccccaggacattatctgctacggtggtaactacggcaccaaggcctacggagacgacaagaaggtcgtcgacaagaagtaa

SEQ ID NO: 34 - DNA, 1158 bp, Kushneria avicenniae
  source 1..1158: mol_type = other DNA
atggccaagattctctgtgttctttatcccgatccggtgaccggctatccaccgcgttatgcgcgtgacgatatccccgaaatcacgggctatcccaacggccagacagcgccttctccggagggagatccggggttttcccccggcgaattgatcggctgcgtctccggtgagctggggctgcgttcgtgggcgcaggccaacggtcatgagcttatcgtgaccagtgacaaggagggcgtcgattcggtcttcgagcgccatctgccggacgctgatgtcgtgatttctcagccgttctggccggcctacctcagcgcggaacgcattgcccgggcaccgaagctcaagctggcgctgacggccggtatcggctcggatcacgtcgatctcaaggcggcggccgagcacggtatcacggtggccgaggtgaccttctccaactcgatcagtgtggccgagcacgtggtcatgaccgcgctgacgctggtccgcaatttcgtgccgtcgcaccagattgccatcaacggcggctggaacatcgctgactgcgtatcacgcagctacgacatcgaagggatgcagtttggtaccctcggtgccggtcgtatcggccttgcggtcctgcgtcgcatgaagcccttcgatacaccgctgcattactacgatccgcaccgtctgtccgaggatatcgagtgcgagctcggtctgacctatcacgactcgccggaatcgctggtgagcgtctgcgacatcgtcaacgttcagacgccgctttacccctcgaccgaagggttcatcaacgacgcgctgctgtcgaaattcaagcgtggcgcctatctggtcaacaccgcgcgcggcgggctttgcgaccgtgaggcagtggtcagggcgctggagtccgggcagctggcaggttacgggggcgatgtgtggtacccgcagcccgcgccggtggatcatccgtggcggcgcatgcccaatcatgcgatgacgccacatatctccggcacctcgctgtcggcccagacccgctatgccgccggaacgctggaaatcctgcagaatttcctcgccggcaagtcgctgcgtgaagagtatctcatcgtcgatgccgggaacctggccggcgccggggccaatgcctatcagctttga

SEQ ID NO: 35 - DNA, 942 bp, Zea mays
  source 1..942: mol_type = other DNA
atggtagagtccacaccattgctggtttgcttgctagacacaaacactaaactggagaagcacattgaagacatgcatgtcctgattaccaccccattccacccagcttatgtcactgcagagaggataaagaaggcgaagaatcttgagcttcttctcacggcaggaatcggctccgatcatattgatcttccagcagctgctgctgcgggcttaactgtggctgaggtcactggaagcaacactgtctctgtggcagaagatgagctcttgcgcattctgattctgctcaggaacttcttgcctgggtaccaacaggtagttcaaggcgaatggaacgtagcaggcattgcccacagagcttatgatcttgaaggaaaaactgttgggactgttggagctggtcgtattggcaggctcttgcttcagcgtcttaagccctttaactgcaacctactttaccatgacagacttcagattgacccagagcttgagaaagaaattggggccaaatttgaggaggacctggatgctatgcttccaaagtttgatgtgattgtgatcaacacacctcttacggagaaaacaagaggcatgtttaacaaagagaggattgcaaagatgaagaaaggtgtaattgttgtgaataatgctcgaggagcaatcatggatgctcgagcagtcgtagatgcatgttctagcggtcacattgctggatatggtggtgatgtgtggttcccacagccagcaccaaaggatcacccctggcgctacatgcctaaccatgccatgacccctcacatctctaggactacaattgatgcgcaggtgcggtatgcggatggtgtgagggacatgctggataggtatttcaagggcgaggacttcccggtgcagaactacatcgtcaaggaaggccagctcgccagtcagtacaagtga

SEQ ID NO: 36 - DNA, 1242 bp, Dactylellina haptotyla
  source 1..1242: mol_type = other DNA
atggtacttcgagccacatctacccttgcgcgttgcctcgccaaaccaaccctccaggcctccacactgggtgctcgtgttgttcggccagcgtatcgccagccattgaccgcaattcaaaccagaaaactgacaactactttccaacctcaggtcaaagttcttgctgtcttgtatgatggtggcaagcacgcccaggatgttccagctctgctaggaaccacagagaacgagctcggtattagaaaatggcttgaggatcaaggacacactttggtcaccacctctgacaaggaaggcgagaactcaaaattcgaccaagagttggttgatgctgaagttatcatcacaactcccttccgccctggatatttgactgctgagcgtcttgcaaaagccaagaaattgaagcttgctatcactgccggaattggatctgaccatgtggacctcaatgctgccaataagacgaatggtggagtcactgttgctgaagtcactggatcgaatgtcgtcagtgtagctgaacatgttgttatgacgatcttgatcttggtcaggaattttgtcccagctcatgaccaaatcagaaatggtgactgggaagttgccgctgttgccaaggatgaatatgacctcgaagataagattgttggaactgttggtgttggtagaatcggcgagcgtgtcctgcgaagattgaagccattcgactgcaaggaactgctttacttcgactaccaaccactcaatgcagcagttgaaaaagaaatcggctgccgccgtgtcgagagtttagaggagatggttgcacagtgcgatgttgtaacgatcaactgtccactccacgagtccacccacggattattcaacaagaatttgatctccaagatgaagaagggttcttggttggttaatactgctcgtggcgccatcgttgtcaaggaagacgttgccgaagccgtcaaatctggccaccttcgaggctatggtggcgatgtttggttcccacaaccggcccctaaagatcacccactgcgatacgccgtaaacccattcggtggcggtaatgccatggtcccgcatatgtcaggaacttctcttgatgctcagaaacgatacgccgatggcaccaagaaaatcctcgaaagctacctttctggtcgccacgactatcagccacatgacctcattgtccacaagggagattacgcgaccaaggcatacggtcagcgcaaatga

SEQ ID NO: 37 - DNA, 1194 bp, Segniliparus rugosus
  source 1..1194: mol_type = other DNA
atggcgaaaatcgttgccgtcctttacccggacccggtggacgggtatcccacgacgtacgcccgcgacgacatcccggtcatcgcgagctaccccggagggcagagcgcgccgaccccgtccgcgatcgacttcgtccccggccagctgctgggcagcgtgaccggcgagctgggcttgcggaagttcctcgaagcccagggccacacgctcgtggtcacctcggacaaggaggggccggattccgcgctcgaccgcgagctggccgacgcggacgtggtgatctcgcagccgttctggcccgcctacctcaccgccgagcggatcgcggcggctccgaacctcaagctcgcgatcacggcgggcatcggctcggaccacgtggacctcaacgccgccatcgcgcgcggcatcaccgtcgccgaggtcaccttcagcaactcgatcagcgtcgccgaacacgcggtcatgcagatccttgctttggtgcgcaactacctgccgtcgcacgcgtgggtcgccgagaagcggggctggaacatcgccgactcggtggagcgcgcctacgacctcgaaggcatgaccgtcggcgtgatcgcggcgggccggatcggcctcgccgtcttgcgcaggctcaagcccttcgacgtgaagctggcgtacaccgacaagcaccgcctgtccgccgaagtcgaggaggagctgggcctcacgttctacccggacatcccctcgctggtgcgcgcggtggacatcgtggacgtccacgcgccgctcgtgcccgagacccaccacctcttcgacgacgcgctcatcggcacgatgaagcgcggctcgtacatcgtgaacaccgccagaggcgcgatcatggacaccggcgcggtggtccgcgcgctcgagtccggccagctcgccgggtacgcgggcgacgtctggtacccgcagccgcccgcacccgaccacccgtggcgcacgatgccgtggagcgggatgaccccgcacgtctcgggcacgacgctctcggcgcaggcccggtacgccgcagggactcgggagatcctcgagtcctggctcgcgggcgcgccgatccgcgacgagtacctcatcgtggacggcggcaagctcgcgggcaccggagcccgttcctacagcaccagtggcagcgggtcgagcccaggatcgagcgggaagtag

SEQ ID NO: 38 - DNA, 1260 bp, Aspergillus indologenus
  source 1..1260: mol_type = other DNA
atggtatttatgcgctctttctcccgtcaacttcgacggcctgcgacttctcttctttcaagcagaggccctctcgcatcggctggcagcagtcccttgcggacctcgcctctggccaactccattgctggtgcacgtacgctaaccgcctcggccaatctacagggcaagattcttatggttctttatgacggcggtgagcacgccaagcagcagcccggtcttctgggtaccaccgagaacgagcttggtttgagaaagtggctggaggaccagggccacactttggttaccacctctgacaaggagggtgagaactccactttcgacaaggagctcgttgatgccgaagtcatcatcaccacccccttccaccccggttaccttactgcggagcgtttggccaaggccaagaaccttaagatcgccgtcactgccggtgttggttccgaccacgtcgacctcaacgccgccaacaagaccaatggcggtatcactgtggccgaagtcactggctgcaatgttgtctccgttgcggagcacgttgtcatgacgatcctgaccctggtccgcaacttcgttcccgcccacgagcagatccgtaacggcgagtgggacgttgctgctgtcgccaagaacgagtacgatttggagaacaaggttgtcggcactgtcgctgtcggccgcatcggtgagcgtgtcctgcgccgtctcaagcctttcgactgcaaggagctgctctactacgactaccagcctctctcccctgaggtcgagaaggagatcggctgccgccgtgttgacgacctcgaggagatgctcgctcagtgtgatgtcgttaccatcaactgccctctacacgagaagactcgtggtctgttcaacaaggacctcatctccaagatgaagaagggctcgtggctcatcaacaccgctcgcggtgccatcgttgtcaaggaggacgtcgccgaggccgtcaagtctggtcacttgagaggctacggaggtgatgtctggttccctcagcccgcccccaaggatcaccctctgcgctacgtccagggcccctggggcggcggcaacgccatggttccccacatgtccggtacctcgatcgacgcccagatccgttacgccaacggcaccaaggctatcctcgagagctacttctccggtcgccacgactacagaaacgaggacctcatcgtccacaacggtgactatgtcactcgcgcctacggtcagcgcaacaaggcttaa

SEQ ID NO: 39 - DNA, 1248 bp, Friedmanniomyces endolithicus
  source 1..1248: mol_type = other DNA
atggtattcgctcgctccgcactccgcctcgcacgcccggcgagctcgctgttctcgcagcgggcggcgccagccttcgcccagcgcggggttccagtcgccaagagggccctgtcagcgactgcgtcgcaacagggaaaagtcctcttggtgctctacgatggccaccaacacgcccaggacgagcctcgattgctcgggacgaccgagaacgagctcgggatccggaagtggattgaggaccagggccacactcttgtgacgacctccgacaaagacggcgagaacagcaagttcgaccaagagcttgttgacgccgaggtcatcatcaccacacccttccaccccggctacctcaccaaggagcgcctcgccaaggcgaagaagctcaagatcgccgtgacggccggtattggttccgaccacgtcgacctcgacgccgccaacacgaccaacggcggtatcaccgtcgccgaagtcaccggctccaacgtggtgtccgtcgccgagcacgtcatcatgaccatgctcgtcctggtccgcaacttcgtccccgcccacgagatgatcgccgccggcaactgggacgtcgcggccgtcgccaagaacgagtacgacttggagaacaaggtcgtcggtaccgtggctgtcggccggatcggcgagcgtgtcctccgcagactgaaggccttcgactgcaaggagctgctgtactttgactaccagggtctgactcccgagaaggagaaggagattggctgcaggcgggtggagaacctcgaggacatgctggcgcagtgcgatgtcgtcaccatcaactgcccgctgcacgagaagacgcgcggcatgttcaacaaggagctgctgagcaagatgaagaagggctcctggctcgtcaacacggcccgcggcgccatcgtcgtcaaggaagacgtggccgaggccctcaagagcggccagctccgcggctacggcggcgacgtctggttcccccagccagcccccaaggaccacccgctccgctacgcctcctactccacctggggcggcggcaacgccatggtgccccacatgtccggcacctccatcgacgcgcaggagcgctacgccgccggcaccaaggcgattttggaggagtacttcagcgggcgcgagaactacaagcccgaggatttgattgtgcataagggggattacgccactaaggcgtatgggcagcgggaggggaagaagcatgctaatgcttag

SEQ ID NO: 40 - DNA, 2307 bp, Actinidia chinensis
  source 1..2307: mol_type = other DNA
atggcgatgaagcgtgctgctgcctctgcagtccgtgctcttgcttcgtcggggaattcgaattcgtcgattttgaccagagagctccatacttctcctgggagcaaaaagatagttggagtgttctataaggcaaatgaatatgctgcaatgaatcccaattttttgggctgtgtggaaagagcattgggcataggtgactggttgaaatcacaagggcaccaatacatcgtcacagatgacaaagagggacctgattgtgaactcgagaaacacatttctgatatgcacgtcctcataactaccccatttcatccggcctatgttactgcggaaaggatcaagaaggctaaaaatctgcaactattgctcacagctggaattggctctgaccatgtcgatctgaaggctgcagctgctgccgggttaacagttgctgaggtcactggaagcaatgtggtttcggtagcagaggacgagctcatgagagttctcattctcactcgaaatttcttacctggataccatcaggttattaaaggggactggaatgttgcagctattgctcacagggcctacgatctggaaggaaagactgttggaactgtcggtgctgggcgtattggcaggctgttactccaaagactgaagcctttcaactgtcatctcctctatcatgaccggatcaagatggacctggagttggagaatcaaatcggtgcaaagtttgaggaggatcttgatgtgatgcttccaaaatgtgacattattgtcatcaacatgcctcttactgagaaaacaaaaggtatgtttaacaaggagaggatcgctaagttgaagaagggagttctcattgttaacaatgctagaggagctataatggatacacaagcagttgttgatgcctgttccagtggacatattgcaggctacagtggggatgtttggaatccacaaccagctccaaaggaccacccatggcgatacatgccaaaccaagccatgacccctcatatttctgggactacaatcgatgcacagttgcgatatgcagcaggagttaaggacatgttggataggtatttcaagggagaagagtttcctccacagcattacattgtcaaggaggggcccacccgttgtggaggaaatgcgattgggtgggggcagtgcgagaaagtaaaaatgggactactggagaatcacttgggagaaagtgtcagagttgtcacaaagtgctctacttctcctgggagcaaaaagatagttggagtgttctataaggcaaatgaatatgctgcaatgaatcccaattttttgggctgtgtggaaagagcattgggcataggtgactggttgaaatcacaagggcaccaatacatcgtcacagatgacaaagagggacctgattgtgaactcgagaaacacatttctgatatgcacgtcctcataactaccccatttcatccggcctatgttactgcggaaaggatcaagaaggctaaaaatctgcaactattgctcacagctggaattggctctgaccatgtcgatctgaaggctgcagctgctgctgggttaacagttgctgaggtcactggaagcaatgtggtttcggtagcagaggacgagctcatgagagttctcattctcactcgaaatttcttacctggataccatcaggttattaaaggggactggaatgttgcagctattgctcacagggcctacgatctggaaggaaagactgttggaactgtcggtgctgggcgtattggcaggctgttactccaaagactgaagcctttcaactgtcatctcctctatcatgaccggatcaagatggacctggagttggagaatcaaatcggtgcaaagtttgaggaggatcttgatgtgatgcttccaaaatgtgacattattgtcatcaacatgcctcttactgagaaaacaaaaggtatgtttaacaaggagaggatcgctaagttgaagaagggagttctcattgttaacaatgctagaggagctataatggatacacaagcagttgttgatgcctgttccagtggacatattgcaggctacagtggggatgtttggaatccacaaccagctccaaaggaccacccatggcgatacatgccaaaccaagccatgacccctcatatttctgggactacaatcgatgcacagttgcgatatgcagcaggagttaaggacatgttggataggtatttcaagggagaagagtttcctccacagcattacattgtcaaggagggtgaactcgctagccaataccgttaa

SEQ ID NO: 41 - DNA, 1257 bp, Aspergillus kawachii
  source 1..1257: mol_type = other DNA
atggtatttatgcgctctttctcccgccaacttcggcgacctgccacctctcttctctccaagggagccttcgcgcccgcggccagcagcccattccgggctgctcctctggctaactcgatcgctggtgcacgtacgctaaccgcctccgcgaatctgcagggcaaggttcttatggtcctctatgacggcggtgagcacgccaagcagcagcccggtcttctgggtaccaccgagaacgagctcggcttgaggaagtggctcgaggagcagggtcacactctggtcaccacctccgacaaggagggcgagaactcgaccttcgacaaggagctcgtcgacgctgaagtcatcatcaccactcccttccaccccggttacctcactgctgagcgtctggccaaggccaagaacctcaagatcgccgtcactgctggtgtcggttccgaccacgttgacctgaacgctgccaacaagaccaatggcggtatcaccgtcgccgaggtcaccggctgcaacgtcgtctccgtcgctgagcacgttgtcatgaccatcctgaccctggtccgcaacttcgttcccgcccacgagcagatccgccgcggcgagtgggacgttgccgctgtcgctaagaacgagttcgacctcgagggtaaggtcgtcggtaccgttgccgttggccgtatcggagagcgtgtcctccgccgtctcaagcccttcgactgcaaggagctcctctactacgactaccagcccctttcccccgaggttgagaaggagatcggctgccgccgcgtcgacgacctcgaggagatgctcgctcagtgcgatgttgtcaccatcaactgccccctgcacgagaagacccgcggtctgttcaacaaggagctcatctccaagatgaagaagggctcttggctcgtcaacaccgctcgtggtgccatcgttgtcaaggaggatgtcgccgaggccgtcaagtctggccacctccgcggatacggtggtgacgtctggttcccccagcccgcccccgccgaccaccctctgcgcaccgtccagggcccctggggcggcggcaacgccatggttcctcacatgtccggtacctccatcgatgcccagatccgttacgccaacggtaccaaggccatcctcgagagctacttctccggccgccacgactaccgcccccaggacctcattgtccacggcggtgactacgttaccaaggcctacggccagcgcaacaaggcctaa

SEQ ID NO: 42 - DNA, 1056 bp, Candidatus Nitrosocosmicus oleophilus
  source 1..1056: mol_type = other DNA
atgaaaattgttgcagtgttatatcctggaggagaaatagcaaaaaggacaccggcgattcttggttcggcagagaatgctcttggcttgacaaattttctgaaagataaaggacatgaatttattgtattaacagataaagaggaggaacttgataaacacatttctactactgatattctgatcactacccccttttggccggcctatgttaccaaagaaagaatttccaaagcttctaacctcaagctcatcttaactgcaggcgtaggatcagatcacatagatttggctgcagcagcaagtagtaaaataactgttgcagaaattacaggtagtaatgtggtaagtgtagctgaacaggttgtgatgcatatcttggctttagtgcgaaactacataccagcttataaacaggttatagaaggtagatgggatatagcagagatagcatcaaacgcgcatgatctagaagacaaggttgttggaattatcggaatgggaagaattggacaaagggtttgtgaaaggttgaaagcatttgatgttaaaatgctttatcatgatcaaattcctctacggactgcagaagaatttgtgttgggtgtcagatacacccctagagatcagatcgtagaacaggctgatgtgattacaataaatacgcctttaacgccagagaccgatggtatgtttaatcgagatctactttttaaaatgaaaaaaggtgcatatcttgttaatacagctcgaggaaagatcgtagatactcatgctcttgtagaatctttggaggaaggtcatttagcaggatatgctggcgatgtttggtatccacaaccggccacaggagatcatccttggagacatatgcctaatcatgcgatggtccctcattattctggaacaaccctcgaagcacaaaacagatatgcgaatggaataaaggattgtcttatgcggtttctagaaaaccgtcctcttgaacaacaataccttattgtcgataaaggtggcgtggtaagcccgagttacagctacgctttcaagaaataa

SEQ ID NO: 43 - DNA, 1023 bp, Blastomyces gilchristii
  source 1..1023: mol_type = other DNA
atgggtaaagtactcttggttttgtatgatggaggacaacatgcgaaggaccagcctggtttgctcggaacgactgagaacgagctgggcctgaggaagtggttggaagagaagggccatacccttgtaaccacctctgacaaagaaggtgccaactctaaattcgaccaggagcttgtcgatgctgaggttatcatcactaccccattccacccaggatatctgaccgcagaacgcctggctaaagccaagaacctgaaactggccgtcacggccggtgtgggatccgaccacgtcgacctgaacgccgccaacaagacgaatggcggcatcaccgttgccgaagtgacgggctgcaatgtcgtctccgtcgccgagcacgtcgtcatgaccattctcgtcctggtccgcaactttgtcccttcacacgagcaggttgccagcggcgattggaacgttgccgccgtggcgaagaacgagtatgacctcgagggaaaagtcgtcggcaccgttgccgtgggccgtattggcgagcgtgtcctgcgacgactgaagccgtttgactgcaaggagctgctgtactatgattaccagccgctgacgccagaggtcgagaaggagatcggatgccgccgcgtggagaatctggaggaaatgctcgcgcagtgtgatgttgtcaccatcaactgccccctgcacgagaagactcgcggcttgtttaacaaagacctcatctcgaagatgaagaagaggctcctggctcatcaacaccgcccgcggcgccatcgtcgtcaaagaagacgtcgccgacgccatcaaatccggccatctgcgcggctacggcggcgacgtgtggttcccgcagcccgcgcccaaggaccatccgctgcgctacgtccaggggccctggggcggcggaaacgcgatggtgccgcacatgtccggcagctccatcgatgcgcaggtgcgctacgcggagggcacgaaggcgattctcgagtcgtatttctccggcaggcatga

SEQ ID NO: 44 - DNA, 1272 bp, Paraburkholderia caribensis
  source 1..1272: mol_type = other DNA
atgtatcacgtcgatatgtcgatagagcgccggcatgaaaatgcgctcgaccagactggagcgagacgaatggctaaggttctttgtgtgctgtatgacgaccccgtcaagggaatgccgaaacagtacgcccgcgacggcgtacccgaaatcacgcgttatcacgacggtcaaacggccccgacgccgaagggcatcgacttcacgccgggcgaattgctgggcagcgtatcgggcgaactggggctgcggaaatacctcgaatcgctagggcatacgctcgtcgtgacatccgacaaggatggtccgaactcgcggttcgagcgcgagcttgccgacgccgaagtcgtgatttcccaaccgttctggcccgcgtacctgacggcggaacgcatcgcgaaagcgccgaagctcaagctcgcgttgacggcgggcatcggctcggatcacgtcgatttgcaggcggccatcgagcgcggcgtgacggtggccgaagtgacgtattgcaacagcatcagcgtcgccgaacacgtcgtgatgatgattctcgggctggtgcgcaattacctgccgtcgcatgaatgggtgaagaagggcggctggaacatcgccgactgcgtcgagcgtgcttacgatctcgaagccatgcacgtaggcacggttgcggcgggccggatcggcgcggccgtgttgcgcagactgaagccgttcgacgtgaagctgcactatacggatcgtcatcgcctgccgctcgatgtcgagaaagaactcggcgcgacgtggcacccggacgtcgagtcgatggtgaaagcgtgcgacgtggtgacgatcaactgtccgctgcacccggaaacggaaaatctcttcaatgaaaagctgatcgcgaaaatgaagcgcggcgcgtatatcgtgaacacggcgcgcggcaagatcgtcgatcgcgatgcgattgtgcgggcgctcgaatcgggacaactggcgggctatgcgggcgacgtgtggttcccgcagccggcgccgcgcgatcatccgtggcgcacgatgccgcacaacggcatgacgccgcatatttcgggcactacgctgtcggcacaggcgcgttatgcggctggcacgcgcgagatcctcgaatgctggttcgagaagcggcctatccgcgacgagtatctgatcgtcgacggcggcaagctggcgggcgtcggcgcgcactcgtatagcgcgggcaatgcgacgtcgggatcggaagaggcggcgcgattcaagactgcgtaa

SEQ ID NO: 45 - DNA, 1212 bp, Mesorhizobium sp.
  source 1..1212: mol_type = other DNA
atcaaaatggcaaaggtcgtttgcgtcctctatgacgatccggttgacggttatccgtcaaattatgcacgggatgggctgccgaagctcgaccgctatcccggcggccagacgcttcccacgccgaaggccatcgacttcgaaccgggggttctgctcggcagcgtgacgggcgagctcggcctgcggccatttctcgaaagcgccgggcacagtctggtcgtgacctcggacaaggacggtcccgacagcgttttcgaacgcgaactcgctgacgccgaaatcgtcatctcgcagccgttctggccggcctatctgaccaacgaacggatcgccaaggcagaccaacttaagcttgcgatcaccgccggcatcggatccgaccatgtcgatctgcaggccgcgatggaccgcggcatcaccgtcgccgaggtgacctactgcaactcgatcagcgtgtccgagcatgttgtgatgatgatcctggcgctggtgcgaaactacatcccctcttaccagtgggtcatcgacggtggctggaatatcgccgactgtgtcgcgcgctcctacgatgtcgaggggatgcagatcggaaccgtcggcgccggccggatcggtagcgcggtgctgcaccggctgcagccgttcgacgtcaaattgcattacaccgaccgccaccgcctgtccgaggcagtcgagaaacaccttggcgtcaccttccacccagacgccgcgtccatggtcggcatctgcgatgtggtgacgatcaatgcacctctgcatccggagacggagaacctgttcgacgaggcgatgatcgccaggatgaaacggggcgcctatctggtgaacacggcgcgcggcaagatctgcaatcgcgacgccgtcgcccgtgcgctggagagcggccagctcgccggctatgccggcgatgtgtggttcccgcagccggcccccaaggaccatccctggcgttcgatgccgcatcacggcatgacgccgcatatttcaggatcttcgctctcggcccaggcgcgctacgccgccggcacccgcgaaatcctggagtgctggttcgaaggccggccgatccgggaggaatacctgatcgtcgatggcggcaagctggccggcgccggcgcgcattcctatagcgcaggcgacgccacaagcggttcggaggaggcggcgcgcttcaaggccaaggcgtga

SEQ ID NO: 46 - DNA, 1275 bp, Helicocarpus griseus
  source 1..1275: mol_type = other DNA
atggtatttctgcgctctacatcccgtctcctctcccggccaatctcctctctgttggctggtcgagcccttccccgcgcctcagctgccttgcccctatacaacacgcagctctgcgggccaacgtcccgtttgcctttcactggagtcagaacactaacagcgagtcctaaactccagggtaaagttctgcttgtcttgtacgatggagaagagcatgcgaagcagcagcccggcctgcttggaaccactgagaacgagctcggattgagaaaatggctcgaagatcagggacataccctggttaccacctccgataaagagggagagaactcaaaattcgaccaggagctcgtcgacgctgaagttatcatcactactcctttccaccccggttacctcactgcggagcgtctcgccaaagctaagaacctcaagcttgccatcacagctggtgttggctccgaccacgtcgatctgaacgccgccaacaagactaacggcggtatcaccgtcgctgaagtcactggctgcaatgtcgtctctgtcgctgagcacgtcgtcatgactatccttgttttggtccgcaacttcgtccccgcacacgagcaggttgccagtggagactggaacgttgctgccgtggccaagaatgagttcgacttggagggcaaggtcgttggtaccgtcgctgttggccgcattggcgagcgtgttctccgacgactcaagccattcgactgcaaggagctgctctactacgactaccagcccttgaagcctgaggttgagaaggagattggatgccgtcgcgtcgagaacctcgaggagatgctggctcagtgtgacgttgtcaccatcaactgccctctccacgagaagacccgtggtctgttcaacaaggagctcatctcgaagatgaagaaaggctcctggctcgtcaacactgcccgtggcgccatcgtcgtcaaggaggacgttgccgaggccgttaagagcggccacctccgcggctacggaggcgacgtctggttcccccagcccgcgcccaaggaccacccactccgctacgcccagggcccatggggcggcggaaacgccatggttcctcacatgtccggtacctccatcgacgcccaggtccgctatgctgagggcaccaagaggatcttggactcgtacttctctggcagacacgactataggcctgaggatttgatcctgttcggtggtgattatgctaccaaggcatatggacagcggactaaggcttag

SEQ ID NO: 47 - DNA, 1179 bp, Streptomyces albidoflavus
  source 1..1179: mol_type = other DNA
atggcgaagatcgtctgcgttctctacccggacccggtcaccggcttcccgccccagtacgcgcgcacctccctccctgcgctcgacggctaccccggcggccagaccctgccgactcccgagggcctcgacttcacccccggcgaactggtgggcagcgtctccggggagctggggttgcgggaattcctggagtcccggggccacaccctggtcgtcacctccgacaaggagggccccgactccgagctggaccgtgaattggccgacgccgacgtggtgatatcccagccgttctggcccgcctacctcaccgccgaacgcatcgcccgcgccccgaagctgaagctggcgctgaccgccggcatcggctccgaccacgtcgacctggacgccgccatcgcccgcggcatcaccgtcgccgaggtcacgtacagcaacagcatcagcgtcgccgagcacgctgtgatgcagatcctggccctggtccgcaactacctgccctcccacaagatcgcggccgagggtggctggaacatcgccgactgcgtctcgcacgcctacgacctggagggcatggacgtcggagtcatcgccgccggccgcatcggccaggccgtgctgcgccgcctcaagcccttcggtgtccgcctccactacaccgacaagcgccggctgccgcgcgaggtcgaggaggagctgggcctgaccttccacgcgagtgcccaggaactcgcccgcaacatcgacgtggtctccatccacgccccgctccaccccgagacgcagaacctcttcgacgagaagctgctggcgaccatgcggccgggctcctacatcgtcaacaccgcccgcgcccagatcgtcgaccgcgacgcgatcgtccgcgccctggagtcgggacagctcgccggttacgcgggcgacgtctggtacccccagcccgcccccgccgaccacccctggcgcaccatgccccacaacggcatgaccccccacatctccggcaccaccctcaccgcccaggcccgctacgcggccggcacccgcgagatcctggaggactggctgcagggcacgccgatccgcgaggaatacctcatcgtcgacggcggccgcctcgccggaacgggcgccgcgtcgtacacggcgggagaaaagacggcagaaaagagctga

SEQ ID NO: 48 - DNA, 1095 bp, synthetic construct
  source 1..1095: mol_type = other DNA
atgaagatcgttttagtcttatacgattgtggtaagcacgctgccgatgaagaaaaattatacggttgtactgaaaacaaattaggtattgccaattggttgaaagatcaaggacatgaattaatcaccacgtctgataaagaaggcggaaacagtgtgttggatcaacatataccagatgccgatattatcattacaactcctttccatcctgcttatatcactaaggaaagaatcgacaaggctaaaaaattgaaattagttgttgtcgctggtgtcggttctgatcatattgatttggattatatcaaccaaaccggtaagaaaatctccgttttggaagttaccggttctaatgttgtctctgttgcagaacacgttgtcatgaccatgcttgtcttggttagaaattttgttccagctcacgaacaaatcattaaccacgattgggaggttgctgctatcgctaaggatgcttacgatatcgaaggtaaaactatcgccaccattggtgccggtagaattggttacagagtcttggaaagattagtcccattcaatcctaaagaattattatactacgattatcaagctttaccaaaagatgctgaagaaaaagttggtgctagaagggttgaaaatattgaagaattggttgcccaagctgatatagttacagttaatgctccattacacgctggtacaaaaggtttaattaacaaggaattattgtctaaattcaagaaaggtgcttggttagtcaatactgcaagaggtgccatttgtgttgccgaagatgttgctgcagctttagaatctggtcaattaagaggttatggtggtgatgtttggttcccacaaccagctccaaaagatcacccatggagagatatgagaaacaaatatggtgctggtaacgccatgactcctcattactctggtactactttagatgctcaaactagatacgctcaaggtactaaaaatatcttggagtcattctttactggtaagtttgattacagaccacaagatatcatcttattaaacggtgaatacgttaccaaagcttacggtaagcacgataagaaataa

SEQ ID NO: 49 - protein, 396 aa, synthetic construct
MSKILCVLYPDPVTGFPPVYARDSIPDIRCYXXXXXXXXXAGXXXXXXXXLXGXXXXXLGJXXXLXXQGHELIXTSDKEGXXSXXXXHJPDADXXIXXPFXPAYJTXERJXKAKXLKLXXXAGXGSDHXDLXXXXZXGXXIXVXEVTXSNXXSVAEHVVMTXLXLVRNXXPAHZXXXNXXWXXAXXXXXXYDXEGXXXXTJGAGRIGXXVLXRLXPFBXXXLXYYDXXXLXXEXEXXXGXXXXXXXEXLVXXXDXVXXXXPLXXXTXGXXNXXXLXKFKXGAXLXNTARGAJCXXXXXXXALEXGQLXGYGGDVWFPQPAPXDHPWRXMXXXXXXXXXMTPHXSGTXLXXQXRYAXGTXXILZXFXXGKXXXRXQXXJJXBGXXXXXAXXXXXKXA